SEQUENCE LISTING (221 sequences)

SEQ ID NO: 1 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 4 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 6 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 226 aa, synthetic construct
DKTHTCPPCPAPELLGGPSVFLFPPKPKDTLMASRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 8 - protein, 226 aa, synthetic construct
DKTHTCPPCPAPELLGGPSVFLFPPKPKDTLMASRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 230 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREELTKNQVSLWCLVKGFYPSDIAVEWESSGQPENNYKTTPPVLDSDGSFFLYSKLTVDKSRWQQGNIFSCSVMHEALHNRFTQKSLSLSPGGSPG

SEQ ID NO: 10 - protein, 230 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPGGSPG

SEQ ID NO: 11 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 12 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 13 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 14 - protein, 133 aa, synthetic construct
APTSSSTKKTQLQLEHLLLDLQMILNGINNYKNPKLTAMLTAKFAMPKKATELKHLQCLEEALKPLEEVLNLAQSKNFHLRPRDLISNINVIVLELKGSETTFMCEYADETATIVEFLNRWITFAQSIISTLT

SEQ ID NO: 15 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 16 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 17 - protein, 114 aa, synthetic construct
NWVNVISDLKKIEDLIQSMHIAATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 18 - protein, 114 aa, synthetic construct
NWVNVISDLKKIEDLIQSMHIDATLYTESDVHPSCKVTAMKCFLLALQVISLESGDASIHDTVENLIILANNSLSSNGNVTESGCKECEELEEKNIKEFLQSFVHIVQMFINTS

SEQ ID NO: 19 - protein, 114 aa, synthetic construct
NWVNVISDLKKIEDLIQSMHIDATLYTESDVHPSCKVTAMKCFLLALQVISLASGDASIHDTVENLIILANNSLSSNGNVTESGCKECEELEEKNIKEFLQSFVHIVQMFINTS

SEQ ID NO: 20 - protein, 114 aa, synthetic construct
NWVNVISDLKKIEDLIQSMHIDATLYTESDVHPSCKVTAMKCFLLRLQVISLRSGDASIHDTVENLIILANNSLSSNGNVTESGCKECEELEEKNIKEFLQSFVHIVQMFINTS

SEQ ID NO: 21 - protein, 114 aa, synthetic construct
NWVNVISDLKKIEDLIQSMHIDATLYTESDVHPSCKVTAMKCFLLSLQVISLSSGDASIHDTVENLIILANNSLSSNGNVTESGCKECEELEEKNIKEFLQSFVHIVQMFINTS

SEQ ID NO: 22 - protein, 114 aa, synthetic construct
NWVNVISDLKKIEDLIQSMHIDATLYTESDVHPSCKVTAMKCFLLELQVISLASGDASIHDTVENLIILANNSLSSNGNVTESGCKECEELEEKNIKEFLQSFVHIVQMFINTS

SEQ ID NO: 23 - protein, 18 aa, synthetic construct
NWVNVISDLKKIEDLIQS

SEQ ID NO: 24 - protein, 79 aa, synthetic construct
KVTAMKCFLLELQVISLESGDASIHDTVENLIILANNSLSSNGNVTESGCKECEELEEKNIKEFLQSFVHIVQMFINTS

SEQ ID NO: 25 - protein, 114 aa, synthetic construct
NWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFINTS

SEQ ID NO: 26 - protein, 114 aa, synthetic construct
NWVNVISDLKKIEDLIQSMHIDATLYTESDVHPSCKVTAMKCFLLELQVISLESGDASIHDTVENLIILANNSLSSNGQVTESGCKECEELEEKNIKEFLQSFVHIVQMFINTS

SEQ ID NO: 27 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIQTS

SEQ ID NO: 28 - protein, 114 aa, synthetic construct
NWVNVISDLKKIEDLIQSMHIDATLYTESDVHPSCKVTAMKCFLLELQVISLESGDASIHDTVENLIILAQNSLSSNGQVTESGCKECEELEEKNIKEFLQSFVHIVQMFINTS

SEQ ID NO: 29 - protein, 114 aa, synthetic construct
NWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFIQTS

SEQ ID NO: 30 - protein, 114 aa, synthetic construct
NWVNVISDLKKIEDLIQSMHIDATLYTESDVHPSCKVTAMKCFLLELQVISLESGDASIHDTVENLIILANNSLSSNGQVTESGCKECEELEEKNIKEFLQSFVHIVQMFIQTS

SEQ ID NO: 31 - protein, 114 aa, synthetic construct
NWVNVISDLKKIEDLIQSMHIDATLYTESDVHPSCKVTAMKCFLLELQVISLESGDASIHDTVENLIILAQNSLSSNGQVTESGCKECEELEEKNIKEFLQSFVHIVQMFIQTS

SEQ ID NO: 32 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 33 - protein, 219 aa, synthetic construct
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 34 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 35 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 36 - protein, 306 aa, synthetic construct; identical to SEQ ID NO: 34

SEQ ID NO: 37 - protein, 10 aa, synthetic construct
KSKREKKDRV

SEQ ID NO: 38 - protein, 303 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKDNTERVFTDKTSATVICRKNASISVRAQDRYYSSSWSEWASVPCS

SEQ ID NO: 39 - protein, 304 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKDNTEGRVFTDKTSATVICRKNASISVRAQDRYYSSSWSEWASVPCS

SEQ ID NO: 40 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCS

SEQ ID NO: 41 - protein, 8 aa, synthetic construct
KDNTEGRV

SEQ ID NO: 42 - protein, 304 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKDNTEGRVFTDKTSATVICRKNASISVRAQDRYYSSSWSEWASVPCS

SEQ ID NO: 43 - protein, 5 aa, synthetic construct
GGGGS

SEQ ID NO: 44 - protein, 15 aa, synthetic construct
GGGGSGGGGSGGGGS

SEQ ID NO: 45 - protein, 420 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMA

SEQ ID NO: 46 - protein, 515 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKDNTERVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 47 - protein, 516 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKDNTEGRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 48 - protein, 518 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 49 - protein, 420 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKDNTEGRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALC

SEQ ID NO: 50 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 51 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRSNISWEISQASHYFERHLEFEARTLSPGHTWEEAPLLTLKQKQEWISLETLTPDTQYEFQVRVKPLQGEFTTWSPWSQPLAFRTKPAALGKD

SEQ ID NO: 52 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRVNISWEISQASHYFERHLEFEARTLSPGHTWEEAPLLTLKQKQEWIVLETLTPDTQYEFQVRVKPLQGEFTTWSPWSQPLAFRTKPAALGKD

SEQ ID NO: 53 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWIVLETLTPDTQYEFQVRVKPLQGEFTTWSPWSQPLAFRTKPAALGKD

SEQ ID NO: 54 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWIVLETLTPDTQYEFQVRVKPLQGEFTTWSPWSQPLAFRTKPAALGKD

SEQ ID NO: 55 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRVNISWEISQASHYFERHLEFEARTLSPGHTWEEAPLLTLKQKQEWIALETLTPDTQYEFQVRVKPLQGEFTTWSPWSQPLAFRTKPAALGKD

SEQ ID NO: 56 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRACISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 57 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRVCISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 58 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWIALETLTPDTQYEFQVRVKPLQGEFTTWSPWSQPLAFRTKPAALGKD

SEQ ID NO: 59 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHCETHRCQISWEISQASHYFERHLEFEARTLSPGHTWEEAPLLTLKQKQEWIALETLTPDTQYEFQVRVKPLQGEFTTWSPWSQPLAFRTKPAALGKD

SEQ ID NO: 60 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCQISWEISQASHYFERHLEFEARTLSPGHTWEEAPLLTLKQKQEWIACETLTPDTQYEFQVRVKPLQGEFTTWSPWSQPLAFRTKPAALGKD

SEQ ID NO: 61 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRCMAPISLQVVHVETHRANISWEISQASHYFERHLEFEARTLSPGHTWEEAPLLTLKQKQEWIALETLTPDTQYEFQVRVKPLQGEFTTWCPWSQPLAFRTKPAALGKD

SEQ ID NO: 62 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRCMAPISLQVVHVETHRANISWEISQASHYFERHLEFEARTLSPGHTWEEAPLLTLKQKQEWIALETLTPDTQYEFQVRCKPLQGEFTTWSPWSQPLAFRTKPAALGKD

SEQ ID NO: 63 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWIALETLTPDTQYEFQVRCKPLQGEFTTWCPWSQPLAFRTKPAALGKD

SEQ ID NO: 64 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWIALETLTPDTCYEFQVRVKPLQGEFTTWSPWSQPLAFCTKPAALGKD

SEQ ID NO: 65 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRCMAPISLQVVHVETHRVNISWEISQASHYFERHLEFEARTLSPGHTWEEAPLLTLKQKQEWIVLETLTPDTQYEFQVRVKPLQGEFTTWCPWSQPLAFRTKPAALGKD

SEQ ID NO: 66 - protein, 213 aa, synthetic construct
AVNGTSQCTCFYNSRANISCVWSQDGALQDTSCQVHAWPDRRRWNQTCELLPVSQASWACNLILGAPDSQKLTTVDIVTLRVLCREGVRWRVMCIQDFKPFENLRLMAPISLQVVHVETHRVNISWEISQASHYFERHLEFEARTLSPGHTWEEAPLLTLKQKQEWIVLETLTPDTQYEFQVRVKPLQGEFTTWSPWSQPLAFRTKPAALGKD

SEQ ID NO: 67 - protein, 662 aa, synthetic construct
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 68 - protein, 862 aa, synthetic construct
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 69 - protein, 213 aa, synthetic construct
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENP

SEQ ID NO: 70 - protein, 522 aa, synthetic construct
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSD

SEQ ID NO: 71 - protein, 189 aa, synthetic construct
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSL

SEQ ID NO: 72 - protein, 199 aa, synthetic construct
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIV

SEQ ID NO: 73 - protein, 296 aa, synthetic construct
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEE

SEQ ID NO: 74 - protein, 296 aa, synthetic construct
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRSTLYWRDEGLVLLNRLRYRPSNSRLWNMVNVTKAKGRHDLLDLKPFTEYEFQISSKLHLYKGSWSDWSESLRAQTPEE

SEQ ID NO: 75 - protein, 540 aa, synthetic construct
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKER

SEQ ID NO: 76 - protein, 204 aa, synthetic construct
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPP

SEQ ID NO: 77 - protein, 120 aa, synthetic construct [sequence omitted]

SEQ ID NO: 78 - protein, 223 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 79 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - protein, 8 aa, synthetic construct
GYTFTNYY

SEQ ID NO: 81 - protein, 8 aa, synthetic construct
INPSNGGT

SEQ ID NO: 82 - protein, 13 aa, synthetic construct
ARRDYRFDMGFDY

SEQ ID NO: 83 - protein, 119 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKTSENLYFQ

SEQ ID NO: 84 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 10 aa, synthetic construct
KGVSTSGYSY

SEQ ID NO: 86 - skipped

SEQ ID NO: 87 - protein, 9 aa, synthetic construct
QHSRDLPLT

SEQ ID NO: 88 - protein, 5 aa, synthetic construct
PGSGS

SEQ ID NO: 89 - protein, 17 aa, synthetic construct
GGSSPPGGGSSGGGSGP

SEQ ID NO: 90 - protein, 10 aa, synthetic construct
GGGGSGGGGS

SEQ ID NO: 91 - protein, 13 aa, synthetic construct
GGSGGGSGGGGGS

SEQ ID NO: 92 - protein, 18 aa, synthetic construct
GGSGGSGGSGGSGGSSGP

SEQ ID NO: 93 - protein, 6 aa, synthetic construct
PGGSGP

SEQ ID NO: 94 - protein, 5 aa, synthetic construct
GGSPG

SEQ ID NO: 95 - protein, 8 aa, synthetic construct
VPLSLYSG

SEQ ID NO: 96 - protein, 8 aa, synthetic construct
LTKNQVSL

SEQ ID NO: 97 - protein, 8 aa, synthetic construct
QQGNVFSC

SEQ ID NO: 98 - protein, 8 aa, synthetic construct
NHYTQKSL

SEQ ID NO: 99 - protein, 8 aa, synthetic construct
IHVTLKSL

SEQ ID NO: 100 - protein, 8 aa, synthetic construct
NSYTIKGL

SEQ ID NO: 101 - protein, 8 aa, synthetic construct
TQKSLSLS

SEQ ID NO: 102 - protein, 8 aa, synthetic construct
SNESLSLS

SEQ ID NO: 103 - protein, 8 aa, synthetic construct
QKSLSLSP

SEQ ID NO: 104 - protein, 8 aa, synthetic construct
QVSSSLSP

SEQ ID NO: 105 - protein, 8 aa, synthetic construct
PTSTSLSP

SEQ ID NO: 106 - protein, 8 aa, synthetic construct
KSLSLSPG

SEQ ID NO: 107 - protein, 8 aa, synthetic construct
ESLSLSEE

SEQ ID NO: 108 - protein, 8 aa, synthetic construct
ASLSLAPV

SEQ ID NO: 109 - protein, 8 aa, synthetic construct
SQESLSLS

SEQ ID NO: 110 - protein, 4 aa, synthetic construct
PLGL

SEQ ID NO: 111 - DNA, 24 bp, synthetic construct
  source 1..24: mol_type = other DNA
attcatgtgaccctgaaaagcctg

SEQ ID NO: 112 - DNA, 24 bp, synthetic construct
  source 1..24: mol_type = other DNA
aacagctataccattaaaggcctg

SEQ ID NO: 113 - DNA, 24 bp, synthetic construct
  source 1..24: mol_type = other DNA
agcaacgaaagcctgagcctgagc

SEQ ID NO: 114 - DNA, 24 bp, synthetic construct
  source 1..24: mol_type = other DNA
caggtgagcagcagcctgagcccg

SEQ ID NO: 115 - DNA, 24 bp, synthetic construct
  source 1..24: mol_type = other DNA
ccgaccagcaccagcctgagcccg

SEQ ID NO: 116 - DNA, 24 bp, synthetic construct
  source 1..24: mol_type = other DNA
gaaagcctgagcctgagcgaagaa

SEQ ID NO: 117 - DNA, 24 bp, synthetic construct
  source 1..24: mol_type = other DNA
gcgagcctgagcctggcgccggtg

SEQ ID NO: 118 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 119 - protein, 10 aa, synthetic construct
APAGLIVPYN

SEQ ID NO: 120 - protein, 9 aa, synthetic construct
GGSSPPGGG

SEQ ID NO: 121 - protein, 119 aa, synthetic construct
EVQLVESGGGLVQPGGSLRLSCAASGSIFSINVMGWYRQAPGKQRELVAAISSGGSTNYADSVKGRFTISRDNAKNTVYLQMNSLKPEDTAVYYCMYASSWYEDETDYWGQGTQVTVSS

SEQ ID NO: 122 - protein, 8 aa, synthetic construct
PVSLRSGS

SEQ ID NO: 123 - protein, 9 aa, synthetic construct
PANLVAPDP

SEQ ID NO: 124 - protein, 121 aa, synthetic construct
QLQLQESGPGLVKPSQTLSLTCTVSGGSISSGGYYWSWIRQHPGKGLEWIGYIYKSGSAYYSPSLKSRVTISVDTSKNQFSLKLSSVTAADTAVYYCARTPTVTGDWFDPWGRGTLVTVSS

SEQ ID NO: 125 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQTYDSIDVYFGGGTKLTVL

SEQ ID NO: 126 - protein, 7 aa, synthetic construct
SGGYYWS

SEQ ID NO: 127 - protein, 19 aa, synthetic construct
GYIYKSGSAYYSPSLKSRV

SEQ ID NO: 128 - protein, 11 aa, synthetic construct
TPTVTGDWFDP

SEQ ID NO: 129 - protein, 13 aa, synthetic construct
TRSSGSIASNYVQ

SEQ ID NO: 130 - protein, 7 aa, synthetic construct
EDNQRPS

SEQ ID NO: 131 - protein, 9 aa, synthetic construct
QTYDSIDVY

SEQ ID NO: 132 - protein, 10 aa, synthetic construct
GSIFSINVMG

SEQ ID NO: 133 - protein, 16 aa, synthetic construct
AISSGGSTNYADSVKG

SEQ ID NO: 134 - protein, 11 aa, synthetic construct
ASSWYEDETDY

SEQ ID NO: 135 - protein, 8 aa, synthetic construct
MPYDLYHP

SEQ ID NO: 136 - protein, 10 aa, synthetic construct
ISSGLLSGRS

SEQ ID NO: 137 - protein, 8 aa, synthetic construct
RAAAVKSP

SEQ ID NO: 138 - protein, 8 aa, synthetic construct
RPLALWRS

SEQ ID NO: 139 - protein, 8 aa, synthetic construct
TQKPLGLS

SEQ ID NO: 140 - protein, 18 aa, synthetic construct
GGSSGSGGSGGGSGSGGG

SEQ ID NO: 141 - protein, 21 aa, synthetic construct
GGGGGSGGGGSGGGGSGGGGS

SEQ ID NO: 142 - protein, 252 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQTYDSIDVYFGGGTKLTVLGGGGGSGGGGSGGGGSGGGGSQLQLQESGPGLVKPSQTLSLTCTVSGGSISSGGYYWSWIRQHPGKGLEWIGYIYKSGSAYYSPSLKSRVTISVDTSKNQFSLKLSSVTAADTAVYYCARTPTVTGDWFDPWGRGTLVTVSS

SEQ ID NO: 143 - protein, 8 aa, synthetic construct
RSKYLATA

SEQ ID NO: 144 - protein, 8 aa, synthetic construct
GRPRHQGV

SEQ ID NO: 145 - protein, 4 aa, synthetic construct
GLFG

SEQ ID NO: 146 - protein, 4 aa, synthetic construct
GFLG

SEQ ID NO: 147 - protein, 7 aa, synthetic construct
AGRRAAK

SEQ ID NO: 148 - protein, 5 aa, synthetic construct
FRLWA

SEQ ID NO: 149 - protein, 5 aa, synthetic construct
FRLWS

SEQ ID NO: 150 - protein, 8 aa, synthetic construct
NFFGVGGE

SEQ ID NO: 151 - protein, 8 aa, synthetic construct
PMKRLTLA

SEQ ID NO: 152 - protein, 8 aa, synthetic construct
FPLATYAP

SEQ ID NO: 153 - protein, 8 aa, synthetic construct
FLVGGASL

SEQ ID NO: 154 - protein, 8 aa, synthetic construct
KPMQFLGD

SEQ ID NO: 155 - protein, 8 aa, synthetic construct
GIVRAKGV

SEQ ID NO: 156 - protein, 8 aa, synthetic construct
ALFKSSFP

SEQ ID NO: 157 - protein, 8 aa, synthetic construct
SGRRSPGG

SEQ ID NO: 158 - protein, 8 aa, synthetic construct
SLGRRPGG

SEQ ID NO: 159 - protein, 8 aa, synthetic construct
SLSGRRGG

SEQ ID NO: 160 - protein, 8 aa, synthetic construct
SLSLGRRG

SEQ ID NO: 161 - protein, 8 aa, synthetic construct
SLSLSGRR

SEQ ID NO: 162 - protein, 6 aa, synthetic construct
GGPRRL

SEQ ID NO: 163 - protein, 6 aa, synthetic construct
GGPLRL

SEQ ID NO: 164 - protein, 6 aa, synthetic construct
GGPKLL

SEQ ID NO: 165 - protein, 6 aa, synthetic construct
GGPRNL

SEQ ID NO: 166 - protein, 6 aa, synthetic construct
GGPRML

SEQ ID NO: 167 - protein, 8 aa, synthetic construct
EHLRSPGG

SEQ ID NO: 168 - protein, 8 aa, synthetic construct
FRSGVPGG

SEQ ID NO: 169 - protein, 8 aa, synthetic construct
SLLLRTGN

SEQ ID NO: 170 - protein, 8 aa, synthetic construct
AGLRSPGG

SEQ ID NO: 171 - protein, 8 aa, synthetic construct
SLFRSAGP

SEQ ID NO: 172 - protein, 8 aa, synthetic construct
SLFRAPGP

SEQ ID NO: 173 - protein, 8 aa, synthetic construct
WLFRSPLG

SEQ ID NO: 174 - protein, 8 aa, synthetic construct
SRLRSPQG

SEQ ID NO: 175 - protein, 8 aa, synthetic construct
SLVLSGRR

SEQ ID NO: 176 - protein, 8 aa, synthetic construct
KQLRHMRG

SEQ ID NO: 177 - protein, 8 aa, synthetic construct
LSGRSDNH

SEQ ID NO: 178 - protein, 4 aa, synthetic construct
LSGK

SEQ ID NO: 179 - protein, 4 aa, synthetic construct
LSGR

SEQ ID NO: 180 - protein, 8 aa, synthetic construct
RQARVVGG

SEQ ID NO: 181 - protein, 8 aa, synthetic construct
RQRRVVGG

SEQ ID NO: 182 - protein, 8 aa, synthetic construct
RQYRVVGG

SEQ ID NO: 183 - protein, 8 aa, synthetic construct
SKGRSLIG

SEQ ID NO: 184 - protein, 8 aa, synthetic construct
PRFKIIGG

SEQ ID NO: 185 - protein, 8 aa, synthetic construct
KQLRVVNG

SEQ ID NO: 186 - protein, 8 aa, synthetic construct
IQPRITGG

SEQ ID NO: 187 - protein, 8 aa, synthetic construct
KQSRKFVP

SEQ ID NO: 188 - protein, 8 aa, synthetic construct
GRQSRAGG

SEQ ID NO: 189 - protein, 8 aa, synthetic construct
SGRSSPGG

SEQ ID NO: 190 - protein, 8 aa, synthetic construct
SSGRSPGG

SEQ ID NO: 191 - protein, 8 aa, synthetic construct
SLSGRSGG

SEQ ID NO: 192 - protein, 8 aa, synthetic construct
SLSSGRSG

SEQ ID NO: 193 - protein, 8 aa, synthetic construct
KLSLSGRS

SEQ ID NO: 194 - protein, 7 aa, synthetic construct
PLRLSRA

SEQ ID NO: 195 - protein, 7 aa, synthetic construct
PLKLSRA

SEQ ID NO: 196 - protein, 8 aa, synthetic construct
PLGLSGRS

SEQ ID NO: 197 - protein, 8 aa, synthetic construct
PLGLRSRA

SEQ ID NO: 198 - protein, 8 aa, synthetic construct
PLGLKSRA

SEQ ID NO: 199 - protein, 6 aa, synthetic construct
RGSRAG

SEQ ID NO: 200 - protein, 6 aa, synthetic construct
RLSRGK

SEQ ID NO: 201 - protein, 6 aa, synthetic construct
RGSRGG

SEQ ID NO: 202 - protein, 6 aa, synthetic construct
KGSRAG

SEQ ID NO: 203 - protein, 6 aa, synthetic construct
KLSRGK

SEQ ID NO: 204 - protein, 6 aa, synthetic construct
GRSRAG

SEQ ID NO: 205 - protein, 6 aa, synthetic construct
LRSRGK

SEQ ID NO: 206 - protein, 6 aa, synthetic construct
GRSRGG

SEQ ID NO: 207 - protein, 6 aa, synthetic construct
GKSRAG

SEQ ID NO: 208 - protein, 6 aa, synthetic construct
LKSRGK

SEQ ID NO: 209 - protein, 9 aa, synthetic construct
VPLSLYSGP

SEQ ID NO: 210 - protein, 24 aa, synthetic construct
GGSSGSGGSGGGSGSGGGSGGSGG

SEQ ID NO: 211 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 212 - protein, 213 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 213 - protein, 113 aa, synthetic construct [sequence omitted]

SEQ ID NO: 214 - protein, 216 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 215 - protein, 107 aa, synthetic construct [sequence omitted]

SEQ ID NO: 216 - protein, 8 aa, synthetic construct
GITFSNSG

SEQ ID NO: 217 - protein, 17 aa, synthetic construct
VIWYDGSKRYYADSVKG

SEQ ID NO: 218 - protein, 6 aa, synthetic construct
ATNDDY

SEQ ID NO: 219 - protein, 6 aa, synthetic construct
QSVSSY

SEQ ID NO: 220 - skipped

SEQ ID NO: 221 - protein, 9 aa, synthetic construct
QQSSNWPRT

(19) United States
(12) Patent Application Publication
(10) Pub. No.: US 2024/0141007 A1
(43) Pub. Date: May 2, 2024

(54) ENGINEERED CLEAVABLE CARRIERS AND METHODS OF USE THEREOF

(71) Applicant: Xilio Development, Inc., Waltham, MA (US)

(72) Inventors: Ertan Eryilmaz, Waltham, MA (US); Carl Uli Bialucha, Waltham, MA (US); Parker Johnson, Waltham, MA (US); Dheeraj Tomar, Waltham, MA (US)

(21) Appl. No.: 18/352,762

(22) Filed: Jul. 14, 2023

Related U.S. Application Data
(60) Provisional application No. 63/389,476, filed on Jul. 15, 2022.
(60) Provisional application No. 63/448,943, filed on Feb. 28, 2023.

(51) Int. Cl. C07K 14/55; A61P 35/00; C07K 16/24

(52) U.S. Cl. CPC C07K 14/55; A61P 35/00; C07K 16/246; A61K 38/00; C07K 2317/569; C07K 2317/622; C07K 2317/71; C07K 2319/30; C07K 2319/50

(57) ABSTRACT

The present disclosure relates to cleavable carriers and cleavable carrier-linked cytokine prodrugs wherein the cleavable carrier is an engineered Fc domain comprising at least one tumor-associated protease cleavage site. Upon cleavage at the cleavage site of the carrier Fc domain, the cytokine is released from a masking moiety. The platform provides enzymatically induced prodrug activation. The present disclosure further provides pharmaceutical compositions comprising said cleavable carrier-linked cytokine prodrugs, their use as a medication as well as methods of treatment of diseases and administration.

### CROSS REFERENCE TO RELATED APPLICATIONS

This application claims priority to, and the benefit of, U.S. provisional application No. 63/389,476, filed on Jul. 15, 2022, and U.S. provisional application No. 63/448,943, filed on Feb. 28, 2023, the contents of each of which is hereby incorporated by reference in its entirety.

### CROSS REFERENCE TO SEQUENCE LISTING

The instant application is being filed with an electronically filed Sequence Listing in XML format. The sequence listing file entitled XTX_UNIVFC_US1_SL.XML, was created on Jul. 14, 2023, and is 210,539 bytes in size; the information in electronic format of the Sequence Listing is incorporated herein by reference in its entirety.

### BACKGROUND

Cancer is the second leading cause of death in the United States, accounting for more deaths than the next five leading causes (chronic respiratory disease, stroke, accidents, Alzheimer's disease, and diabetes). While great strides have been made especially with targeted therapies, there remains a great deal of work to do in this space. Immunotherapy and a branch of this field, immuno-oncology, is creating viable and exciting therapeutic options for treating malignancies. Specifically, it is now recognized that one hallmark of cancer is immune evasion and significant efforts have identified targets and developed therapies to these targets to reactivate the immune system to recognize and treat cancer.

One effective strategy for stimulating the immune system to induce anti-tumor cytotoxicity is a cytokine therapy. Unfortunately, cytokines that are administered to patients generally have a very short half-life, thereby requiring frequent dosing. For instance, the product label of aldesleukin, marketed under the brand name Proleukin, states that the drug was shown to have a half-life of 85 minutes in patients who received a 5-minute intravenous (IV) infusion. In addition, administration of high doses of cytokine can cause adverse health outcomes, such as vascular leakage, through systemic immune activation. To extend half-life and reduce toxicity, carrier moieties such as an Fc domain, albumin, and PEG have been fused to the cytokine through a cleavable linker, which allows release of an active cytokine once the fused molecule reaches a tumor microenvironment.

### SUMMARY OF THE INVENTION

The present invention provides, among other things, compositions and methods for use in the precision treatment of cancer based on cleavable carriers. In particular, the present invention is based on cleavable carriers (e.g., cleavable Fc domains) that can be linked to therapeutic agents, particularly masked therapeutic agents, thereby to deliver and activate the linked therapeutic agents in specific targets such as different tumor microenvironments. For example, a cleavable carrier may be an Fc domain engineered to contain a cleavage site cleavable by a tumor specific protease. Such an engineered cleavable Fc domain is linked to a cytokine or a masking moiety of a cytokine such that the cytokine is only released from its masking moiety upon cleavage of the Fc domain by a tumor specific protease. Prior to the present invention, existing tumor specific therapeutics typically include a cleavable linker. The inventive approach described herein allows more flexible linker designs without constrains of cleavage sites for more effective masking and activation. Thus, the present invention is a significant improvement in the precision medicine field, promising safer and more efficacious therapeutics for cancer patients.

In one aspect of the present invention, provided is a masked therapeutic agent comprising a therapeutic molecule, a masking moiety and a cleavable carrier moiety that comprises an engineered tumor-associated protease cleavage site such that the carrier moiety is cleavable. The cleavable carrier moiety is fused to the therapeutic molecule and/or the masking moiety and upon cleavage of the engineered tumor-associated protease site within the cleavable carrier moiety, the therapeutic molecule is released from the masking moiety. In some embodiments, the cleavable carrier moiety is fused to the therapeutic moiety and/or the masking moiety via a non-cleavable linker. In other embodiments, the cleavable carrier moiety is fused to the therapeutic moiety and/or the masking moiety via a cleavable linker.

In some embodiments, the cleavable carrier moiety is a cleavable Fc domain. The cleavable Fc domain comprises a first Fc domain and a second Fc domain wherein the one of the first or second Fc domain comprises an engineered tumor-associated protease cleavage site. In some embodiments, the carrier moiety may be a half-life extension domain selected from the group consisting of albumin, transferrin and a tissue factor. In other embodiments, the carrier moiety may be an antigen targeting domain selected from the group consisting of immunoglobulin, Fab, F(ab)2, scFv, VHH, ScAb, nanobody, VH, VL, single domain antibody, CL and CK; wherein a tumor-associated protease cleavage site is engineered within the antigen targeting domain.

In one aspect, the present invention provides, among other things, an engineered Fc domain comprising one or more amino acid substitutions as compared to a wild-type Fc domain, wherein the engineered Fc domain comprises a protease cleavage site.

In some embodiments, the protease cleavage site is a tissue selective protease cleavage site. In some embodiments, the protease cleavage site is a tumor associated protease cleavage site. In some embodiments, the protease cleavage site is an inflammation associated protease cleavage site.

In one embodiment, the cleavable carrier moiety is an engineered cleavable Fc domain comprising at least one tumor-associated protease cleavage site. The Fc domain is a human Fc domain.

The therapeutic molecule may be any agent that has a therapeutic effect such as suppression of tumor growth, invasion and progression. In some embodiments, the therapeutic molecule is a cytokine, a variant thereof, or a functional fragment thereof.

In one embodiment, provided in the present disclosure is a masked cytokine. The masked cytokine comprises a cytokine molecule, a masking moiety and a carrier moiety comprising an engineered tumor-associated protease cleavage site, wherein the carrier moiety is fused to the cytokine and/or the masking moiety and upon cleavage of the engineered tumor-associated protease site, the cytokine being masked is released from the masking moiety. In one preferred embodiment, the present invention provide a masked cytokine comprising a cytokine molecule, a masking moiety and an engineered Fe domain comprising a tumor-associated cleavage site, wherein the engineered Fc domain is fused to the cytokine moiety or the masking moiety such that the masking moiety binds to the cytokine moiety and upon cleavage of the tumor associated protease cleavage site on the engineered Fc domain, the cytokine molecule is released from the masking moiety. The engineered Fc domain is fused to the cytokine molecule and/or the masking moiety via a non-cleavable linker. In some embodiments, the engineered Fc domain is directly fused to the cytokine molecule and/or the masking moiety.

In some embodiments, the protease cleavage site is a tissue selective protease cleavage site. In some embodiments, the protease cleavage site is an inflammation associated protease cleavage site.

In some embodiments, the protease cleavage site is a tumor-associated protease cleavage site. Accordingly, in some embodiments, the protease cleavage site in the engineered Fc domain is a tumor-associated protease cleavage site. The tumor-associated protease cleavage site can be recognized by a tumor-associated protease such that the Fc domain is cleaved. As non-limiting examples, the tumor-associated protease is a matrix metalloproteinase (MMP), selected from the group consisting of MMP1, MMP2, MMP3, MMP7, MMP8, MMP9, MMP10, MMP11, MMP12, MMP13, MMP14, MMP15, MMP16, MMP17, MMP19, MMP20, MMP21, MMP23A, MMP23B, MMP24, MMP25, MMP27, and MMP28. In one embodiment, the tumor-associated protease is MMP2. In another embodiment, the tumor-associated protease is MMP3. In yet another embodiment, the tumor-associated protease is MMP9. In yet another embodiment, the tumor-associated protease is MMP10. Other disease associated and tissue selective proteases include but are not limited to Cathepsins (Cathepsin B, cathepsin D, cathepsin F, cathepsin K, cathepsin L, cathepsin V, cathepsin S, cathepsin W), ADAM, ADAMTS, Kallikreins 1 to 15, HTRA1-2-3, HGFAc, PRSS, TMPRSS, elastase, PR-3, granzymes (granzyme A, B, M, H and K), fibroblast activation proteins (FAP), plasmin, urokinase plasminogen activator (uPA), Tryptase, Caspase, Thrombin, Legumain, Chymase, Collagenase, napsin A, and matripatsel-2. In one embodiment, the disease-associated and tissue selective protease is Cathepsin B. In another embodiment, the disease-associated and tissue selective protease is matriptase.

In accordance with the present disclosure, the cleavable Fc domain is engineered to comprise at least one protease cleavage site. As non-limiting examples, the engineered Fc domain comprises one or more amino acid substitutions as compared to a wild-type Fc domain, wherein the substitutions create a protease cleavage site. In some embodiments, the engineered Fc domain comprises one or more amino acid substitutions in the hinge region, in the CH2 domain, in the CH3 domain and/or in the CH2-CH3 linker region, such that the engineered Fc domain comprises a protease cleavage site in the hinge region, in the CH2 domain, in the CH3 domain or in the CH2-CH3 linker region, In some embodiments, the engineered Fc domain comprises one or amino acid substitutions in positions 439-446 according to EU numbering; such that the substitutions create a protease cleavage site in positions 439-446 of the Fc domain.

In one aspect, the present invention provides, among other thing, an engineered Fc domain comprising one or more amino acid substitutions in the hinge region, in the CH2 domain, in the CH3 domain, and/or in the CH2-CH3 linker, such that the engineered Fc domain comprises a protease cleavage site.

In some embodiments, the one or more amino acid substitutions are in the hinge region. In some embodiments, the one or more amino acid substitutions are in the hinge region. In some embodiments, the one or more amino acid substitutions are in the CH2 domain. In some embodiments, the one or more amino acid substitutions are in the CH3 domain. In some embodiments, the one or more amino acid substitutions are in the CH2-CH3 linker.

In one aspect, the present invention provides, among other things, an engineered Fc domain comprising one or more amino acid substitutions in positions 436-447 according to EU numbering, such that the engineered Fc domain comprises a protease cleavage site.

In some embodiments, the protease cleavage site is located between positions 438 and 445, between positions 439 and 446, between positions 440 and 447, between positions 438 and 447, between positions 437 and 444, between positions 440 and 443, between positions 441 and 444, between positions 442 and 445, between positions 444 and 447, between positions 441 and 447, between positions 443 and 447, between positions 436 and 443, or between positions 442 and 447 by EU numbering.

In some embodiments, the engineered Fc domain comprises at least one set of the combined mutations, including: N434I, Y346V and Q438L mutations; H435S, Q438I and S440G mutations; T437S, Q438N and K438E mutations; K439V and L441S mutations; Q438P, K439T and L441T mutations; K439E, P445E and G446E mutations; and K439A, S444A and G446V mutations.

In some embodiments, the engineered Fc domain comprises a cleavage substrate in G-strand region. In some embodiments, the engineered Fc domain comprises a cleavage substrate in F-strand region. In some embodiments, the engineered Fc domain comprises a cleavage substrate in FG-loop region.

In another aspect, the present invention provides, among other things, an engineered Fc domain comprising one or more amino acid substitutions in positions 416-425 according to EU numbering, such that the engineered Fc domain comprises a protease cleavage site.

In further another aspect, the present invention provides, among other things, an engineered Fc domain comprising one or more amino acid substitutions in positions 426-437 according to EU numbering, such that the engineered Fc domain comprises a protease cleavage site. As non-limiting examples, provided in the present disclosure is an engineered cleavable Fc domain comprising the tumor-associated protease cleavage site having amino acid sequence of VPLSLYSG (SEQ ID NO: 95). As non-limiting examples, provided in the present disclosure is an engineered cleavable Fc domain comprising the tumor-associated protease cleavage site having amino acid sequence of VPLSLYSGP (SEQ ID NO: 209). In one embodiment, the present engineered Fc domain comprising the tumor-associated protease cleavage site having amino acid sequence of IHVTLKSL (SEQ ID NO: 99). In one embodiment, the present engineered Fc domain comprising the tumor-associated protease cleavage site having amino acid sequence of NSYTIKGL (SEQ ID NO.: 100). In one embodiment, the present engineered Fc domain comprising the tumor-associated protease cleavage site having amino acid sequence of SNESLSLS (SEQ ID NO.: 102). In one embodiment, the present engineered Fc domain comprising the tumor-associated protease cleavage site having amino acid sequence of QVSSSLSP (SEQ ID NO.: 104). In one embodiment, the present engineered Fc domain comprising the tumor-associated protease cleavage site having amino acid sequence of PTSTSLSP (SEQ ID NO.: 105). In one embodiment, the present engineered Fc domain comprising the tumor-associated protease cleavage site having amino acid sequence of ESLSLSEE (SEQ ID NO.: 107). In one embodiment, the present engineered Fc domain comprising the tumor-associated protease cleavage site having amino acid sequence of ASLSLAPV (SEQ ID NO.: 108). In one embodiment, the present engineered Fc domain comprising the tumor-associated protease cleavage site having amino acid sequence of SQESLSLS (SEQ ID NO.: 109). In one embodiment, the present engineered Fc domain comprising the tumor-associated protease cleavage site having amino acid sequence of PLGL (SEQ ID NO.: 110).

In some embodiments, the protease site comprises any one of SEQ ID NOs: 95, 97, 99-100, 102, 104, 105, 107-110, 119, 122, 123, 135-139, 143-208, and 209.

In some embodiments, engineered Fe domain of any one of preceding claims, wherein the engineered Fc domain comprises any one of amino acid substitution sets listed in Table 5, Table 8a, Table 11, Table 11a, Table 11b or Table 11c.

In some embodiments, the engineered Fc domain comprises at least one set of the combined substitutions in Table 5.

In some embodiments, the engineered Fc domain comprises at least one set of the combined substitutions in Table 8a.

In some embodiments, the engineered Fc domain comprises at least one set of the combined substitutions in Table 11.

In some embodiments, the engineered Fc domain comprises at least one set of the combined substitutions in Table 11a.

In some embodiments, the engineered Fc domain comprises at least one set of the combined substitutions in Table 11b.

In some embodiments, the engineered Fc domain comprises at least one set of the combined substitutions in Table 11c.

In some embodiments, the engineered cleavable Fc domain comprises a first Fc polypeptide and a second Fc polypeptide, wherein the one of the first or the second Fc polypeptide comprises a protease cleavage site. In some examples, the one of the first or the second Fc polypeptide comprises a tumor associated protease cleavage site as described herein.

In some embodiments, the first and/or the second Fc polypeptides each contain one or more modifications that promote the non-covalent association of the first and the second Fc polypeptides.

In some embodiments, the first Fc polypeptide is a first IgG1, IgG2, or IgG4 Fc domain, or a fragment thereof. In some embodiments, the second Fc polypeptide is a second IgG1, IgG2, or IgG4 Fc domain, or a fragment thereof

In some embodiments, the cytokine molecule of the present masked and/or targeted cytokine is IL-1, IL-2, IL-3, IL-4, IL-5, IL-6, IL-7, IL-9, IL-10, IL-11, IL-12, IL-13, IL-14, IL-15, IL-16, IL-17, IL-18, IL-20, IL-22, IL-34, TNF-α, TNF-β, CXCL8 (IL-8), G-CSF, GM-CSF, LIF, OSM, IFN-α, IFN-β, IFN-γ, CD154, LT-β, 4-1BBL, APRIL, CD70, CD153, CD178, GITRL, LIGHT, OX40L, TALL-1, TRAIL, TWEAK, TRANCE, TGF-β, M-CSF, or MSP, or a variant thereof, or a fragment thereof. As non-limiting examples, the cytokine molecule of the present masked or targeted cytokine is IL-2, IL-15 or IL-12, or a variant thereof, or a fragment thereof.

In one embodiment, the cytokine molecule is IL-2, wherein the IL-2 is a modified IL-2 cytokine or functional fragment thereof compared to the sequence of a mature IL-2 having the amino acid sequence of SEQ ID NO: 13. As non-limiting examples, the modified IL-2 cytokine or functional fragment thereof comprises modifications R38A, F42A, Y45A, and E62A relative to the sequence of a mature IL-2 having the amino acid sequence of SEQ ID NO: 13. In some examples, the modified IL-2 cytokine or functional fragment thereof comprises the modification C125A relative to the sequence of a mature IL-2 having SEQ ID NO: 13. In other examples, the modified IL-2 cytokine or functional fragment thereof comprises R38A, F42A, Y45A, E62A and C125A relative to the sequence of a mature IL-2 having SEQ ID NO: 13.

In another embodiment, the cytokine molecule is IL-15, wherein the IL-15 polypeptide comprises an amino acid sequence of SEQ ID NO: 16 or an amino acid sequence having at least one amino acid modification as compared to the amino acid sequence of SEQ ID NO: 16.

In another embodiment, the cytokine molecule is IL-12, wherein the IL-12 polypeptide or functional fragment thereof comprises an IL-12p40 polypeptide or functional fragment thereof covalently linked to an IL-12p35 polypeptide or functional fragment thereof. The IL-12p40-IL-12p35 linker is between 5 and 20 amino acids in length. In some examples, The IL-12p40-IL-12p35 linker is rich in amino acid residues G and S.

In accordance with the present disclosure, the present masked cytokine comprises a masking moiety that binds to the cytokine molecule.

In some embodiments, the masking moiety is a receptor of the cytokine, or a variant thereof, or a fragment thereof. As non-limiting examples, the masking moiety is CD121, IL-18Rα, IL-18Rβ, CD25, CD122, CD132, CD124, CD213a13, CD132, CD127, IL-9R, CD213a1, CD213a2, CD1243, CD132, IL-15Ra, CDw131, CDw125, CD131, CD116, CD126, CD130, IL-11Ra, CD114, CD212, LIFR, OSMR, IL-20Rα, IL-20Rβ, IL-14R, CD4, CDw127, CD118, CDw119, CD40, LTOR, CD120a, CD120b, CDw137, BCMA, TACI, CD27, CD30, CD95, GITR, LTbR, HVEM, OX40, TRAILR1-4, Apo3, RANK, OPG, TGF-βR1, TGF-βR2, TGF-βR3, CD 115, or CDw136, or a variant thereof, or a fragment thereof.

In one embodiment, the masking moiety is CD122. In some examples, the CD122 is an engineered CD122 polypeptide or a fragment thereof comprising one or more mutations relative to a wild-type CD122 amino acid sequence. The engineered CD122 comprises one or more mutations selected from the group consisting of F8C, A94C, L106C, C122S, C122V, C122A, N123C, N123Q, C168V, C168A, C168S, L169C, Q177C, V184C, S195C, and R204C.

In some embodiments, the masking moiety is a peptide, a polypeptide, a protein, a ligand, or an agent that specifically binds to the cytokine, or the variant thereof, or the fragment thereof.

In some embodiments, the masking moiety comprises a Fab, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), a single heavy chain antibody (HcAb), an antibody, or a combination thereof.

In some embodiments, the masking moiety comprises an anti-IL-2 scFv. In some embodiments, the masking moiety comprises a scFv having SEQ ID NO: 124. In some embodiments, the masking moiety comprises a scFv having SEQ ID NO: 125. In some embodiments, the masking moiety comprises a scFv having SEQ ID NO: 124 and SEQ ID NO: 125. In some embodiments, the masking moiety comprises a scFv having SEQ ID NO: 124 linked to SEQ ID NO: 125 by a linker. In some embodiments, the masking moiety comprises a scFv having SEQ ID NO: 142.

In some embodiments, the masking moiety comprises an anti-IL-2 VHH. In some embodiments, the masking moiety comprises a VHH having a hCDR1 of SEQ ID NO: 132, hCDR2 of SEQ ID NO: 133, and hCDR3 of SEQ ID NO: 134. In some embodiments, the masking moiety comprises a VHH having SEQ ID NO: 121.

In some embodiments, the masked cytokine of the present disclosure comprises an engineered cleavable Fc domain moiety that is further modified. In some embodiments, the first and the second Fc polypeptides of the present engineered cleavable Fc domain moiety further comprise the same amino acid substitutions described herein. In other embodiments, the first and the second Fc polypeptides of the present engineered cleavable Fc domain moiety comprise different amino acid substitutions described herein. As non-limiting examples, the present engineered Fc domain comprising a first Fc polypeptide comprising Y349C, T366S, L368A, Y407V, and N297A mutations and a second Fc polypeptide comprising S354C, T366W and N297A mutations. Alternatively, the present engineered Fc domain comprising a first Fc polypeptide comprising S354C, T366W and N297A mutations and a second Fc polynucleotide comprising Y349C, T366S, L368A, Y407V, and N297A mutations. In another example, the present engineered Fc domain comprising a first Fc polypeptide comprising Y349C, T366S, L368A, Y407V, N297A and I253A mutations and a second Fc polypeptide comprising S354C, T366W, N297A and I253A mutations. Alternatively, the present engineered Fc domain comprising a first Fc polypeptide comprising S354C, T366W, N297A and I253A mutations and a second Fc polynucleotide comprising Y349C, T366S, L368A, Y407V, N297A and I253A mutations.

The masked cytokine of the present disclosure comprises an engineered cleavable Fc domain moiety that is further modified. In some embodiments, the first Fc polypeptide of the engineered Fc domain comprises a CH3 domain comprising a modification that reduces or eliminates binding to Protein A, and the second Fc domain comprises a CH3 domain that binds to Protein A. In some embodiments, the CH3 domain that binds to Protein A is a human IgG1, IgG2 or IgG4 sequence. The second CH3 domain may be a human IgG1, IgG2 or IgG4 sequence comprising a modification at position H435 and/or Y436 according to Kabat numbering. As a non-limiting example, the second CH3 domain comprises the modifications of H435R and Y436F according to Kabat numbering.

In one embodiment, the first CH3 domain comprises a human IgG3 sequence.

In some embodiments, the present masked cytokine further comprises a targeting moiety; the targeting moiety comprises one or more antigen binding domains, peptide, a polypeptide, a protein, a ligand, or an agent that specifically binds to an antigen or a ligand. As non-limiting examples, the targeting moiety comprises an antigen binding domain selected from the group consisting of Fab, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), an antibody, and a combination thereof.

In one embodiment, the targeting moiety comprises a first antigen binding domain and a second antigen binding domain. In some examples, the first and second antigen binding domains specifically bind to the same target. The first and second antigen binding domains may comprise the same amino acid sequence. In other examples, the first and second antigen binding domains specifically bind to different targets. The first and second antigen binding domains comprise different amino acid sequences.

The targeting moiety may bind to a molecule specific to a target, therefore targets the masked cytokine to the target, e.g., a targeted cell, tissue and organ. In some embodiments, the targeting moiety specifically binds PD-1, PD-L1, PD-L2, CTLA-4, TIGIT, TIM-3, LAG-3, CD25, CD16a, CD16b, NKG2A, NKG2D, NKP44, NKP30, CD19, CD20, CD30, CD38, BCMA, human epidermal growth factor receptor 2 (HER2), human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGF R1), vascular endothelial growth factor receptor 2 (VEGF R2), Nectin-4, Liv-1, glycoprotein NMB (GPNMB), prostate specific membrane antigen (PSMA), Trop-2, carbonic anhydrase IX (CA9), endothelin B receptor (ETBR), six transmembrane epithelial antigen of the prostate 1 (STEAP1), folate receptor alpha (FR-a), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD22, CD33, CD40, CD56, CD66e, CD70, CD74, CD79b, CD94, CD96, CD98, CD 123, CD 138, CD352, CD47, signal-regulatory protein alpha (SIRPa), Claudin 18.2, Claudin 6, 5T4, fibroblast activation protein alpha (FAPa), the melanoma-associated chondroitin sulfate proteoglycan (MCSP), epithelial cellular adhesion molecule (EPCAM), or combinations thereof.

In one preferred embodiment, the targeting moiety binds to PD-1.

In accordance with the present disclosure, the moieties of the present masked cytokine are covalently linked. In some embodiments, the targeting moiety is linked to the engineered Fc domain through one or both of the first and second Fc polypeptides. In one example, the targeting moiety is linked to the N-terminus of the first Fc polypeptide of the engineered Fc domain and the C-terminus of the first Fc polypeptide is linked to the N-terminus of the cytokine or a fragment thereof. In another example, the C-terminus of the targeting moiety is linked to the N-terminus of the second Fc polypeptide of the engineered Fc domain and the C-terminus of the second Fc polypeptide is linked to the N-terminus of the masking moiety.

In accordance with the present disclosure, the protease cleavage site has an in vitro cleavage efficiency yielding at least 10% active cytokine. In some embodiments, about 10% to 30% active cytokine is released after cleavage.

In some embodiments, provided in the present disclosure include nucleic acid molecules that encode the engineered Fc domain comprising a protease cleavage site. Provided in the present disclosure also include nucleic acid molecules that encode the masked cytokine.

In some embodiments, provided in the present disclosure are vectors, host cells comprising nucleic acid molecules encoding the engineered Fc domain comprising a protease cleavage site and the masked cytokine as described herein. Methods of producing the present masked cytokine are provided herein; the methods comprise culturing the host cell comprising a nucleic acid molecule encoding the engineered Fc domain comprising a protease cleavage site or the masked cytokine, under a condition that produces the engineered Fc domain or the masked cytokine.

Provided in the present disclosure further include compositions, pharmaceutical compositions and kits that comprise the engineered Fc domain or the masked cytokine. The pharmaceutical composition comprising the engineered cleavable Fc domain and/or the masked cytokine further comprises a pharmaceutically acceptable excipient. In some embodiments, the pharmaceutical composition comprising the engineered cleavable Fc domain and/or the masked cytokine is formulated for administration via a defined route, e.g., intravenous (IV) infusion.

In another aspect of the present disclosure, methods of use of the engineered cleavable Fc domain and the masked cytokine, are provided. In some embodiments, the present disclosure provides a method for treating or preventing a neoplastic disease in a subject; the method comprises administering to the subject an effective amount of the masked cytokine, compositions or pharmaceutical compositions that comprise the present masked cytokine.

In some embodiments, the present masked cytokine, compositions or pharmaceutical compositions that comprise the present masked cytokine is used in a method of treating or preventing an inflammatory or autoimmune disease in a subject; the method comprises administering to the subject an effective amount of the masked cytokine, compositions or pharmaceutical compositions that comprise the present masked cytokine.

In some embodiments, the present engineered cleavable Fc domain is used, alone or in combination with other therapeutic agents for treating a neoplastic disease, an inflammatory disease, and/or an autoimmune disease.

In one aspect, the present invention provides, among other things, a masked cytokine comprising a cytokine moiety, a masking moiety, and an engineered Fc domain comprising a tumor-associated protease cleavage site, wherein the engineered Fc domain is fused to the cytokine moiety or the masking moiety such that the masking moiety binds to the cytokine moiety and upon cleavage of the tumor-associated protease cleavage site on the engineered Fc domain, the cytokine moiety is released from the masking moiety.

In one aspect, the present invention provides, among other things, a masked therapeutically active molecule comprising a therapeutically active domain, a masking moiety, and an engineered Fc domain comprising a tumor-associated cleavage site, wherein the engineered Fc domain is fused to the therapeutically active domain or the masking moiety such that the masking moiety binds to the therapeutically active domain and upon cleavage of the tumor-associated protease cleavage site on the engineered Fc domain, the therapeutically active domain is released from the masking moiety.

In some embodiments, the engineered Fc domain is fused to the therapeutically active domain or the masking moiety via a non-cleavable linker. In some embodiments, the engineered Fc domain is directly fused to the therapeutically active domain or the masking moiety.

In some embodiments, a therapeutically active domain is a cell engager. In some embodiments, a therapeutically active domain is a co-stimulatory domain.

### BRIEF DESCRIPTION OF THE DRAWINGS

The drawings are for illustration purposes only, not for limitation.

FIG. 1A is a presentative schematic demonstrating the design of a masked cytokine with a cleavable Fc domain comprising a protease cleavage site (protease substrate). FIG. 1B is an exemplary schematic of a masked cytokine comprising a protease cleavage site in the linker.

FIG. 2 illustrates two exemplary universal cleavable Fc platforms displaying two different protease substrates (VPLSLYSG and MMP7-specific substrate).

FIGS. 3A and 3B illustrate the structure of the human Fc beta strands and loop regions. FIG. 3C lists the sequences of hFc where a protease cleavage site is incorporated.

FIG. 4 illustrates the fusion of a mutated IL-12 (muIL-12) to the C-terminal of the knob chain of Fc.

FIGS. 5A and 5B are SDS-PAGE images, showing the cleavage of several constructs with VPLSLYSG substrates (except #19) by MMP10: full cleavage of constructs #12 (FIG. 5A) and #23 (FIG. 5B), and partial cleavage of constructs #7, 9, 10, and 11 (shown in FIG. 5A), and #19, 20, and 21 (shown in FIG. 5B).

FIGS. 6A and 6B are SDS-PAGE images, showing the cleavage of several constructs with VPLSLYSG substrates (except #19) by MMP2, MMP3 and MMP9: nearly full cleavage of constructs #13, 15, 16 and 17 (FIG. 6A), and partial cleavage of constructs #19 and 20 (FIG. 6B).

FIG. 7A is a series of exemplary EC50 graphs of masked IL-2 cytokines with cleavable Fc domain in the absence of matrix metalloprotease (MMP) as performed in a cell-based reporter assay (HEK Blue IL-2 assay) which determines % calculated active cytokine. Recombinant human IL-2 (rhIL-2) and CM3 (unmasked, Fc domain fused to IL-2) were used as a positive control. FIG. 7B is a series of exemplary EC50 graphs of masked IL-2 cytokines with cleavable Fc domain in the presence of MMPs, as performed in a cell-based reporter assay (HEK Blue IL-2 assay) which determines % calculated active cytokine.

FIG. 8 is an exemplary schematic of a targeted cytokine construct containing a cleavable Fc domain. The cross indicates the location of a cleavage site. One Fc polypeptide is fused to a cytokine via a non-cleavable linker, and the other Fc polypeptide is fused to a masking moiety (e.g., anti-cytokine VHH) via a non-cleavable linker. Each Fc polypeptide also contains a targeting moiety (e.g., Fab) that can specifically bind to a target of interest (e.g., PD-1).

FIG. 9A illustrates the mechanism of the PD-1/PD-L1 Blockade Bioassay used in Example 8. This assay is a biologically relevant MOA-based assay that can be used to measure the potency and stability of antibodies and other biologics designed to block the PD-1/PD-L1 interaction. When the two cell types are co-cultured, the PD-1/PD-L1 interaction inhibits TCR signaling and NFAT-mediated luciferase activity. Addition of an antibody that blocks either PD-1 or PD-L1 releases the inhibitory signal and results in TCR signaling and NFAT-mediated luciferase activity. FIG. 9B is an exemplary schematic of a targeted “unmasked” cytokine construct that is used as a positive control. As compared to a targeted cytokine as represented in FIG. 8, the “unmasked” version does not contain a masking moiety.

FIG. 10 is a series of exemplary graphs showing effective blocking of the PD-1/PD-L1 interaction by PD-1 targeted cytokines incorporating cleavable Fc domains.

FIGS. 11A and 11B are series of exemplary EC50 graphs of targeted IL-2 cytokines with cleavable Fc domain in absence and presence of matrix metalloprotease (MMP) as performed in a cell-based reporter assay (HEK Blue IL-2 assay) which determines % calculated active cytokine.

FIG. 12 is a series of exemplary graphs illustrating body weight % change, tumor growth inhibition, and overall survival of mice treated with the targeted cytokine of the present invention, demonstrating in vivo efficacy.

FIGS. 13A to 13E are exemplary graphs illustrating cleavage kinetics of masked cytokines comprising cleavable Fc domains by MMP7, MMP8, MMP14, MMP9 and MMP10, respectively.

FIGS. 14A to 14C are exemplary graphs illustrating cleavage kinetics of UCM-75-UCM84 by MMP7, MMP9 and MMP14, respectively.

FIG. 15A shows % cleavage of cleavable Fc molecules UCM75-UCM84 by human tumors ex vivo. FIG. 15B shows that cleavable Fc molecules UCM75-UCM84 are not cleaved by human plasma.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

It is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a,” “an,” and “the” include plural referents unless the content clearly dictates otherwise. Thus, for example, reference to “an IL-2 polypeptide” optionally includes a combination of two or more such polypeptides, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

As used herein, the term “and/or” refers to any one of the items, any combination of the items, or all of the items with which the term is associated. For instance, the phrase “A, B, and/or C” is intended to encompass each of the following embodiments: A, B, and C; A, B, or C; A or B; A or C; B or C; A and B; A and C; B and C; A and B or C; B and A or C; C and A or B; A (alone); B (alone); and C (alone).

Antibody: As used herein, the term “antibody” is used in the broadest sense, including polyclonal antibodies, monoclonal antibodies (including full length antibodies which have an immunoglobulin Fc region), antibody compositions with polyepitopic specificity, multispecific antibodies (e.g., bispecific antibodies, diabodies, and single-chain molecules (e.g., scFv and heavy chain only antibody (HcAb)), as well as antibody fragments (e.g., Fab, F(ab′)2, and Fv). The term “immunoglobulin” (Ig) is used interchangeably with “antibody” herein. An “antibody” can be naturally occurring or man-made.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the a and y chains and four CH domains for p and s isotypes. Each L chain has at the N-terminus, a variable domain (VL) followed by a constant domain at its other end. The VL is aligned with the VH and the CL is aligned with the first constant domain of the heavy chain (CHI). Particular amino acid residues are believed to form an interface between the light chain and heavy chain variable domains. The pairing of a VH and VL together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, C T, 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated a, 8, e, y and p, respectively. The y and a classes are further divided into subclasses on the basis of relatively minor differences in the CH sequence and function, e.g., humans express the following subclasses: IgGl, IgG2, IgG3, IgG4, IgA1 and IgA2. IgGl antibodies can exist in multiple polymorphic variants termed allotypes (reviewed in Jefferis and Lefranc 2009. mAbs Vol 1 Issue (4): 1-7) any of which are suitable for use in the invention. Common allotypic variants in human populations are those designated by the letters a,f,n,and z.

An “isolated” antibody is one that has been identified, separated and/or recovered from a component of its production environment (e.g., naturally or recombinantly). In some embodiments, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In some embodiments, the polypeptide is purified: (1) to greater than 95% by weight of antibody as determined by, for example, the Lowry method, and in some embodiments, to greater than 99% by weight; (1) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or silver stain. Isolated antibody includes the antibody in situ within recombinant cells since at least one component of the antibody's natural environment will not be present. Ordinarily, however, an isolated polypeptide or antibody is prepared by at least one purification step.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. In some embodiments, monoclonal antibodies have a C-terminal cleavage at the heavy chain and/or light chain. For example, 1, 2, 3, 4, or 5 amino acid residues are cleaved at the C-terminus of heavy chain and/or light chain. In some embodiments, the C-terminal cleavage removes a C-terminal lysine from the heavy chain. In some embodiments, monoclonal antibodies have an N-terminal cleavage at the heavy chain and/or light chain. For example, 1, 2, 3, 4, or 5 amino acid residues are cleaved at the N-terminus of heavy chain and/or light chain. In some embodiments truncated forms of monoclonal antibodies can be made by recombinant techniques. In some embodiments, monoclonal antibodies are highly specific, being directed against a single antigenic site. In some embodiments, monoclonal antibodies are highly specific, being directed against multiple antigenic sites (such as a bispecific antibody or a multispecific antibody). The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including, for example, the hybridoma method, recombinant DNA methods, phage-display technologies, and technologies for producing human or human-like antibodies in animals that have parts or all of the human immunoglobulin loci or genes encoding human immunoglobulin sequences.

The terms “full-length antibody,” “intact antibody” or “whole antibody” are used interchangeably to refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, such as the antigen binding region and/or the variable region of the intact antibody, and/or the constant region of the intact antibody. Examples of an antibody fragment include the Fc region of the antibody, a portion of the Fc region, or a portion of the antibody comprising the Fc region. Examples of antigen-binding antibody fragments include domain antibodies (dAbs), Fab, Fab′, F(ab′)2 and Fv fragments; diabodies; linear antibodies (see U.S. Pat. No. 5,641,870, Example 2; Zapata et al., Protein Eng. 8(10): 1057-1062 [1995]); single-chain antibody molecules, and multispecific antibodies formed from antibody fragments, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), and a combination thereof. Single heavy chain antibodies or single light chain antibodies can be engineered, or in the case of the heavy chain, can be isolated from camelids, shark, libraries or mice engineered to produce single heavy chain molecules.

Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CHI). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having different antigen-binding activity and is still capable of cross-linking antigen. Fab′ fragments differ from Fab fragments by having a few additional residues at the carboxy terminus of the CHI domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known. The Fc fragment comprises the carboxy-terminal portions of both H chains held together by disulfides. The effector functions of antibodies are determined by sequences and glycan in the Fc region, the region which is also recognized by Fc receptors (FcR) found on certain types of cells.

The term “diabodies” refers to small antibody fragments with two antigen-binding sites, which comprise a heavy chain variable (VH) domain connected to a light chain variable (VL) domain in the same polypeptide chain (VH-VL).

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative subshtuyions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y
where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

Antibody “effector functions” refer to those biological activities attributable to the Fc region (a native sequence Fc region or amino acid sequence variant Fc region) of an antibody and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptors); and B cell activation.

Amino acid: As used herein, term “amino acid,” in its broadest sense, refers to any compound and/or substance that can be incorporated into a polypeptide chain. In some embodiments, an amino acid has the general structure H2N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

At risk: As used herein, an individual “at risk” of developing a disorder may or may not have detectable disease or symptoms of disease and may or may not have displayed detectable disease or symptoms of disease prior to the treatment methods described herein. “At risk” denotes that an individual has one or more risk factors, which are measurable parameters that correlate with development of the disease, as known in the art. An individual having one or more of these risk factors has a higher probability of developing the disorder than an individual without one or more of these risk factors.

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active.

Binding affinity: As used herein the term “binding affinity” refers to the strength of the non-covalent interactions between a single binding site of a molecule (e.g., a cytokine) and its binding partner (e.g., a cytokine receptor). In some embodiments, the affinity of a binding protein (e.g., a cytokine) can generally be represented by a dissociation constant (Kd). Affinity can be measured by common methods known in the art, including those described herein.

Pharmaceutical carriers: As used herein, the term “pharmaceutical carriers” includes pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

Chronic: As used herein, a “chronic” administration refers to administration of the medicament(s) in a continuous as opposed to acute mode, so as to main the initial therapeutic effect (activity) for an extended period of time. “Intermittent” administration is treatment that is not consecutively done without interruption, but rather is cyclic in nature.

Disease: As used herein the term “disease” is a pathological condition, for example, one that can be identified by symptoms or other identifying factors as diverging from a healthy or a normal state. The term “disease” includes disorders, syndromes, conditions, and injuries. Diseases include, but are not limited to, proliferative, inflammatory, immune, metabolic, infectious, and ischemic diseases.

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery).

Dosing interval: As used herein, the term “dosing interval” in the context of a method for treating a disease is the frequency of administering a therapeutic composition in a subject (mammal) in need thereof, for example an mRNA composition, at an effective dose of the mRNA, such that one or more symptoms associated with the disease is reduced; or one or more biomarkers associated with the disease is reduced, at least for the period of the dosing interval. Dosing frequency and dosing interval may be used interchangeably in the current disclosure.

Effective amount: As used herein, an “effective amount” refers to at least an amount effective, at dosages and for periods of time necessary, to achieve the desired or indicated effect, including a therapeutic or prophylactic result. An effective amount can be provided in one or more administrations. A “therapeutically effective amount” is at least the minimum concentration required to effect a measurable improvement of a particular disorder. A therapeutically effective amount herein may vary according to factors such as the disease state, age, sex, and weight of the patient, and the ability of the antibody to elicit a desired response in the individual. A therapeutically effective amount may also be one in which any toxic or detrimental effects of the targeted cytokine are outweighed by the therapeutically beneficial effects. A “prophylactically effective amount” refers to an amount effective, at the dosages and for periods of time necessary, to achieve the desired prophylactic result. Typically, but not necessarily, since a prophylactic dose is used in subjects prior to or at the earlier stage of disease, the prophylactically effective amount can be less than the therapeutically effective amount.

Effective dose: As used herein, an effective dose is a dose of the mRNA in the pharmaceutical composition which when administered to the subject in need thereof, hereby a mammalian subject, according to the methods of the invention, is effective to bring about an expected outcome in the subject, for example reduce a symptom associated with the disease.

Host cell: As used herein, the term “host cell” is an individual cell or a cell culture which can be or has been a recipient of any recombinant vector(s) or isolated polynucleotide(s), such as a vector or polynucleotide for an engineered cleavable Fc domain, or a masked cytokine of the present disclosure. A host cell includes cells transfected or infected in vivo or in vitro with a recombinant vector or a polynucleotide of the present invention. In some cases, the host cell is a mammalian host cell.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In conjunction with: As used herein, “in conjunction with” or “in combination with” refers to administration of one treatment modality in addition to another treatment modality. As such, “in conjunction with” or “in combination with” refers to administration of one treatment modality before, during or after administration of the other treatment modality to the individual.

Individual: As used herein, an “individual” or a “subject” is a mammal. A “mammal” for purposes of treatment includes humans, domestic and farm animals, and zoo, sports, or pet animals, such as dogs, horses, rabbits, cattle, pigs, hamsters, gerbils, mice, ferrets, rats, cats, etc. In some embodiments, the individual or subject is human.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, an “isolated” nucleic acid molecule encoding the cytokine polypeptides described herein is a nucleic acid molecule that is identified and separated from at least one contaminant nucleic acid molecule with which it is ordinarily associated in the environment in which it was produced. In some embodiments, the isolated nucleic acid is free of association with all components associated with the production environment. The isolated nucleic acid molecules encoding the polypeptides and cytokine polypeptides herein is in a form other than in the form or setting in which it is found in nature. Isolated nucleic acid molecules therefore are distinguished from nucleic acid encoding the polypeptides and cytokine polypeptides herein existing naturally in cells.

Pharmaceuticalformulation: As used herein, the term “pharmaceutical formulation” refers to a preparation that is in such form as to permit the biological activity of the active ingredient to be effective, and that contains no additional components that are unacceptably toxic to a subject to which the formulation would be administered.

Moiety: As used herein, the term “moiety” refers to sub-structures which are part of a molecule.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4 alkyl)4 salts. Representative alkali or alkaline earth metal salts include sodium, lithium, potassium, calcium, magnesium, and the like. Further pharmaceutically acceptable salts include, when appropriate, nontoxic ammonium. quaternary ammonium, and amine cations formed using counterions such as halide, hydroxide, carboxylate, sulfate, phosphate, nitrate, sulfonate and aryl sulfonate. Further pharmaceutically acceptable salts include salts formed from the quarternization of an amine using an appropriate electrophile, e.g., an alkyl halide, to form a quarternized alkylated amino salt.

Polypeptide: As used herein, the terms “Polypeptide,” “peptide,” and “protein” are used interchangeably herein to refer to a polymer of amino acid residues, e.g., a polymer of at least 3, 4, 5, 6, 7, 8, 9, lo, or more amino acids. Throughout the present disclosure, standard three letter or single letter designations for amino acids are used. As used herein, the terms encompass amino acid chains of any length, including full-length proteins, wherein the amino acid residues are linked by covalent peptide bonds.

Polynucleotide: As used herein, the term “polynucleotide” refers to a polymeric form of nucleotides of at least 5, 6, 7, 8, 9 or 10 bases or base pairs in length, either ribonucleotides or deoxynucleotides or a modified form of either type of nucleotide and is meant to include single and double stranded forms of DNA and/or RNA. Throughout the present disclosure, the term is used interchangeably with “nucleic acid molecule”.

Prevention: As used herein, the term “prevention” includes providing prophylaxis with respect to occurrence or recurrence of a disease in an individual. An individual may be predisposed to, susceptible to a disorder, or at risk of developing a disorder, but has not yet been diagnosed with the disorder. In some embodiments, targeted cytokines described herein are used to delay development of a disorder.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target tissues: As used herein, the term “target tissues” refers to any tissue that is affected by a disease to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treat: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. It can refer to any clinical intervention designed to alter the natural course of the individual or cell being treated during the course of clinical pathology. Treatment may be administered to a subject who does not exhibit signs of a disease and/or exhibits only early signs of the disease for the purpose of decreasing the risk of developing pathology associated with the disease. An individual is successfully “treated”, for example, if one or more symptoms associated with a disorder (e.g., a neoplastic disease) are mitigated or eliminated. For example, an individual is successfully “treated” if treatment results in increasing the quality of life of those suffering from a disease, decreasing the dose of other medications required for treating the disease, reducing the frequency of recurrence of the disease, lessening severity of the disease, delaying the development or progression of the disease, and/or prolonging survival of individuals.

Vector: As used herein, the term “vector” refers to a macromolecule or complex of molecules comprising a polynucleotide to be delivered to a host cell or organism, either in vitro or in vivo, typically a virus or a plasmid.

Various aspects of the invention are described in detail in the following sections. The use of sections is not meant to limit the invention. Each section can apply to any aspect of the invention. In this application, the use of “or” means “and/or” unless stated otherwise.

### DETAILED DESCRIPTION

### I. Introduction

Specific delivery and activation of therapeutic agents, such as cytokines, to tumor cells is desirable to increase safety of such treatment (e.g., to avoid targeting normal cells). The present invention provides compositions and methods for such precision treatment of cancer. In particular, the present invention relates to a universal cleavable carrier platform, together with the invented masking moieties, for the purpose of the precision treatment of tumor, among other things. The present cleavable carrier can be linked to a therapeutic agent (e.g., a cytokine) for tumor specific delivery and activation.

In general, the present disclosure relates to a cleavable carrier moiety that comprises an engineered tumor-associated protease cleavage site such that the carrier moiety is cleavable. The cleavable carrier moiety is fused to a therapeutic molecule and/or the masking moiety to form a masked therapeutic agent. For example, the carrier moiety is fused to the therapeutic molecule and/or the masking moiety via a non-cleavable linker. The masked therapeutic agent comprising such cleavable carrier moiety is capable of releasing the active therapeutic molecule from the masking moiety, upon cleavage of the engineered tumor-associated protease site within the cleavable carrier moiety.

As used herein, the term “cleavable carrier” refers to any agent that is cleavable from the present composition enzymatically or non-enzymatically. For example, the cleavable carrier may be a protein, a polypeptide, and a domain thereof. In the context of the present disclosure, the terms “cleavable carrier” and “cleavable domain” are used interchangeably. The present cleavable carrier moiety provides enzymatically induced prodrug activation, characterized in that the cleavage of the carrier moiety controls release of active therapeutics.

The cleavable carrier moiety may be a half-life extension domain derived from albumin, transferrin or a tissue factor. The cleavable carrier moiety may be an antigen targeting domain selected from the group consisting of immunoglobulin, Fab, F(ab)2, scFv, VHH, ScAb and nanobody. A tumor-associated protease cleavage site is engineered within the antigen targeting domain. The carrier moiety may be an Fc domain in which at least one tumor-associated protease cleavage site is engineered, e.g., by amino acid substitutions at particular positions of the Fc domain.

In addition to the aforementioned cytokines, the therapeutic molecule may include any therapeutic agent that has functional effects for a disease, such as therapeutic proteins, cell engagers, and co-stimulatory domains.

The masking moiety within the present masked therapeutic agent binds to the therapeutic molecule, masking it from being released and/or activated. The masking moiety can be any agent that binds to the therapeutic molecule. For example, the masking moiety is a peptide, a polypeptide, a protein, a ligand, or an agent that specifically binds to the cytokine, or the variant thereof, or the fragment thereof.

The masked therapeutic agent may further comprise a targeting moiety; the targeting moiety can specifically binds to an antigen, a ligand, or a biomarker of a targeted cell, tissue and/or organ. For example, the targeting moiety comprises one or more antigen binding domains, peptide, a polypeptide, a protein, a ligand, or an agent that specifically binds to an antigen, a ligand or a biomarker. In this context, the cytokine is also referred to as a targeted cytokine.

In accordance with the present disclosure, a masked therapeutic agent may be construed as a fusion polypeptide. Upon the cleavage of the engineered tumor-associated protease site in the carrier moiety, the polypeptide is cleaved, and the masked therapeutic molecule is then released and activated for its function in a targeted cell, tissue and/or organ (e.g., in a tumor tissue). The fusion polypeptide may be created by recombinant technologies known in the art.

In particular, the present disclosure provides, among other things, an engineered Fc domain comprising a tumor-associated protease cleavage site. The engineered Fc domain with the tumor-associated protease cleavage site provides a universal cleavable platform that can be linked to a therapeutic agent such as a masked cytokine, to control its activation. The engineered cleavable Fc domain of the present invention can be fused to a cytokine, a masking moiety, and/or a targeting moiety to produce a prodrug. Prodrugs comprising an engineered Fc domain of the present disclosure become active at the site of disease and are able to specifically target a cell of interest for effective treatment of cancer without causing undesired side effects.

The present disclosure also provides, among other things, therapeutic agents and pharmaceutical compositions and formulations thereof, comprising the engineered Fc domain and/or the targeted cytokine. Methods of use such agents and compositions and formulations for treatment of a disease (e.g., cancer) are provided as well.

### II. Compositions

Provided in the present invention include a cleavable carrier, and a composition comprising the cleavable carrier, among other things. As a non-limiting example, the cleavable carrier is a cleavable Fc domain. The present composition comprises a cleavable Fc domain. Among the compositions described herein, provided is a masked cytokine. The masked cytokine comprises a cytokine moiety, a masking moiety and an engineered cleavable Fc domain comprising a tumor-associated cleavage site, wherein the engineered cleavable Fe domain is fused to the cytokine moiety or the masking moiety such that the masking moiety binds to the cytokine moiety and upon cleavage of the tumor associated protease cleavage site of the engineered Fc domain, the cytokine moiety is released from the masking moiety. The engineered cleavable Fc domain is fused to the cytokine molecule and/or the masking moiety via a non-cleavable linker. The cleavable Fc domain-linked cytokine prodrugs increase therapeutic potency of cytokines in vivo.

Cleavable Carriers

As used herein, the term “cleavable carrier” refers to any agent that is cleavable from the present composition enzymatically or non-enzymatically. For example, the cleavable carrier may be a protein, a polypeptide, and a domain thereof. In the context of the present disclosure, the terms “cleavable carrier” and “cleavable domain” are used interchangeably. The present cleavable carrier moiety provides enzymatically induced prodrug activation, characterized in that the cleavage of the carrier moiety controls release of active therapeutics.

The cleavable carrier moiety may be a half-life extension domain derived from albumin, transferrin or a tissue factor. The cleavable carrier moiety may be an antigen targeting domain selected from the group consisting of immunoglobulin, Fab, F(ab)2, scFv, VHH, ScAb and nanobody. A tumor-associated protease cleavage site is engineered within the antigen targeting domain. The carrier moiety may be an Fc domain in which at least one tumor-associated protease cleavage site is engineered, e.g., by amino acid substitutions at particular positions of the Fc domain.

Engineered Cleavable Fc Domain

In accordance with the present disclosure, the carrier moiety is an Fc domain derived from an immunoglobulin, such as IgM, IgG1, IgG2, IgG3, IgG4, IgD, IgE and IgA, or variant thereof, or fragment thereof. Accordingly, the Fc domain is genetically engineered to be enzymatically cleavable. The engineered Fc domain is referred to a “cleavable Fc domain”.

As used herein, the term “Fc domain” refers to a polypeptide or a fragment from an immunoglobulin. The Fc domain may be any antibody or fragment thereof. The Fc domain is derived from an antibody or fragment thereof that naturally occur in an animal (e.g., a mammal) or is made by any technologies known in the art. In some embodiments, the Fc domain is a human Fc domain.

In some embodiments, the Fc domain comprises a first Fc polypeptide and a second Fc polypeptide. In some embodiments, the Fc domain is from any antibody or fragment thereof comprising either a heavy chain Fc polypeptide or a light chain Fc polypeptide. In some embodiments, the Fc domain comprises a portion of either a heavy chain polypeptide or a light chain polypeptide. In some embodiments, the antibody or fragment thereof comprises an Fc domain or fragment thereof. In some embodiments, the Fe domain derived from an antibody or fragment thereof comprises a hinge region, a CH2 domain and a CH3 domain or a fragment thereof. In some embodiments, the Fe domain comprises only the constant domain of the heavy chain polypeptide. In some embodiments, the Fc domain comprises only the constant domain of the light chain polypeptide. In some embodiments, the Fc domain comprises a first Fc polypeptide having the CH2 and CH3 domains and a second Fc polypeptide having the CH2 and CH3 domains. In some embodiments, the Fc domain comprises a first Fc polypeptide having the CH3 domain and a second Fc polypeptide having the CH3 domain. In some embodiments, the Fc domain comprises a first Fc polypeptide having the CH2 and CH3 domains and a second Fc polypeptide having the CH3 domain. In some embodiments, the Fc domain comprises a first Fc polypeptide having the CH3 domain and a second Fc polypeptide having the CH2 and CH3 domains.

In some embodiments, the first Fc polypeptide and the second Fc polypeptide are linked via a linker such as a short peptide linker. In some embodiments, the linker is non-cleavable.

An engineered cleavable Fc domain comprises one or more cleavable sites. As used herein, a “cleavage site” refers to a recognizable site for cleavage of a portion of the cleavable peptide found in any of the Fc domain. A “cleavage site” can be an amino acid sequence, such as a short peptide motif, that is recognized and cleaved by a cleaving agent. The cleavage sites may be the amino acid sequences naturally in the Fc domain. Additionally and/or alternatively, the cleavage sites may be introduced into the cleavable portion of the Fc domain by mutations (e.g., amino acid insertions, substitutions and deletions). The mutations do not change other activities of Fc domain. In some embodiments, one or more cleavage peptide motifs may be engineered in the cleavable Fc domain as described herein. The cleaving agent may be an enzyme, for example a protease. In some embodiments, the cleavage sites are protease cleavage sites such that the cleavable Fc domain is proteolytically cleavable. Proteases are enzymes that cleave and hydrolyse the peptide bonds between two specific amino acid residues of target substrate proteins. Proteases general recognize a specific peptide motif and cleave the peptide bonds between two specific amino acid residues within the short peptide motif.

The Fc domain may be engineered to include one or more peptide motifs that can be recognized by one or more protease such as tumor associated proteases, tissue selective proteases and diseases (e.g., inflammation) associated proteases. As non-limiting examples, the cleavable Fc domain as described herein may be cleaved by a disease associated or tissue selective protease selected from matrix metalloproteases (MMP1, MMP2, MMP3, MMP7, MMP8, MMP9, MMP10, MMP11, MMP12, MMP13, MMP14, MMP15, MMP16, MMP17, MMP19, MMP20, MMP21, MMP23A, MMP23B, MMP24, MMP25, MMP27 and MMP28), Cathepsins (Cathepsin B, cathepsin D, cathepsin F, cathepsin K, cathepsin L, cathepsin V, cathepsin S and cathepsin W), ADAM, ADAMTS, Kallikreins 1 to 15, HTRA1-2-3, HGFAc, PRSS, TMPRSS, elastase, PR-3, granzymes (granzyme A, B, M, H and K), fibroblast activation proteins (FAP), plasmin, urokinase plasminogen activator (uPA), Tryptase, Caspase, Thrombin, Legumain, Chymase, Collagenase, napsin A, and matriptasel-2.

In some embodiments, the cleavable Fc domain as described herein comprises at least one engineered tumor-associated protease cleavage site. A “tumor-associated protease cleavage site” as provided herein is an amino acid sequence recognized by a protease whose expression is specific or upregulated for a tumor cell or tumor cell environment thereof. In some embodiments, the tumor-associated protease is a matrix metalloproteinase (MMP), selected from the group consisting of MMP1, MMP2, MMP3, MMP7, MMP8, MMP9, MMP10, MMP11, MMP12, MMP13, MMP14, MMP15, MMP16, MMP17, MMP19, MMP20, MMP21, MMP23A, MMP23B, MMP24, MMP25, MMP27, and MMP28. In one embodiment, the tumor-associated protease is MMP2. In another embodiment, the tumor-associated protease is MMP3. In yet another embodiment, the tumor-associated protease is MMP9. In yet another embodiment, the tumor-associated protease is MMP10. In some embodiments, the protease is Cathepsin B. In other embodiments, the protease is matriptase.

An advantage of such engineered cleavable Fc domain provides a universal design architecture; such that the fusion of an engineered cleavable Fc domain to a therapeutic agent and the subsequent cleavage at the cleavage site allows release of the agent at a particular environment, e.g., a tumor microenvironment.

The tumor cell environment is complex and can comprise multiple different proteases. As such, the precise site at which the Fc domain will be cleaved in the tumor cell environment may vary between tumor types, between patients with the same tumor type and even between cleavage products formed in the same tumor dependent on the specific tumor cell environment. Moreover, even after cleavage, further modification of the initial cleavage product, e.g., by removal of one or two terminal amino acids, may occur by the further action of proteases in the tumor cell environment. A distribution of cleavage products is expected to form in the tumor cell environment of a patient following administration of a single structure of a targeted cytokine as described herein.

It will be understood that a cleavage site as referred to herein refers to a site between two specific amino acid residues within the cleavable peptide that are a target for a protease known to be associated with a tumor cell environment. In this sense, there may be more than one cleavage site present in a cleavable peptide as described herein where different proteases cleave the cleavable peptide at different cleavage sites. It is also possible that more than one protease may act on the same cleavage site within a cleavable peptide. Discussion of protease cleavage sites can be found in the art.

Thus, the cleavable Fc domain disclosed herein may be cleaved by one or more proteases. In some embodiments, the cleavage sites of the cleavable Fc domain are cleavage sites of one or more tumor associated proteases. In some embodiments, the cleavage sites of the cleavable Fc domain are cleavage sites of one or more tissue selective proteases. In some embodiments, the cleavage sites of the cleavable Fc domain are cleavage sites of one or more inflammation associated proteases. In other embodiments, the cleavable Fc domain comprises one or more cleavage sites of other disease-associated proteases.

In some embodiments, the cleavable Fc domain is a substrate for a protease that is co-localized in a region or a tissue or an organ expressing the cytokine receptor.

The cleavable peptide motif in the Fc domain comprises at least 3 amino acid residues. In some embodiments, the cleavable peptide motif is a 3-mer (i.e. peptide 3 amino acids in length), 4-mer (i.e. peptide 4 amino acids in length), 5-mer (i.e. peptide 5 amino acids in length), 6-mer (i.e. peptide 6 amino acids in length), 7-mer (i.e. peptide 7 amino acids in length), 8-mer (i.e. peptide 8 amino acids in length), 9-mer (i.e. peptide 9 amino acids in length), 10-mer (i.e. peptide 10 amino acids in length), 11-mer (i.e. peptide 11 amino acids in length), 12-mer (i.e. peptide 12 amino acids in length), 13-mer (i.e. peptide 13 amino acids in length), 14-mer (i.e. peptide 14 amino acids in length), 15-mer (i.e. peptide 15 amino acids in length), 16-mer (i.e. peptide 16 amino acids in length), 17-mer (i.e. peptide 17 amino acids in length), or 18-mer (i.e. peptide 18 amino acids in length).

In some embodiments, the cleavable peptide motif in the Fe domain is from 3 to 18 amino acids in length. In some embodiments, the cleavable peptide motif in the Fc domain is from 5 to 10 amino acids in length, or from 5 to 8 amino acids in length, or from 6 to 10 amino acids in length, or from 7 to 10 amino acids in length, or from 6 to 12 amino acids in length. In some embodiments, the cleavable peptide motif in the Fc domain is 3 amino acids in length. In some embodiments, the cleavable peptide motif in the Fc domain is 4 amino acids in length. In some embodiments, the cleavable peptide motif in the Fc domain is 5 amino acids in length. In some embodiments, the cleavable peptide motif in the Fc domain is 6 amino acids in length. In some embodiments, the cleavable peptide motif in the Fc domain is 7 amino acids in length. In some embodiments, the cleavable peptide motif in the Fc domain is 8 amino acids in length. In some embodiments, the cleavable peptide motif in the Fc domain is 9 amino acids in length. In some embodiments, the cleavable peptide motif in the Fc domain is 10 amino acids in length.

In some embodiments, the protease cleavage site within the engineered cleavable Fc domain is in the hinge region, in the CH2 domain, in the CH3 domain and/or in the CH2-CH3 domain linker region. In some embodiments, the protease cleavage site within the engineered cleavable Fc domain is in the F strand region. In some embodiments, the protease cleavage site within the engineered cleavable Fc domain is in the FG-loop region. In some embodiments, the protease cleavage site within the engineered cleavable Fc domain is in the G-strand region.

In some embodiments, the cleavable sites within the engineered cleavable Fc domain may be created based on the short amino acid motifs close to a cleavage site of a protease.

Based on the short peptide sequence of the Fc domain, one or more mutations (e.g., amino acid substitutions) may be introduced to the peptide sequences to create a protease cleavage site. As non-limiting examples, one or more protease cleavage sites may be created based on the short peptide sequences of SEQ ID NOs: 98, 101, 103 and 106.

In some embodiments, a protease cleavage site that can be recognized by MMP2 is created. In some embodiments, a protease cleavage site that can be recognized by MMP3 is created. In some embodiments, a protease cleavage site that can be recognized by MMP9 is created. In some embodiments, a protease cleavage site that can be recognized by MMP10 is created. In some embodiments, a protease cleavage site that can be recognized by Cathepsin B is created. In some embodiments, a protease cleavage site that can be recognized by matriptase is created.

As non-limiting examples, the cleavable peptide motif within the engineered cleavable Fe domain comprises an amino acid sequence selected from the group consisting of SEQ ID NOs: 95, 97, 99, 100, 102, 104, 105, 107, 108, 109, 110, 119, 122, 123, 135-139, 143-208, and 209. In some embodiments, the cleavable peptide motif within the engineered cleavable Fc domain comprises VPLSLYSG (SEQ ID NO: 95). In some embodiments, the cleavable peptide motif within the engineered cleavable Fc domain comprises VPLSLYSGP (SEQ ID NO: 209). In some embodiments, the cleavable peptide motif within the engineered cleavable Fc domain comprises QQGNVFSC (SEQ ID NO.: 97). In some embodiments, the cleavable peptide motif within the engineered cleavable Fc domain comprises IHVTLKSL (SEQ ID NO.: 99). In some embodiments, the cleavable peptide motif within the engineered cleavable Fc domain comprises NSYTIKGL (SEQ ID NO: 100). In some embodiments, the cleavable peptide motif within the engineered cleavable Fc domain comprises SNESLSLS (SEQ ID NO: 102). In some embodiments, the cleavable peptide motif within the engineered cleavable Fc domain comprises QVSSSLSP (SEQ ID NO: 104). In some embodiments, the cleavable peptide motif within the engineered cleavable Fc domain comprises PTSTSLSP (SEQ ID NO.: 105). In some embodiments, the cleavable peptide motif within the engineered cleavable Fc domain comprises ESLSLSEE (SEQ ID NO: 107). In some embodiments, the cleavable peptide motif within the engineered cleavable Fc domain comprises ASLSLAPV (SEQ ID NO.: 108). In some embodiments, the cleavable peptide motif within the engineered cleavable Fc domain comprises SQESLSLS (SEQ ID NO: 109). In some embodiments, the cleavable peptide motif within the engineered cleavable Fc domain comprises PLGL (SEQ ID NO: 110). In some embodiments, the cleavable peptide motif within the engineered cleavable Fc domain comprises MPYDLYHP (SEQ ID NO: 135). In some embodiments, the cleavable peptide motif within the engineered cleavable Fc domain comprises ISSGLLSGRS (SEQ ID NO: 136). In some embodiments, the cleavable peptide motif within the engineered cleavable Fc domain comprises RAAAVKSP (SEQ ID NO: 137). In some embodiments, the cleavable peptide motif within the engineered cleavable Fc domain comprises RPLALWRS (SEQ ID NO: 138). In some embodiments, the cleavable peptide motif within the engineered cleavable Fc domain comprises TQKPLGLS (SEQ ID NO: 139). In some embodiments, the cleavable peptide motif within the engineered cleavable Fc domain comprises APAGLIVPYN (SEQ ID NO: 119). In some embodiments, the cleavable peptide motif within the engineered cleavable Fc domain comprises PVSLRSGS (SEQ ID NO: 122). In some embodiments, the cleavable peptide motif within the engineered cleavable Fc domain comprises PANLVAPDP (SEQ ID NO: 123). In some embodiments, the cleavable peptide motif within the engineered cleavable Fe domain comprises RSKYLATA (SEQ ID NO: 143). In some embodiments, the cleavable peptide motif within the engineered cleavable Fe domain comprises GRPRHQGV (SEQ ID NO: 144). In some embodiments, the cleavable peptide motif within the engineered cleavable Fe domain comprises GLFG (SEQ ID NO: 145). In some embodiments, the cleavable peptide motif within the engineered cleavable Fe domain comprises GFLG (SEQ ID NO: 146). In some embodiments, the cleavable peptide motif within the engineered cleavable Fe domain comprises AGRRAAK (SEQ ID NO: 147). In some embodiments, the cleavable peptide motif within the engineered cleavable Fe domain comprises FRLWA (SEQ ID NO: 148). In some embodiments, the cleavable peptide motif within the engineered cleavable Fe domain comprises FRLWS (SEQ ID NO: 149). In some embodiments, the cleavable peptide motif within the engineered cleavable Fe domain comprises NFFGVGGE (SEQ ID NO: 150). In some embodiments, the cleavable peptide motif within the engineered cleavable Fe domain comprises PMKRLTLA (SEQ ID NO: 151). In some embodiments, the cleavable peptide motif within the engineered cleavable Fe domain comprises FPLATYAP (SEQ ID NO: 152). In some embodiments, the cleavable peptide motif within the engineered cleavable Fe domain comprises FLVGGASL (SEQ ID NO: 153). In some embodiments, the cleavable peptide motif within the engineered cleavable Fe domain comprises KPMQFLGD (SEQ ID NO: 154). In some embodiments, the cleavable peptide motif within the engineered cleavable Fe domain comprises GIVRAKGV (SEQ ID NO: 155). In some embodiments, the cleavable peptide motif within the engineered cleavable Fe domain comprises ALFKSSFP (SEQ ID NO: 156). In some embodiments, the cleavable peptide motif within the engineered cleavable Fe domain comprises SGRRSPGG (SEQ ID NO: 157). In some embodiments, the cleavable peptide motif within the engineered cleavable Fe domain comprises SLGRRPGG (SEQ ID NO: 158). In some embodiments, the cleavable peptide motif within the engineered cleavable Fe domain comprises SLSGRRGG (SEQ ID NO: 159). In some embodiments, the cleavable peptide motif within the engineered cleavable Fe domain comprises SLSLGRRG (SEQ ID NO: 160). In some embodiments, the cleavable peptide motif within the engineered cleavable Fe domain comprises SLSLSGRR (SEQ ID NO: 161). In some embodiments, the cleavable peptide motif within the engineered cleavable Fe domain comprises GGPRRL (SEQ ID NO: 162). In some embodiments, the cleavable peptide motif within the engineered cleavable Fe domain comprises GGPLRL (SEQ ID NO: 163). In some embodiments, the cleavable peptide motif within the engineered cleavable Fe domain comprises GGPKLL (SEQ ID NO: 164). In some embodiments, the cleavable peptide motif within the engineered cleavable Fe domain comprises GGPRNL (SEQ ID NO: 165). In some embodiments, the cleavable peptide motif within the engineered cleavable Fe domain comprises GGPRML (SEQ ID NO: 166). In some embodiments, the cleavable peptide motif within the engineered cleavable Fe domain comprises EHLRSPGG (SEQ ID NO: 167). In some embodiments, the cleavable peptide motif within the engineered cleavable Fe domain comprises FRSGVPGG (SEQ ID NO: 168). In some embodiments, the cleavable peptide motif within the engineered cleavable Fe domain comprises SLLLRTGN (SEQ ID NO: 169). In some embodiments, the cleavable peptide motif within the engineered cleavable Fe domain comprises AGLRSPGG (SEQ ID NO: 170). In some embodiments, the cleavable peptide motif within the engineered cleavable Fe domain comprises SLFRSAGP (SEQ ID NO: 171). In some embodiments, the cleavable peptide motif within the engineered cleavable Fe domain comprises SLFRAPGP (SEQ ID NO: 172). In some embodiments, the cleavable peptide motif within the engineered cleavable Fe domain comprises WLFRSPLG (SEQ ID NO: 173). In some embodiments, the cleavable peptide motif within the engineered cleavable Fe domain comprises SRLRSPQG (SEQ ID NO: 174). In some embodiments, the cleavable peptide motif within the engineered cleavable Fe domain comprises SLVLSGRR (SEQ ID NO: 175). In some embodiments, the cleavable peptide motif within the engineered cleavable Fe domain comprises KQLRHMRG (SEQ ID NO: 176). In some embodiments, the cleavable peptide motif within the engineered cleavable Fe domain comprises LSGRSDNH (SEQ ID NO: 177). In some embodiments, the cleavable peptide motif within the engineered cleavable Fe domain comprises LSGK (SEQ ID NO: 178). In some embodiments, the cleavable peptide motif within the engineered cleavable Fe domain comprises LSGR (SEQ ID NO: 179). In some embodiments, the cleavable peptide motif within the engineered cleavable Fe domain comprises RQARVVGG (SEQ ID NO: 180). In some embodiments, the cleavable peptide motif within the engineered cleavable Fe domain comprises RQRRVVGG (SEQ ID NO: 181). In some embodiments, the cleavable peptide motif within the engineered cleavable Fe domain comprises RQYRVVGG (SEQ ID NO: 182). In some embodiments, the cleavable peptide motif within the engineered cleavable Fe domain comprises SKGRSLIG (SEQ ID NO: 183). In some embodiments, the cleavable peptide motif within the engineered cleavable Fe domain comprises PRFKIIGG (SEQ ID NO: 184). In some embodiments, the cleavable peptide motif within the engineered cleavable Fe domain comprises KQLRVVNG (SEQ ID NO: 185). In some embodiments, the cleavable peptide motif within the engineered cleavable Fe domain comprises IQPRITGG (SEQ ID NO: 186). In some embodiments, the cleavable peptide motif within the engineered cleavable Fe domain comprises KQSRKFVP (SEQ ID NO: 187). In some embodiments, the cleavable peptide motif within the engineered cleavable Fe domain comprises GRQSRAGG (SEQ ID NO: 188). In some embodiments, the cleavable peptide motif within the engineered cleavable Fe domain comprises SGRSSPGG (SEQ ID NO: 189). In some embodiments, the cleavable peptide motif within the engineered cleavable Fe domain comprises SSGRSPGG (SEQ ID NO: 190). In some embodiments, the cleavable peptide motif within the engineered cleavable Fe domain comprises SLSGRSGG (SEQ ID NO: 191). In some embodiments, the cleavable peptide motif within the engineered cleavable Fe domain comprises SLSSGRSG (SEQ ID NO: 192). In some embodiments, the cleavable peptide motif within the engineered cleavable Fe domain comprises KLSLSGRS (SEQ ID NO: 193). In some embodiments, the cleavable peptide motif within the engineered cleavable Fe domain comprises PLRLSRA (SEQ ID NO: 194). In some embodiments, the cleavable peptide motif within the engineered cleavable Fe domain comprises PLKLSRA (SEQ ID NO: 195). In some embodiments, the cleavable peptide motif within the engineered cleavable Fe domain comprises PLGLSGRS (SEQ ID NO: 196). In some embodiments, the cleavable peptide motif within the engineered cleavable Fe domain comprises PLGLRSRA (SEQ ID NO: 197). In some embodiments, the cleavable peptide motif within the engineered cleavable Fe domain comprises PLGLKSRA (SEQ ID NO: 198). In some embodiments, the cleavable peptide motif within the engineered cleavable Fe domain comprises RGSRAG (SEQ ID NO: 199). In some embodiments, the cleavable peptide motif within the engineered cleavable Fe domain comprises RLSRGK (SEQ ID NO: 200). In some embodiments, the cleavable peptide motif within the engineered cleavable Fe domain comprises RGSRGG (SEQ ID NO: 201). In some embodiments, the cleavable peptide motif within the engineered cleavable Fe domain comprises KGSRAG (SEQ ID NO: 202). In some embodiments, the cleavable peptide motif within the engineered cleavable Fe domain comprises KLSRGK (SEQ ID NO: 203). In some embodiments, the cleavable peptide motif within the engineered cleavable Fe domain comprises GRSRAG (SEQ ID NO: 204). In some embodiments, the cleavable peptide motif within the engineered cleavable Fe domain comprises LRSRGK (SEQ ID NO: 205). In some embodiments, the cleavable peptide motif within the engineered cleavable Fe domain comprises GRSRGG (SEQ ID NO: 206). In some embodiments, the cleavable peptide motif within the engineered cleavable Fe domain comprises GKSRAG (SEQ ID NO: 207). In some embodiments, the cleavable peptide motif within the engineered cleavable Fe domain comprises LKSRGK (SEQ ID NO: 208). In some embodiments, the cleavable peptide motif within the engineered cleavable Fe domain comprises VPLSLYSGP (SEQ ID NO: 209).

Incorporation of Cleavable Substrate in G-Strand of the Fc Domain

In some embodiments, an engineered cleavable Fe domain comprises one or more substitutions in the CH3 domain. In some embodiments, an engineered cleavable Fe domain comprises one or more substitutions in the C-terminal region within the Fe domain. In some embodiments, an engineered cleavable Fe domain comprises one or more substitutions in the G-strand within the Fe domain. In some embodiments, an engineered cleavable Fe domain comprises one or more substitutions in positions between 436-447 by EU numbering. In some embodiments, an engineered cleavable Fe domain comprises one or more substitutions in positions between 438 and 447 by EU numbering.

In some embodiments, the cleavable peptide motif is located between positions 438 and 445 by EU numbering. In some embodiments, the cleavable peptide motif is located between positions 439 and 446 by EU numbering. In some embodiments, the cleavable peptide motif is located between positions 440 and 447 by EU numbering. In some embodiments, the cleavable peptide motif is located between positions 438 and 447 by EU numbering. In some embodiments, the cleavable peptide motif is located between positions 437 and 444 by EU numbering. In some embodiments, the cleavable peptide motif is located between positions 440 and 443 by EU numbering. In some embodiments, the cleavable peptide motif is located between positions 441 and 444 by EU numbering. In some embodiments, the cleavable peptide motif is located between positions 442 and 445 by EU numbering. In some embodiments, the cleavable peptide motif is located between positions 444 and 447 by EU numbering. In some embodiments, the cleavable peptide motif is located between positions 441 and 447 by EU numbering. In some embodiments, the cleavable peptide motif is located between positions 443 and 447 by EU numbering. In some embodiments, the cleavable peptide motif is located between positions 436 and 443 by EU numbering. In some embodiments, the cleavable peptide motif is located between positions 442 and 447 by EU numbering.

In some embodiments, an engineered cleavable Fe domain comprises Q438V, K439P, S440L, L441S, S442L, L443Y, P445G substitutions by EU numbering. In some embodiments, an engineered cleavable Fe domain comprises K439V, S440P, S444Y, P445S substitutions by EU numbering. In some embodiments, an engineered cleavable Fe domain comprises S440V, L441P, S442L, L443S, S444L, P445Y, G446S substitutions by EU numbering. In some embodiments, an engineered cleavable Fe domain comprises Q438M, K439P, S440Y, L441D, S442L, L443Y, S444H substitutions by EU numbering. In some embodiments, an engineered cleavable Fe domain comprises K439M, S440P, L441Y, S442D, S444Y, P445H, G446P substitutions by EU numbering. In some embodiments, an engineered cleavable Fe domain comprises S440M, L441P, S442Y, L443D, S444L, P445Y, G446H, G447P substitutions by EU numbering. In some embodiments, an engineered cleavable Fe domain comprises Q438R, K439A, S440A, L441A, S442V, L443K substitutions by EU numbering. In some embodiments, an engineered cleavable Fe domain comprises K439R, S440A, L441A, S442A, L443V, S444K, P445S, G446P substitutions by EU numbering. In some embodiments, an engineered cleavable Fe domain comprises S440R, L441A, S442A, L443A, S444V, P445K, G446S, G447P substitutions by EU numbering. In some embodiments, an engineered cleavable Fe domain comprises K439R, S440P, S442A, S444W, P445R, G446S substitutions by EU numbering. In some embodiments, an engineered cleavable Fe domain comprises Q438A, K439P, S440A, L441G, S442L, L443I, S444V, G446Y, G447N substitutions by EU numbering. In some embodiments, an engineered cleavable Fe domain comprises K439P, S440V, L441S, S442L, L443R, P445G, G446S substitutions by EU numbering. In some embodiments, an engineered cleavable Fe domain comprises Q438P, K439A, S440N, S442V, L443A, S444P, P445D, G446P substitutions by EU numbering. In some embodiments, an engineered cleavable Fe domain comprises S440P, S442G substitutions by EU numbering. In some embodiments, an engineered cleavable Fe domain comprises K439G, S440P, S442G substitutions by EU numbering. In some embodiments, an engineered cleavable Fe domain comprises L441P, S442L, L443G, S444L substitutions by EU numbering. In some embodiments, an engineered cleavable Fe domain comprises S440G, L441P, S442L, L443G, S444L substitutions by EU numbering. In some embodiments, an engineered cleavable Fe domain comprises S442P, S444G, P445L substitutions by EU numbering. In some embodiments, an engineered cleavable Fe domain comprises S444P, P445L, G447L substitutions by EU numbering. In some embodiments, an engineered cleavable Fe domain comprises L443G, S444P, P445L, G447L substitutions by EU numbering. In some embodiments, an engineered cleavable Fe domain comprises L443G, S444P, P445L, G447L substitutions by EU numbering. In some embodiments, an engineered cleavable Fe domain comprises S440M, L441P, S442Y, L443D, S444L, P445Y, G446H, G447P substitutions by EU numbering. In some embodiments, an engineered cleavable Fe domain comprises S442G, L443G, S444P, P445L, G447L substitutions by EU numbering.

In some embodiments, an engineered cleavable Fe domain comprises S440R, L441S, S442K, L443Y, S444L, P445A, G446T, G447A substitutions by EU numbering. In some embodiments, an engineered cleavable Fe domain comprises S440G, L441R, S442P, L443R, S444H, P445Q, G447V substitutions by EU numbering. In some embodiments, an engineered cleavable Fe domain comprises S444G, P445L, G446F substitutions by EU numbering. In some embodiments, an engineered cleavable Fe domain comprises S444G, P445F, G446L substitutions by EU numbering. In some embodiments, an engineered cleavable Fe domain comprises L441A, S442G, L443R, S444R, P445A, G446A, G447K substitutions by EU numbering. In some embodiments, an engineered cleavable Fe domain comprises L443F, S444R, P445L, G446W, G447A substitutions by EU numbering. In some embodiments, an engineered cleavable Fe domain comprises L443F, S444R, P445L, G446W, G447S substitutions by EU numbering. In some embodiments, an engineered cleavable Fe domain comprises S440N, L441F, S442F, L443G, S444V, P445G, G447E substitutions by EU numbering. In some embodiments, an engineered cleavable Fe domain comprises S440P, L441M, S442K, L443R, S444L, P445T, G446L, G447A substitutions by EU numbering. In some embodiments, an engineered cleavable Fe domain comprises K439P, S440M, L441K, S442R, S444T, P445L, G446A substitutions by EU numbering. In some embodiments, an engineered cleavable Fe domain comprises Q438P, K439M, S440K, L441R, S442L, L443T, S444L, P445A substitutions by EU numbering. In some embodiments, an engineered cleavable Fe domain comprises S440F, L441P, S442L, L443A, S444T, P445Y, G446A, G447P substitutions by EU numbering. In some embodiments, an engineered cleavable Fe domain comprises S440F, S442V, L443G, S444G, P445A, G446S, G447L substitutions by EU numbering. In some embodiments, an engineered cleavable Fe domain comprises S440K, L441P, S442M, L443Q, S444F, P445L, G447D substitutions by EU numbering. In some embodiments, an engineered cleavable Fe domain comprises S440G, L441I, S442V, L443R, S444A, P445K, G447V substitutions by EU numbering. In some embodiments, an engineered cleavable Fe domain comprises S440A, S442F, L443K, P445S, G446F, G447P substitutions by EU numbering. In some embodiments, an engineered cleavable Fe domain comprises L441G, S442R, L443R substitutions by EU numbering. In some embodiments, an engineered cleavable Fe domain comprises S442G, L443R, S444R substitutions by EU numbering. In some embodiments, an engineered cleavable Fe domain comprises L443G, S444R, P445R substitutions by EU numbering. In some embodiments, an engineered cleavable Fe domain comprises S444G, P445R, G446R substitutions by EU numbering. In some embodiments, an engineered cleavable Fe domain comprises P445G, G446R, G447R substitutions by EU numbering. In some embodiments, an engineered cleavable Fe domain comprises S442G, L443G, S444P, P445R, G446R, G447L substitutions by EU numbering. In some embodiments, an engineered cleavable Fe domain comprises S442G, L443G, S444P, P445L, G446R, G447L substitutions by EU numbering. In some embodiments, an engineered cleavable Fe domain comprises S442G, L443G, S444P, P445K, G446L, G447L substitutions by EU numbering. In some embodiments, an engineered cleavable Fe domain comprises S442G, L443G, S444P, P445R, G446N, G447L substitutions by EU numbering. In some embodiments, an engineered cleavable Fe domain comprises S442G, L443G, S444P, P445R, G446M, G447L substitutions by EU numbering. In some embodiments, an engineered cleavable Fe domain comprises S440E, L441H, S442L, L443R substitutions by EU numbering. In some embodiments, an engineered cleavable Fe domain comprises S440F, L441R, L443G, S444V substitutions by EU numbering. In some embodiments, an engineered cleavable Fe domain comprises S442L, S444R, P445T, G447N substitutions by EU numbering. In some embodiments, an engineered cleavable Fe domain comprises S440A, L441G, S442L, L443R substitutions by EU numbering. In some embodiments, an engineered cleavable Fe domain comprises S442F, L443R, P445A, G447P substitutions by EU numbering. In some embodiments, an engineered cleavable Fe domain comprises S442F, L443R, S444A, G447P substitutions by EU numbering. In some embodiments, an engineered cleavable Fe domain comprises S440W, S442F, L443R, G446L substitutions by EU numbering. In some embodiments, an engineered cleavable Fe domain comprises L441R, S442L, L443R, G446Q substitutions by EU numbering. In some embodiments, an engineered cleavable Fe domain comprises S442V, P445G, G446R, G447R substitutions by EU numbering. In some embodiments, an engineered cleavable Fe domain comprises S440K, L441Q, S442L, L443R, S444H, P445M, G446R substitutions by EU numbering. In some embodiments, an engineered cleavable Fe domain comprises S440L, L441S, S442G, L443R, P445D, G446N, G447H substitutions by EU numbering. In some embodiments, an engineered cleavable Fe domain comprises S444L, P445S, G447K substitutions by EU numbering. In some embodiments, an engineered cleavable Fe domain comprises S444L, P445S, G447R substitutions by EU numbering. In some embodiments, an engineered cleavable Fe domain comprises S440R, L441Q, S442A, L443R, S444V, P445V substitutions by EU numbering. In some embodiments, an engineered cleavable Fe domain comprises S440R, L441Q, S442R, L443R, S444V, P445V substitutions by EU numbering. In some embodiments, an engineered cleavable Fe domain comprises K439R, S440Q, L441R, S442R, L443V, S444V, P445G substitutions by EU numbering. In some embodiments, an engineered cleavable Fe domain comprises Q438R, K439Q, S440R, L441R, S442V, L443V, S444G, P445G substitutions by EU numbering. In some embodiments, an engineered cleavable Fe domain comprises S440R, L441Q, S442Y, L443R, S444V, P445V substitutions by EU numbering. In some embodiments, an engineered cleavable Fe domain comprises L441K, S442G, L443R, P445L, G446I substitutions by EU numbering. In some embodiments, an engineered cleavable Fe domain comprises S440P, L441R, S442F, L443K, S444I, P445I substitutions by EU numbering. In some embodiments, an engineered cleavable Fe domain comprises S440K, L441Q, S442L, L443R, S444V, P445V, G446N substitutions by EU numbering. In some embodiments, an engineered cleavable Fe domain comprises S440I, L441Q, S442P, L443R, S444I, P445T substitutions by EU numbering. In some embodiments, an engineered cleavable Fe domain comprises S440K, L441Q, L443R, S444K, P445F, G446V, G447P substitutions by EU numbering. In some embodiments, an engineered cleavable Fe domain comprises S440G, L441R, S442Q, L443S, S444R, P445A substitutions by EU numbering. In some embodiments, an engineered cleavable Fe domain comprises L441G, S442R, L443S substitutions by EU numbering. In some embodiments, an engineered cleavable Fe domain comprises L441S, S442G, L443R substitutions by EU numbering. In some embodiments, an engineered cleavable Fe domain comprises L443G, S444R, P445S substitutions by EU numbering. In some embodiments, an engineered cleavable Fe domain comprises L443S, S444G, P445R, G446S substitutions by EU numbering. In some embodiments, an engineered cleavable Fe domain comprises P445G, G446R, G447S substitutions by EU numbering. In some embodiments, an engineered cleavable Fe domain comprises L441P, S442L, L443R, S444L, P445S, G446R, G447A substitutions by EU numbering. In some embodiments, an engineered cleavable Fe domain comprises L441P, S442L, L443K, S444L, P445S, G446R, G447A substitutions by EU numbering. In some embodiments, an engineered cleavable Fe domain comprises S440P, S442G, P445G, G446R, G447S substitutions by EU numbering. In some embodiments, an engineered cleavable Fe domain comprises S440P, S442G, S444R, P445S, G446R, G447A substitutions by EU numbering. In some embodiments, an engineered cleavable Fe domain comprises S440P, S442G, S444K, P445S, G446R, G447A substitutions by EU numbering. In some embodiments, an engineered cleavable Fc domain comprises S442R, L443G, P445R, G446A substitutions by EU numbering. In some embodiments, an engineered cleavable Fc domain comprises S442R, P445R, G447K substitutions by EU numbering. In some embodiments, an engineered cleavable Fc domain comprises S442R, L443G, P445R substitutions by EU numbering. In some embodiments, an engineered cleavable Fc domain comprises S442K, L443G, P445R, G446A substitutions by EU numbering. In some embodiments, an engineered cleavable Fc domain comprises S442K, P445R, G447K substitutions by EU numbering. In some embodiments, an engineered cleavable Fc domain comprises S442G, L443R, P445R, G446A substitutions by EU numbering. In some embodiments, an engineered cleavable Fc domain comprises S442L, L443R, P445R, G447K substitutions by EU numbering. In some embodiments, an engineered cleavable Fc domain comprises S442G, L443R, P445R substitutions by EU numbering. In some embodiments, an engineered cleavable Fc domain comprises S442G, L443K, P445R, G446A substitutions by EU numbering. In some embodiments, an engineered cleavable Fc domain comprises S442L, L443K, P445R, G447K substitutions by EU numbering.

In some embodiments, an engineered cleavable Fc domain comprises one or more substitutions shown in Table 5, Table 8a, or Table 11.

TABLE 11
Exemplary Engineered Fc domain comprising cleavable substrates in G-Strand

| Cleavage | | Cleavage Substrate |
|---|---|---|
| Substrate | Fc Mutations | location |
| RSKYLATA | S440R, L441S, S442K, L443Y, S444L, P445A, G446T, | 440-RSKYLATA-447 |
| | G447A | |
| GRPRHQGV | S440G, L441R, S442P, L443R, S444H, P445Q, G447V | 440-GRPRHQGV-447 |
| GLFG | S444G, P445L, G446F | 444-GLFG-447 |
| GFLG | S444G, P445F, G446L | 444-GFLG-447 |
| AGRRAAK | L441A, S442G, L443R, S444R, P445A, G446A, | 441-AGRRAAK-447 |
| | G447K | |
| FRLWA | L443F, S444R, P445L, G446W, G447A | 443-FRLWA-447 |
| FRLWS | L443F, S444R, P445L, G446W, G447S | 443-FRLWS-447 |
| NFFGVGGE | S440N, L441F, S442F, L443G, S444V, P445G, G447E | 440-NFFGVGGE-447 |
| PMKRLTLA | S440P, L441M, S442K, L443R, S444L, P445T, G446L, | 440-PMKRLTLA-447 |
| | G447A | |
| PMKRLTLA | K439P, S440M, L441K, S442R, S444T, P445L, G446A | 438-PMKRLTLA-445 |
| PMKRLTLA | Q438P, K439M, S440K, L441R, S442L, L443T, | 436-PMKRLTLA-443 |
| | S444L, P445A | |
| FPLATYAP | S440F, L441P, S442L, L443A, S444T, P445Y, G446A, | 440-FPLATYAP-447 |
| | G447P | |
| FLVGGASL | S440F, S442V, L443G, S444G, P445A, G446S, G447L | 440-FLVGGASL-447 |
| KPMQFLGD | S440K, L441P, S442M, L443Q, S444F, P445L, G447D | 440-KPMQFLGD-447 |
| GIVRAKGV | S440G, L441I, S442V, L443R, S444A, P445K, G447V | 440-GIVRAKGV-447 |
| ALFKSSFP | S440A, S442F, L443K, P445S, G446F, G447P | 440-ALFKSSFP-447 |
| SGRRSPGG | L441G, S442R, L443R | 440-SGRRSPGG-447 |
| SLGRRPGG | S442G, L443R, S444R | 440-SLGRRPGG-447 |
| SLSGRRGG | L443G, S444R, P445R | 440-SLSGRRGG-447 |
| SLSLGRRG | S444G, P445R, G446R | 440-SLSLGRRG-447 |
| SLSLSGRR | P445G, G446R, G447R | 440-SLSLSGRR-447 |
| GGPRRL | S442G, L443G, S444P, P445R, G446R, G447L | 442-GGPRRL-447 |
| GGPLRL | S442G, L443G, S444P, P445L, G446R, G447L | 442-GGPLRL-447 |
| GGPKLL | S442G, L443G, S444P, P445K, G446L, G447L | 442-GGPKLL-447 |
| GGPRNL | S442G, L443G, S444P, P445R, G446N, G447L | 442-GGPRNL-447 |
| GGPRML | S442G, L443G, S444P, P445R, G446M, G447L | 442-GGPRML-447 |
| EHLRSPGG | S440E, L441H, S442L, L443R | 440-EHLRSPGG-447 |
| FRSGVPGG | S440F, L441R, L443G, S444V | 440-FRSGVPGG-447 |
| SLLLRTGN | S442L, S444R, P445T, G447N | 440-SLLLRTGN-447 |
| AGLRSPGG | S440A, L441G, S442L, L443R | 440-AGLRSPGG-447 |
| SLFRSAGP | S442F, L443R, P445A, G447P | 440-SLFRSAGP-447 |
| SLFRAPGP | S442F, L443R, S444A, G447P | 440-SLFRAPGP-447 |
| WLFRSPLG | S440W, S442F, L443R, G446L | 440-WLFRSPLG-447 |
| SRLRSPQG | L441R, S442L, L443R, G446Q | 440-SRLRSPQG-447 |
| SLVLSGRR | S442V, P445G, G446R, G447R | 440-SLVLSGRR-447 |
| KQLRHMRG | S440K, L441Q, S442L, L443R, S444H, P445M, G446R | 440-KQLRHMRG-447 |
| LSGRSDNH | S440L, L441S, S442G, L443R, P445D, G446N, G447H | 440-LSGRSDNH-447 |
| LSGK | S444L, P445S, G447K | 444-LSGK-447 |
| LSGR | S444L, P445S, G447R | 444-LSGR-447 |
| RQARVVGG | S440R, L441Q, S442A, L443R, S444V, P445V | 440-RQARVVGG-447 |
| RQRRVVGG | S440R, L441Q, S442R, L443R, S444V, P445V | 440-RQRRVVGG-447 |
| RQRRVVGG | K439R, S440Q, L441R, S442R, L443V, S444V, P445G | 438-RQRRVVGG-445 |
| RQRRVVGG | Q438R, K439Q, S440R, L441R, S442V, L443V, | 436-RQRRVVGG-443 |
| | S444G, P445G | |
| RQYRVVGG | S440R, L441Q, S442Y, L443R, S444V, P445V | 440-RQYRVVGG-447 |
| SKGRSLIG | L441K, S442G, L443R, P445L, G446I | 440-SKGRSLIG-447 |
| PRFKIIGG | S440P, L441R, S442F, L443K, S444I, P445I | 440-PRFKIIGG-447 |
| KQLRVVNG | S440K, L441Q, S442L, L443R, S444V, P445V, G446N | 440-KQLRVVNG-447 |
| IQPRITGG | S440I, L441Q, S442P, L443R, S444I, P445T | 440-IQPRITGG-447 |
| KQSRKFVP | S440K, L441Q, L443R, S444K, P445F, G446V, G447P | 440-KQSRKFVP-447 |
| GRQSRAGG | S440G, L441R, S442Q, L443S, S444R, P445A | 440-GRQSRAGG-447 |
| SGRSSPGG | L441G, S442R, L443S | 440-SGRSSPGG-447 |
| SSGRSPGG | L441S, S442G, L443R | 440-SSGRSPGG-447 |
| SLSGRSGG | L443G, S444R, P445S | 440-SLSGRSGG-447 |
| SLSSGRSG | L443S, S444G, P445R, G446S | 440-SLSSGRSG-447 |
| KLSLSGRS | P445G, G446R, G447S | 440-SLSLSGRS-447 |
| PLRLSRA | L441P, S442L, L443R, S444L, P445S, G446R, G447A | 441-PLRLSRA-447 |
| PLKLSRA | L441P, S442L, L443K, S444L, P445S, G446R, G447A | 441-PLKLSRA-447 |
| PLGLSGRS | S440P, S442G, P445G, G446R, G447S | 440-PLGLSGRS-447 |
| PLGLRSRA | S440P, S442G, S444R, P445S, G446R, G447A | 440-PLGLRSRA-447 |
| PLGLKSRA | S440P, S442G, S444K, P445S, G446R, G447A | 440-PLGLKSRA-447 |
| RGSRAG | S442R, L443G, P445R, G446A | 442-RGSRAG-447 |
| RLSRGK | S442R, P445R, G447K | 442-RLSRGK-447 |
| RGSRGG | S442R, L443G, P445R | 442-RGSRGG-447 |
| KGSRAG | S442K, L443G, P445R, G446A | 442-KGSRAG-447 |
| KLSRGK | S442K, P445R, G447K | 442-KLSRGK-447 |
| GRSRAG | S442G, L443R, P445R, G446A | 442-GRSRAG-447 |
| LRSRGK | S442L, L443R, P445R, G447K | 442-LRSRGK-447 |
| GRSRGG | S442G, L443R, P445R | 442-GRSRGG-447 |
| GKSRAG | S442G, L443K, P445R, G446A | 442-GKSRAG-447 |
| LKSRGK | S442L, L443K, P445R, G447K | 442-LKSRGK-447 |

In some embodiments, an engineered cleavable Fe domain further comprises one or more substitutions that allow heterodimerization of two Fc polypeptides. In some embodiments, an engineered cleavable Fe domain further comprises Y349C; T366S; L368A; and Y407V substitutions. In some embodiments, an engineered cleavable Fe domain further comprises S354C and T366W substitutions. In some embodiments, an engineered cleavable Fe domain further comprises Y349C; T366S; L368A; Y407V, and N297Asubstitutions. In some embodiments, an engineered cleavable Fe domain further comprises S354C, T366W and N297Asubstitutions. In some embodiments, an engineered cleavable Fe domain further comprises Y349C; T366S; L368A; Y407V, N297A and I253A substitutions. In some embodiments, an engineered cleavable Fe domain further comprises S354C, T366W, N297A and I253A substitutions.

In some embodiments, the engineered cleavable Fe domain is used as a carrier moiety of a cytokine for cancer treatment. The engineered cleavable Fe domain is fused to a masked cytokine molecule such that upon cleavage of the engineered protease cleavage site such as the engineered tumor-associated protease cleavage site in the cleavable Fe domain, the masked cytokine is released from the masking moiety.

In some embodiments, the cleavable Fe domain, in addition to incorporation of one or more protease cleavage sites, may comprise further mutations described herein.

Incorporation of Cleavable Substrate in F-Strand of the Fc Domain

In some embodiments, an engineered cleavable Fe domain comprises one or more substitutions in the F-strand of the CH3 domain. In some embodiments, an engineered cleavable Fe domain comprises one or more substitutions in the F-strand within the Fe domain. In some embodiments, an engineered cleavable Fe domain comprises one or more substitutions in positions between 416 and 425 by EU numbering.

In some embodiments, the cleavable peptide motif is located between positions 416 and 423 by EU numbering. In some embodiments, the cleavable peptide motif is located between positions 416 and 425 by EU numbering. In some embodiments, the cleavable peptide motif is located between positions 416 and 423 by EU numbering. In some embodiments, the cleavable peptide motif is located between positions 417 and 425 by EU numbering. In some embodiments, the cleavable peptide motif is located between positions 417 and 424 by EU numbering. In some embodiments, the cleavable peptide motif is located between positions 418 and 425 by EU numbering. In some embodiments, the cleavable peptide motif is located between positions 419 and 422 by EU numbering. In some embodiments, the cleavable peptide motif is located between positions 419 and 426 by EU numbering. In some embodiments, the cleavable peptide motif is located between positions 420 and 423 by EU numbering. In some embodiments, the cleavable peptide motif is located between positions 421 and 424 by EU numbering.

In some embodiments, an engineered cleavable Fc domain comprises R416A, W417P, Q418A, Q419G, G420L, N421I, F423P, S424Y, C425N substitutions by EU numbering. In some embodiments, an engineered cleavable Fc domain comprises R416I, W417S, Q418S, Q419G, G420L, N421L, V422S, F423G, S424R, C425S substitutions by EU numbering. In some embodiments, an engineered cleavable Fc domain comprises R416V, W417P, Q418L, Q419S, G420L, N421Y, V422S, F423G, C425G substitutions by EU numbering. In some embodiments, an engineered cleavable Fc domain comprises W417P, Q418A, Q419N, G420L, N421V, V422A, F423P, S424D, C425P substitutions by EU numbering. In some embodiments, an engineered cleavable Fc domain comprises W417V, Q418P, Q419L, G420S, N421L, V422Y, F423S, S424G, C425G substitutions by EU numbering. In some embodiments, an engineered cleavable Fc domain comprises Q418M, Q419P, G420Y, N421D, V422L, F423Y, S424H, C425P substitutions by EU numbering. In some embodiments, an engineered cleavable Fc domain comprises Q418P, Q419V, G420S, N421L, V422R, F423S, S424G, C425S substitutions by EU numbering. In some embodiments, an engineered cleavable Fc domain comprises Q418R, Q419A, G420A, N421A, F423K, C425P substitutions by EU numbering. In some embodiments, an engineered cleavable Fc domain comprises Q418R, Q419P, G420L, N421A, V422L, F423W, S424R, C425S substitutions by EU numbering. In some embodiments, an engineered cleavable Fc domain comprises Q418V, Q419P, G420L, N421S, V422L, F423Y, C425G substitutions by EU numbering.

In some embodiments, an engineered cleavable Fc domain comprises Q419P, G420L, N421G, V422L, C425G substitutions by EU numbering. In some embodiments, an engineered cleavable Fc domain comprises Q418V, Q419P, G420L, N421S, V422L, F423Y, C425G substitutions by EU numbering. In some embodiments, an engineered cleavable Fc domain comprises G420P, N421L, V422G, F423L, C425G substitutions by EU numbering. In some embodiments, an engineered cleavable Fe domain comprises N421P, V422L, F423G, S424L, C425G substitutions by EU numbering.

In some embodiments, an engineered cleavable Fe domain comprises substitutions shown in Table 11a.

TABLE 11a
Exemplary Engineered Fc domain comprising cleavable substrates in F-Strand

| Protease | | |
|---|---|---|
| Cleavage Site | Fc Mutations | Cleavage Substrate location |
| VPLSLYSG | Q418V, Q419P, G420L, N421S, V422L, | 418-VPLSLYSG-425 |
| | F423Y, C425G | |
| RAAAVKSP | Q418R, Q419A, G420A, N421A, F423K, | 418-RAAAVKSP-425 |
| | C425P | |
| MPYDLYHP | Q418M, Q419P, G420Y, N421D, V422L, | 418-MPYDLYHP-425 |
| | F423Y, S424H, C425P | |
| VPLSLYSG | W417V, Q418P, Q419L, G420S, N421L, | 417-VPLSLYSG-424 |
| | V422Y, F423S, S424G, C425G | |
| VPLSLYSG | R416V, W417P, Q418L, Q419S, G420L, | 416-VPLSLYSG-423 |
| | N421Y, V422S, F423G, C425G | |
| ISSGLLSGRS | R416I, W417S, Q418S, Q419G, G420L, | 416-ISSGLLSGRS-425 |
| | N421L, V422S, F423G, S424R, C425S | |
| RPLALWRS | Q418R, Q419P, G420L, N421A, V422L, | 418-RPLALWRS-425 |
| | F423W, S424R, C425S | |
| TQKPLGLS | Q418T, G420K, N421P, V422L, F423G, | 418-TQKPLGLS-425 |
| | S424L, C425S | |
| APAGLIVPYN | R416A, W417P, Q418A, Q419G, G420L, | 416-APAGLIVPYN-425 |
| | N421I, F423P, S424Y, C425N | |
| PVSLRSGS | Q418P, Q419V, G420S, N421L, V422R, | 418-PVSLRSGS-425 |
| | F423S, S424G, C425S | |
| PANLVAPDP | W417P, Q418A, Q419N, G420L, N421V, | 417-PANLVAPDP-425 |
| | V422A, F423P, S424D, C425P | |
| PLGL | N421P, V422L, F423G, S424L, C425G | 421-PLGL-424 |
| PLGL | G420P, N421L, V422G, F423L, C425G | 420-PLGL-423 |
| PLGL | Q419P, G420L, N421G, V422L, C425G | 419-PLGL-422 |
| VPLSLYSG | S375C, P396C, Q418V, Q419P, G420L, | 418-VPLSLYSG-425 |
| | N421S, V422L, F423Y, C425G | |
| VPLSLYSG | Q418V, Q419P, G420L, N421S, V422L, | 418-VPLSLYSG-425 |
| | F423Y, C425G, L432C, T437C | |
| VPLSLYSG | Q418V, Q419P, G420L, N421S, V422L, | 418-VPLSLYSG-425 |
| | F423Y, C425G | |
| VPLSLYSG | Q418V, Q419P, G420L, N421S, V422L, | 418-VPLSLYSG-425 |
| | F423Y, C425G | |
| VPLSLYSG | Q418V, Q419P, G420L, N421S, V422L, | 418-VPLSLYSG-425 |
| | F423Y, C425G | |
| VPLSLYSG | Q418V, Q419P, G420L, N421S, V422L, | 418-VPLSLYSG-425 |
| | F423Y, C425G | |
| VPLSLYSG | Q418V, Q419P, G420L, N421S, V422L, | 419-VPLSLYSG-426 |
| | F423Y, C425G | |

In some embodiments, an engineered cleavable Fe domain further comprises substitutions that allow heterodimerization of two Fc polypeptides. In some embodiments, the engineered cleavable Fe domain comprises substitutions shown in Table 11a and comprises a serine at position 367 by EU numbering. In some embodiments, the engineered cleavable Fe domain comprises substitutions shown in Table 11a and further comprises knob mutations. In some embodiments, the engineered cleavable Fe domain comprises substitutions shown in Table 11a and further comprises hole mutations.

In some embodiments, an engineered cleavable Fe domain comprises a combination of mutations listed in Table 11a and mutations that stabilize the Fe domain. In some embodiments, an engineered cleavable Fe domain comprises a combination of mutations listed in Table 11a and C367S. In some embodiments, an engineered cleavable Fe domain comprises a combination of mutations listed in Table 11a and C425G. In some embodiments, an engineered cleavable Fe domain comprises a combination of mutations listed in Table 11a and S375C. In some embodiments, an engineered cleavable Fe domain comprises a combination of mutations listed in Table 11a and P396C. In some embodiments, an engineered cleavable Fe domain comprises a combination of mutations listed in Table 11a and 432C. In some embodiments, an engineered cleavable Fe domain comprises a combination of mutations listed in Table 11a and T437C. In some embodiments, an engineered cleavable Fe domain comprises a combination of mutations listed in Table 11a and S408C. In some embodiments, an engineered cleavable Fe domain comprises a combination of mutations listed in Table 11a and 379C. In some embodiments, an engineered cleavable Fe domain comprises a combination of mutations listed in Table 11a and W381C. In some embodiments, an engineered cleavable Fe domain comprises a combination of mutations listed in Table 11a and L410C. In some embodiments, an engineered cleavable Fe domain comprises a combination of mutations listed in Table 11a and K370C. In some embodiments, an engineered cleavable Fe domain comprises a combination of mutations listed in Table 11a and F405C. In some embodiments, an engineered cleavable Fe domain comprises a combination of mutations listed in Table 11a and 371C. In some embodiments, an engineered cleavable Fc domain comprises a combination of mutations listed in Table 11a and S403C.

In some embodiments, an engineered cleavable Fc domain further comprises Y349C; T366S; L368A; and Y407V substitutions. In some embodiments, an engineered cleavable Fc domain further comprises S354C and T366W substitutions. In some embodiments, an engineered cleavable Fc domain further comprises Y349C; T366S; L368A; Y407V, and N297A substitutions. In some embodiments, an engineered cleavable Fc domain further comprises S354C, T366W and N297A substitutions. In some embodiments, an engineered cleavable Fc domain further comprises Y349C; T366S; L368A; Y407V, N297A and I253A substitutions. In some embodiments, an engineered cleavable Fc domain further comprises S354C, T366W, N297A and I253A substitutions.

Incorporation of Cleavable Substrate in FG-Loop of the Fc Domain

In some embodiments, an engineered cleavable Fc domain comprises one or more substitutions in the FG-loop within the Fc domain. In some embodiments, an engineered cleavable Fc domain comprises one or more substitutions in positions between 426 and 437 by EU numbering.

In some embodiments, the cleavable peptide motif is located between positions 430 and 437 by EU numbering. In some embodiments, the cleavable peptide motif is located between positions 428 and 434 by EU numbering. In some embodiments, the cleavable peptide motif is located between positions 428 and 433 by EU numbering. In some embodiments, the cleavable peptide motif is located between positions 428 and 437 by EU numbering. In some embodiments, the cleavable peptide motif is located between positions 429 and 437 by EU numbering. In some embodiments, the cleavable peptide motif is located between positions 434 and 437 by EU numbering. In some embodiments, the cleavable peptide motif is located between positions 433 and 436 by EU numbering. In some embodiments, the cleavable peptide motif is located between positions 432 and 435 by EU numbering. In some embodiments, the cleavable peptide motif is located between positions 431 and 434 by EU numbering. In some embodiments, the cleavable peptide motif is located between positions 430 and 433 by EU numbering. In some embodiments, the cleavable peptide motif is located between positions 429 and 432 by EU numbering. In some embodiments, the cleavable peptide motif is located between positions 428 and 431 by EU numbering.

In some embodiments, an engineered cleavable Fc domain comprises E430V, A431P, H433S, N434L, H435Y, Y436S, T437G substitutions by EU numbering. In some embodiments, an engineered cleavable Fc domain comprises E430R, L432A, H433A, N434V, H435K, Y436S, T437P substitutions by EU numbering. In some embodiments, an engineered cleavable Fc domain comprises E430M, A431P, L432Y, H433D, N434L, H435Y, Y436H, T437P substitutions by EU numbering. In some embodiments, an engineered cleavable Fc domain comprises 428-ins-VP, H429L, E430S, A431L, L432Y, H433S, N434G substitutions by EU numbering. In some embodiments, an engineered cleavable Fc domain comprises 428-ins-RA, H429A, E430A, A431V, L432K, H433S, N434P substitutions by EU numbering. In some embodiments, an engineered cleavable Fc domain comprises 428-ins-MP, H429Y, E430D, A431L, L432Y, N434P substitutions by EU numbering. In some embodiments, an engineered cleavable Fc domain comprises 428-ins-VPL, H429S, E430L, A431Y, L432S, H433G substitutions by EU numbering. In some embodiments, an engineered cleavable Fc domain comprises 428-ins-RAA, H429A, E430V, A431K, L432S, H433P substitutions by EU numbering. In some embodiments, an engineered cleavable Fc domain comprises 428-ins-MPY, H429D, E430L, A431Y, L432H, H433P substitutions by EU numbering. In some embodiments, an engineered cleavable Fc domain comprises 428-ins-I, H429S, E430S, A431G, H433L, N434S, H435G, Y436R, T437S substitutions by EU numbering. In some embodiments, an engineered cleavable Fc domain comprises E430R, A431P, H433A, N434L, H435W, Y436R, T437S substitutions by EU numbering. In some embodiments, an engineered cleavable Fc domain comprises E430T, A431Q, L432K, H433P, N434L, H435G, Y436L, T437S substitutions by EU numbering. In some embodiments, an engineered cleavable Fc domain comprises 428-ins-A, H429P, E430A, A431G, H433I, N434V, H435P, T437N substitutions by EU numbering. In some embodiments, an engineered cleavable Fc domain comprises E430P, A431V, L432S, H433L, N434R, H435S, Y436G, T437S substitutions by EU numbering. In some embodiments, an engineered cleavable Fc domain comprises H429P, E430A, A431N, H433V, N434A, H435P, Y436D, T437P substitutions by EU numbering. In some embodiments, an engineered cleavable Fc domain comprises N434P, H435L, Y436G, T437L substitutions by EU numbering. In some embodiments, an engineered cleavable Fc domain comprises H433P, N434L, H435G, Y436L substitutions by EU numbering. In some embodiments, an engineered cleavable Fc domain comprises L432P, H433L, N434G, H435L substitutions by EU numbering. In some embodiments, an engineered cleavable Fe domain comprises A43P, H433G, N434L substitutions by EU numbering. In some embodiments, an engineered cleavable Fe domain comprises E430G, A431P, H433G, N434L substitutions by EU numbering. In some embodiments, an engineered cleavable Fe domain comprises E430P, A431L, L432G, H433L substitutions by EU numbering. In some embodiments, an engineered cleavable Fe domain comprises H429G, E430P, A431L, L432G, H433L substitutions by EU numbering. In some embodiments, an engineered cleavable Fe domain comprises 428-ins-G, H429P, E430L, A43G, H433G substitutions by EU numbering. In some embodiments, an engineered cleavable Fe domain comprises 428-ins-GGP, H429L, E430G, A431L, L432G substitutions by EU numbering.

In some embodiments, an engineered cleavable Fe domain comprises protease cleavage sites in Table 11b. The engineered cleavable Fe domain may comprise substitutions shown in Table 11b.

TABLE 11b
Exemplary Engineered Fc domain comprising cleavable substrates in FG-Loop

| Protease cleavage site | Substitutes | Location |
|---|---|---|
| VPLSLYSG | E430V, A431P, H433S, N434L, | 430-VPLSLYSG-437 |
| | H435Y, Y436S, T437G, | |
| RAAAVKSP | E430R, L432A, H433A, N434V, | 430-RAAAVKSP-437 |
| | H435K, Y436S, T437P | |
| MPYDLYHP | E430M, A431P, L432Y, H433D, | 430-MPYDLYHP-437 |
| | N434L, H435Y, Y436H, T437P | |
| VPLSLYSG | 428-ins-VP, H429L, E430S, A431L, | 428-ins-VPLSLYSG-434 |
| | L432Y, H433S, N434G | |
| RAAAVKSP | 428-ins-RA, H429A, E430A, A431V, | 428-ins-RAAAVKSP-434 |
| | L432K, H433S, N434P | |
| MPYDLYHP | 428-ins-MP, H429Y, E430D, A431L, | 428-ins-MPYDLYHP-434 |
| | L432Y, N434P | |
| VPLSLYSG | 428-ins-VPL, H429S, E430L, | 428-ins-VPLSLYSG-433 |
| | A431Y, L432S, H433G | |
| RAAAVKSP | 428-ins-RAA, H429A, E430V, | 428-ins-RAAAVKSP-433 |
| | A431K, L432S, H433P | |
| MPYDLYHP | 428-ins-MPY, H429D, E430L, | 428-ins-MPYDLYHP-433 |
| | A431Y, L432H, H433P | |
| ISSGLLSGRS | 428-ins-I, H429S, E430S, A431G, | 428-ins-ISSGLLSGRS- |
| | H433L, N434S, H435G, Y436R, | 437 |
| | T437S | |
| RPLALWRS | E430R, A431P, H433A, N434L, | 430-RPLALWRS-437 |
| | H435W, Y436R, T437S | |
| TQKPLGLS | E430T, A431Q, L432K, H433P, | 430-TQKPLGLS-437 |
| | N434L, H435G, Y436L, T437S | |
| APAGLIVPYN | 428-ins-A, H429P, E430A, A431G, | 428-ins-APAGLIVPYN- |
| | H433I, N434V, H435P, T437N | 437 |
| PVSLRSGS | E430P, A431V, L432S, H433L, | 430-PVSLRSGS-437 |
| | N434R, H435S, Y436G, T437S | |
| PANLVAPDP | H429P, E430A, A431N, H433V, | 429-PANLVAPDP-437 |
| | N434A, H435P, Y436D, T437P | |
| PLGL | N434P, H435L, Y436G, T437L | 434-PLGL-437 |
| PLGL | H433P, N434L, H435G, Y436L | 433-PLGL-436 |
| PLGL | L432P, H433L, N434G, H435L | 432-PLGL-435 |
| PLGL | A431P, H433G, N434L | 431-PLGL-434 |
| PLGL | E430G, A431P, H433G, N434L | 431-PLGL-434 |
| PLGL | E430P, A431L, L432G, H433L | 430-PLGL-433 |
| PLGL | H429G, E430P, A431L, L432G, | 430-PLGL-433 |
| | H433L | |
| PLGL | 428-ins-G, H429P, E430L, A431G, | 429-PLGL-432 |
| | H433G | |
| PLGL | 428-ins-GGP, H429L, E430G, | 428-ins-PLGL-431 |
| | A431L, L432G | |

In some embodiments, an engineered cleavable Fe domain further comprises substitutions that allow heterodimerization of two Fe polypeptides. In some embodiments, an engineered cleavable Fe domain further comprises substitutions that can extend half-life of a polypeptide comprising an engineered cleavable Fe domain.

In some embodiments, the engineered cleavable Fe domain comprises substitutions shown in Table 11b and comprises a serine at position 367 by EU numbering. In some embodiments, the engineered cleavable Fe domain comprises substitutions shown in Table 11b and further comprises knob mutations. In some embodiments, the engineered cleavable Fe domain comprises substitutions shown in Table 11b and further comprises hole mutations.

In some embodiments, an engineered cleavable Fe domain further comprises Y349C; T366S; L368A; and Y407V substitutions. In some embodiments, an engineered cleavable Fe domain further comprises S354C and T366W substitutions. In some embodiments, an engineered cleavable Fe domain further comprises Y349C; T366S; L368A; Y407V, and N297A substitutions. In some embodiments, an engineered cleavable Fe domain further comprises S354C, T366W and N297A substitutions. In some embodiments, an engineered cleavable Fe domain further comprises Y349C; T366S; L368A; Y407V, N297A and I253A substitutions. In some embodiments, an engineered cleavable Fe domain further comprises S354C, T366W, N297A and I253A substitutions.

In some embodiments, an engineered cleavable Fe domain comprising one or more substitutions between positions 416 and 416, or between positions 428 to 437 has reduced binding to protein A.

Exemplary Engineered Cleavable Fc Domains with Cathepsin B or Matriptase Substrates

In some embodiments, an engineered cleavable Fe domain comprises a non-MMP protease cleavage site in Table 11c. In one embodiment, the non-MMP protease is Cathepsin B. In another embodiment, the non-MMP protease is Matriptase. The engineered cleavable Fe domain may comprise any one set of the substitutions in Table 11c.

In some embodiments, an engineered cleavable Fe domain comprises one or more substitutions in positions between 436 and 447 by EU numbering to incorporate a cleavage site for Cathepsin B or Matriptase.

In some embodiments, the cleavable peptide motif is located between positions 440 and 447 by EU numbering. In some embodiments, the cleavable peptide motif is located between positions 444 and 447 by EU numbering. In some embodiments, the cleavable peptide motif is located between positions 441 and 447 by EU numbering. In some embodiments, the cleavable peptide motif is located between positions 443 and 447 by EU numbering. In some embodiments, the cleavable peptide motif is located between positions 438 and 445 by EU numbering. In some embodiments, the cleavable peptide motif is located between positions 436 and 443 by EU numbering. In some embodiments, the cleavable peptide motif is located between positions 442 and 447 by EU numbering.

TABLE 11c
Exemplary Engineered Fc domain comprising Non-MMP cleavable substrates

| Protease | Substitutes | Location |
|---|---|---|
| RSKYLATA | S440R, L441S, S442K, L443Y, | 440-RSKYLATA-447 |
| | S444L, P445A, G446T, G447A | |
| GRPRHQGV | S440G, L441R, S442P, L443R, | 440-GRPRHQGV-447 |
| | S444H, P445Q, G447V | |
| GLFG | S444G, P445L, G446F | 444-GLFG-447 |
| GFLG | S444G, P445F, G446L | 444-GFLG-447 |
| AGRRAAK | L441A, S442G, L443R, S444R, | 441-AGRRAAK-447 |
| | P445A, G446A, G447K | |
| FRLWA | L443F, S444R, P445L, G446W, | 443-FRLWA-447 |
| | G447A | |
| FRLWS | L443F, S444R, P445L, G446W, | 443-FRLWS-447 |
| | G447S | |
| NFFGVGGE | S440N, L441F, S442F, L443G, | 440-NFFGVGGE-447 |
| | S444V, P445G, G447E | |
| PMKRLTLA | S440P, L441M, S442K, L443R, | 440-PMKRLTLA-447 |
| | S444L, P445T, G446L, G447A | |
| PMKRLTLA | K439P, S440M, L441K, S442R, | 438-PMKRLTLA-445 |
| | S444T, P445L, G446A | |
| PMKRLTLA | Q438P, K439M, S440K, L441R, | 436-PMKRLTLA-443 |
| | S442L, L443T, S444L, P445A | |
| FPLATYAP | S440F, L441P, S442L, L443A, | 440-FPLATYAP-447 |
| | S444T, P445Y, G446A, G447P | |
| FLVGGASL | S440F, S442V, L443G, S444G, | 440-FLVGGASL-447 |
| | P445A, G446S, G447L | |
| KPMQFLGD | S440K, L441P, S442M, L443Q, | 440-KPMQFLGD-447 |
| | S444F, P445L, G447D | |
| GIVRAKGV | S440G, L441I, S442V, L443R, | 440-GIVRAKGV-447 |
| | S444A, P445K, G447V | |
| ALFKSSFP | S440A, S442F, L443K, P445S, | 440-ALFKSSFP-447 |
| | G446F, G447P | |
| SGRRSPGG | L441G, S442R, L443R | 440-SGRRSPGG-447 |
| SLGRRPGG | S442G, L443R, S444R | 440-SLGRRPGG-447 |
| SLSGRRGG | L443G, S444R, P445R | 440-SLSGRRGG-447 |
| SLSLGRRG | S444G, P445R, G446R | 440-SLSLGRRG-447 |
| SLSLSGRR | P445G, G446R, G447R | 440-SLSLSGRR-447 |
| GGPRRL | S442G, L443G, S444P, P445R, | 442-GGPRRL-447 |
| | G446R, G447L | |
| GGPLRL | S442G, L443G, S444P, P445L, | 442-GGPLRL-447 |
| | G446R, G447L | |
| GGPKLL | S442G, L443G, S444P, P445K, | 442-GGPKLL-447 |
| | G446L, G447L | |
| GGPRNL | S442G, L443G, S444P, P445R, | 442-GGPRNL-447 |
| | G446N, G447L | |
| GGPRML | S442G, L443G, S444P, P445R, | 442-GGPRML-447 |
| | G446M, G447L | |
| EHLRSPGG | S440E, L441H, S442L, L443R | 440-EHLRSPGG-447 |
| FRSGVPGG | S440F, L441R, L443G, S444V | 440-FRSGVPGG-447 |
| SLLLRTGN | S442L, S444R, P445T, G447N | 440-SLLLRTGN-447 |
| AGLRSPGG | S440A, L441G, S442L, L443R | 440-AGLRSPGG-447 |
| SLFRSAGP | S442F, L443R, P445A, G447P | 440-SLFRSAGP-447 |
| SLFRAPGP | S442F, L443R, S444A, G447P | 440-SLFRAPGP-447 |
| WLFRSPLG | S440W, S442F, L443R, G446L | 440-WLFRSPLG-447 |
| SRLRSPQG | L441R, S442L, L443R, G446Q | 440-SRLRSPQG-447 |
| SLVLSGRR | S442V, P445G, G446R, G447R | 440-SLVLSGRR-447 |
| KQLRHMRG | S440K, L441Q, S442L, L443R, | 440-KQLRHMRG-447 |
| | S444H, P445M, G446R | |
| LSGRSDNH | S440L, L441S, S442G, L443R, | 440-LSGRSDNH-447 |
| | P445D, G446N, G447H | |
| LSGK | S444L, P445S, G447K | 444-LSGK-447 |
| LSGR | S444L, P445S, G447R | 444- LSGR -447 |
| RQARVVGG | S440R, L441Q, S442A, L443R, | 440-RQARVVGG-447 |
| | S444V, P445V | |
| RQRRVVGG | S440R, L441Q, S442R, L443R, | 440-RQRRVVGG-447 |
| | S444V, P445V | |
| RQRRVVGG | K439R, S440Q, L441R, S442R, | 438-RQRRVVGG-445 |
| | L443V, S444V, P445G | |
| RQRRVVGG | Q438R, K439Q, S440R, L441R, | 436-RQRRVVGG-443 |
| | S442V, L443V, S444G, P445G | |
| RQYRVVGG | S440R, L441Q, S442Y, L443R, | 440-RQYRVVGG-447 |
| | S444V, P445V | |
| SKGRSLIG | L441K, S442G, L443R, P445L, | 440-SKGRSLIG-447 |
| | G446I | |
| PRFKIIGG | S440P, L441R, S442F, L443K, | 440-PRFKIIGG-447 |
| | S444I, P445I | |
| KQLRVVNG | S440K, L441Q, S442L, L443R, | 440-KQLRVVNG-447 |
| | S444V, P445V, G446N | |
| IQPRITGG | S440I, L441Q, S442P, L443R, S444I, | 440-IQPRITGG-447 |
| | P445T | |
| KQSRKFVP | S440K, L441Q, L443R, S444K, | 440-KQSRKFVP-447 |
| | P445F, G446V, G447P | |
| GRQSRAGG | S440G, L441R, S442Q, L443S, | 440-GRQSRAGG-447 |
| | S444R, P445A | |
| SGRSSPGG | L441G, S442R, L443S | 440-SGRSSPGG-447 |
| SSGRSPGG | L441S, S442G, L443R | 440-SSGRSPGG-447 |
| SLSGRSGG | L443G, S444R, P445S | 440-SLSGRSGG-447 |
| SLSSGRSG | L443S, S444G, P445R, G446S | 440-SLSSGRSG-447 |
| SLSLSGRS | P445G, G446R, G447S | 440-SLSLSGRS-447 |
| PLRLSRA | L441P, S442L, L443R, S444L, | 441-PLRLSRA-447 |
| | P445S, G446R, G447A | |
| PLKLSRA | L441P, S442L, L443K, S444L, | 441-PLKLSRA-447 |
| | P445S, G446R, G447A | |
| PLGLSGRS | S440P, S442G, P445G, G446R, | 440-PLGLSGRS-447 |
| | G447S | |
| PLGLRSRA | S440P, S442G, S444R, P445S, | 440-PLGLRSRA-447 |
| | G446R, G447A | |
| PLGLKSRA | S440P, S442G, S444K, P445S, | 440-PLGLKSRA-447 |
| | G446R, G447A | |
| RGSRAG | S442R, L443G, P445R, G446A | 442-RGSRAG-447 |
| RLSRGK | S442R, P445R, G447K | 442-RLSRGK-447 |
| RGSRGG | S442R, L443G, P445R | 442-RGSRGG-447 |
| KGSRAG | S442K, L443G, P445R, G446A | 442-KGSRAG-447 |
| KLSRGK | S442K, P445R, G447K | 442-KLSRGK-447 |
| GRSRAG | S442G, L443R, P445R, G446A | 442-GRSRAG-447 |
| LRSRGK | S442L, L443R, P445R, G447K | 442-LRSRGK-447 |
| GRSRGG | S442G, L443R, P445R | 442-GRSRGG-447 |
| GKSRAG | S442G, L443K, P445R, G446A | 442-GKSRAG-447 |
| LKSRGK | S442L, L443K, P445R, G447K | 442-LKSRGK-447 |

In some embodiments, an engineered cleavable Fe domain comprises one or more substitutions in positions between 416 and 425 by EU numbering to incorporate a cleavage site in Table 11c. In some embodiments, an engineered cleavable Fc domain comprises one or more substitutions in positions between 426 and 437 by EU numbering to incorporate a cleavage site in Table 11c.

In some embodiments, an engineered cleavable Fc domain further comprises substitutions that allow heterodimerization of two Fc polypeptides. In some embodiments, an engineered cleavable Fc domain further comprises substitutions that can extend half-life of a polypeptide comprising an engineered cleavable Fc domain.

In some embodiments, the engineered cleavable Fc domain comprises substitutions shown in Table 11c and comprises a serine at position 367 by EU numbering. In some embodiments, the engineered cleavable Fc domain comprises substitutions shown in Table 11c and further comprises knob mutations. In some embodiments, the engineered cleavable Fc domain comprises substitutions shown in Table 11c and further comprises hole mutations.

In some embodiments, an engineered cleavable Fc domain further comprises Y349C; T366S; L368A; and Y407V substitutions. In some embodiments, an engineered cleavable Fc domain further comprises S354C and T366W substitutions. In some embodiments, an engineered cleavable Fe domain further comprises Y349C; T366S; L368A; Y407V, and N297A substitutions. In some embodiments, an engineered cleavable Fe domain further comprises S354C, T366W and N297A substitutions. In some embodiments, an engineered cleavable Fe domain further comprises Y349C; T366S; L368A; Y407V, N297A and I253A substitutions. In some embodiments, an engineered cleavable Fe domain further comprises S354C, T366W, N297A and I253A substitutions.

Fc Engineering to Promote Heterodimerization or to Increase Half-Life

An Fe domain or a fragment thereof that is capable of FcRn-mediated recycling, can be reduce or otherwise delay clearance of the targeted cytokine from a subject, thereby prolonging the half-life of the administered targeted cytokine. In some embodiments, the cleavable Fe domain or a fragment thereof is any antibody or fragment thereof that is capable of FcRn-mediated recycling, such as any heavy chain polypeptide or portion thereof (e.g., Fe domain or fragment thereof) that is capable of FcRn-mediated recycling.

The cleavable Fe domain or a fragment thereof may be derived from any antibody or fragment thereof. However, in some embodiments, either a first Fc polypeptide or a second Fc polypeptide may does not bind to the FcRn receptor, such as a light chain polypeptide. For example, in some embodiments, a first Fc polypeptide does not directly interact with the FcRn receptor, but the targeted cytokine nonetheless has an extended half-life due to comprising a second Fc polypeptide that is capable of interacting with the FcRn receptor, such as by comprising a heavy chain polypeptide. It is recognized in the art that FcRn-mediated recycling requires binding of the FcRn receptor to the Fc region of the antibody or fragment thereof. For instance, studies have shown that residues 1253, S254, H435, and Y436 (numbering according to the Kabat EU index numbering system) are important for the interaction between the human Fc region and the human FcRn complex. See, e.g., Firan, M., et al., Int. Immunol. 13 (2001) 993-1002; Shields, R. L., et al, J. Biol. Chem. 276 (2001) 6591-6604). Various mutants of residues 248-259, 301-317, 376-382, and 424-437 (numbering according to the Kabat EU index numbering system) have also been examined and reported. Yeung, Y. A., et al. (J. Immunol. 182 (2009) 7667-7671.

In some embodiments, the first and/or second Fc polypeptides of the cleavable Fe domains each contain one or more modifications that promote the non-covalent association of the first and the second Fc polypeptides. In some embodiments, the first Fc polypeptide comprises an IgG1 Fc domain or fragment thereof including the mutations Y349C; T366S; L368A; and Y407V to form a ‘hole’ in the first half-life extension domain and the second Fc polypeptide comprises an IgG1 Fc domain or fragment thereof including the mutations S354C and T366W to form the ‘knob’ in the second half-life extension domain.

In some embodiments, the first and second Fc polypeptides are each an IgG1, IgG2 or IgG4 Fc domain or fragment thereof. In some embodiments, the first and second Fc polypeptides are each an IgG1 Fc domain or fragment thereof. Human IgG1 Immunoglobulin heavy constant gamma 1 has the sequence:

(SEQ ID NO: 1)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPG

In some embodiments, the first and second Fc polypeptides are derived from the sequence for human IgG1 Immunoglobulin heavy constant gamma 1 having SEQ ID NO: 1 (the ‘parent sequence’), such that the first and second Fc polypeptides each comprise SEQ ID NO: 1 or fragment thereof, with one or more amino acid modifications.

In some embodiments, the first and Fc polypeptides each comprise the portion of SEQ ID NO: 1 shown in bold above, optionally with one or more amino acid modifications, i.e.:

(SEQ ID NO: 2)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPG

In some embodiments, the first and second Fc polypeptides comprise SEQ ID NO: 2 with amino substitutions to promote association of the first and second Fc polypeptides according to the ‘knob into holes’ approach. In some embodiments, the sequence SEQ ID NO: 2 contains mutations Y349C; T366S; L368A; and Y407V (numbered according to the Kabat EU numbering system) to form the ‘hole’ in the first Fc polypeptide and mutations S354C and T366W (numbered according to the Kabat EU numbering system) to form the ‘knob’ in the second Fc polypeptide. These modified sequences have SEQ ID Nos. 3 and 4 shown below:

First Fc polypeptide (Y349C; T366S; L368A; and
Y407V) SEQ ID NO. 3:
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVCTLPPSRDELTKNQVSLSC
AVKGFYPSDIAVEWESNGQPENNYKTTPPVLDSDGSFFLVSKLTVDKSR
WQQGNVFSCSVMHEALHNHYTQKSLSLSPG
Second Fc polypeptide (S354C and T366W) SEQ ID
NO. 4:
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPCRDELTKNQVSLWC
LVKGFYPSDIAVEWESNGQPENNYKTTPPVLDSDGSFFLYSKLTVDKSR
WQQGNVFSCSVMHEALHNHYTQKSLSLSPG

In some embodiments, the first and second half-life extension domains each further comprise amino substitution N297A, numbered according to the Kabat EU numbering system:

First Fc polypeptide (Y349C; T366S; L368A; Y407V
and N297A) SEQ ID NO. 5:
DKTHTCPPCPAPELLGGPSVFLFPPKPKDTLMISRTPEVTCVVVDVSHE
DPEVKFNWYVDGVEVHNAKTKPREEQYASTYRVVSVLTVLHQDWLNGKE
YKCKVSNKALPAPIEKTISKAKGQPREPQVCTLPPSRDELTKNQVSLSC
AVKGFYPSDIAVEWESNGQPENNYKTTPPVLDSDGSFFLVSKLTVDKSR
WQQGNVFSCSVMHEALHNHYTQKSLSLSPG
Second Fc polypeptide (S354C, T366W and N297A)
SEQ ID NO. 6:
DKTHTCPPCPAPELLGGPSVFLFPPKPKDTLMISRTPEVTCVVVDVSHE
DPEVKFNWYVDGVEVHNAKTKPREEQYASTYRVVSVLTVLHQDWLNGKE
YKCKVSNKALPAPIEKTISKAKGQPREPQVYTLPPCRDELTKNQVSLWC
LVKGFYPSDIAVEWESNGQPENNYKTTPPVLDSDGSFFLYSKLTVDKSR
WQQGNVFSCSVMHEALHNHYTQKSLSLSPG

In some embodiments, the first and second Fc polypeptides each further comprise the amino substitution I253A, numbered according to the Kabat EU numbering system.

In some embodiments, the first and second Fc polypeptides each further comprise both the amino substitutions N297A and I253A, numbered according to the Kabat EU numbering system.

First Fc polypeptide (Y349C; T366S; L368A; Y407V,
N297A and I253A) SEQ ID NO. 7:
DKTHTCPPCPAPELLGGPSVFLFPPKPKDTLMASRTPEVTCVVVDVSHE
DPEVKFNWYVDGVEVHNAKTKPREEQYASTYRVVSVLTVLHQDWLNGKE
YKCKVSNKALPAPIEKTISKAKGQPREPQVCTLPPSRDELTKNQVSLSC
AVKGFYPSDIAVEWESNGQPENNYKTTPPVLDSDGSFFLVSKLTVDKSR
WQQGNVFSCSVMHEALHNHYTQKSLSLSPG
Second Fc polypeptide (S354C, T366W, N297A and
I253A) SEQ ID NO. 8:
DKTHTCPPCPAPELLGGPSVFLFPPKPKDTLMASRTPEVTCVVVDVSHE
DPEVKFNWYVDGVEVHNAKTKPREEQYASTYRVVSVLTVLHQDWLNGKE
YKCKVSNKALPAPIEKTISKAKGQPREPQVYTLPPCRDELTKNQVSLWC
LVKGFYPSDIAVEWESNGQPENNYKTTPPVLDSDGSFFLYSKLTVDKSR
WQQGNVFSCSVMHEALHNHYTQKSLSLSPG

In some embodiments, the first Fc polypeptide comprises an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequences of any one of SEQ ID NOs: 2-8.

In some embodiments, the second Fc polypeptide comprises an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequences of any one of SEQ ID NOs: 2-8.

In some embodiments, the first Fc polypeptide comprises an amino acid sequence having one or more modifications, such as one or more amino acid substitutions, additions, or deletions, as compared to the amino acid sequence of any one of SEQ ID NOs: 2-8. In some embodiments, the second Fc polypeptide comprises an amino acid sequence having one or more modifications, such as one or more amino acid substitutions, additions, or deletions, as compared to the amino acid sequence of any one of SEQ ID NOs: 2-8. The one or more modifications can be any modifications or alterations described herein, including, in some embodiments, any modifications or alterations disclosed herein that promote heterodimerization of polypeptide chains and/or suppresses homodimerization of polypeptide chains, alter effector function, or enhance effector function.

In some embodiments, the protease cleavage sites as described herein may be introduced into any of SEQ ID NOs: 2-8.

In some embodiments, the cleavable Fc domain may further comprise one or more amino acid substitutions altering effector function. In some embodiments, the half-life extension domain is an IgG1 Fc domain or fragment thereof and comprises one or more amino acid substitutions selected from the group consisting of N297A, N297G, N297Q, L234A, L235A, C220S, C226S, C229S, P238S, E233P, L234V, L234F, L235E, P331S, S267E, L328F, D265A, and P329G, numbered according to the Kabat EU numbering system. In some embodiments, the half-life extension domain is an IgG2 Fc domain or fragment thereof and comprises the amino substitution(s): V234A and G237A; H268Q, V309L, A330S, and A331S; and/or V234A, G237A, P238S, H268A, V309L, and A330S, numbered according to the Kabat EU numbering system. In some embodiments, the half-life extension domain is an IgG2 Fc domain or fragment thereof and comprises one or more amino acid substitutions selected from the group consisting of V234A, G237A, H268Q, V309L, A330S, A331S, P238S, H268A, and V309L, numbered according to the Kabat EU numbering system. In some embodiments, the half-life extension domain is an IgG4 Fc domain or fragment thereof and comprises the amino substitution(s): L235A, G237A, and E318A; S228P, L234A, and L235A; H268Q, V309L, A330S, and P331S; and/or S228P and L235A, numbered according to the Kabat EU numbering system. In some embodiments, the half-life extension domain is an IgG2 Fc domain or fragment thereof and comprises one or more amino acid substitutions selected from the group consisting of L235A, G237A, E318A, S228P, L234A, H268Q, V309L, A330S, and P331S, numbered according to the Kabat EU numbering system.

In some embodiments, the cleavable Fc domain further comprises one or more amino acid substitutions enhancing effector function. In some embodiments, the half-life extension domain is an IgG1 Fc domain or fragment thereof and comprises the amino acid substitution(s): S298A, E333A, and K334A; S239D and I332E; S239D, A330L, and I332E; P247I and A339D or A339Q; D280H and K290S; D280H, K290S, and either S298D or S298V; F243L, R292P, and Y300L; F243L, R292P, Y300L, and P396L; F243L, R292P, Y300L, V305I, and P396L; G236A, S239D, and 1332E; K326A and E333A; K326W and E333S; K290E, S298G, and T299A; K290E, S298G, T299A, and K326E; K290N, S298G, and T299A; K290N, S298G, T299A, and K326E; K334V; L235S, S239D, and K334V; K334V and Q331M, S239D, F243V, E294L, or S298T; E233L, Q311M, and K334V; L234I, Q311M, and K334V; K334V and S298T, A330M, or A330F; K334V, Q311M, and either A330M or A330F; K334V, S298T, and either A330M or A330F; K334V, S239D, and either A330M or S298T; L234Y, Y296W, and K290Y, F243V, or E294L; Y296W and either L234Y or K290Y; S239D, A330S, and 1332E, V264I; F243L and V264I; L328M; 1332E; L328M and 1332E; V264I and 1332E; S239E and 1332E; S239Q and 1332E; S239E; A330Y; I332D; L328I and 1332E; L328Q and 1332E; V264T; V240I; V266I; S239D; S239D and I332D; S239D and I332N; S239D and I332Q; S239E and 1332D; S239E and I332N; S239E and I332Q; S239N and I332D; S239N and 1332E; S239Q and 1332D; A330Y and 1332E; V264I, A330Y, and 1332E; A330L and 1332E; V264I, A330L, and 1332E; L234E, L234Y, or L234I; L235D, L235S, L235Y, or L235I; S239T; V240M; V264Y; A330I; N325T; 1332E and L328D, L328V, L328T, or L328I; V264I, I332E, and either S239E or S239Q; S239E, V264I, A330Y, and 1332E; A330Y, 1332E, and either S239D or S239N; A330L, 1332E, and either S239D or S239N; V264I, S298A, and 1332E; S298A, 1332E, and either S239D or S239N; S239D, V264I, and 1332E; S239D, V264I, S298A, and 1332E; S239D, V264I, A330L, and 1332E; S239D, 1332E, and A330I; P230A; P230A, E233D, and 1332E; E272Y; K274T, K274E, K274R, K274L, or K274Y; F275W; N276L; Y278T; V302I; E318R; S324D, S324I or S324V; K326I or K326T; T335D, T335R, or T335Y; V240I and V266I; S239D, A330Y, 1332E, and L234I; S239D, A330Y, 1332E, and L235D; S239D, A330Y, 1332E, and V240I; S239D, A330Y, 1332E, and V264T; and/or S239D, A330Y, 1332E, and either K326E or K326T, numbered according to the Kabat EU numbering system. In some embodiments, the cleavable Fc domain is an IgG1 Fc domain or fragment thereof and comprises one or more amino acid substitution(s) selected from the group consisting of: P230A, E233D, L234E, L234Y, L234I, L235D, L235S, L235Y, L235I, S239D, S239E, S239N, S239Q, S239T, V240I, V240M, F243L, V264I, V264T, V264Y, V266I, E272Y, K274T, K274E, K274R, K274L, K274Y, F275W, N276L, Y278T, V302I, E318R, S324D, S324I, S324V, N325T, K326I, K326T, L328M, L328I, L328Q, L328D, L328V, L328T, A330Y, A330L, A330I, I332D, I332E, I332N, I332Q, T335D, T335R, and T335Y.

In some embodiments, the cleavable Fc domain further comprises one or more amino acid substitution(s) that enhance binding of the half-life extension domain to FcRn. In some embodiments, the one or more amino acid substitution(s) increase binding affinity of an Fc-containing polypeptide (e.g., a heavy chain polypeptide or an Fc domain or fragment thereof to FcRn at acidic pH. In some embodiments, the half-life extension domain comprises one or more amino acid substitution(s) selected from the group consisting of M428F; T250Q and M428F; M252Y, S254T, and T256E; P257I and N434H; D376V and N434H; P257I and Q3111; N434A; N434W; M428F and N434S; V259I and V308F; M252Y, S254T, and T256E; V259I, V308F and M428F; T307Q and N434A; T307Q and N434S; T307Q, E380A, and N434A; V308P and N434A; N434H; and V308P.

Knobs-into-Holes Approach

One strategy for promoting heterodimerization of two Fc polypeptides is an approach termed the “knobs-into-holes”.

In some embodiments, the cleavable Fc domain comprises a first Fc polypeptide and a second Fc polypeptide, each of which comprises a CH3 domain. In some embodiments, the Fc polypeptide comprising a CH3 domain is a heavy chain polypeptide or a fragment thereof (e.g., an Fc domain or fragment thereof). The CH3 domains of the two Fc polypeptides can be altered by the “knobs-into-holes” technology, which is described in detail with several examples in, e.g., WO 1996/027011; Ridgway, J. B. et al, Protein Eng. (1996) 9(7): 617-621; Merchant, A. M., et al, Nat. Biotechnol. (1998) 16(7): 677-681. See also Klein et al. (2012), MAbs, 4(6): 653-663. Using the knob-into-holes method, the interaction surfaces of the two CH3 domains are altered to increase the heterodimerization of the two half-life extension domains containing the two altered CH3 domains. This occurs by introducing a bulky residue into the CH3 domain of one of the half-life extension domains, which acts as the “knob.” Then, in order to accommodate the bulky residue, a “hole” is formed in the other half-life extension domain that can accommodate the knob. Either of the altered CH3 domains can be the “knob” while the other can be the “hole.” The introduction of a disulfide bridge further stabilizes the heterodimers (Merchant, A. M., et al, Nat. Biotechnol. (1998) 16(7); Atwell, S., et al, J. Mol. Biol. (1997) 270(1): 26-35) as well as increases yield.

It has been reported that heterodimerization yields above 97% can be achieved by introducing the S354C and T366W mutations in a heavy chain to create the “knob” and by introducing the Y349C, T366S, L368A, and Y407V mutations in a heavy chain to create the “hole” (numbering of the residues according to the Kabat EU numbering system). Carter et al. (2001), J. Immunol. Methods, 248: 7-15; Klein et al. (2012), MAbs, 4(6): 653-663.

In some embodiments comprising a first Fc polypeptide and a second Fc polypeptide, the first half-life Fc polypeptide comprises a heavy chain polypeptide or portion thereof (e.g., an Fc domain or fragment thereof) that comprises the amino acid mutations S354C and T366W (numbered according to the Kabat EU numbering system), and the second Fc polypeptide comprises a heavy chain polypeptide or portion thereof (e.g., an Fc domain or fragment thereof) that comprises the amino acid mutations Y349C, T366S, L368A, and Y407V (numbered according to the Kabat EU numbering system). In some embodiments comprising a first Fc polypeptide and a second Fc polypeptide, the first Fc polypeptide comprises a heavy chain polypeptide or portion thereof (e.g., an Fc domain or fragment thereof) that comprises the amino acid mutations Y349C, T366S, L368A, and Y407V (numbered according to the Kabat EU numbering system), and the second Fc polypeptide comprises a heavy chain polypeptide or portion thereof (e.g., an Fc domain or fragment thereof) that comprises the amino acid mutations S354C and T366W (numbered according to the Kabat EU numbering system).

Additional examples of substitutions that can be made to form knobs and holes include those described in US20140302037A1, the contents of which are herein incorporated by reference. For example, in some embodiments, any of the following amino acid substitutions can be made to a first Fc polypeptide (“first domain”) and a paired second Fc polypeptide (“second domain”) that each contain an Fc domain: (a) Y407T in the first domain and T366Y in the second domain; (b) Y407A in the first domain and T366W in the second domain; (c) F405A in the first domain and T394W in the second domain; (d) F405W in the first domain and T394S in the second domain; (e) Y407T in the first domain and T366Y in the second domain; (f) T366Y and F405A in the first domain and T394W and Y407T in the second domain; (g) T366W and F405W in the first domain and T394S and Y407A in the second domain; (h) F405W and Y407A in the first domain and T366W and T394S in the second domain; or (i) T366W in the first domain and T366S, L368A, and Y407V in the second domain, numbered according to the Kabat EU numbering system.

In some embodiments, any of the following amino acid substitutions can be made to a first Fc polypeptide (“first domain”) and a paired second Fc polypeptide (“second domain”) that each contain an Fc domain: (a) Y407T in the second domain and T366Y in the first domain; (b) Y407A in the second domain and T366W in the first domain; (c) F405A in the second domain and T394W in the first domain; (d) F405W in the second domain and T394S in the first domain; (e) Y407T in the second domain and T366Y in the first domain; (f) T366Y and F405A in the second domain and T394W and Y407T in the first domain; (g) T366W and F405W in the second domain and T394S and Y407A in the first domain; (h) F405W and Y407A in the second domain and T366W and T394S in the first domain; or (i) T366W in the second domain and T366S, L368A, and Y407V in the first domain, numbered according to the Kabat EU numbering system.

In embodiments comprising a first Fc polypeptide and a second Fc polypeptide that each comprise an Fc domain, any of the heterodimerizing alterations described herein can be used in the Fc domains to promote heterodimerization of any of the targeted cytokines described herein.

RF Mutation or CH3 Domain Swap for Heterodimeric Protein Purification

Two immunoglobulin heavy chains that differ by at least one amino acid allows isolation of the antigen-binding protein based on a differential affinity of an immunoglobulin heavy chain and a modified or mutated immunoglobulin heavy chain toward an affinity reagent. The antigen-binding proteins that have IgG CH2 and CH3 regions with different affinities with respect to Protein A allow rapid isolation by differential binding of the IgG regions to Protein A.

In one embodiment, a second Fc polypeptide comprises a 95R modification (by IMGT exon numbering; 435R by EU numbering) in the CH3 region. In another embodiment, a second Fc polypeptide further comprises a 96F modification (IMGT; 436F by EU). In some embodiments, a first Fc polypeptide comprises wild-type CH2 and CH3 domains derived from IgG1 or IgG4, and a second Fc polypeptide comprises 95R/96F modifications by IMGT exon numbering. In some embodiments, a first Fc polypeptide comprises wild-type CH2 and CH3 domains derived from IgG1 or IgG4, and a second Fc polypeptide comprises 435R/436F modifications by EU numbering.

In some embodiments, a first Fc polypeptide comprises wild-type CH2 and CH3 domains derived from IgG1 or IgG4, and a second Fc polypeptide comprises CH3 domain derived from IgG3.

In some embodiments, a first Fc polypeptide or a second Fc polypeptide comprises amino acid sequence of SEQ ID NO: 9. SEQ ID NO: 9 comprises “knob mutations” with CH3 domain from IgG3.

(SEQ ID NO. 9)
DKTHTCPPCPAPELLGGPSVFLFPPKPKDTLMISRTPEVTCVVVDVSHE
DPEVKFNWYVDGVEVHNAKTKPREEQYASTYRVVSVLTVLHQDWLNGKE
YKCKVSNKALPAPIEKTISKAKGQPREPQVYTLPPCREELTKNQVSLWC
LVKGFYPSDIAVEWESSGQPENNYKTTPPVLDSDGSFFLYSKLTVDKSR
WQQGNIFSCSVMHEALHNRFTQKSLSLSPGGSPG

In some embodiments, a first Fc polypeptide or a second Fc polypeptide comprises amino acid sequence of SEQ ID NO: 10. SEQ ID NO: 10 comprises “knob mutations” with “RF mutations (435R/436F).

(SEQ ID NO. 10)
DKTHTCPPCPAPELLGGPSVFLFPPKPKDTLMISRTPEVTCVVVDVSHE
DPEVKFNWYVDGVEVHNAKTKPREEQYASTYRVVSVLTVLHQDWLNGKE
YKCKVSNKALPAPIEKTISKAKGQPREPQVYTLPPCRDELTKNQVSLWC
LVKGFYPSDIAVEWESNGQPENNYKTTPPVLDSDGSFFLYSKLTVDKSR
WQQGNVFSCSVMHEALHNRFTQKSLSLSPGGSPG

In some embodiments, a first Fc polypeptide or a second Fc polypeptide comprises amino acid sequence of SEQ ID NO: 11.

(SEQ ID NO. 11)
DKTHTCPPCPAPELLGGPSVFLFPPKPKDTLMISRTPEVTCVVVDVSHE
DPEVKFNWYVDGVEVHNAKTKPREEQYASTYRVVSVLTVLHQDWLNGKE
YKCKVSNKALPAPIEKTISKAKGQPREPQVYTLPPCRDELTKNQVSLWC
LVKGFYPSDIAVEWESNGQPENNYKTTPPVLDSDGSFFLYSKLTVDKSR
WQQGNVFSCSVMHEALHNRFTQKSLSLSPG

In some embodiments, the protease cleavage sites as described herein may be introduced into any one of SEQ ID NOs: 9-11.

In some embodiments, a first Fc polypeptide or a second Fc polypeptide comprises amino acid sequence having at least about 85% identity to SEQ ID NO: 11. In some embodiments, a first Fc polypeptide or a second Fc polypeptide comprises amino acid sequence having at least about 90% identity to SEQ ID NO: 11. In some embodiments, a first Fc polypeptide or a second Fc polypeptide comprises amino acid sequence having at least about 91% identity to SEQ ID NO: 11. In some embodiments, a first Fc polypeptide or a second Fc polypeptide comprises amino acid sequence having at least about 92% identity to SEQ ID NO: 11. In some embodiments, a first Fc polypeptide or a second Fc polypeptide comprises amino acid sequence having at least about 93% identity to SEQ ID NO: 11. In some embodiments, a first Fc polypeptide or a second Fc polypeptide comprises amino acid sequence having at least about 94% identity to SEQ ID NO: 11. In some embodiments, a first Fc polypeptide or a second Fc polypeptide comprises amino acid sequence having at least about 95% identity to SEQ ID NO: 11. In some embodiments, a first Fc polypeptide or a second Fc polypeptide comprises amino acid sequence having at least about 96% identity to SEQ ID NO: 11. In some embodiments, a first Fc polypeptide or a second Fc polypeptide comprises amino acid sequence having at least about 97% identity to SEQ ID NO: 11. In some embodiments, a first Fc polypeptide or a second Fc polypeptide comprises amino acid sequence having at least about 98% identity to SEQ ID NO: 11. In some embodiments, a first Fc polypeptide or a second Fc polypeptide comprises amino acid sequence having at least about 99% identity to SEQ ID NO: 11.

In some embodiments, a first Fc polypeptide or a second Fc polypeptide comprises amino acid sequence having at least about 85% identity to SEQ ID NO: 5. In some embodiments, a first Fc polypeptide or a second Fc polypeptide comprises amino acid sequence having at least about 90% identity to SEQ ID NO: 5. In some embodiments, a first Fc polypeptide or a second Fc polypeptide comprises amino acid sequence having at least about 91% identity to SEQ ID NO: 5. In some embodiments, a first Fc polypeptide or a second Fc polypeptide comprises amino acid sequence having at least about 92% identity to SEQ ID NO: 5. In some embodiments, a first Fc polypeptide or a second Fc polypeptide comprises amino acid sequence having at least about 93% identity to SEQ ID NO: 5. In some embodiments, a first Fc polypeptide or a second Fc polypeptide comprises amino acid sequence having at least about 94% identity to SEQ ID NO: 5. In some embodiments, a first Fc polypeptide or a second Fc polypeptide comprises amino acid sequence having at least about 95% identity to SEQ ID NO: 5. In some embodiments, a first Fc polypeptide or a second Fc polypeptide comprises amino acid sequence having at least about 96% identity to SEQ ID NO: 5. In some embodiments, a first Fc polypeptide or a second Fc polypeptide comprises amino acid sequence having at least about 97% identity to SEQ ID NO: 5. In some embodiments, a first Fc polypeptide or a second Fc polypeptide comprises amino acid sequence having at least about 98% identity to SEQ ID NO: 5. In some embodiments, a first Fc polypeptide or a second Fc polypeptide comprises amino acid sequence having at least about 99% identity to SEQ ID NO: 5.

Masked or Targeted Cytokine

In accordance with the present disclosure, the engineered cleavable Fc domain comprising a tumor-associated protease cleavage site can be fused to a masked cytokine comprising a cytokine molecule and a masking moiety. Alternatively, the engineered cleavable Fc domain comprising a tumor-associated protease cleavage site can be fused to a targeted cytokine comprising a cytokine molecule, a masking moiety, and a targeting moiety. Upon the cleavage of the Fc domain, e.g., at the protease cleavage site, the masked and targeted cytokines are released and become active at the site of disease, and are able to specifically target a cell of interest for effective treatment of cancer without causing undesired side effects.

In some embodiments, a cytokine is linked to an engineered cleavable Fc domain comprising a tumor-associated protease cleavage site, via a non-cleavable linker. In some embodiments, a masking moiety is linked to an engineered cleavable Fc domain via a non-cleavable linker. In some embodiments, a targeting moiety is linked to an engineered Fc domain with or without non-cleavable linker.

In other embodiments, the engineered cleavable Fc domain of the present invention is directly linked to a cytokine, a masking moiety, or a targeting moiety without a linker.

Cytokines

The immune system is skilled in communication and designed to respond quickly, specifically and globally to protect an organism against foreign invaders and disease. The cytokine superfamily of proteins is an integral part of the signalling network between cells and is essential in generating and regulating the immune system. These interacting biological signals have remarkable capabilities, such as influencing growth and development, hematopoiesis, lymphocyte recruitment, T cell subset differentiation and inflammation.

Cytokines can be part of a bigger immune program, e.g., T cell subset differentiation. Mature CD4 and CD8 T cells leave the thymus with a naive phenotype and produce a variety of cytokines. In the periphery, these T cells encounter antigen presenting cells (APCs) displaying either major histocompatibility complex (MHC) class I molecules (present peptides generated in the cytosol to CD8 T cells) or MHC class II molecules (present peptides degraded in intracellular vesicles to CD4 T cells). Following activation, characteristic cytokine and chemokine secretion profiles allow the classification of CD4 T helper (Th) cells into two major subpopulations in mice and humans.3-7Th1 cells secrete mainly IL-2, interferon-7 (IFN-7) and tumor necrosis factor-O (TNF-0), whereas Th2 cells secrete mainly IL-4, IL-5, IL-6, IL-10 and IL-13. Th1 cells support cell-mediated immunity and as a consequence promote inflammation, cytotoxicity and delayed-type hypersensitivity (DTH). Th2 cells support humoral immunity and serve to downregulate the inflammatory actions of Th1 cells. This paradigm is a great example of an integrated biological network and is very useful in simplifying our understanding of typical immune responses and those that turn pathogenic. For example, the failure to communicate “self” can lead to a loss of tolerance to our own antigens and prompt destructive immune responses to self-tissues and autoimmune disease. Autoimmunity, the major focus of this book, is the underlying mechanism of a set of conditions, such as type 1 diabetes mellitus, multiple sclerosis and rheumatoid arthritis. Autoimmune diseases may be caused in part by cytokine- and chemokine-mediated dysregulation of Th cell subset differentiation. The main factors affecting the development of Th subsets, aside from the context in which the antigen and costimulatory signals are presented, are the cytokines and chemokines in the stimulatory milieu. A better understanding of the properties and interactions of the individual cytokines and chemokines that play a role in Th cell activation may lead to more advanced treatments for autoimmune disease.

The targeted cytokine of the present invention can comprise any cytokine or a variant thereof that is known in the art. See, e.g., Cameron MJ, Kelvin DJ. Cytokines, Chemokines and Their Receptors. In: Madame Curie Bioscience Database. Austin (TX): Landes Bioscience; 2000-2013, the contents of which is incorporated in its entirety. For example, a cytokine incorporated in the targeted cytokine can be IL-1, IL-2, IL-3, IL-4, IL-5, IL-6, IL-7, IL-9, IL-10, IL-11, IL-12, IL-13, IL-14, IL-15, IL-16, IL-17, IL-18, IL-20, IL-21, IL-33, TNF-α, TNF-0, CXCL8 (IL-8), G-CSF, GM-CSF, LIF, OSM, IFN-α, IFN-0, IFN-7, CD154, LT-0, 4-1BBL, APRIL, CD70, CD153, CD178, GITRL, LIGHT, OX40L, TALL-1, TRAIL, TWEAK, TRANCE, TGF-β, M-CSF, or MSP or a fragment thereof.

In some embodiments, a cytokine is IL-1 or a variant thereof. In some embodiments, a cytokine is IL-2 or a variant thereof. In some embodiments, a cytokine is IL-3 or a variant thereof. In some embodiments, a cytokine is IL-4 or a variant thereof. In some embodiments, a cytokine is IL-5 or a variant thereof. In some embodiments, a cytokine is IL-6 or a variant thereof. In some embodiments, a cytokine is IL-7 or a variant thereof. In some embodiments, a cytokine is IL-9 or a variant thereof. In some embodiments, a cytokine is IL-10 or a variant thereof. In some embodiments, a cytokine is IL-11 or a variant thereof. In some embodiments, a cytokine is IL-12 or a variant thereof. In some embodiments, a cytokine is IL-13 or a variant thereof. In some embodiments, a cytokine is IL-14 or a variant thereof. In some embodiments, a cytokine is IL-15 or a variant thereof. In some embodiments, a cytokine is IL-16 or a variant thereof. In some embodiments, a cytokine is IL-17 or a variant thereof. In some embodiments, a cytokine is IL-18 or a variant thereof. In some embodiments, a cytokine is IL-20 or a variant thereof. In some embodiments, a cytokine is TNF-α or a variant thereof. In some embodiments, a cytokine TNF-0 or a variant thereof. In some embodiments, a cytokine is CXCL8 (IL-8) or a variant thereof. In some embodiments, a cytokine is G-CSF or a variant thereof. In some embodiments, a cytokine is GM-CSF or a variant thereof. In some embodiments, a cytokine is LIF or a variant thereof. In some embodiments, a cytokine is OSM or a variant thereof. In some embodiments, a cytokine is IFN-α or a variant thereof. In some embodiments, a cytokine is IFN-β or a variant thereof. In some embodiments, a cytokine is IFN-7 or a variant thereof. In some embodiments, a cytokine is CD154 or a variant thereof. In some embodiments, a cytokine is LT-0 or a variant thereof. In some embodiments, a cytokine is 4-1BBL or a variant thereof. In some embodiments, a cytokine is APRIL or a variant thereof. In some embodiments, a cytokine is CD153 or a variant thereof. In some embodiments, a cytokine is CD70 or a variant thereof. In some embodiments, a cytokine is CD178 or a variant thereof. In some embodiments, a cytokine is GITRL or a variant thereof. In some embodiments, a cytokine is LIGHT or a variant thereof. In some embodiments, a cytokine is OX40L or a variant thereof. In some embodiments, a cytokine is TALL-1 or a variant thereof. In some embodiments, a cytokine is TRAIL or a variant thereof. In some embodiments, a cytokine is TWEAK or a variant thereof. In some embodiments, a cytokine is TRANCE or a variant thereof. In some embodiments, a cytokine is TGF-β or a variant thereof. In some embodiments, a cytokine is M-CSF or a variant thereof. In some embodiments, a cytokine is MSP or a variant thereof.

Interleukin 2 (IL-2)

Provided herein is an IL-2 cytokine or functional fragment thereof for use in a targeted cytokine or cleavage product thereof. A cytokine plays a role in cellular signalling, particularly in cells of the immune system. IL-2 is an interleukin, which is a type of cytokine signalling molecule in the immune system that regulates activities of white blood cells. Suitable IL-2 cytokines for use in the present invention can be any IL-2 or functional fragment thereof. In some embodiments, the IL-2 is naturally occurring IL-2, an IL-2 comprising one or more substitutions (e.g., an IL-2 mutein, or IL-2 variant), or truncated IL-2. In some embodiments, the IL-2 is polypeptide that retains at least one property of IL-2 biological activity.

In some embodiments, the IL-2 is naturally occurring IL-2. In some embodiments, the IL-2 comprises C125A substitution of mature IL-2 (SEQ ID NO: 13).

In some embodiments, the amino acid substitutions reduce the affinity of the IL-2 polypeptide or functional fragment thereof for CD25 (IL-2Rα).

In some embodiments, the IL-2 polypeptide or functional fragment thereof comprises an amino acid sequence produced by introducing one or more amino acid substitutions into the amino acid sequence of the IL-2 polypeptide or functional fragment thereof that increases the affinity of the IL-2 polypeptide or functional fragment thereof for IL-2Rb or IL-2Rγ. In some embodiments, the IL-2 polypeptide or functional fragment thereof comprises an amino acid sequence having one or more amino acid substitutions compared to the amino acid sequence of wildtype IL-2 that enhances the affinity of the IL-2 polypeptide or functional fragment thereof for IL-2Rb (CD 122). In some embodiments, the IL-2 polypeptide or functional fragment thereof comprises an amino acid sequence having one or more amino acid substitutions compared to the amino acid sequence of wildtype IL-2 that reduces the affinity of the IL-2 peptide or functional fragment thereof for IL-2Ra (CD25), and one or more amino acid substitutions compared to the amino acid sequence of wildtype IL-2 that enhances the affinity of the IL-2 polypeptide or functional fragment thereof for IL-2R (CD122).

In some embodiments, the IL-2 binds to IL-2Ra with an affinity similar to or higher than wildtype IL-2. In some embodiments, the IL-2 preferentially binds to CD25 (e.g., alpha-biased). In some embodiments, the IL-2 has reduced affinity for CD122 and/or CD132. In some embodiments, the IL-2 comprises N88D and C125A relative to SEQ ID NO: 13.

In eukaryotic cells, naturally occurring IL-2 is synthesized as a precursor polypeptide of 153 amino acids, which has SEQ ID NO: 12.

(SEQ ID NO: 12)
MYRMQLLSCIALSLALVTNSAPTSSSTKKTQLQLEHLLLDLQMILNGIN
NYKNPKLTRMLTFKFYMPKKATELKHLQCLEEELKPLEEVLNLAQSKNF
HLRPRDLISNINVIVLELKGSETTFMCEYADETATIVEFLNRWITFCQS
IISTLT

This is then processed into mature IL-2 by the removal of amino acid residues 1-20. This results in a mature form of IL-2 consisting of 133 amino acids (amino acid residues 21-153), which has the sequence presented by SEQ ID NO: 13.

(SEQ ID NO: 13)
APTSSSTKKTQLQLEHLLLDLQMILNGINNYKNPKLTRMLTFKFYMPKK
ATELKHLQCLEEELKPLEEVLNLAQSKNFHLRPRDLISNINVIVLELKG
SETTFMCEYADETATIVEFLNRWITFAQSIISTLT

“Functional fragments” of an IL-2 cytokine comprise a portion of a full length cytokine protein which retains or has modified cytokine receptor binding capability (e.g., within at least 50%, 80%, 90%, 95%, 96%, 97%, 98%, 99% or 100% activity compared to the full length cytokine protein). Cytokine receptor binding capability can be shown, for example, by the capability of a cytokine to bind to the cytokine's cognate receptor or a component thereof (e.g., one or more chain(s) of a heterotrimeric receptor complex).

In some embodiments, the IL-2 cytokine or functional fragment thereof is any naturally occurring interleukin-2 (IL-2) protein or modified variant thereof capable of binding to an interleukin-2 receptor, particularly the IL-2Rα chain. In the context of IL-2 cytokine binding, the target protein could be IL-2R (comprising the IL-2Rα, IL-2Rβ, and IL-2Rγ chains), the IL-2Rα chain, the IL-2Rβ chain, or the IL-2Rα/β dimeric complex. In some embodiments, the IL-2 cytokine or functional fragment thereof comprises the amino acid sequence of amino acid residues 21-153 of SEQ ID NO: 13. In some embodiments, the IL-2 polypeptide or functional fragment thereof comprises the amino acid sequence of mature IL-2, SEQ ID NO: 13.

In some embodiments, the IL-2 cytokine or functional fragment thereof comprises an amino acid sequence having at least one amino acid modification as compared to the amino acid sequence of SEQ ID NO: 13. Each of the at least one amino acid modifications can be any amino acid modification, such as a substitution, insertion, or deletion. In some embodiments, the IL-2 cytokine or functional fragment thereof comprises an amino acid sequence having at least 1, at least 2, at least 3, at least 4, at least 5, at least 6, at least 7, at least 8, at least 9, or at least 10 amino acid substitutions as compared to the amino acid sequence of SEQ ID NO: 13. In some embodiments, the IL-2 cytokine or functional fragment thereof comprises an amino acid sequence having at least 5 amino acid substitutions as compared to the amino acid sequence of SEQ ID NO: 13.

In some embodiments, the IL-2 cytokine or functional fragment thereof comprises an amino acid sequence having at least 80% sequence identity to SEQ ID NO: 13. In some embodiments, the IL-2 cytokine or functional fragment thereof comprises an amino acid sequence having at least 85% sequence identity to SEQ ID NO: 13. In some embodiments, the IL-2 cytokine or functional fragment thereof comprises an amino acid sequence having at least 90% sequence identity to SEQ ID NO: 13. In some embodiments, the IL-2 cytokine or functional fragment thereof comprises an amino acid sequence having at least 91% sequence identity to SEQ ID NO: 13. In some embodiments, the IL-2 cytokine or functional fragment thereof comprises an amino acid sequence having at least 92% sequence identity to SEQ ID NO: 13. In some embodiments, the IL-2 cytokine or functional fragment thereof comprises an amino acid sequence having at least 93% sequence identity to SEQ ID NO: 13. In some embodiments, the IL-2 cytokine or functional fragment thereof comprises an amino acid sequence having at least 94% sequence identity to SEQ ID NO: 13. In some embodiments, the IL-2 cytokine or functional fragment thereof comprises an amino acid sequence having at least 95% sequence identity to SEQ ID NO: 13. In some embodiments, the IL-2 cytokine or functional fragment thereof comprises an amino acid sequence having at least 96% sequence identity to SEQ ID NO: 13. In some embodiments, the IL-2 cytokine or functional fragment thereof comprises an amino acid sequence having at least 97% sequence identity to SEQ ID NO: 13. In some embodiments, the IL-2 cytokine or functional fragment thereof comprises an amino acid sequence having at least 98% sequence identity to SEQ ID NO: 13. In some embodiments, the IL-2 cytokine or functional fragment thereof comprises an amino acid sequence having at least 99% sequence identity to SEQ ID NO: 13.

In some embodiments, the IL-2 cytokine or functional fragment thereof comprises an amino acid sequence having one or more amino acid substitutions as compared to the amino acid sequence of wild-type IL-2 of SEQ ID NO: 13 that reduces the affinity of the IL-2 peptide or functional fragment thereof for IL-2Rα (CD25). In some embodiments, the IL-2 cytokine or functional fragment thereof comprises an amino acid sequence having one or more amino acid substitutions as compared to the amino acid sequence of SEQ ID NO: 13, such that one or more of amino acid residues 38, 42, 45, and 62 is an alanine (A). In some embodiments, the IL-2 cytokine or functional fragment thereof comprises an amino acid sequence having one or more amino acid substitutions as compared to the amino acid sequence of SEQ ID NO: 13, such that amino acid residues 38, 42, 45, and 62 are an alanine (A).

In some embodiments, the IL-2 cytokine or functional fragment thereof comprises amino acid sequence substitution C125A as compared to the amino acid sequence of SEQ ID NO: 13.

In some embodiments, the IL-2 cytokine or functional fragment thereof comprises an amino acid sequence having one or more amino acid substitutions as compared to the amino acid sequence of SEQ ID NO: 13, such that amino acid residues 38, 42, 45, and 62 are an alanine (A) and amino acid residue 125 is a alanine (A). In some embodiments, the IL-2 cytokine or functional fragment thereof comprises an amino acid sequence having amino acid residues R38, F42, Y45, and E62 substituted for alanine in the amino acid sequence of SEQ ID NO: 13. In some embodiments, the IL-2 cytokine or functional fragment thereof comprises an amino acid sequence having amino acid residues R38, F42, Y45, and E62 substituted for alanine (A) and amino acid residue C125 substituted for alanine (A) in the amino acid sequence of SEQ ID NO: 13.

In some embodiments, the IL-2 cytokine or functional fragment thereof comprises the amino acid sequence of SEQ ID NO: 14.

(SEQ ID NO: 14)
APTSSSTKKTQLQLEHLLLDLQMILNGINNYKNPKLTAMLTAKFAMPKK
ATELKHLQCLEEALKPLEEVLNLAQSKNFHLRPRDLISNINVIVLELKG
SETTFMCEYADETATIVEFLNRWITFAQSIISTLT

In some embodiments, the IL-2 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 14. In some embodiments, the IL-2 cytokine or functional fragment thereof comprises an amino acid sequence having at least about 80% identity to SEQ ID NO: 14. In some embodiments, the IL-2 cytokine or functional fragment thereof comprises an amino acid sequence having at least about 85% identity to SEQ ID NO: 14. In some embodiments, the IL-2 cytokine or functional fragment thereof comprises an amino acid sequence having at least about 90% identity to SEQ ID NO: 14. In some embodiments, the IL-2 cytokine or functional fragment thereof comprises an amino acid sequence having at least about 92% identity to SEQ ID NO: 14. In some embodiments, the IL-2 cytokine or functional fragment thereof comprises an amino acid sequence having at least about 95% identity to SEQ ID NO: 14. In some embodiments, the IL-2 cytokine or functional fragment thereof comprises an amino acid sequence having at least about 97% identity to SEQ ID NO: 14. In some embodiments, the IL-2 cytokine or functional fragment thereof comprises an amino acid sequence having at least about 99% identity to SEQ ID NO: 14.

In some embodiments, the IL-2 cytokine or functional fragment thereof comprises an amino acid sequence having one or more amino acid substitutions as compared to the amino acid sequence of SEQ ID NO: 13, such that amino acid residue 88 is an aspartic acid (D) and amino acid residue 125 is an alanine (A). In some embodiments, the IL-2 cytokine or functional fragment thereof comprises an amino acid sequence having amino acid residue N88 substituted for aspartic acid in the amino acid sequence of SEQ ID NO: 13.

In some embodiments, the IL-2 cytokine or functional fragment thereof comprises the amino acid sequence of SEQ ID NO: 118.

(SEQ ID NO: 118)
APTSSSTKKTQLQLEHLLLDLQMILNGINNYKNPKLTRMLTFKFYMPKK
ATELKHLQCLEEELKPLEEVLNLAQSKNFHLRPRDLISDINVIVLELKG
SETTFMCEYADETATIVEFLNRWITFAQSIISTLT

In some embodiments, the IL-2 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 118. In some embodiments, the IL-2 cytokine or functional fragment thereof comprises an amino acid sequence having at least about 80% identity to SEQ ID NO: 118. In some embodiments, the IL-2 cytokine or functional fragment thereof comprises an amino acid sequence having at least about 85% identity to SEQ ID NO: 118. In some embodiments, the IL-2 cytokine or functional fragment thereof comprises an amino acid sequence having at least about 90% identity to SEQ ID NO: 118. In some embodiments, the IL-2 cytokine or functional fragment thereof comprises an amino acid sequence having at least about 92% identity to SEQ ID NO: 118. In some embodiments, the IL-2 cytokine or functional fragment thereof comprises an amino acid sequence having at least about 95% identity to SEQ ID NO: 118. In some embodiments, the IL-2 cytokine or functional fragment thereof comprises an amino acid sequence having at least about 97% identity to SEQ ID NO: 118. In some embodiments, the IL-2 cytokine or functional fragment thereof comprises an amino acid sequence having at least about 99% identity to SEQ ID NO: 118.

In some embodiments, the IL-2 cytokine or functional fragment thereof has one or more amino acid residues e.g. residues 1-3 removed as compared to the amino acid sequence of the mature IL-2 of SEQ ID NO: 13, for the purpose of removing an O-glycosylation site. In some embodiments, the IL-2 cytokine or functional fragment thereof has one or more amino acid residues substituted as compared to the amino acid sequence of the mature IL-2 of SEQ ID NO: 13, for the purpose of removing an O-glycosylation site. In some embodiments, the IL-2 cytokine or functional fragment thereof has one or more amino acid residues inserted, e.g. in the region of residues 1-3, as compared to the amino acid sequence of the mature IL-2 of SEQ ID NO: 13, for the purpose of removing an O-glycosylation site. In some embodiments, the IL-2 cytokine or functional fragment thereof does not have an O-glycosylation site within residues 1-3.

Interleukin 15 (IL-15)

Provided herein is an IL-15 cytokine or functional fragment thereof for use in a targeted cytokine or cleavage product thereof. A cytokine plays a role in cellular signalling, particularly in cells of the immune system. IL-15 is an interleukin, which is a type of cytokine signalling molecule in the immune system that regulates activities of white blood cells.

In eukaryotic cells, IL-15 is synthesized as a precursor polypeptide of 162 amino acids (SEQ ID NO: 15), which is then processed into mature IL-15 by the removal of amino acid residues 1-48. This results in a mature form of IL-15 consisting of 114 amino acids (amino acid residues 49-162) that is secreted in a mature, active form (see SEQ ID NO: 16).

IL-15 precursor polypeptide (SEQ ID NO: 15):
MRISKPHLRSISIQCYLCLLLNSHFLTEAGIHVFILGCFSAGLPKTEAN
WVNVISDLKKIEDLIQSMHIDATLYTESDVHPSCKVTAMKCFLLELQVI
SLESGDASIHDTVENLIILANNSLSSNGNVTESGCKECEELEEKNIKEF
LQSFVHIVQMFINTS
IL-15 mature polypeptide (SEQ ID NO: 16):
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS

The term “IL-15” or “IL-15 polypeptide” as used herein refers to any interleukin-15 (IL-15) protein, or a functional fragment or variant thereof. The term encompasses any native IL-15 from any vertebrate source, including mammals such as primates (e.g., humans) and rodents (e.g., rats and mice). The term encompasses unprocessed IL-15 (e.g., a full length, precursor form of IL-15 that consists of amino acid residues 1-162) as well as any form of IL-15 that results from processing in the cell (e.g., a mature form of IL-15 that consists of amino acid residues 49-162). As such, the term encompasses a protein encoded by the amino acid sequence of SEQ ID NO: 16, as well as sequence variants thereof. The term also encompasses naturally occurring variants of IL-15. The term also encompasses non-naturally occurring variants of IL-15, such as truncations, deletions, forms where IL-15 is linked to another molecule, and variants caused by at least one amino acid change to the amino acid sequence (e.g., by substitution, addition, or deletion). In some aspects, the variants or homologs have at least 90%, 95%, 96%, 97%, 98%, 99% or 100% amino acid sequence identity across the whole sequence or a portion of the sequence (e.g., a 50, 100, or 114 continuous amino acid portion) compared to a naturally occurring IL-15 polypeptide, such as an IL-15 polypeptide encoded by the amino acid sequence of SEQ ID NO: 15 or 16. As such, the term “IL-15” or “IL-15 polypeptide” includes an IL-15 protein comprising the amino acid sequence of SEQ ID NO: 15 or 16, including variants thereof, such as variants created by one or more amino acid substitutions to the amino acid sequence of SEQ ID NO: 15 or 16.

“Functional fragments” of an IL-15 cytokine comprise a portion of a full length cytokine protein which retains or has modified cytokine receptor binding capability (e.g., within at least 50%, 80%, 90%, 95%, 96%, 97%, 98%, 99% or 100% activity compared to the full length cytokine protein). Cytokine receptor binding capability can be shown, for example, by the capability of a cytokine to bind to the cytokine's cognate receptor or a component thereof (e.g., one or more chain(s) of a heterotrimeric receptor complex).

In some embodiments, the IL-15 cytokine or functional fragment thereof is any naturally occurring interleukin-2 (IL-15) protein or modified variant thereof capable of binding to an interleukin-2 receptor, particularly the IL-15Rα chain.

In some embodiments, the IL-15 cytokine or fragment thereof comprises SEQ ID NO: 16 or a functional fragment thereof.

In some embodiments, the IL-15 cytokine or functional fragment thereof comprises an amino acid sequence of SEQ ID NO: 16.

In some embodiments, the IL-15 cytokine or functional fragment thereof comprises an amino acid sequence having at least one amino acid modification as compared to the amino acid sequence of SEQ ID NO: 16. Each of the at least one amino acid modifications can be any amino acid modification, such as a substitution, insertion, or deletion. In some embodiments, the IL-15 cytokine or functional fragment thereof comprises an amino acid sequence having at least 1, at least 2, at least 3, at least 4, at least 5, at least 6, at least 7, at least 8, at least 9, or at least 10 amino acid substitutions as compared to the amino acid sequence of SEQ ID NO: 16. In some embodiments, the IL-15 cytokine or functional fragment thereof comprises an amino acid sequence having at least 5 amino acid substitutions as compared to the amino acid sequence of SEQ ID NO: 16. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 16.

In some embodiments, the IL-15 cytokine or functional fragment thereof comprises an amino acid sequence having at least about 85% sequence identity to SEQ ID NO: 16. In some embodiments, the IL-15 cytokine or functional fragment thereof comprises an amino acid sequence having at least about 90% sequence identity to SEQ ID NO: 16. In some embodiments, the IL-15 cytokine or functional fragment thereof comprises an amino acid sequence having at least about 92% sequence identity to SEQ ID NO: 16. In some embodiments, the IL-15 cytokine or functional fragment thereof comprises an amino acid sequence having at least about 93% sequence identity to SEQ ID NO: 16. In some embodiments, the IL-15 cytokine or functional fragment thereof comprises an amino acid sequence having at least about 94% sequence identity to SEQ ID NO: 16. In some embodiments, the IL-15 cytokine or functional fragment thereof comprises an amino acid sequence having at least about 95% sequence identity to SEQ ID NO: 16. In some embodiments, the IL-15 cytokine or functional fragment thereof comprises an amino acid sequence having at least about 96% sequence identity to SEQ ID NO: 16. In some embodiments, the IL-15 cytokine or functional fragment thereof comprises an amino acid sequence having at least about 97% sequence identity to SEQ ID NO: 16. In some embodiments, the IL-15 cytokine or functional fragment thereof comprises an amino acid sequence having at least about 98% sequence identity to SEQ ID NO: 16. In some embodiments, the IL-15 cytokine or functional fragment thereof comprises an amino acid sequence having at least about 99% sequence identity to SEQ ID NO: 16.

In some embodiments, the IL-15 cytokine or functional fragment thereof comprises an amino acid sequence having one or more amino acid substitutions as compared to the amino acid sequence of SEQ ID NO: 16.

In some embodiments, the IL-15 cytokine or functional fragment thereof comprises an amino acid sequence having one or more amino acid substitutions at positions D22, E46, E53 as compared to the amino acid sequence of SEQ ID NO: 16. In some embodiments, the IL-15 cytokine or functional fragment thereof comprises an amino acid sequence having one or more amino acid substitutions at positions D22, E46, E53, N71, N79, and N112 as compared to the amino acid sequence of SEQ ID NO: 16.

In some embodiments, the IL-15 cytokine or functional fragment thereof comprises an amino acid sequence having an amino acid substitution at position D22 as compared to the amino acid sequence of SEQ ID NO: 16.

In some embodiments, the IL-15 cytokine or functional fragment thereof comprises an amino acid sequence having an amino acid substitution at position E46 as compared to the amino acid sequence of SEQ ID NO: 16.

In some embodiments, the IL-15 cytokine or functional fragment thereof comprises an amino acid sequence having an amino acid substitution at position E53 as compared to the amino acid sequence of SEQ ID NO: 16.

In some embodiments, the IL-15 cytokine or functional fragment thereof comprises an amino acid sequence having an amino acid substitution at position N71 as compared to the amino acid sequence of SEQ ID NO: 16.

In some embodiments, the IL-15 cytokine or functional fragment thereof comprises an amino acid sequence having an amino acid substitution at position N79 as compared to the amino acid sequence of SEQ ID NO: 16.

In some embodiments, the IL-15 cytokine or functional fragment thereof comprises an amino acid sequence having an amino acid substitution at position N112 as compared to the amino acid sequence of SEQ ID NO: 16.

In some embodiments, the IL-15 cytokine or functional fragment thereof comprises an amino acid sequence having amino acid substitutions at positions E46 and E53 as compared to the amino acid sequence of SEQ ID NO: 16.

In some embodiments, the IL-15 cytokine or functional fragment thereof comprises an amino acid sequence having an amino acid substitution at position N71 and N79 as compared to the amino acid sequence of SEQ ID NO: 16.

In some embodiments, the IL-15 cytokine or functional fragment thereof comprises an amino acid sequence having an amino acid substitution at position N71 and N112 as compared to the amino acid sequence of SEQ ID NO: 16.

In some embodiments, the IL-15 cytokine or functional fragment thereof comprises an amino acid sequence having an amino acid substitution at position N79 and N112 as compared to the amino acid sequence of SEQ ID NO: 16.

In some embodiments, the IL-15 cytokine or functional fragment thereof comprises an amino acid sequence having an amino acid substitution at position N71, N79 and N112 as compared to the amino acid sequence of SEQ ID NO: 16.

In some embodiments, the amino acid substitution at position D22 is D22A. In some embodiments, the amino acid substitution at position E46 is E46A. In some embodiments, the amino acid substitution at position E46 is E46R. In some embodiments, the amino acid substitution at position E46 is E46S. In some embodiments, the amino acid substitution at position E53 is E53A, E53R, or E53S. In some embodiments, the amino acid substitution at position N71 is N71Q. In some embodiments, the amino acid substitution at position N79 is N79Q. In some embodiments, the amino acid substitution at position N112 is N112Q.

In some embodiments, the IL-15 cytokine or functional fragment thereof comprises an amino acid sequence having an amino acid substitution D22A as compared to the amino acid sequence of SEQ ID NO: 16.

In some embodiments, the IL-15 cytokine or functional fragment thereof comprises an amino acid sequence of SEQ ID NO: 17.

(SEQ ID NO: 17)
NWVNVISDLKKIEDLIQSMHIAATLYTESDVHPSCKVTAMKCFLLELQV
ISLESGDASIHDTVENLIILANNSLSSNGNVTESGCKECEELEEKNIKE
FLQSFVHIVQMFINTS

In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 17.

In some embodiments, the IL-15 cytokine or functional fragment thereof comprises an amino acid sequence having an amino acid substitution E46A as compared to the amino acid sequence of SEQ ID NO: 16. In some embodiments, the IL-15 cytokine or functional fragment thereof comprises an amino acid sequence of SEQ ID NO: 18.

(SEQ ID NO: 18)
NWVNVISDLKKIEDLIQSMHIDATLYTESDVHPSCKVTAMKCFLLALQV
ISLESGDASIHDTVENLIILANNSLSSNGNVTESGCKECEELEEKNIKE
FLQSFVHIVQMFINTS

In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 18.

In some embodiments, the IL-15 cytokine or functional fragment thereof comprises an amino acid sequence having amino acid substitutions E46A and E53A as compared to the amino acid sequence of SEQ ID NO: 16. In some embodiments, the IL-15 cytokine or functional fragment thereof comprises an amino acid sequence of SEQ ID NO: 19. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 19.

(SEQ ID NO: 19)
NWVNVISDLKKIEDLIQSMHIDATLYTESDVHPSCKVTAMKCFLLALQV
ISLASGDASIHDTVENLIILANNSLSSNGNVTESGCKECEELEEKNIKE
FLQSFVHIVQMFINTS

In some embodiments, the IL-15 cytokine or functional fragment thereof comprises an amino acid sequence having amino acid substitutions E46R and E53R as compared to the amino acid sequence of SEQ ID NO: 16. In some embodiments, the IL-15 cytokine or functional fragment thereof comprises an amino acid sequence of SEQ ID NO: 20. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 20.

(SEQ ID NO: 20)
NWVNVISDLKKIEDLIQSMHIDATLYTESDVHPSCKVTAMKCFLLRLQV
ISLRSGDASIHDTVENLIILANNSLSSNGNVTESGCKECEELEEKNIKE
FLQSFVHIVQMFINTS

In some embodiments, the IL-15 cytokine or functional fragment thereof comprises an amino acid sequence having amino acid substitutions E46S and E53S as compared to the amino acid sequence of SEQ ID NO: 16. In some embodiments, the IL-15 cytokine or functional fragment thereof comprises an amino acid sequence of SEQ ID NO: 21. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 21.

(SEQ ID NO: 21)
NWVNVISDLKKIEDLIQSMHIDATLYTESDVHPSCKVTAMKCFLLSLQV
ISLSSGDASIHDTVENLIILANNSLSSNGNVTESGCKECEELEEKNIKE
FLQSFVHIVQMFINTS

In some embodiments, the IL-15 cytokine or functional fragment thereof comprises an amino acid sequence having an amino acid substitution E53A as compared to the amino acid sequence of SEQ ID NO: 16. In some embodiments, the IL-15 cytokine or functional fragment thereof comprises an amino acid sequence of SEQ ID NO: 22. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 22.

(SEQ ID NO: 22)
NWVNVISDLKKIEDLIQSMHIDATLYTESDVHPSCKVTAMKCFLLELQV
ISLASGDASIHDTVENLIILANNSLSSNGNVTESGCKECEELEEKNIKE
FLQSFVHIVQMFINTS

In some embodiments, the IL-15 cytokine or functional fragment thereof comprises an amino acid sequence of SEQ ID NO: 23 and an amino acid sequence of SEQ ID NO: 24. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 23 and an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 24.

(SEQ ID NO: 23)
NWVNVISDLKKIEDLIQS
(SEQ ID NO: 24)
KVTAMKCFLLELQVISLESGDASIHDTVENLIILANNSLSSNGNVTESG
CKECEELEEKNIKEFLQSFVHIVQMFINTS

In some embodiments, the IL-15 cytokine or functional fragment thereof comprises an amino acid sequence having an amino acid substitution N71Q as compared to the amino acid sequence of SEQ ID NO: 16. In some embodiments, the IL-15 cytokine or functional fragment thereof comprises an amino acid sequence of SEQ ID NO: 102. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 25.

(SEQ ID NO: 25)
NWVNVISDLKKIEDLIQSMHIDATLYTESDVHPSCKVTAMKCFLLELQV
ISLESGDASIHDTVENLIILAQNSLSSNGNVTESGCKECEELEEKNIKE
FLQSFVHIVQMFINTS

In some embodiments, the IL-15 cytokine or functional fragment thereof comprises an amino acid sequence having an amino acid substitution N79Q as compared to the amino acid sequence of SEQ ID NO: 16. In some embodiments, the IL-15 cytokine or functional fragment thereof comprises an amino acid sequence of SEQ ID NO: 26. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 26.

(SEQ ID NO: 26)
NWVNVISDLKKIEDLIQSMHIDATLYTESDVHPSCKVTAMKCFLLELQV
ISLESGDASIHDTVENLIILANNSLSSNGQVTESGCKECEELEEKNIKE
FLQSFVHIVQMFINTS

In some embodiments, the IL-15 cytokine or functional fragment thereof comprises an amino acid sequence having an amino acid substitution N112Q as compared to the amino acid sequence of SEQ ID NO: 16. In some embodiments, the IL-15 cytokine or functional fragment thereof comprises an amino acid sequence of SEQ ID NO: 27. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 27.

(SEQ ID NO: 27)
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFIQTS

In some embodiments, the IL-15 cytokine or functional fragment thereof comprises an amino acid sequence having an amino acid substitution N71Q and N79Q as compared to the amino acid sequence of SEQ ID NO: 16. In some embodiments, the IL-15 cytokine or functional fragment thereof comprises an amino acid sequence of SEQ ID NO: 28. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 28.

(SEQ ID NO: 28)
NWVNVISDLKKIEDLIQSMHIDATLYTESDVHPSCKVTAMKCFLLELQV
ISLESGDASIHDTVENLIILAQNSLSSNGQVTESGCKECEELEEKNIKE
FLQSFVHIVQMFINTS

In some embodiments, the IL-15 cytokine or functional fragment thereof comprises an amino acid sequence having an amino acid substitution N71Q and N112Q as compared to the amino acid sequence of SEQ ID NO: 16. In some embodiments, the IL-15 cytokine or functional fragment thereof comprises an amino acid sequence of SEQ ID NO: 29. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 29.

(SEQ ID NO: 29)
NWVNVISDLKKIEDLIQSMHIDATLYTESDVHPSCKVTAMKCFLLELQV
ISLESGDASIHDTVENLIILAQNSLSSNGNVTESGCKECEELEEKNIKE
FLQSFVHIVQMFIQTS

In some embodiments, the IL-15 cytokine or functional fragment thereof comprises an amino acid sequence having an amino acid substitution N79Q and N112Q as compared to the amino acid sequence of SEQ ID NO: 16. In some embodiments, the IL-15 cytokine or functional fragment thereof comprises an amino acid sequence of SEQ ID NO: 30. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 30.

(SEQ ID NO: 30)
NWVNVISDLKKIEDLIQSMHIDATLYTESDVHPSCKVTAMKCFLLELQV
ISLESGDASIHDTVENLIILANNSLSSNGQVTESGCKECEELEEKNIKE
FLQSFVHIVQMFIQTS

In some embodiments, the IL-15 cytokine or functional fragment thereof comprises an amino acid sequence having an amino acid substitution N71Q, N79Q and N112Q as compared to the amino acid sequence of SEQ ID NO: 16. In some embodiments, the IL-15 cytokine or functional fragment thereof comprises an amino acid sequence of SEQ ID NO: 31. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 31.

(SEQ ID NO: 31)
NWVNVISDLKKIEDLIQSMHIDATLYTESDVHPSCKVTAMKCFLLELQV
ISLESGDASIHDTVENLIILAQNSLSSNGQVTESGCKECEELEEKNIKE
FLQSFVHIVQMFIQTS

In some embodiments, an additional mutation may be included in any of the sequences above at position N71. In some embodiments, the mutation is N71A, N71R, N71W, N71F, N71P, N71M, N71L, N71T, N71S, or N71Y.

In some embodiments, an additional mutation may be included in any of the sequences above at position S73. In some embodiments, the mutation is S73A, S73W, S73V, or S73M.

In some embodiments, an additional mutation may be included in any of the sequences above at one or more of amino acid positions N72, N79, V80, T81, and N112. In some embodiments, one or more additional mutations selected from N72A, N79A, V80A, T81A and N112R may be included in any of the sequences above.

In some embodiments, an additional mutation may be included in any of the sequences above at one or more of amino acid positions N72, S73, N79, V80, T81, and N112. In some embodiments, one or more additional mutations N72A, S73A, N79A, V80A, T81A, and N112 may be included in any of the sequences above.

In some embodiments, the IL-15 cytokine or functional fragment thereof has one or more amino acid residues e.g., residues 1-3 s removed as compared to the amino acid sequence of the mature IL-15 of SEQ ID NO: 16, for the purpose of removing an O-glycosylation site. In some embodiments, the IL-15 cytokine or functional fragment thereof has one or more amino acid residues substituted as compared to the amino acid sequence of the mature IL-15 of SEQ ID NO: 16, for the purpose of removing an O-glycosylation site. In some embodiments, the IL-15 cytokine or functional fragment thereof has one or more amino acid residues inserted, e.g. in the region of residues 1-3, as compared to the amino acid sequence of the mature IL-15 of SEQ ID NO: 16, for the purpose of removing an O-glycosylation site. In some embodiments, the IL-15 cytokine or functional fragment thereof does not have an O-glycosylation site within residues 1-3.

Interleukin 12 (IL-12)

Provided herein is an IL-12 cytokine or functional fragment thereof for use in a targeted cytokine or cleavage product thereof. A cytokine plays a role in cellular signalling, particularly in cells of the immune system. IL-12 is an interleukin, which is a type of cytokine signalling molecule in the immune system that regulates activities of white blood cells.

Endogenous IL-12 exists as two distinct molecules IL-12 p40 and IL-12 p35 that dimerize in the cell during biosynthesis.

The full sequences of IL-12 p40 and IL-12 p35 are (pro-peptides cleaved off during biosynthesis are shown) in bold):

IL-12 p40 subunit:
(SEQ ID NO: 32)
MCHQQLVISWFSLVFLASPLVAIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCS
IL-12 p35 subunit:
(SEQ ID NO: 33)
MCPARSLLLVATLVLLDHLSLARNLPVATPDPGMFPCLHHSQNLLRAVS
NMLQKARQTLEFYPCTSEEIDHEDITKDKTSTVEACLPLELTKNESCLN
SRETSFITNGSCLASRKTSFMMALCLSSIYEDLKMYQVEFKTMNAKLLM
DPKRQIFLDQNMLAVIDELMQALNFNSETVPQKSSLEEPDFYKTKIKLC
ILLHAFRIRAVTIDRVMSYLNAS

The mature forms are as follows:

IL-12 p40 subunit:
(SEQ ID NO: 34)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFCVQVQGKSKREKKDRVFTDKTSATVICRKNASISVRAQDRYYS
SSWSEWASVPCS
IL-12 p35 subunit:
(SEQ ID NO: 35)
RNLPVATPDPGMFPCLHHSQNLLRAVSNMLQKARQTLEFYPCTSEEIDH
EDITKDKTSTVEACLPLELTKNESCLNSRETSFITNGSCLASRKTSFMM
ALCLSSIYEDLKMYQVEFKTMNAKLLMDPKRQIFLDQNMLAVIDELMQA
LNFNSETVPQKSSLEEPDFYKTKIKLCILLHAFRIRAVTIDRVMSYLNA
S

They are expressed as two chains that covalently dimerize during biosynthesis through a disulfide bound between the two subunits: Cysteine C199 of the p40 subunit associates with Cysteine C96 of the p35 subunit.

“Functional fragments” of an IL-12 cytokine comprise a portion of a full length cytokine protein which retains or has modified cytokine receptor binding capability (e.g., within at least 50%, 80%, 90%, 95%, 96%, 97%, 98%, 99% or 100% activity compared to the full length cytokine protein). Cytokine receptor binding capability can be shown, for example, by the capability of a cytokine to bind to the cytokine's cognate receptor or a component thereof.

In some embodiments, the IL-12 cytokine or functional fragment thereof is any naturally occurring interleukin-2 (IL-12) protein or modified variant thereof capable of binding to an interleukin-12 receptor.

In some embodiments, the IL-12 polypeptide or functional fragment thereof comprises an IL-12p40 polypeptide or functional fragment thereof covalently linked to an IL-12p35 polypeptide or functional fragment thereof.

The IL-12p40 polypeptide or functional fragment thereof may be attached to the first half life extension domain such that the first polypeptide chain comprises:

N′HL1-L1-MM C′

and the second polypeptide chain comprises:

N′HL2-L2-[IL-12p40-linker-IL-12p35]C′

where ‘IL-12p40’ is the IL-12p40 polypeptide or functional fragment thereof and ‘IL-12p35’ is the IL-12p35 polypeptide or functional fragment thereof.

In some embodiments, the IL-12p40 polypeptide comprises SEQ ID NO: 34. In some embodiments, the IL-12p40 polypeptide comprises an amino acid sequence having at least one amino acid modification as compared to the amino acid sequence of SEQ ID NO: 34. Each of the at least one amino acid modifications can be any amino acid modification, such as a substitution, insertion, or deletion. In some embodiments, the IL-12 cytokine or functional fragment thereof comprises an amino acid sequence having at least 1, at least 2, at least 3, at least 4, at least 5, at least 6, at least 7, at least 8, at least 9, or at least 10 amino acid substitutions as compared to the amino acid sequence of SEQ ID NO: 34. In some embodiments, the IL-12 cytokine or functional fragment thereof comprises an amino acid sequence having at least 5 amino acid substitutions as compared to the amino acid sequence of SEQ ID NO: 34.

The IL-12p40 polypeptide comprises a glycosaminoglycan (GAG)-binding domain. GAGs, such as heparin and heparan sulphate, have been shown to bind numerous growth factors and cytokines, including IL-12. The physiological significance of this binding is two-fold. First, GAGs can serve as co-receptors on cell surfaces to maintain high, local concentrations of cytokines. Second, GAGs can regulate bioactivities of growth factors and cytokines through multiple mechanisms including dimerization and protection from proteolytic degradation.

The GAG-binding domain in the mature form of the IL-12 p40 subunit is shown below in bold:

(SEQ ID NO: 36)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFCVQVQGKSKREKKDRVFTDKTSATVICRKNASISVRAQDRYYS
SSWSEWASVPCS

Modifications to the GAG-binding domain KSKREKKDRV (SEQ ID NO: 37) has been shown herein to increase the PK profile of constructs comprising an IL-12 cytokine with a mutated GAG-binding domain, without any decrease in cytokine activity. Thus, in some embodiments, the IL-12p40 polypeptide comprises at least one amino acid modification to the GAG-binding domain. In some embodiments, the modification to the GAG-binding domain is a deletion mutation. In some embodiments, the modification to the GAG-binding domain is a deletion mutation and at least one substitution mutation.

In some embodiments, the GAG-binding domain comprises the amino acid sequence KDNTERV. In some embodiments, the IL-12p40 polypeptide comprises the amino acid sequence SEQ ID NO: 38.

(SEQ ID NO: 38)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFCVQVQGKDNTERVFTDKTSATVICRKNASISVRAQDRYYSSSW
SEWASVPCS

In some embodiments, the GAG-binding domain comprises the amino acid sequence KDNTEGRV. In some embodiments, the IL-12p40 polypeptide comprises the amino acid sequence SEQ ID NO: 39.

(SEQ ID NO: 39)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFCVQVQGKDNTEGRVFTDKTSATVICRKNASISVRAQDRYYSSS
WSEWASVPCS

In some embodiments, the GAG-binding domain consists of the amino acid sequence KDNTERV. In some embodiments, the IL-12p40 polypeptide comprises the amino acid sequence SEQ ID NO: 38. In some embodiments, the GAG-binding domain consists of the amino acid sequence KDNTEGRV. In some embodiments, the IL-12p40 polypeptide comprises the amino acid sequence SEQ ID NO: 39.

In some embodiments, the IL-12p40 polypeptide comprises an amino acid sequence having one or more cysteine substitutions as compared to the amino acid sequence of SEQ ID NO: 34. In some embodiments, the IL-12p40 polypeptide comprises an amino acid sequence having an amino acid substitution at position C252 as compared to the amino acid sequence of SEQ ID NO: 34. In some embodiments, the amino acid substitution at position C252 is C252S. In some embodiments, the IL-12p40 polypeptide comprises an amino acid sequence of SEQ ID NO: 40. In some embodiments, the IL-12p40 polypeptide comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 40. In some embodiments, the IL-12p40 polypeptide consists of an amino acid sequence of SEQ ID NO: 40.

(SEQ ID NO: 40)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFSVQVQGKSKREKKDRVFTDKTSATVICRKNASISVRAQDRYYS
SSWSEWASVPCS

In some embodiments, the IL-12p40 polypeptide comprises an amino acid sequence having one or more cysteine substitutions as compared to the amino acid sequence of SEQ ID NO: 34, and at least one amino acid modification to the GAG-binding domain. In some embodiments, the IL-12p40 polypeptide comprises an amino acid substitution at position C252S as compared to the amino acid sequence of SEQ ID NO: 34, and the GAG-binding domain comprises the amino acid sequence KDNTERV. In some embodiments, the IL-12p40 polypeptide comprises an amino acid substitution at position C252S as compared to the amino acid sequence of SEQ ID NO: 34, and the GAG-binding domain comprises the amino acid sequence KDNTEGRV (SEQ ID NO: 41). In some embodiments, the IL-12p40 polypeptide comprises an amino acid sequence of SEQ ID NO: 42. In some embodiments, the IL-12p40 polypeptide comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 42. In some embodiments, the IL-12p40 polypeptide consists of an amino acid sequence of SEQ ID NO: 42.

(SEQ ID NO: 42)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFSVQVQGKDNTEGRVFTDKTSATVICRKNASISVRAQDRYYSSS
WSEWASVPCS

In some embodiments, the IL-12p35 polypeptide comprises SEQ ID NO: 35. In some embodiments, the IL-12p35 polypeptide comprises an amino acid sequence having at least one amino acid modification as compared to the amino acid sequence of SEQ ID NO: 35. Each of the at least one amino acid modifications can be any amino acid modification, such as a substitution, insertion, or deletion. In some embodiments, the IL-12 cytokine or functional fragment thereof comprises an amino acid sequence having at least 1, at least 2, at least 3, at least 4, at least 5, at least 6, at least 7, at least 8, at least 9, or at least 10 amino acid substitutions as compared to the amino acid sequence of SEQ ID NO: 35. In some embodiments, the IL-12 cytokine or functional fragment thereof comprises an amino acid sequence having at least 5 amino acid substitutions as compared to the amino acid sequence of SEQ ID NO: 35.

In some embodiments, the IL-12p40-IL-12p35 linker is between 5 and 20 amino acids in length.

In some embodiments, the IL-12p40-IL-12p35 linker is rich in amino acid residues G and S.

In some embodiments, the IL-12p40-IL-12p35 linker only includes amino acid residue types selected from the group consisting of G and S.

In some embodiments, the IL-12p40-IL-12p35 linker includes [(G)nS], where n=4 or 5.

In some embodiments, the IL-12p40-IL-12p35 linker includes a (GGGGS) (SEQ ID NO: 43) repeat.

In some embodiments, IL-12p40-IL-12p35 linker comprises SEQ ID NO: 44. (GGGGSGGGGSGGGGS)

In some embodiments, the IL-12 cytokine or functional fragment thereof comprises SEQ ID NO: 45. In some embodiments, the IL-12 cytokine or functional fragment thereof comprises an amino acid sequence having at least one amino acid modification as compared to the amino acid sequences of SEQ ID NO: 34 and 35. Each of the at least one amino acid modifications can be any amino acid modification, such as a substitution, insertion, or deletion. In some embodiments, the IL-12 cytokine or functional fragment thereof comprises an amino acid sequence having at least 1, at least 2, at least 3, at least 4, at least 5, at least 6, at least 7, at least 8, at least 9, or at least 10 amino acid substitutions as compared to the amino acid sequences of SEQ ID NO: 34 and 35. In some embodiments, the IL-12 cytokine or functional fragment thereof comprises an amino acid sequence having at least 5 amino acid substitutions as compared to the amino acid sequences of SEQ ID NO: 34 and 35.

(SEQ ID NO: 45)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFCVQVQGKSKREKKDRVFTDKTSATVICRKNASISVRAQDRYYS
SSWSEWASVPCSGGGGSGGGGSGGGGSRNLPVATPDPGMFPCLHHSQNL
LRAVSNMLQKARQTLEFYPCTSEEIDHEDITKDKTSTVEACLPLELTKN
ESCLNSRETSFITNGSCLASRKTSFMMA

In some embodiments, the IL-12 cytokine or functional fragment thereof comprises the amino acid sequence of SEQ ID NO: 45. In some embodiments, the IL-12 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 45.

In some embodiments, the IL-12 cytokine or functional fragment thereof comprises the amino acid sequence of SEQ ID NO: 46. In some embodiments, the IL-12 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 46.

(SEQ ID NO: 46)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFCVQVQGKDNTERVFTDKTSATVICRKNASISVRAQDRYYSSSW
SEWASVPCSGGGGSGGGGSGGGGSRNLPVATPDPGMFPCLHHSQNLLRA
VSNMLQKARQTLEFYPCTSEEIDHEDITKDKTSTVEACLPLELTKNESC
LNSRETSFITNGSCLASRKTSFMMALCLSSIYEDLKMYQVEFKTMNAKL
LMDPKRQIFLDQNMLAVIDELMQALNENSETVPQKSSLEEPDFYKTKIK
LCILLHAFRIRAVTIDRVMSYLNAS

In some embodiments, the IL-12 cytokine or functional fragment thereof comprises the amino acid sequence of SEQ ID NO: 47. In some embodiments, the IL-12 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 47.

(SEQ ID NO: 47)
IWELKKDVYVVELDWYPDAPGEMVVLTCDTPEEDGITWTLDQSSEVLGSGKTLTIQVK
EFGDAGQYTCHKGGEVLSHSLLLLHKKEDGIWSTDILKDQKEPKNKTFLRCEAKNYSGR
FTCWWLTTISTDLTFSVKSSRGSSDPQGVTCGAATLSAERVRGDNKEYEYSVECQEDSA
CPAAEESLPIEVMVDAVHKLKYENYTSSFFIRDIIKPDPPKNLQLKPLKNSRQVEVSWEY
PDTWSTPHSYFSLTFCVQVQGKDNTEGRVFTDKTSATVICRKNASISVRAQDRYYSSSW
SEWASVPCSGGGGSGGGGSGGGGSRNLPVATPDPGMFPCLHHSQNLLRAVSNMLQKA
RQTLEFYPCTSEEIDHEDITKDKTSTVEACLPLELTKNESCLNSRETSFITNGSCLASRKTS
FMMALCLSSIYEDLKMYQVEFKTMNAKLLMDPKRQIFLDQNMLAVIDELMQALNENSE
TVPQKSSLEEPDFYKTKIKLCILLHAFRIRAVTIDRVMSYLNAS

In some embodiments, the IL-12 cytokine or functional fragment thereof comprises the amino acid sequence of SEQ ID NO: 48. In some embodiments, the IL-12 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 48.

(SEQ ID NO: 48)
IWELKKDVYVVELDWYPDAPGEMVVLTCDTPEEDGITWTLDQSSEVLGSGKTLTIQVK
EFGDAGQYTCHKGGEVLSHSLLLLHKKEDGIWSTDILKDQKEPKNKTFLRCEAKNYSGR
FTCWWLTTISTDLTFSVKSSRGSSDPQGVTCGAATLSAERVRGDNKEYEYSVECQEDSA
CPAAEESLPIEVMVDAVHKLKYENYTSSFFIRDIIKPDPPKNLQLKPLKNSRQVEVSWEY
PDTWSTPHSYFSLTFSVQVQGKSKREKKDRVFTDKTSATVICRKNASISVRAQDRYYSSS
WSEWASVPCSGGGGSGGGGSGGGGSRNLPVATPDPGMFPCLHHSQNLLRAVSNMLQK
ARQTLEFYPCTSEEIDHEDITKDKTSTVEACLPLELTKNESCLNSRETSFITNGSCLASRKT
SFMMALCLSSIYEDLKMYQVEFKTMNAKLLMDPKRQIFLDQNMLAVIDELMQALNENS
ETVPQKSSLEEPDFYKTKIKLCILLHAFRIRAVTIDRVMSYLNAS

In some embodiments, the IL-12 cytokine or functional fragment thereof comprises the amino acid sequence of SEQ ID NO: 49. In some embodiments, the IL-12 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 49.

(SEQ ID NO: 49)
IWELKKDVYVVELDWYPDAPGEMVVLTCDTPEEDGITWTLDQSSEVLGSGKTLTIQVK
EFGDAGQYTCHKGGEVLSHSLLLLHKKEDGIWSTDILKDQKEPKNKTFLRCEAKNYSGR
FTCWWLTTISTDLTFSVKSSRGSSDPQGVTCGAATLSAERVRGDNKEYEYSVECQEDSA
CPAAEESLPIEVMVDAVHKLKYENYTSSFFIRDIIKPDPPKNLQLKPLKNSRQVEVSWEY
PDTWSTPHSYFSLTFSVQVQGKDNTEGRVFTDKTSATVICRKNASISVRAQDRYYSSSW
SEWASVPCSGGGGSGGGGSGGGGSRNLPVATPDPGMFPCLHHSQNLLRAVSNMLQKA
RQTLEFYPCTSEEIDHEDITKDKTSTVEACLPLELTKNESCLNSRETSFITNGSCLASRKTS
FMMALC

Masking Moiety

Provided herein is a masking moiety for use in a masked or targeted cytokine or any other masked therapeutically active molecule. It will be understood that the masking moiety is cleaved from the targeted cytokine to form the cleavage product thereof. A masking moiety binds to the cytokine moiety and inhibits a biological activity of the cytokine. Upon cleavage, a masking moiety is released from a cytokine, activating the function of cytokine in a target of interest.

In some embodiments, a masking moiety comprises an agent, a peptide, or a polypeptide that binds to a cytokine. In some embodiments, a masking moiety comprises a cyclic peptide that binds a cytokine. In some embodiments, a masking moiety comprises a linear peptide that binds a cytokine.

In some embodiments, a masking moiety comprises a Fab, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), an antibody, or a combination thereof. In some embodiments, a masking moiety comprises a Fab that binds to a cytokine. In some embodiments, a masking moiety comprises a single chain Fv (scFv) that binds to a cytokine. In some embodiments, a masking moiety comprises a single domain antibody (VHH) that binds to a cytokine. In some embodiments, a masking moiety comprises one or more CDRs that bind to a cytokine. In some embodiments, a masking moiety comprises a variable heavy chain (VH) that binds to a cytokine. In some embodiments, a masking moiety comprises a variable light chain (VL) that binds to a cytokine. In some embodiments, a masking moiety comprises a Fab-like bispecific antibodies (bsFab) that binds to a cytokine. In some embodiments, a masking moiety comprises a single-domain antibody-linked Fab (s-Fab) that binds to a cytokine. In some embodiments, a masking moiety comprises an antibody or a fragment thereof that binds to a cytokine. In some embodiments, the masking moiety comprises an antibody against the cytokine or a binding fragment of the antibody.

In some embodiments, the masking moiety is a receptor of the cytokine. In some embodiments, the masking moiety is a fragment of a receptor of the cytokine. In some embodiments, the masking moiety is an extracellular domain (ECD) of a receptor of the cytokine.

In some embodiments, the cytokine is an IL-1α or an IL-1β; and wherein the masking moiety is a Fab, a single chain Fv (scFv), or a single domain antibody against IL-1α or an IL-1β. In some embodiments, the cytokine is an IL-1α or an IL-1β; and wherein the masking moiety is CD121a, CDwl21b, or a fragment thereof.

In some embodiments, the cytokine is an IL-2; and wherein the masking moiety is a Fab, a single chain Fv (scFv), or a single domain antibody against IL-2. In some embodiments, the cytokine is an IL-2; and wherein the masking moiety is IL-2Rα, IL-2Rβ, CD25, CD122, CD132, or a fragment thereof.

In some embodiments, the cytokine is an IL-18; and wherein the masking moiety is a Fab, a single chain Fv (scFv), or a single domain antibody against IL-18. In some embodiments, the cytokine is an IL-18; and wherein the masking moiety is IL-18Rα, IL-18Rβ, or a fragment thereof.

In some embodiments, the cytokine is an IL-4; and wherein the masking moiety is a Fab, a single chain Fv (scFv), or a single domain antibody against IL-4. In some embodiments, the cytokine is an IL-4; and wherein the masking moiety is CD124, CD213a13, CD132, or a fragment thereof.

In some embodiments, the cytokine is an IL-7; and wherein the masking moiety is a Fab, a single chain Fv (scFv), or a single domain antibody against IL-7. In some embodiments, the cytokine is an IL-7; and wherein the masking moiety is CD127, CD132, or a fragment thereof.

In some embodiments, the cytokine is an IL-9; and wherein the masking moiety is a Fab, a single chain Fv (scFv), or a single domain antibody against IL-9. In some embodiments, the cytokine is an IL-9, and wherein the masking moiety is IL-9R, CD132, or a fragment thereof.

In some embodiments, the cytokine is an IL-13; and wherein the masking moiety is a Fab, a single chain Fv (scFv), or a single domain antibody against IL-13. In some embodiments, the cytokine is an IL-13; and wherein the masking moiety is CD213a1, CD213a2, or a fragment thereof.

In some embodiments, the cytokine is an IL-15; and wherein the masking moiety is a Fab, a single chain Fv (scFv), or a single domain antibody against IL-15. In some embodiments, the cytokine is an IL-15; and wherein the masking moiety is IL-15Ra, CD122, CD132, or a fragment thereof.

In some embodiments, the cytokine is an IL-3; and wherein the masking moiety is a Fab, a single chain Fv (scFv), or a single domain antibody against IL-3. In some embodiments, the cytokine is an IL-3; and wherein the masking moiety is CD123, CDw131, or a fragment thereof.

In some embodiments, the cytokine is an IL-5; and wherein the masking moiety is a Fab, a single chain Fv (scFv), or a single domain antibody against IL-5. In some embodiments, the cytokine is an IL-5; and wherein the masking moiety is CDw125, CD131, or a fragment thereof.

In some embodiments, the cytokine is a GM-CSF; and wherein the masking moiety is a Fab, a single chain Fv (scFv), or a single domain antibody against GM-CSF. In some embodiments, the cytokine is a GM-CSF; and wherein the masking moiety is CD116, CDw131, or a fragment thereof.

In some embodiments, the cytokine is an IL-6; and wherein the masking moiety is a Fab, a single chain Fv (scFv), or a single domain antibody against IL-6. In some embodiments, the cytokine is an IL-6; and wherein the masking moiety is CD126, CD130, or a fragment thereof.

In some embodiments, the cytokine is an IL-11; and wherein the masking moiety is a Fab, a single chain Fv (scFv), or a single domain antibody against IL-11. In some embodiments, the cytokine is an IL-11; and wherein the masking moiety is IL-11Ra, CD130, or a fragment thereof.

In some embodiments, the cytokine is a G-CSF; and wherein the masking moiety is a Fab, a single chain Fv (scFv), or a single domain antibody against G-CSF. In some embodiments, the cytokine is a G-CSF; and wherein the masking moiety is CD114, or a fragment thereof.

In some embodiments, the cytokine is an IL-12; and wherein the masking moiety is a Fab, a single chain Fv (scFv), or a single domain antibody against IL-12. In some embodiments, the cytokine is an IL-12; and wherein the masking moiety is CD212, or a fragment thereof.

In some embodiments, the cytokine is an LIF; and wherein the masking moiety is a Fab, a single chain Fv (scFv), or a single domain antibody against LIF. In some embodiments, the cytokine is an LIF; and wherein the masking moiety is LIFR, CD130 or a fragment thereof.

In some embodiments, the cytokine is an OSM; and wherein the masking moiety is a Fab, a single chain Fv (scFv), or a single domain antibody against OSM. In some embodiments, the cytokine is an OSM; and wherein the masking moiety is OSMR, CD130, or a fragment thereof.

In some embodiments, the cytokine is an IL-10; and wherein the masking moiety is a Fab, a single chain Fv (scFv), or a single domain antibody against IL-10. In some embodiments, the cytokine is an IL-10; and wherein the masking moiety is CDw210 or a fragment thereof.

In some embodiments, the cytokine is an IL-20; and wherein the masking moiety is a Fab, a single chain Fv (scFv), or a single domain antibody against IL-20. In some embodiments, the cytokine is an IL-20; and wherein the masking moiety is IL-20Rα, IL-20Rβ, or a fragment thereof.

In some embodiments, the cytokine is an IL-14; and wherein the masking moiety is a Fab, a single chain Fv (scFv), or a single domain antibody against IL-14. In some embodiments, the cytokine is an IL-14; and wherein the masking moiety is IL-14R, or a fragment thereof.

In some embodiments, the cytokine is an IL-16; and wherein the masking moiety is a Fab, a single chain Fv (scFv), or a single domain antibody against IL-16. In some embodiments, the cytokine is an IL-16; and wherein the masking moiety is CD4, or a fragment thereof.

In some embodiments, the cytokine is an IL-17; and wherein the masking moiety is a Fab, a single chain Fv (scFv), or a single domain antibody against IL-17. In some embodiments, the cytokine is an IL-17; and wherein the masking moiety is CDw217, or a fragment thereof.

In some embodiments, the cytokine is an IFN-α; and wherein the masking moiety is a Fab, a single chain Fv (scFv), or a single domain antibody against IFN-α. In some embodiments, the cytokine is an IFN-α; and wherein the masking moiety is CD118, or a fragment thereof.

In some embodiments, the cytokine is an IFN-β; and wherein the masking moiety is a Fab, a single chain Fv (scFv), or a single domain antibody against IFN-β. In some embodiments, the cytokine is an IFN-β; and wherein the masking moiety is CD118, or a fragment thereof.

In some embodiments, the cytokine is an IFN-γ; and wherein the masking moiety is a Fab, a single chain Fv (scFv), or a single domain antibody against IFN-γ. In some embodiments, the cytokine is an IFN-γ; and wherein the masking moiety is CDw119, or a fragment thereof.

In some embodiments, the cytokine is a CD154; and wherein the masking moiety is a Fab, a single chain Fv (scFv), or a single domain antibody against CD154. In some embodiments, the cytokine is a CD154; and wherein the masking moiety is CD40, or a fragment thereof.

In some embodiments, the cytokine is an LT-β; and wherein the masking moiety is a Fab, a single chain Fv (scFv), or a single domain antibody against LT-β. In some embodiments, the cytokine is an LT-β; and wherein the masking moiety is LT-OR, or a fragment thereof.

In some embodiments, the cytokine is a TNF-α; and wherein the masking moiety is a Fab, a single chain Fv (scFv), or a single domain antibody against TNF-α. In some embodiments, the cytokine is a TNF-α; and wherein the masking moiety is CD120a, CD120b, or a fragment thereof.

In some embodiments, the cytokine is a TNF-β; and wherein the masking moiety is a Fab, a single chain Fv (scFv), or a single domain antibody against TMF-β. In some embodiments, the cytokine is a TNF-β; and wherein the masking moiety is CD120a, CD120b, or a fragment thereof.

In some embodiments, the cytokine is a 4-1BBL; and wherein the masking moiety is a Fab, a single chain Fv (scFv), or a single domain antibody against 4-1BBL. In some embodiments, the cytokine is a 4-1BBL; and wherein the masking moiety is CDw137, 4-1BB, or a fragment thereof.

In some embodiments, the cytokine is an APRIL; and wherein the masking moiety is a Fab, a single chain Fv (scFv), or a single domain antibody against APRIL. In some embodiments, the cytokine is an APRIL; and wherein the masking moiety is BCMA, TACI, or a fragment thereof.

In some embodiments, the cytokine is a CD70; and wherein the masking moiety is a Fab, a single chain Fv (scFv), or a single domain antibody against CD70. In some embodiments, the cytokine is a CD70; and wherein the masking moiety is CD27, or a fragment thereof.

In some embodiments, the cytokine is a CD153; and wherein the masking moiety is a Fab, a single chain Fv (scFv), or a single domain antibody against CD153. In some embodiments, the cytokine is a CD153; and wherein the masking moiety is CD30, or a fragment thereof.

In some embodiments, the cytokine is a CD178; and wherein the masking moiety is a Fab, a single chain Fv (scFv), or a single domain antibody against CD178. In some embodiments, the cytokine is a CD178; and wherein the masking moiety is CD95, or a fragment thereof.

In some embodiments, the cytokine is a GITRL; and wherein the masking moiety is a Fab, a single chain Fv (scFv), or a single domain antibody against GITRL. In some embodiments, the cytokine is a GITRL; and wherein the masking moiety is GITR, or a fragment thereof.

In some embodiments, the cytokine is a LIGHT; and wherein the masking moiety is a Fab, a single chain Fv (scFv), or a single domain antibody against LIGHT. In some embodiments, the cytokine is a LIGHT; and wherein the masking moiety is LTβR, HVEM, or a fragment thereof.

In some embodiments, the cytokine is an OX40; and wherein the masking moiety is a Fab, a single chain Fv (scFv), or a single domain antibody against OX40. In some embodiments, the cytokine is an OX40; and wherein the masking moiety is OX40, or a fragment thereof.

In some embodiments, the cytokine is a TALL-1; and wherein the masking moiety is a Fab, a single chain Fv (scFv), or a single domain antibody against TALL-1. In some embodiments, the cytokine is a TALL-1; and wherein the masking moiety is BCMA, TACI, or a fragment thereof.

In some embodiments, the cytokine is a TRAIL; and wherein the masking moiety is a Fab, a single chain Fv (scFv), or a single domain antibody against TRAIL. In some embodiments, the cytokine is a TRAIL; and wherein the masking moiety is TRAILR1-4, or a fragment thereof.

In some embodiments, the cytokine is a TWEAK; and wherein the masking moiety is a Fab, a single chain Fv (scFv), or a single domain antibody against TWEAK. In some embodiments, the cytokine is a TWEAK; and wherein the masking moiety is Apo3, or a fragment thereof.

In some embodiments, the cytokine is a TRANCE; and wherein the masking moiety is a Fab, a single chain Fv (scFv), or a single domain antibody against TRANCE. In some embodiments, the cytokine is a TRANCE; and wherein the masking moiety is RANK, OPG, or a fragment thereof.

In some embodiments, the cytokine is a TGF-β1; and wherein the masking moiety is a Fab, a single chain Fv (scFv), or a single domain antibody against TGF-β1. In some embodiments, the cytokine is a TGF-β1; and wherein the masking moiety is TGF-βR1, or a fragment thereof.

In some embodiments, the cytokine is a TGF-β2; and wherein the masking moiety is a Fab, a single chain Fv (scFv), or a single domain antibody against TGF-β2. In some embodiments, the cytokine is a TGF-β2; and wherein the masking moiety is TGF-βR2, or a fragment thereof.

In some embodiments, the cytokine is a TGF-β3; and wherein the masking moiety is a Fab, a single chain Fv (scFv), or a single domain antibody against TGF-β3. In some embodiments, the cytokine is a TGF-β3; and wherein the masking moiety is TGF-βR3, or a fragment thereof.

In some embodiments, the cytokine is an Epo; and wherein the masking moiety is a Fab, a single chain Fv (scFv), or a single domain antibody against Epo. In some embodiments, the cytokine is an Epo; and wherein the masking moiety is EpoR, or a fragment thereof.

In some embodiments, the cytokine is a Tpo; and wherein the masking moiety is a Fab, a single chain Fv (scFv), or a single domain antibody against Tpo. In some embodiments, the cytokine is a Tpo; and wherein the masking moiety is TpoR, or a fragment thereof.

In some embodiments, the cytokine is an Flt-3L; and wherein the masking moiety is a Fab, a single chain Fv (scFv), or a single domain antibody against Flt-3L. In some embodiments, the cytokine is an Flt-3L; and wherein the masking moiety is Flt-3, or a fragment thereof.

In some embodiments, the cytokine is an SCF; and wherein the masking moiety is a Fab, a single chain Fv (scFv), or a single domain antibody against SCF. In some embodiments, the cytokine is an SCF; and wherein the masking moiety is CD117, or a fragment thereof.

In some embodiments, the cytokine is an M-CSF; and wherein the masking moiety is a Fab, a single chain Fv (scFv), or a single domain antibody against M-CSF. In some embodiments, the cytokine is an M-CSF; and wherein the masking moiety is CD115, or a fragment thereof.

In some embodiments, the cytokine is an MSP; and wherein the masking moiety is a Fab, a single chain Fv (scFv), or a single domain antibody against MSP. In some embodiments, the cytokine is an MSP; and wherein the masking moiety is CDw136, or a fragment thereof.

In some embodiments, the cytokine is an IL-15 agonist polypeptide; and wherein the masking moiety is a Fab, a single chain Fv (scFv), or a single domain antibody against IL-15. In some embodiments, the cytokine moiety comprises an IL-15 agonist polypeptide, wherein the chimeric molecule further comprises the sushi domain of IL-15 receptor a (IL-15Ra sushi domain).

In some embodiments, a cytokine is an IL-2 agonist polypeptide; and wherein the masking moiety is the extracellular domain of IL-21 receptor a (IL-21Ra ECD) or a functional analog thereof. In some embodiments, the cytokine is an IL-2 agonist polypeptide or an IL-15 agonist polypeptide; and wherein the masking moiety is the extracellular domain of IL-2 receptor b (IL-2R β ECD). In some embodiments, the cytokine is an IL-21 agonist polypeptide; and wherein the masking moiety is a Fab, a single chain Fv (scFv) or a single domain antibody against IL-21.

In some embodiments, a cytokine is an IL-2 agonist polypeptide; and wherein the masking moiety is the extracellular domain of IL-21 receptor a (IL-21Ra ECD) or a functional analog thereof. In some embodiments, the cytokine is an IL-2 agonist polypeptide or an IL-15 agonist polypeptide; and wherein the masking moiety is the extracellular domain of IL-2 receptor b (IL-2R β ECD). In some embodiments, the cytokine is an IL-21 agonist polypeptide; and wherein the masking moiety is a Fab, a single chain Fv (scFv) or a single domain antibody against IL-21.

Antibody or Antigen Binding Fragment

The masking moiety masks a cytokine or a therapeutically active domain in the masked cytokine or the masked therapeutically active molecule thereby reducing or preventing binding of the cytokine or a therapeutically active domain to its cognate receptor or ligand.

In some embodiments, a masking moiety is an antibody or an antigen binding fragment thereof that prevents a therapeutically active domain from binding to its cognate ligand or receptor. In some embodiments, a masking moiety is an antibody or an antigen binding fragment thereof that specifically binds a therapeutically active domain. In some embodiments, a masking moiety is an antibody or an antigen binding fragment thereof that prevents a cytokine from binding to its cognate ligand or receptor. In some embodiments, a masking moiety is an antibody or an antigen binding fragment thereof that specifically binds a cytokine.

In some embodiments, a masking moiety comprises an antibody or an antigen binding fragment thereof that specifically binds a therapeutically active domain. In some embodiments, a masking moiety comprises an antibody or an antigen binding fragment thereof that specifically binds a cytokine. In some embodiments, a masking moiety comprises an antibody or an antigen binding fragment thereof that specifically binds an IL-2 cytokine. In some embodiments, a masking moiety comprises an antibody or an antigen binding fragment thereof that specifically binds an IL-15 cytokine. In some embodiments, a masking moiety comprises an antibody or an antigen binding fragment thereof that specifically binds an IL-12 cytokine. In some embodiments, a masking moiety comprises an antibody or an antigen binding fragment thereof that specifically binds an IL-18 cytokine.

In some embodiments, a masking moiety comprises a scFv that specifically binds an IL-2 cytokine. In some embodiments, a masking moiety comprises a scFv that specifically binds an IL-15 cytokine. In some embodiments, a masking moiety comprises a scFv that specifically binds an IL-12 cytokine. In some embodiments, a masking moiety comprises a scFv that specifically binds an IL-18 cytokine.

In some embodiments, a masking moiety comprises a VHH that specifically binds an IL-2 cytokine. In some embodiments, a masking moiety comprises a VHH that specifically binds an IL-15 cytokine. In some embodiments, a masking moiety comprises a VHH that specifically binds an IL-12 cytokine. In some embodiments, a masking moiety comprises a VHH that specifically binds an IL-18 cytokine.

Anti-IL-2 scFv

In some embodiments, a masking moiety comprises an anti-IL-2 scFv having a variable heavy chain (VH) of SEQ ID NO: 124. In some embodiments, a masking moiety comprises an anti-IL-2 scFv having a variable light chain (VL) of SEQ ID NO: 125. In some embodiments, a masking moiety comprises an anT-IL-2 scFv having a VH of SEQ ID NO: 124 and a NVL of SEQ ID NO: 125.

In some embodiments, a masking moiety comprises an anti-IL-2 scFv having a hCDR1 of SEQ ID NO: 126, a hCDR2 of SEQ ID NO: 127, and hCDR3 of SEQ ID NO: 128. In some embodiments, a masking moiety comprises an anRV-IL-2 scFv having a CDR1 of SEQ ID NO: 129, a CDR2 of SEQ ID NO: 130, and lCDR3 of SEQ ID NO: 131. In some embodiments, a masking moiety comprises an and-IL-2 scFv having a hCDR1 of SEQ ID NO: 126, a hCDR2 of SEQ ID NO: 127, hCDR3 of SEQ ID NO: 128, a 3CDR1 ofSEQ ID NO: 129, a 0CDR2 of SEQ ID NO: 130, and CDR3 of SEQ ID NO: 131.

An Exemplary Anti-IL-2 scFv Sequence

| | | |
|---|---|---|
| VH | QLQLQESGPGLVKPSQTLSLTCTVSGGSISSGGYYWS | SEQ ID NO: 124 |
| | WIRQHPGKGLEWIGYIYKSGSAYYSPSLKSRVTISVDT | |
| | SKNQFSLKLSSVTAADTAVYYCARTPTVTGDWFDPW | |
| | GRGTLVTVSS | |
| VL | NFMLTQPHSVSESPGKTVTISCTRSSGSIASNYVQWYQ | SEQ ID NO: 125 |
| | QRPGSSPTTVIYEDNQRPSGVPDRFSGSIDSSSNSASLTI | |
| | SGLKTEDEADYYCQTYDSIDVYFGGGTKLTVL | |
| hCDR1 | SGGYYWS | SEQ ID NO: 126 |
| hCDR2 | GYIYKSGSAY YSPSLKSRV | SEQ ID NO: 127 |
| hCDR3 | TPTVTGDWFDP | SEQ ID NO: 128 |
| lCDR1 | TRSSGSIASNYVQ | SEQ ID NO: 129 |
| lCDR2 | EDNQRPS | SEQ ID NO: 130 |
| lCDR3 | QTYDSIDVY | SEQ ID NO: 131 |
| scFv | NFMLTQPHSVSESPGKTVTISCTRSSGSIASNYVQWYQ | SEQ ID NO: 142 |
| | QRPGSSPTTVIYEDNQRPSGVPDRFSGSIDSSSNSASLTI | |
| | SGLKTEDEADYYCQTYDSIDVYFGGGTKLTVLGGGG | |
| | GSGGGGSGGGGSGGGGSQLQLQESGPGLVKPSQTLSL | |
| | TCTVSGGSISSGGYYWSWIRQHPGKGLEWIGYIYKSG | |
| | SAYYSPSLKSRVTISVDTSKNQFSLKLSSVTAADTAVY | |
| | YCARTPTVTGDWFDPWGRGTLVTVSS | |

In some embodiments, a masking moiety comprises an amino acid sequence having at least 80% identity to SEQ ID NO: 124. In some embodiments, a masking moiety comprises an amino acid sequence having at least 85% identity to SEQ ID NO: 124. In some embodiments, a masking moiety comprises an amino acid sequence having at least 87% identity to SEQ ID NO: 124. In some embodiments, a masking moiety comprises an amino acid sequence having at least 88% identity to SEQ ID NO: 124. In some embodiments, a masking moiety comprises an amino acid sequence having at least 90% identity to SEQ ID NO: 124. In some embodiments, a masking moiety comprises an amino acid sequence having at least 92% identity to SEQ ID NO: 124. In some embodiments, a masking moiety comprises an amino acid sequence having at least 93% identity to SEQ ID NO: 124. In some embodiments, a masking moiety comprises an amino acid sequence having at least 94% identity to SEQ ID NO: 124. In some embodiments, a masking moiety comprises an amino acid sequence having at least 95% identity to SEQ ID NO: 124. In some embodiments, a masking moiety comprises an amino acid sequence having at least 96% identity to SEQ ID NO: 124. In some embodiments, a masking moiety comprises an amino acid sequence having at least 97% identity to SEQ ID NO: 124. In some embodiments, a masking moiety comprises an amino acid sequence having at least 98% identity to SEQ ID NO: 124. In some embodiments, a masking moiety comprises an amino acid sequence having at least 99% identity to SEQ ID NO: 124.

In some embodiments, a masking moiety comprises an amino acid sequence having at least 80% identity to SEQ ID NO: 125. In some embodiments, a masking moiety comprises an amino acid sequence having at least 85% identity to SEQ ID NO: 125. In some embodiments, a masking moiety comprises an amino acid sequence having at least 87% identity to SEQ ID NO: 125. In some embodiments, a masking moiety comprises an amino acid sequence having at least 88% identity to SEQ ID NO: 125. In some embodiments, a masking moiety comprises an amino acid sequence having at least 90% identity to SEQ ID NO: 125. In some embodiments, a masking moiety comprises an amino acid sequence having at least 92% identity to SEQ ID NO: 125. In some embodiments, a masking moiety comprises an amino acid sequence having at least 93% identity to SEQ ID NO: 125. In some embodiments, a masking moiety comprises an amino acid sequence having at least 94% identity to SEQ ID NO: 125. In some embodiments, a masking moiety comprises an amino acid sequence having at least 95% identity to SEQ ID NO: 125. In some embodiments, a masking moiety comprises an amino acid sequence having at least 96% identity to SEQ ID NO: 125. In some embodiments, a masking moiety comprises an amino acid sequence having at least 97% identity to SEQ ID NO: 125. In some embodiments, a masking moiety comprises an amino acid sequence having at least 98% identity to SEQ ID NO: 125. In some embodiments, a masking moiety comprises an amino acid sequence having at least 99% identity to SEQ ID NO: 125.

In some embodiments, a masking moiety comprises a VL linked to a VH via a non-cleavable linker. In some embodiments, a masking moiety comprises a VH linked to a VL via a non-cleavable linker. In some embodiments, a non-cleavable linker is

(SEQ ID NO: 141)
GGGGGSGGGGSGGGGSGGGGS

In some embodiments, a masking moiety comprises an amino acid sequence of SEQ ID NO: 142.

(SEQ ID NO: 142)
NFMLTQPHSVSESPGKTVTISCTRSSGSIASNYVQWYQQRPGSSPTTVIYEDNQRPSGVP
DRFSGSIDSSSNSASLTISGLKTEDEADYYCQTYDSIDVYFGGGTKLTVLGGGGGSGGGG
SGGGGSGGGGSQLQLQESGPGLVKPSQTLSLTCTVSGGSISSGGYYWSWIRQHPGKGLE
WIGYIYKSGSAYYSPSLKSRVTISVDTSKNQFSLKLSSVTAADTAVYYCARTPTVTGDW
FDPWGRGTLVTVSS

In some embodiments, a masking moiety comprises an amino acid sequence having at least 80% identity to SEQ ID NO: 142. In some embodiments, a masking moiety comprises an amino acid sequence having at least 85% identity to SEQ ID NO: 142. In some embodiments, a masking moiety comprises an amino acid sequence having at least 87% identity to SEQ ID NO: 142. In some embodiments, a masking moiety comprises an amino acid sequence having at least 88% identity to SEQ ID NO: 142. In some embodiments, a masking moiety comprises an amino acid sequence having at least 90% identity to SEQ ID NO: 142. In some embodiments, a masking moiety comprises an amino acid sequence having at least 92% identity to SEQ ID NO: 142. In some embodiments, a masking moiety comprises an amino acid sequence having at least 93% identity to SEQ ID NO: 142. In some embodiments, a masking moiety comprises an amino acid sequence having at least 94% identity to SEQ ID NO: 142. In some embodiments, a masking moiety comprises an amino acid sequence having at least 95% identity to SEQ ID NO: 142. In some embodiments, a masking moiety comprises an amino acid sequence having at least 96% identity to SEQ ID NO: 142. In some embodiments, a masking moiety comprises an amino acid sequence having at least 97% identity to SEQ ID NO: 142. In some embodiments, a masking moiety comprises an amino acid sequence having at least 98% identity to SEQ ID NO: 142. In some embodiments, a masking moiety comprises an amino acid sequence having at least 99% identity to SEQ ID NO: 142.

In some embodiments, a masking moiety is linked to an engineered Fc domain via a non-cleavable linker of SEQ ID NO: 140 (GGSSGSGGSGGGSGSGGG). In some embodiments, a masking moiety is linked to an engineered Fe domain via a non-cleavable linker of SEQ ID NO: 141 (GGGGGSGGGGSGGGGSGGGGS). In some embodiments, a masking moiety is linked to an engineered Fe domain via a non-cleavable linker of SEQ ID NO: 210 (GGSSGSGGSGGGSGSGGGSGGSGG).

Anti-IL-2 VHH

In some embodiments, a masking moiety comprises an anti-IL-2 VHH having an amino acid sequence of SEQ ID NO: 121. In some embodiments, a masking moiety comprises an anti-IL-2 VHH having a hCDR1 of SEQ ID NO: 132, a hCDR2 of SEQ ID NO: 133, and hCDR3 of SEQ ID NO: 134.

An exemplary anti-IL-2 scFv sequence

| | | |
|---|---|---|
| VHH | EVQLVESGGGLVQPGGSLRLSCAASGSIFSINVMGWYRQA | SEQ ID NO: 121 |
| | PGKQRELVAAISSGGSTNYADSVKGRFTISRDNAKNTVYLQ | |
| | MNSLKPEDTAVYYCMYASSWYEDETDYWGQGTQVTVSS | |
| hCDR1 | GSIFSINVMG | SEQ ID NO: 132 |
| hCDR2 | AISSGGSTNYADSVKG | SEQ ID NO: 133 |
| hCDR3 | ASSWYEDETDY | SEQ ID NO: 134 |

In some embodiments, a masking moiety comprises an amino acid sequence having at least 80% identity to SEQ ID NO: 121. In some embodiments, a masking moiety comprises an amino acid sequence having at least 85% identity to SEQ ID NO: 121. In some embodiments, a masking moiety comprises an amino acid sequence having at least 87% identity to SEQ ID NO: 121. In some embodiments, a masking moiety comprises an amino acid sequence having at least 88% identity to SEQ ID NO: 121. In some embodiments, a masking moiety comprises an amino acid sequence having at least 90% identity to SEQ ID NO: 121. In some embodiments, a masking moiety comprises an amino acid sequence having at least 92% identity to SEQ ID NO: 121. In some embodiments, a masking moiety comprises an amino acid sequence having at least 93% identity to SEQ ID NO: 121. In some embodiments, a masking moiety comprises an amino acid sequence having at least 94% identity to SEQ ID NO: 121. In some embodiments, a masking moiety comprises an amino acid sequence having at least 95% identity to SEQ ID NO: 121. In some embodiments, a masking moiety comprises an amino acid sequence having at least 96% identity to SEQ ID NO: 121. In some embodiments, a masking moiety comprises an amino acid sequence having at least 97% identity to SEQ ID NO: 121. In some embodiments, a masking moiety comprises an amino acid sequence having at least 98% identity to SEQ ID NO: 121. In some embodiments, a masking moiety comprises an amino acid sequence having at least 99% identity to SEQ ID NO: 121.

In some embodiments, a masking moiety is linked to an engineered Fc domain via a non-cleavable linker of SEQ ID NO: 140. In some embodiments, a masking moiety is linked to an engineered Fc domain via a non-cleavable linker of SEQ ID NO: 141. In some embodiments, a masking moiety is linked to an engineered Fc domain via a non-cleavable linker of SEQ ID NO: 210.

CD122 (Masking moiety for IL-2 and IL-15)

The masking moiety masks the cytokine or functional fragment thereof in the masked cytokine thereby reducing or preventing binding of the cytokine or functional fragment thereof to its cognate receptor. In some embodiments, the masking moiety reduces or prevents binding of the IL-2 cytokine or functional fragment thereof to IL-2Rα (CD25). In some embodiments, the masking moiety as provided herein refers to a moiety capable of binding to, or otherwise exhibiting an affinity for the IL-2 cytokine or functional fragment thereof, such as an anti-IL-2 antibody or IL-2 cognate receptor protein. In some embodiments, the masking moiety reduces or prevents binding of the IL-15 cytokine or functional fragment thereof to IL-15Rα. In some embodiments, the masking moiety as provided herein refers to a moiety capable of binding to, or otherwise exhibiting an affinity for the IL-15 cytokine or functional fragment thereof, such as an anti-IL-15 antibody or IL-15 cognate receptor protein. Methods for determining the extent of binding of a protein (e.g., cytokine) to a cognate protein (e.g., cytokine receptor) are well known in the art.

In some embodiments, the masking moiety comprises an IL-2 cytokine receptor, or a subunit or functional fragment thereof.

In some embodiments, the masking moiety comprises CD122 (also referred to as IL-2Rβ) or a fragment, portion, or variant thereof that retains or otherwise demonstrates an affinity to IL-2 and IL-15.

In some embodiments, the masking moiety comprises the amino acid sequence of SEQ ID NO: 50.

(SEQ ID NO: 50)
AVNGTSQFTCFYNSRANISCVWSQDGALQDTSCQVHAWPDRRRWNQTCELLPVSQAS
WACNLILGAPDSQKLTTVDIVTLRVLCREGVRWRVMAIQDFKPFENLRLMAPISLQVVH
VETHRCNISWEISQASHYFERHLEFEARTLSPGHTWEEAPLLTLKQKQEWICLETLTPDT
QYEFQVRVKPLQGEFTTWSPWSQPLAFRTKPAALGKD

In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 50. In some embodiments, the masking moiety comprises an amino acid sequence having the amino acid sequence of SEQ ID NO: 50 with one to four amino acid substitutions. In some embodiments, the masking moiety comprises an amino acid sequence having the amino acid sequence of SEQ ID NO: 50 with one or two amino acid substitutions.

In some embodiments, the CD122 or a fragment, portion or variant thereof has mutation at amino acid position C122 as compared to CD122 of SEQ ID NO: 50.

In some embodiments, the CD122 or a fragment, portion or variant thereof has mutation C122S at amino acid position 122 as compared to CD122 of SEQ ID NO: 50.

In some embodiments, the masking moiety comprises an amino acid sequence of SEQ ID NO: 50 with a C122 mutation.

In some embodiments, the masking moiety comprises an amino acid sequence of SEQ ID NO: 50 with a C122S mutation.

In some embodiments, the CD122 or a fragment, portion or variant thereof has mutation at amino acid position C168 as compared to CD122 of SEQ ID NO: 50.

In some embodiments, the CD122 or a fragment, portion or variant thereof has mutation C168S at amino acid position 168 as compared to CD122 of SEQ ID NO: 50.

In some embodiments, the masking moiety comprises an amino acid sequence of SEQ ID NO: 50 with a C168 mutation.

In some embodiments, the masking moiety comprises an amino acid sequence of SEQ ID NO: 50 with a C168S mutation.

In some embodiments, the CD122 or a fragment, portion or variant thereof has mutation at amino acid positions C122 and C168 as compared to CD122 of SEQ ID NO: 50.

In some embodiments, the CD122 or a fragment, portion or variant thereof has mutation C122S and C168S as compared to CD122 of SEQ ID NO: 50.

In some embodiments, the masking moiety comprises an amino acid sequence of SEQ ID NO: 51.

(SEQ ID NO: 51)
AVNGTSQFTCFYNSRANISCVWSQDGALQDTSCQVHAWPDRRRWNQTCELLPVSQAS
WACNLILGAPDSQKLTTVDIVTLRVLCREGVRWRVMAIQDFKPFENLRLMAPISLQVVH
VETHRSNISWEISQASHYFERHLEFEARTLSPGHTWEEAPLLTLKQKQEWISLETLTPDTQ
YEFQVRVKPLQGEFTTWSPWSQPLAFRTKPAALGKD

In some embodiments, the masking moiety comprises an amino acid sequence having at least about 80% identity to SEQ ID NO: 51. In some embodiments, the masking moiety comprises an amino acid sequence having at least about 85% identity to SEQ ID NO: 51. In some embodiments, the masking moiety comprises an amino acid sequence having at least about 90% identity to SEQ ID NO: 51. In some embodiments, the masking moiety comprises an amino acid sequence having at least about 91% identity to SEQ ID NO: 51. In some embodiments, the masking moiety comprises an amino acid sequence having at least about 92% identity to SEQ ID NO: 51. In some embodiments, the masking moiety comprises an amino acid sequence having at least about 93% identity to SEQ ID NO: 51. In some embodiments, the masking moiety comprises an amino acid sequence having at least about 94% identity to SEQ ID NO: 51. In some embodiments, the masking moiety comprises an amino acid sequence having at least about 95% identity to SEQ ID NO: 51. In some embodiments, the masking moiety comprises an amino acid sequence having at least about 96% identity to SEQ ID NO: 51. In some embodiments, the masking moiety comprises an amino acid sequence having at least about 97% identity to SEQ ID NO: 51. In some embodiments, the masking moiety comprises an amino acid sequence having at least about 98% identity to SEQ ID NO: 51. In some embodiments, the masking moiety comprises an amino acid sequence having at least about 99% identity to SEQ ID NO: 51.

In some embodiments, a masking moiety has a mutation at amino acid positions F8 as compared to CD122 of SEQ ID NO: 50. In some embodiments, a masking moiety has a mutation F8C as compared to CD122 of SEQ ID NO: 50.

In some embodiments, a masking moiety has a mutation at amino acid positions A94 as compared to CD122 of SEQ ID NO: 50. In some embodiments, a masking moiety has a mutation A94C as compared to CD122 of SEQ ID NO: 50.

In some embodiments, a masking moiety has a mutation at amino acid positions L106 as compared to CD122 of SEQ ID NO: 50. In some embodiments, a masking moiety has a mutation L106C as compared to CD122 of SEQ ID NO: 50.

In some embodiments, a masking moiety has a mutation at amino acid positions V117 as compared to CD122 of SEQ ID NO: 50. In some embodiments, a masking moiety has a mutation V117C as compared to CD122 of SEQ ID NO: 50.

In some embodiments, a masking moiety has a mutation at amino acid positions C122 as compared to CD122 of SEQ ID NO: 50. In some embodiments, a masking moiety has a mutation C122S as compared to CD122 of SEQ ID NO: 50. In some embodiments, a masking moiety has a mutation C122V as compared to CD122 of SEQ ID NO: 50. In some embodiments, a masking moiety has a mutation C122A as compared to CD122 of SEQ ID NO: 50.

In some embodiments, a masking moiety has a mutation at amino acid positions N123 as compared to CD122 of SEQ ID NO: 50. In some embodiments, a masking moiety has a mutation N123C as compared to CD122 of SEQ ID NO: 50. In some embodiments, a masking moiety has a mutation N123Q as compared to CD122 of SEQ ID NO: 50.

In some embodiments, a masking moiety has a mutation at amino acid positions C168 as compared to CD122 of SEQ ID NO: 50. In some embodiments, a masking moiety has a mutation C168S as compared to CD122 of SEQ ID NO: 50. In some embodiments, a masking moiety has a mutation C168V as compared to CD122 of SEQ ID NO: 50. In some embodiments, a masking moiety has a mutation C168A as compared to CD122 of SEQ ID NO: 50.

In some embodiments, a masking moiety has a mutation at amino acid positions L169 as compared to CD122 of SEQ ID NO: 50. In some embodiments, a masking moiety has a mutation L169C as compared to CD122 of SEQ ID NO: 50.

In some embodiments, a masking moiety has a mutation at amino acid positions Q177 as compared to CD122 of SEQ ID NO: 50. In some embodiments, a masking moiety has a mutation Q177C as compared to CD122 of SEQ ID NO: 50.

In some embodiments, a masking moiety has a mutation at amino acid positions V184 as compared to CD122 of SEQ ID NO: 50. In some embodiments, a masking moiety has a mutation V184C as compared to CD122 of SEQ ID NO: 50.

In some embodiments, a masking moiety has a mutation at amino acid positions S195 as compared to CD122 of SEQ ID NO: 50. In some embodiments, a masking moiety has a mutation S195C as compared to CD122 of SEQ ID NO: 50.

In some embodiments, a masking moiety has a mutation at amino acid positions R204 as compared to CD122 of SEQ ID NO: 50. In some embodiments, a masking moiety has a mutation R204C as compared to CD122 of SEQ ID NO: 50.

In some embodiments, a masking moiety has mutations C122V/C168V as compared to CD122 of SEQ ID NO: 50.

In some embodiments, a masking moiety comprises an amino acid sequence of SEQ ID NO: 52.

(SEQ ID NO: 52)
AVNGTSQFTCFYNSRANISCVWSQDGALQDTSCQVHAWPDRRRWNQTCELLPVSQAS
WACNLILGAPDSQKLTTVDIVTLRVLCREGVRWRVMAIQDFKPFENLRLMAPISLQVVH
VETHRVNISWEISQASHYFERHLEFEARTLSPGHTWEEAPLLTLKQKQEWIVLETLTPDT
QYEFQVRVKPLQGEFTTWSPWSQPLAFRTKPAALGKD

In some embodiments, a masking moiety has mutations C122A/C168V as compared to CD122 of SEQ ID NO: 50. In some embodiments, a masking moiety comprises an amino acid sequence of SEQ ID NO: 53.

(SEQ ID NO: 53)
AVNGTSQFTCFYNSRANISCVWSQDGALQDTSCQVHAWPDRRRWNQTCELLPVSQAS
WACNLILGAPDSQKLTTVDIVTLRVLCREGVRWRVMAIQDFKPFENLRLMAPISLQVVH
VETHRANISWEISQASHYFERHLEFEARTLSPGHTWEEAPLLTLKQKQEWIVLETLTPDT
QYEFQVRVKPLQGEFTTWSPWSQPLAFRTKPAALGKD

In some embodiments, a masking moiety has a mutations C168V as compared to CD122 of SEQ ID NO: 50. In some embodiments, a masking moiety comprises an amino acid sequence of SEQ ID NO: 54.

(SEQ ID NO: 54)
AVNGTSQFTCFYNSRANISCVWSQDGALQDTSCQVHAWPDRRRWNQTCELLPVSQAS
WACNLILGAPDSQKLTTVDIVTLRVLCREGVRWRVMAIQDFKPFENLRLMAPISLQVVH
VETHRCNISWEISQASHYFERHLEFEARTLSPGHTWEEAPLLTLKQKQEWIVLETLTPDT
QYEFQVRVKPLQGEFTTWSPWSQPLAFRTKPAALGKD

In some embodiments, a masking moiety has mutations C122V/C168A as compared to CD122 of SEQ ID NO: 50. In some embodiments, a masking moiety comprises an amino acid sequence of SEQ ID NO: 55.

(SEQ ID NO: 55)
AVNGTSQFTCFYNSRANISCVWSQDGALQDTSCQVHAWPDRRRWNQTCELLPVSQAS
WACNLILGAPDSQKLTTVDIVTLRVLCREGVRWRVMAIQDFKPFENLRLMAPISLQVVH
VETHRVNISWEISQASHYFERHLEFEARTLSPGHTWEEAPLLTLKQKQEWIALETLTPDT
QYEFQVRVKPLQGEFTTWSPWSQPLAFRTKPAALGKD

In some embodiments, a masking moiety has mutations C122A/N123C as compared to CD122 of SEQ ID NO: 50. In some embodiments, a masking moiety comprises an amino acid sequence of SEQ ID NO: 56.

(SEQ ID NO: 56)
AVNGTSQFTCFYNSRANISCVWSQDGALQDTSCQVHAWPDRRRWNQTCELLPVSQAS
WACNLILGAPDSQKLTTVDIVTLRVLCREGVRWRVMAIQDFKPFENLRLMAPISLQVVH
VETHRACISWEISQASHYFERHLEFEARTLSPGHTWEEAPLLTLKQKQEWICLETLTPDT
QYEFQVRVKPLQGEFTTWSPWSQPLAFRTKPAALGKD

In some embodiments, the masking moiety comprises an amino acid sequence having at least about 80% identity to SEQ ID NO: 56. In some embodiments, the masking moiety comprises an amino acid sequence having at least about 85% identity to SEQ ID NO: 56. In some embodiments, the masking moiety comprises an amino acid sequence having at least about 90% identity to SEQ ID NO: 56. In some embodiments, the masking moiety comprises an amino acid sequence having at least about 91% identity to SEQ ID NO: 56. In some embodiments, the masking moiety comprises an amino acid sequence having at least about 92% identity to SEQ ID NO: 56. In some embodiments, the masking moiety comprises an amino acid sequence having at least about 93% identity to SEQ ID NO: 56. In some embodiments, the masking moiety comprises an amino acid sequence having at least about 94% identity to SEQ ID NO: 56. In some embodiments, the masking moiety comprises an amino acid sequence having at least about 95% identity to SEQ ID NO: 56. In some embodiments, the masking moiety comprises an amino acid sequence having at least about 96% identity to SEQ ID NO: 56. In some embodiments, the masking moiety comprises an amino acid sequence having at least about 97% identity to SEQ ID NO: 56. In some embodiments, the masking moiety comprises an amino acid sequence having at least about 98% identity to SEQ ID NO: 56. In some embodiments, the masking moiety comprises an amino acid sequence having at least about 99% identity to SEQ ID NO: 56.

In some embodiments, a masking moiety has mutations C122V/N123C as compared to CD122 of SEQ ID NO: 50. In some embodiments, a masking moiety comprises an amino acid sequence of SEQ ID NO: 57.

(SEQ ID NO: 57)
AVNGTSQFTCFYNSRANISCVWSQDGALQDTSCQVHAWPDRRRWNQTCELLPVSQAS
WACNLILGAPDSQKLTTVDIVTLRVLCREGVRWRVMAIQDFKPFENLRLMAPISLQVVH
VETHRVCISWEISQASHYFERHLEFEARTLSPGHTWEEAPLLTLKQKQEWICLETLTPDT
QYEFQVRVKPLQGEFTTWSPWSQPLAFRTKPAALGKD

In some embodiments, a masking moiety has mutations C122A/C168A as compared to CD122 of SEQ ID NO: 50. In some embodiments, a masking moiety comprises an amino acid sequence of SEQ ID NO: 58.

(SEQ ID NO: 58)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRLMAPISLQVVHVETHRANISWEISQASHYFERHLEFEARTLS
PGHTWEEAPLLTLKQKQEWIALETLTPDTQYEFQVRVKPLQGEFTTWSP
WSQPLAFRTKPAALGKD

In some embodiments, a masking moiety has mutations V117C/N123Q/C168A as compared to CD122 of SEQ ID NO: 50. In some embodiments, a masking moiety comprises an amino acid sequence of SEQ ID NO: 59.

(SEQ ID NO: 59)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRLMAPISLQVVHCETHRCQISWEISQASHYFERHLEFEARTLS
PGHTWEEAPLLTLKQKQEWIALETLTPDTQYEFQVRVKPLQGEFTTWSP
WSQPLAFRTKPAALGKD

In some embodiments, a masking moiety has mutations N123Q/C168A/L169C as compared to CD122 of SEQ ID NO: 50. In some embodiments, a masking moiety comprises an amino acid sequence of SEQ ID NO: 60.

(SEQ ID NO: 60)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRLMAPISLQVVHVETHRCQISWEISQASHYFERHLEFEARTLS
PGHTWEEAPLLTLKQKQEWIACETLTPDTQYEFQVRVKPLQGEFTTWSP
WSQPLAFRTKPAALGKD

In some embodiments, a masking moiety has mutations L106C/C122A/C168A/S195C as compared to CD122 of SEQ ID NO: 50. In some embodiments, a masking moiety comprises an amino acid sequence of SEQ ID NO: 61.

(SEQ ID NO: 61)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRCMAPISLQVVHVETHRANISWEISQASHYFERHLEFEARTLS
PGHTWEEAPLLTLKQKQEWIALETLTPDTQYEFQVRVKPLQGEFTTWCP
WSQPLAFRTKPAALGKD

In some embodiments, a masking moiety has mutations L106C/C122A/C168A/V184C as compared to CD122 of SEQ ID NO: 50. In some embodiments, a masking moiety comprises an amino acid sequence of SEQ ID NO: 62.

(SEQ ID NO: 62)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRCMAPISLQVVHVETHRANISWEISQASHYFERHLEFEARTLS
PGHTWEEAPLLTLKQKQEWIALETLTPDTQYEFQVRCKPLQGEFTTWSP
WSQPLAFRTKPAALGKD

In some embodiments, a masking moiety has mutations C122A/C168A/V184C/S195C as compared to CD122 of SEQ ID NO: 50. In some embodiments, a masking moiety comprises an amino acid sequence of SEQ ID NO: 63.

(SEQ ID NO: 63)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRLMAPISLQVVHVETHRANISWEISQASHYFERHLEFEARTLS
PGHTWEEAPLLTLKQKQEWIALETLTPDTQYEFQVRCKPLQGEFTTWCP
WSQPLAFRTKPAALGKD

In some embodiments, a masking moiety has mutations C122A/C168A/Q177C/R204C as compared to CD122 of SEQ ID NO: 50. In some embodiments, a masking moiety comprises an amino acid sequence of SEQ ID NO: 64.

(SEQ ID NO: 64)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRLMAPISLQVVHVETHRANISWEISQASHYFERHLEFEARTLS
PGHTWEEAPLLTLKQKQEWIALETLTPDTCYEFQVRVKPLQGEFTTWSP
WSQPLAFCTKPAALGKD

In some embodiments, a masking moiety has mutations L106C/C122V/C168V/S195C as compared to CD122 of SEQ ID NO: 50. In some embodiments, a masking moiety comprises an amino acid sequence of SEQ ID NO: 65.

(SEQ ID NO: 65)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRCMAPISLQVVHVETHRVNISWEISQASHYFERHLEFEARTLS
PGHTWEEAPLLTLKQKQEWIVLETLTPDTQYEFQVRVKPLQGEFTTWCP
WSQPLAFRTKPAALGKD

In some embodiments, a masking moiety has mutations F8C/A94C/C122V/C168V as compared to CD122 of SEQ ID NO: 50. In some embodiments, a masking moiety comprises an amino acid sequence of SEQ ID NO: 66.

(SEQ ID NO: 66)
AVNGTSQCTCFYNSRANISCVWSQDGALQDTSCQVHAWPDRRRWNQTCE
LLPVSQASWACNLILGAPDSQKLTTVDIVTLRVLCREGVRWRVMCIQDF
KPFENLRLMAPISLQVVHVETHRNISWEISQASHYFERHLEFEARTLSP
GHTWEEAPLLTLKQKQEWIVLETLTPDTQYEFQVRVKPLQGEFTTWSPW
SQPLAFRTKPAALGKD

IL-12 Receptor

The masking moiety masks the IL-12 cytokine or functional fragment thereof in the targeted cytokine thereby reducing or preventing binding of the IL-cytokine or functional fragment thereof to its cognate receptor.

The IL-12 receptor, beta 1, or IL-12Rβ1 is a subunit of the IL-12 receptor complex. IL-12Rβ1 is also known as CD212. This protein binds to interleukin-12 (IL-12) with a low affinity. This protein forms a disulfide-linked oligomer, which is required for its IL-12 binding activity. The IL-12 receptor, beta 2, or IL-12Rβ2 is a subunit of the IL-12 receptor complex. The coexpression of IL-12Rβ1 and IL-12Rβ2 protein has been shown to lead to the formation of high-affinity IL-12 binding sites.

In some embodiments, the masking moiety comprises an extracellular domain of an IL-12 cytokine receptor, or a subunit or functional fragment thereof.

Interleukin-12 receptor subunit beta-1, also called CD212 has the sequence:

(SEQ ID NO: 67)
MEPLVTWVVPLLFLFLLSRQGAACRTSECCFQDPPYPDADSGSASGPRD
LRCYRISSDRYECSWQYEGPTAGVSHFLRCCLSSGRCCYFAAGSATRLQ
FSDQAGVSVLYTVTLWVESWARNQTEKSPEVTLQLYNSVKYEPPLGDIK
VSKLAGQLRMEWETPDNQVGAEVQFRHRTPSSPWKLGDCGPQDDDTESC
LCPLEMNVAQEFQLRRRQLGSQGSSWSKWSSPVCVPPENPPQPQVRFSV
EQLGQDGRRRLTLKEQPTQLELPEGCQGLAPGTEVTYRLQLHMLSCPCK
AKATRTLHLGKMPYLSGAAYNVAVISSNQFGPGLNQTWHIPADTHTEPV
ALNISVGTNGTTMYWPARAQSMTYCIEWQPVGQDGGLATCSLTAPQDPD
PAGMATYSWSRESGAMGQEKCYYITIFASAHPEKLTLWSTVLSTYHFGG
NASAAGTPHHVSVKNHSLDSVSVDWAPSLLSTCPGVLKEYVVRCRDEDS
KQVSEHPVQPTETQVTLSGLRAGVAYTVQVRADTAWLRGVWSQPQRFSI
EVQVSDWLIFFASLGSFLSILLVGVLGYLGLNRAARHLCPPLPTPCASS
AIEFPGGKETWQWINPVDFQEEASLQEALVVEMSWDKGERTEPLEKTEL
PEGAPELALDTELSLEDGDRCKAKM

Interleukin-12 receptor subunit beta-2 has the sequence:

(SEQ ID NO: 68)
MAHTFRGCSLAFMFIITWLLIKAKIDACKRGDVTVKPSHVILLGSTVNI
TCSLKPRQGCFHYSRENKLILYKFDRRINFHHGHSLNSQVTGLPLGTTL
FVCKLACINSDEIQICGAEIFVGVAPEQPQNLSCIQKGEQGTVACTWER
GRDTHLYTEYTLQLSGPKNLTWQKQCKDIYCDYLDFGINLTPESPESNF
TAKVTAVNSLGSSSSLPSTFTFLDIVRPLPPWDIRIKFQKASVSRCTLY
WRDEGLVLLNRLRYRPSNSRLWNMVNVTKAKGRHDLLDLKPFTEYEFQI
SSKLHLYKGSWSDWSESLRAQTPEEEPTGMLDVWYMKRHIDYSRQQISL
FWKNLSVSEARGKILHYQVTLQELTGGKAMTQNITGHTSWTTVIPRTGN
WAVAVSAANSKGSSLPTRINIMNLCEAGLLAPRQVSANSEGMDNILVTW
QPPRKDPSAVQEYVVEWRELHPGGDTQVPLNWLRSRPYNVSALISENIK
SYICYEIRVYALSGDQGGCSSILGNSKHKAPLSGPHINAITEEKGSILI
SWNSIPVQEQMGCLLHYRIYWKERDSNSQPQLCEIPYRVSQNSHPINSL
QPRVTYVLWMTALTAAGESSHGNEREFCLQGKANWMAFVAPSICIAIIM
VGIFSTHYFQQKVFVLLAALRPQWCSREIPDPANSTCAKKYPIAEEKTQ
LPLDRLLIDWPTPEDPEPLVISEVLHQVTPVFRHPPCSNWPQREKGIQG
HQASEKDMMHSASSPPPPRALQAESRQLVDLYKVLESRGSDPKPENPAC
PWTVLPAGDLPTHDGYLPSNIDDLPSHEAPLADSLEELEPQHISLSVFP
SSSLHPLTFSCGDKLTLDQLKMRCDSLML

The bold indicates the pro-peptide, the italics with underline indicates the extracellular domain, the italics indicates the transmembrane domain and the bold with underline indicates the cytoplasmic domain.

In some embodiments, the masking moiety comprises the extracellular domain of human IL-12Rβ1 or a fragment, portion, or variant thereof that retains or otherwise demonstrates an affinity to IL-12.

In some embodiments, the masking moiety comprises an amino acid sequence having an amino acid sequence of human IL-12Rβ1 (SEQ ID NO: 68) with one to four amino acid substitutions. In some embodiments, the masking moiety comprises an amino acid sequence having an amino acid sequence of human IL-12Rβ1 with one or two amino acid substitutions.

In some embodiments, the masking moiety comprises residues 24 to 237 of human IL-12Rβ1, namely a sequence having SEQ ID NO: 69 or a fragment, portion, or variant thereof that retains or otherwise demonstrates an affinity to IL-12.

(SEQ ID NO: 69)
CRTSECCFQDPPYPDADSGSASGPRDLRCYRISSDRYECSWQYEGPTAG
VSHFLRCCLSSGRCCYFAAGSATRLQFSDQAGVSVLYTVTLWVESWARN
QTEKSPEVTLQLYNSVKYEPPLGDIKVSKLAGQLRMEWETPDNQVGAEV
QFRHRTPSSPWKLGDCGPQDDDTESCLCPLEMNVAQEFQLRRRQLGSQG
SSWSKWSSPVCVPPENP

In some embodiments, the masking moiety comprises IL-12Rβ1 having SEQ ID NO: 69. In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequence of SEQ ID NO: 69. In some embodiments, the masking moiety comprises an amino acid sequence having at least about 85% sequence identity to SEQ ID NO: 69. In some embodiments, the masking moiety comprises an amino acid sequence having at least about 90% sequence identity to SEQ ID NO: 69. In some embodiments, the masking moiety comprises an amino acid sequence having at least about 92% sequence identity to SEQ ID NO: 69. In some embodiments, the masking moiety comprises an amino acid sequence having at least about 93% sequence identity to SEQ ID NO: 69. In some embodiments, the masking moiety comprises an amino acid sequence having at least about 94% sequence identity to SEQ ID NO: 69. In some embodiments, the masking moiety comprises an amino acid sequence having at least about 95% sequence identity to SEQ ID NO: 69. In some embodiments, the masking moiety comprises an amino acid sequence having at least about 96% sequence identity to SEQ ID NO: 69. In some embodiments, the masking moiety comprises an amino acid sequence having at least about 97% sequence identity to SEQ ID NO: 69. In some embodiments, the masking moiety comprises an amino acid sequence having at least about 98% sequence identity to SEQ ID NO: 69. In some embodiments, the masking moiety comprises an amino acid sequence having at least about 99% sequence identity to SEQ ID NO: 69.

In some embodiments, the masking moiety comprises an amino acid sequence having the amino acid sequence of SEQ ID NO: 69 with one to four amino acid substitutions. In some embodiments, the masking moiety comprises an amino acid sequence having the amino acid sequence of SEQ ID NO: 69 with one or two amino acid substitutions.

In some embodiments, the masking moiety comprises residues 24 to 545 of human IL-12Rβ1, namely a sequence having SEQ ID NO: 70 or a fragment, portion, or variant thereof that retains or otherwise demonstrates an affinity to IL-12.

(SEQ ID NO: 70)
CRTSECCFQDPPYPDADSGSASGPRDLRCYRISSDRYECSWQYEGPTAG
VSHFLRCCLSSGRCCYFAAGSATRLQFSDQAGVSVLYTVTLWVESWARN
QTEKSPEVTLQLYNSVKYEPPLGDIKVSKLAGQLRMEWETPDNQVGAEV
QFRHRTPSSPWKLGDCGPQDDDTESCLCPLEMNVAQEFQLRRRQLGSQG
SSWSKWSSPVCVPPENPPQPQVRFSVEQLGQDGRRRLTLKEQPTQLELP
EGCQGLAPGTEVTYRLQLHMLSCPCKAKATRTLHLGKMPYLSGAAYNVA
VISSNQFGPGLNQTWHIPADTHTEPVALNISVGTNGTTMYWPARAQSMT
YCIEWQPVGQDGGLATCSLTAPQDPDPAGMATYSWSRESGAMGQEKCYY
ITIFASAHPEKLTLWSTVLSTYHFGGNASAAGTPHHVSVKNHSLDSVSV
DWAPSLLSTCPGVLKEYVVRCRDEDSKQVSEHPVQPTETQVTLSGLRAG
VAYTVQVRADTAWLRGVWSQPQRFSIEVQVSD

In some embodiments, the masking moiety comprises IL-12Rβ1 having SEQ ID NO: 70. In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequence of SEQ ID NO: 70. In some embodiments, the masking moiety comprises an amino acid sequence having at least about 85% sequence identity to SEQ ID NO: 70. In some embodiments, the masking moiety comprises an amino acid sequence having at least about 90% sequence identity to SEQ ID NO: 70. In some embodiments, the masking moiety comprises an amino acid sequence having at least about 92% sequence identity to SEQ ID NO: 70. In some embodiments, the masking moiety comprises an amino acid sequence having at least about 93% sequence identity to SEQ ID NO: 70. In some embodiments, the masking moiety comprises an amino acid sequence having at least about 94% sequence identity to SEQ ID NO: 70. In some embodiments, the masking moiety comprises an amino acid sequence having at least about 95% sequence identity to SEQ ID NO: 70. In some embodiments, the masking moiety comprises an amino acid sequence having at least about 96% sequence identity to SEQ ID NO: 70. In some embodiments, the masking moiety comprises an amino acid sequence having at least about 97% sequence identity to SEQ ID NO: 70. In some embodiments, the masking moiety comprises an amino acid sequence having at least about 98% sequence identity to SEQ ID NO: 70. In some embodiments, the masking moiety comprises an amino acid sequence having at least about 99% sequence identity to SEQ ID NO: 70.

In some embodiments, the masking moiety comprises an amino acid sequence having the amino acid sequence of SEQ ID NO: 70 with one to four amino acid substitutions. In some embodiments, the masking moiety comprises an amino acid sequence having the amino acid sequence of SEQ ID NO: 70 with one or two amino acid substitutions.

In some embodiments, the masking moiety comprises the extracellular domain of human IL-12Rβ2 or a fragment, portion, or variant thereof that retains or otherwise demonstrates an affinity to IL-12. In some embodiments, the masking moiety comprises an amino acid sequence having an amino acid sequence of human IL-12Rβ2 (SEQ ID NO: 68) with one to four amino acid substitutions. In some embodiments, the masking moiety comprises an amino acid sequence having an amino acid sequence of human IL-12Rβ2 with one or two amino acid substitutions.

In some embodiments, the masking moiety comprises residues 24 to 212 of human IL-12Rβ2, namely a sequence having SEQ ID NO: 71.

(SEQ ID NO: 71)
KIDACKRGDVTVKPSHVILLGSTVNITCSLKPRQGCFHYSRRNKLILYK
FDRRINFHHGHSLNSQVTGLPLGTTLFVCKLACINSDEIQICGAEIFVG
VAPEQPQNLSCIQKGEQGTVACTWERGRDTHLYTEYTLQLSGPKNLTWQ
KQCKDIYCDYLDFGINLTPESPESNFTAKVTAVNSLGSSSSL

In some embodiments, the masking moiety comprises IL-12Rβ1 having SEQ ID NO: 71. In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequence of SEQ ID NO: 71. In some embodiments, the masking moiety comprises an amino acid sequence having at least about 85% sequence identity to SEQ ID NO: 71. In some embodiments, the masking moiety comprises an amino acid sequence having at least about 90% sequence identity to SEQ ID NO: 71. In some embodiments, the masking moiety comprises an amino acid sequence having at least about 92% sequence identity to SEQ ID NO: 71. In some embodiments, the masking moiety comprises an amino acid sequence having at least about 93% sequence identity to SEQ ID NO: 71. In some embodiments, the masking moiety comprises an amino acid sequence having at least about 94% sequence identity to SEQ ID NO: 71. In some embodiments, the masking moiety comprises an amino acid sequence having at least about 95% sequence identity to SEQ ID NO: 71. In some embodiments, the masking moiety comprises an amino acid sequence having at least about 96% sequence identity to SEQ ID NO: 71. In some embodiments, the masking moiety comprises an amino acid sequence having at least about 97% sequence identity to SEQ ID NO: 71. In some embodiments, the masking moiety comprises an amino acid sequence having at least about 98% sequence identity to SEQ ID NO: 71. In some embodiments, the masking moiety comprises an amino acid sequence having at least about 99% sequence identity to SEQ ID NO: 71.

In some embodiments, the masking moiety comprises an amino acid sequence having the amino acid sequence of SEQ ID NO: 71 with one to four amino acid substitutions. In some embodiments, the masking moiety comprises an amino acid sequence having the amino acid sequence of SEQ ID NO: 71 with one or two amino acid substitutions.

In some embodiments, the masking moiety comprises residues 24 to 222 of human IL-12Rβ2, namely a sequence having SEQ ID NO: 72.

(SEQ ID NO: 72)
KIDACKRGDVTVKPSHVILLGSTVNITCSLKPRQGCFHYSRRNKLILYK
FDRRINFHHGHSLNSQVTGLPLGTTLFVCKLACINSDEIQICGAEIFVG
VAPEQPQNLSCIQKGEQGTVACTWERGRDTHLYTEYTLQLSGPKNLTWQ
KQCKDIYCDYLDFGINLTPESPESNFTAKVTAVNSLGSSSSLPSTFTFL
DIV

In some embodiments, the masking moiety comprises IL-12Rβ1 having SEQ ID NO: 72. In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequence of SEQ ID NO: 72. In some embodiments, the masking moiety comprises an amino acid sequence having at least about 85% sequence identity to SEQ ID NO: 72. In some embodiments, the masking moiety comprises an amino acid sequence having at least about 90% sequence identity to SEQ ID NO: 72. In some embodiments, the masking moiety comprises an amino acid sequence having at least about 92% sequence identity to SEQ ID NO: 72. In some embodiments, the masking moiety comprises an amino acid sequence having at least about 93% sequence identity to SEQ ID NO: 72. In some embodiments, the masking moiety comprises an amino acid sequence having at least about 94% sequence identity to SEQ ID NO: 72. In some embodiments, the masking moiety comprises an amino acid sequence having at least about 95% sequence identity to SEQ ID NO: 72. In some embodiments, the masking moiety comprises an amino acid sequence having at least about 96% sequence identity to SEQ ID NO: 72. In some embodiments, the masking moiety comprises an amino acid sequence having at least about 97% sequence identity to SEQ ID NO: 72. In some embodiments, the masking moiety comprises an amino acid sequence having at least about 98% sequence identity to SEQ ID NO: 72. In some embodiments, the masking moiety comprises an amino acid sequence having at least about 99% sequence identity to SEQ ID NO: 72.

In some embodiments, the masking moiety comprises an amino acid sequence having the amino acid sequence of SEQ ID NO: 72 with one to four amino acid substitutions. In some embodiments, the masking moiety comprises an amino acid sequence having the amino acid sequence of SEQ ID NO: 72 with one or two amino acid substitutions.

In some embodiments, the masking moiety comprises residues 24 to 319 of human IL-12Rβ2, namely a sequence having SEQ ID NO: 73.

(SEQ ID NO: 73)
KIDACKRGDVTVKPSHVILLGSTVNITCSLKPRQGCFHYSRRNKLILYKFD
RRINFHHGHSLNSQVTGLPLGTTLFVCKLACINSDEIQICGAEIFVGVAPE
QPQNLSCIQKGEQGTVACTWERGRDTHLYTEYTLQLSGPKNLTWQKQCKDI
YCDYLDFGINLTPESPESNFTAKVTAVNSLGSSSSLPSTFTFLDIVRPLPP
WDIRIKFQKASVSRCTLYWRDEGLVLLNRLRYRPSNSRLWNMVNVTKAKGR
HDLLDLKPFTEYEFQISSKLHLYKGSWSDWSESLRAQTPEE

In some embodiments, the masking moiety comprises IL-12Rβ2 having SEQ ID NO: 73. In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequence of SEQ ID NO: 73. In some embodiments, the masking moiety comprises an amino acid sequence having at least about 85% sequence identity to SEQ ID NO: 73. In some embodiments, the masking moiety comprises an amino acid sequence having at least about 90% sequence identity to SEQ ID NO: 130. In some embodiments, the masking moiety comprises an amino acid sequence having at least about 92% sequence identity to SEQ ID NO: 73. In some embodiments, the masking moiety comprises an amino acid sequence having at least about 93% sequence identity to SEQ ID NO: 73. In some embodiments, the masking moiety comprises an amino acid sequence having at least about 94% sequence identity to SEQ ID NO: 73. In some embodiments, the masking moiety comprises an amino acid sequence having at least about 95% sequence identity to SEQ ID NO: 73. In some embodiments, the masking moiety comprises an amino acid sequence having at least about 96% sequence identity to SEQ ID NO: 73. In some embodiments, the masking moiety comprises an amino acid sequence having at least about 97% sequence identity to SEQ ID NO: 73. In some embodiments, the masking moiety comprises an amino acid sequence having at least about 98% sequence identity to SEQ ID NO: 73. In some embodiments, the masking moiety comprises an amino acid sequence having at least about 99% sequence identity to SEQ ID NO: 73.

In some embodiments, the masking moiety comprises an amino acid sequence having the amino acid sequence of SEQ ID NO: 73 with one to four amino acid substitutions. In some embodiments, the masking moiety comprises an amino acid sequence having the amino acid sequence of SEQ ID NO: 73 with one or two amino acid substitutions.

In some embodiments, the masking moiety comprises residues 24 to 319 of human IL-12Rβ2, namely a sequence having SEQ ID NO: 73, with one or more cysteine substitutions. In some embodiments, the masking moiety comprises residues 24 to 319 of human IL-12Rβ2, namely a sequence having SEQ ID NO: 73, with an amino acid substitution at position C242. In some embodiments, the amino acid substitution is at position C242 is C242S. In some embodiments, the masking moiety comprises an amino acid sequence of SEQ ID NO: 74.

(SEQ ID NO: 74)
KIDACKRGDVTVKPSHVILLGSTVNITCSLKPRQGCFHYSRRNKLILYKFD
RRINFHHGHSLNSQVTGLPLGTTLFVCKLACINSDEIQICGAEIFVGVAPE
QPQNLSCIQKGEQGTVACTWERGRDTHLYTEYTLQLSGPKNLTWQKQCKDI
YCDYLDFGINLTPESPESNFTAKVTAVNSLGSSSSLPSTFTFLDIVRPLPP
RLWNMVNVTKAKGRHDLLDLKPFTEYEFQISSKLHLYKGSWSDWSESLRAQ
TWDIRIKFQKASVSRSTLYWRDEGLVLLNRLRYRPSNSPEE

In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 74. In some embodiments, the masking moiety consists of an amino acid sequence of SEQ ID NO: 74.

In some embodiments, the masking moiety comprises residues 24 to 622 of human IL-12Rβ2, namely a sequence having SEQ ID NO: 75.

(SEQ ID NO: 75)
KIDACKRGDVTVKPSHVILLGSTVNITCSLKPRQGCFHYSRRNKLILYKFD
RRINFHHGHSLNSQVTGLPLGTTLFVCKLACINSDEIQICGAEIFVGVAPE
QPQNLSCIQKGEQGTVACTWERGRDTHLYTEYTLQLSGPKNLTWQKQCKDI
YCDYLDFGINLTPESPESNFTAKVTAVNSLGSSSSLPSTFTFLDIVRPLPP
WDIRIKFQKASVSRCTLYWRDEGLVLLNRLRYRPSNSRLWNMVNVTKAKGR
HDLLDLKPFTEYEFQISSKLHLYKGSWSDWSESLRAQTPEEEPTGMLDVWY
MKRHIDYSRQQISLFWKNLSVSEARGKILHYQVTLQELTGGKAMTQNITGH
TSWTTVIPRTGNWAVAVSAANSKGSSLPTRINIMNLCEAGLLAPRQVSANS
EGMDNILVTWQPPRKDPSAVQEYVVEWRELHPGGDTQVPLNWLRSRPYNVS
ALISENIKSYICYEIRVYALSGDQGGCSSILGNSKHKAPLSGPHINAITEE
KGSILISWNSIPVQEQMGCLLHYRIYWKER

In some embodiments, the masking moiety comprises IL-12Rβ1 having SEQ ID NO: 75. In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequence of SEQ ID NO: 75. In some embodiments, the masking moiety comprises an amino acid sequence having at least about 85% sequence identity to SEQ ID NO: 75. In some embodiments, the masking moiety comprises an amino acid sequence having at least about 90% sequence identity to SEQ ID NO: 75. In some embodiments, the masking moiety comprises an amino acid sequence having at least about 92% sequence identity to SEQ ID NO: 75. In some embodiments, the masking moiety comprises an amino acid sequence having at least about 93% sequence identity to SEQ ID NO: 75. In some embodiments, the masking moiety comprises an amino acid sequence having at least about 94% sequence identity to SEQ ID NO: 75. In some embodiments, the masking moiety comprises an amino acid sequence having at least about 95% sequence identity to SEQ ID NO: 75. In some embodiments, the masking moiety comprises an amino acid sequence having at least about 96% sequence identity to SEQ ID NO: 75. In some embodiments, the masking moiety comprises an amino acid sequence having at least about 97% sequence identity to SEQ ID NO: 75. In some embodiments, the masking moiety comprises an amino acid sequence having at least about 98% sequence identity to SEQ ID NO: 75. In some embodiments, the masking moiety comprises an amino acid sequence having at least about 99% sequence identity to SEQ ID NO: 75.

In some embodiments, the masking moiety comprises an amino acid sequence having the amino acid sequence of SEQ ID NO: 75 with one to four amino acid substitutions. In some embodiments, the masking moiety comprises an amino acid sequence having the amino acid sequence of SEQ ID NO: 75 with one or two amino acid substitutions.

In some embodiments, the masking moiety comprises residues 24 to 227 of human IL-12Rβ2, namely a sequence having SEQ ID NO: 76.

(SEQ ID NO: 76)
KIDACKRGDVTVKPSHVILLGSTVNITCSLKPRQGCFHYSRRNKLILYKFD
RRINFHHGHSLNSQVTGLPLGTTLFVCKLACINSDEIQICGAEIFVGVAPE
QPQNLSCIQKGEQGTVACTWERGRDTHLYTEYTLQLSGPKNLTWQKQCKDI
YCDYLDFGINLTPESPESNFTAKVTAVNSLGSSSSLPSTFTFLDIVRPLPP

In some embodiments, the masking moiety comprises IL-12Rβ1 having SEQ ID NO: 76. In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequence of SEQ ID NO: 76. In some embodiments, the masking moiety comprises an amino acid sequence having at least about 85% sequence identity to SEQ ID NO: 76. In some embodiments, the masking moiety comprises an amino acid sequence having at least about 90% sequence identity to SEQ ID NO: 76. In some embodiments, the masking moiety comprises an amino acid sequence having at least about 92% sequence identity to SEQ ID NO: 76. In some embodiments, the masking moiety comprises an amino acid sequence having at least about 93% sequence identity to SEQ ID NO: 76. In some embodiments, the masking moiety comprises an amino acid sequence having at least about 94% sequence identity to SEQ ID NO: 76. In some embodiments, the masking moiety comprises an amino acid sequence having at least about 95% sequence identity to SEQ ID NO: 76. In some embodiments, the masking moiety comprises an amino acid sequence having at least about 96% sequence identity to SEQ ID NO: 76. In some embodiments, the masking moiety comprises an amino acid sequence having at least about 97% sequence identity to SEQ ID NO: 76. In some embodiments, the masking moiety comprises an amino acid sequence having at least about 98% sequence identity to SEQ ID NO: 76. In some embodiments, the masking moiety comprises an amino acid sequence having at least about 99% sequence identity to SEQ ID NO: 76.

In some embodiments, the masking moiety comprises an amino acid sequence having the amino acid sequence of SEQ ID NO: 76 with one to four amino acid substitutions. In some embodiments, the masking moiety comprises an amino acid sequence having the amino acid sequence of SEQ ID NO: 76 with one or two amino acid substitutions.

Targeted Cytokine Formats

As discussed, the cleavable Fc domain linked cytokine may further comprise a targeting moiety to form a targeted cytokine. In some embodiments, a targeted cytokine is a bivalent targeting format, comprising 1) a first chain comprising a variable heavy region and an IgG1 or IgG4 heavy constant region with “hole mutations” (Y349C; T366S; L368A; and Y407V); 2) a second chain comprising a variable heavy region, a heavy constant region with “knob mutations” (S354C and T366W) fused to a cytokine or a variant thereof, wherein the CH1 and CH2 domains are from IgG1 or IgG4, and the CH3 domain is from IgG3; and 3) a third chain comprising a variable light region and immunoglobulin kappa or lambda constant region.

In some embodiments, a targeted cytokine is a bivalent targeting format, comprising 1) a first chain comprising a variable heavy region and an IgG1 or IgG4 heavy constant region with “hole mutations” (Y349C; T366S; L368A; and Y407V); 2) a second chain comprising a variable heavy region, a heavy constant region from IgG1 or IgG4 with “knob mutations” (S354C and T366W) and “RF mutations” (H435R and Y436F) fused to a cytokine or a variant thereof; and 3) a third chain comprising a variable light region and immunoglobulin kappa or lambda constant region.

In some embodiments, a targeted cytokine is a bivalent targeting format, comprising 1) a first chain comprising a variable heavy region and an IgG1 or IgG4 heavy constant region with “hole mutations” (Y349C; T366S; L368A; and Y407V) fused to a cytokine or a variant thereof; 2) a second chain comprising a variable heavy region, a heavy constant region with “knob mutations” (S354C and T366W) fused to a masking moiety, wherein the CH1 and CH2 domains are from IgG1 or IgG4, and the CH3 domain is from IgG3; and 3) a third chain comprising a variable light region and immunoglobulin kappa or lambda constant region.

In some embodiments, a targeted cytokine is a bivalent targeting format, comprising 1) a first chain comprising a variable heavy region and an IgG1 or IgG4 heavy constant region with “hole mutations” (Y349C; T366S; L368A; and Y407V) fused to a cytokine or a variant thereof; 2) a second chain comprising a variable heavy region, a heavy constant region from IgG1 or IgG4 with “knob mutations” (S354C and T366W) and “RF mutations” (H435R and Y436F) fused to a masking moiety; and 3) a third chain comprising a variable light region and immunoglobulin kappa or lambda constant region.

In some embodiments, a targeted cytokine is a bivalent targeting format, comprising 1) a first chain comprising a variable heavy region and an IgG1 or IgG4 heavy constant region with “hole mutations” (Y349C; T366S; L368A; and Y407V) fused to a masking moiety; 2) a second chain comprising a variable heavy region, a heavy constant region with “knob mutations” (S354C and T366W) fused to a cytokine or a variant thereof, wherein the CH1 and CH2 domains are from IgG1 or IgG4, and the CH3 domain is from IgG3; and 3) a third chain comprising a variable light region and immunoglobulin kappa or lambda constant region.

In some embodiments, a targeted cytokine is a bivalent targeting format, comprising 1) a first chain comprising a variable heavy region and an IgG1 or IgG4 heavy constant region with “hole mutations” (Y349C; T366S; L368A; and Y407V) fused to a masking moiety; 2) a second chain comprising a variable heavy region, a heavy constant region from IgG1 or IgG4 with “knob mutations” (S354C and T366W) and “RF mutations” (H435R and Y436F) fused to a cytokine or a variant thereof; and 3) a third chain comprising a variable light region and immunoglobulin kappa or lambda constant region.

In some embodiments, a targeted cytokine is a monovalent targeting format, comprising 1) a first chain comprising a Fab fused to a first Fc polypeptide chain from an IgG1 or IgG4 with “hole mutations” (Y349C; T366S; L368A; and Y407V); and 2) a second chain comprising a second Fc polypeptide chain from IgG1 or IgG4 with “knob mutations” (S354C and T366W) and “RF mutations” (H435R and Y436F).

In some embodiments, a targeted cytokine is a monovalent targeting format, comprising 1) a first chain comprising a Fab fused to a first Fc polypeptide chain from an IgG1 or IgG4 with “hole mutations” (Y349C; T366S; L368A; and Y407V); and 2) a second chain comprising a second Fc polypeptide chain with “knob mutations” (S354C and T366W) wherein the CH1 and CH2 domains are from IgG1 or IgG4, and the CH3 domain is from IgG3.

In some embodiments, a targeted cytokine is a monovalent targeting format, comprising 1) a first chain comprising a first Fc polypeptide chain from an IgG1 or IgG4 with “hole mutations” (Y349C; T366S; L368A; and Y407V); and 2) a second chain comprising a Fab fused to a second Fc polypeptide chain from IgG1 or IgG4 with “knob mutations” (S354C and T366W) and “RF mutations” (H435R and Y436F). In some embodiments, a cytokine is fused to a first Fc polypeptide chain. In some embodiments, a cytokine is fused to a second Fc polypeptide chain. In some embodiments, a masking moiety is fused to a first Fc polypeptide chain. In some embodiments, a masking moiety is fused to a second Fc polypeptide chain.

In some embodiments, a targeted cytokine is a monovalent targeting format, comprising 1) a first chain comprising a first Fc polypeptide chain from an IgG1 or IgG4 with “hole mutations” (Y349C; T366S; L368A; and Y407V); and 2) a second chain comprising a Fab fused to a second Fc polypeptide chain with “knob mutations” (S354C and T366W) wherein the CH1 and CH2 domains are from IgG1 or IgG4, and the CH3 domain is from IgG3. In some embodiments, a cytokine is fused to a second Fc polypeptide chain. In some embodiments, a masking moiety is fused to a first Fc polypeptide chain. In some embodiments, a masking moiety is fused to a second Fc polypeptide chain.

In some embodiments, a targeted cytokine is a monovalent targeting format, comprising 1) a first chain comprising a variable light region and IgG kappa or lambda constant region fused to an IgG1 or IgG4 heavy constant region with “hole mutations” (Y349C; T366S; L368A; and Y407V; and 2) a second chain comprising a variable heavy region fused to an IgG1 or IgG4 heavy constant region with “knob mutations” (S354C and T366W) and “RF mutations” (H435R and Y436F), wherein the C-terminal of the CH3 domain is fused to a cytokine or a variant thereof. In some embodiments, a cytokine is fused to a second Fc polypeptide chain. In some embodiments, a masking moiety is fused to a first Fc polypeptide chain. In some embodiments, a masking moiety is fused to a second Fc polypeptide chain.

In some embodiments, a targeted cytokine is a monovalent targeting format, comprising 1) a first chain comprising a variable light region and IgG kappa or lambda constant region fused to an IgG1 or IgG4 heavy constant region with “hole mutations” (Y349C; T366S; L368A; and Y407V; and 2) a second chain comprising a variable heavy region fused to a heavy constant region with “knob mutations” (S354C and T366W) fused to a cytokine or a variant thereof, wherein the CH1 and CH2 domains are from IgG1 or IgG4, and the CH3 domain is from IgG3.

In some embodiments, a targeted cytokine is a monovalent targeting format, comprising 1) a first chain comprising a variable heavy region fused to an IgG1 or IgG4 heavy constant region with “hole mutations” (Y349C; T366S; L368A; and Y407V; and 2) a second chain comprising a variable light region and IgG kappa or lambda constant region fused to an IgG1 or IgG4 heavy constant region with “knob mutations” (S354C and T366W) and “RF mutations” (H435R and Y436F), wherein the C-terminal of the CH3 domain is fused to a cytokine or a variant thereof.

In some embodiments, a targeted cytokine is a monovalent targeting format, comprising 1) a first chain comprising a variable heavy region fused to an IgG1 or IgG4 heavy constant region with “hole mutations” (Y349C; T366S; L638A; and Y407V; and 2) a second chain comprising a light variable region and IgG kappa or lambda constant region fused to a heavy constant region with “knob mutations” (S354C and T366W) fused to a cytokine or a variant thereof, wherein the CH1 and CH2 domains are from IgG1 or IgG4, and the CH3 domain is from IgG3.

Targeting Moiety

In accordance with the present disclosure, a targeted cytokine further comprises a targeting moiety. In some embodiments, a targeting moiety comprises an antigen-binding moiety that binds to an antigen expressed on the surface of a target cell.

In some embodiments, the targeting moiety comprises an antigen-binding moiety, wherein the antigen is expressed on an immune cell. In some embodiments, the targeting moiety comprises an antigen-binding moiety, wherein the antigen is selected from PD-1, PD-L1, CTLA-4, TIGIT, TIM-3, LAG-3, OX40, DR5, ICOS, GITR, CD73, CD39, CD25, CD16a, CD8, KLRC1, KLRD1, KLRB1, CD40, CD137, CD28 and CD16b.

In some embodiments, a targeting moiety specifically binds PD-1, PD-L1, PD-L2, CTLA-4, TIGIT, TIM-3, LAG-3, CD25, CD16a, CD16b, OX40, DR5, ICOS, GITR, NKG2D, KLRC1, KLRD1, KLRB1, NKP44, NKP30, BCMA, human epidermal growth factor receptor 2 (HER2), MICA, DLK1, human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGF R1), vascular endothelial growth factor receptor 2 (VEG FR2), Nectin-4, Liv-1, glycoprotein NMB (GPNMB), prostate specific membrane antigen (PSMA), Trop-2, carbonic anhydrase IX (CA9), endothelin B receptor (ETBR), six transmembrane epithelial antigen of the prostate 1 (STEAP1), NAPI2B, folate receptor alpha (FR-a), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD8, CD20, CD22, CD28, CD33, CD39, CD40, CD56, CD66e, CD70, CD73, CD74, CD79b, CD98, CD123, CD137, CD138, CD352, CD47, signal-regulatory protein alpha (SIRPa), Claudin 18.2, Claudin 6, 5T4, fibroblast activation protein alpha (FAPa), fibronectin, the melanoma-associated chondroitin sulfate proteoglycan (MCSP), epithelial cellular adhesion molecule (EPCAM), or combinations thereof.

In some embodiments, a targeting moiety specifically binds PD-1. In some embodiments, a targeting moiety binds PD-L1.

In some embodiments, a targeting moiety comprises an agent, a peptide, or a polypeptide that specifically binds to a target.

In some embodiments, a targeting moiety comprises a Fab, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), an antibody, or a combination thereof. In some embodiments, a targeting moiety comprises a Fab. In some embodiments, a targeting moiety comprises a single chain Fv (scFv). In some embodiments, a targeting moiety comprises a single domain antibody (VHH). In some embodiments, a targeting moiety comprises one or more CDRs. In some embodiments, a targeting moiety comprises a variable heavy chain (VH). In some embodiments, a targeting moiety comprises a variable light chain (VL). In some embodiments, a targeting moiety comprises a Fab-like bispecific antibodies (bsFab). In some embodiments, a targeting moiety comprises a single-domain antibody-linked Fab (s-Fab). In some embodiments, a targeting moiety comprises an antibody or a fragment thereof.

In some embodiments, a targeting moiety is an anti-PD-1 Fab. In some embodiments, a targeting moiety is an anti-PD-1 scFV.

In some embodiments, a targeting moiety comprises a heavy chain variable region of

(SEQ ID NO: 77)
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSS.

In some embodiments, a targeting moiety comprises an amino acid sequence having at least about 80% sequence identity to SEQ ID NO: 77. In some embodiments, a targeting moiety comprises an amino acid sequence having at least about 85% sequence identity to SEQ ID NO: 77. In some embodiments, a targeting moiety comprises an amino acid sequence having at least about 90% sequence identity to SEQ ID NO: 77. In some embodiments, a targeting moiety comprises an amino acid sequence having at least about 92% sequence identity to SEQ ID NO: 77. In some embodiments, a targeting moiety comprises an amino acid sequence having at least about 95% sequence identity to SEQ ID NO: 77. In some embodiments, a targeting moiety comprises an amino acid sequence having at least about 96% sequence identity to SEQ ID NO: 77. In some embodiments, a targeting moiety comprises an amino acid sequence having at least about 97% sequence identity to SEQ ID NO: 77. In some embodiments, a targeting moiety comprises an amino acid sequence having at least about 98% sequence identity to SEQ ID NO: 77. In some embodiments, a targeting moiety comprises an amino acid sequence having at least about 99% sequence identity to SEQ ID NO: 77.

In some embodiments, a targeting moiety comprises a heavy chain of

(SEQ ID NO: 78)
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSC.

In some embodiments, a targeting moiety comprises an amino acid sequence having at least about 80% sequence identity to SEQ ID NO: 78. In some embodiments, a targeting moiety comprises an amino acid sequence having at least about 85% sequence identity to SEQ ID NO: 78. In some embodiments, a targeting moiety comprises an amino acid sequence having at least about 90% sequence identity to SEQ ID NO: 78. In some embodiments, a targeting moiety comprises an amino acid sequence having at least about 92% sequence identity to SEQ ID NO: 78. In some embodiments, a targeting moiety comprises an amino acid sequence having at least about 95% sequence identity to SEQ ID NO: 78. In some embodiments, a targeting moiety comprises an amino acid sequence having at least about 96% sequence identity to SEQ ID NO: 78. In some embodiments, a targeting moiety comprises an amino acid sequence having at least about 97% sequence identity to SEQ ID NO: 78. In some embodiments, a targeting moiety comprises an amino acid sequence having at least about 98% sequence identity to SEQ ID NO: 78. In some embodiments, a targeting moiety comprises an amino acid sequence having at least about 99% sequence identity to SEQ ID NO: 78.

In some embodiments, a targeting moiety comprises a heavy chain of

(SEQ ID NO: 79)
QVQLVQSGVEVKKPGASVKVSCKASGYTFTNYYMYWVRQAPGQGLEWMGGI
NPSNGGTNFNEKFKNRVTLTTDSSTTTAYMELKSLQFDDTAVYYCARRDYR
FDMGFDYWGQGTTVTVSSASTKGPSVFPLAPCSRSTSESTAALGCLVKDYF
PEPVTVSWNSGALTSGVHTFPAVLQSSGLYSLSSVVTVPSSSLGTKTYTCN
VDHKPSNTKVDKRVESKYGPPCPPCPAPEFLGGPSVFLFPPKPKDTLMISR
TPEVTCVVVDVSQEDPEVQFNWYVDGVEVHNAKTKPREEQFNSTYRVVSVL
TVLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLYS
RLTVDKSRWQEGNVFSCSVMHEALHNHYTQKSLSLSLGK

In some embodiments, a targeting moiety comprises an amino acid sequence having at least about 80% sequence identity to SEQ ID NO: 79. In some embodiments, a targeting moiety comprises an amino acid sequence having at least about 85% sequence identity to SEQ ID NO: 79. In some embodiments, a targeting moiety comprises an amino acid sequence having at least about 90% sequence identity to SEQ ID NO: 79. In some embodiments, a targeting moiety comprises an amino acid sequence having at least about 92% sequence identity to SEQ ID NO: 79. In some embodiments, a targeting moiety comprises an amino acid sequence having at least about 95% sequence identity to SEQ ID NO: 79. In some embodiments, a targeting moiety comprises an amino acid sequence having at least about 96% sequence identity to SEQ ID NO: 79. In some embodiments, a targeting moiety comprises an amino acid sequence having at least about 97% sequence identity to SEQ ID NO: 79. In some embodiments, a targeting moiety comprises an amino acid sequence having at least about 98% sequence identity to SEQ ID NO: 79. In some embodiments, a targeting moiety comprises an amino acid sequence having at least about 99% sequence identity to SEQ ID NO: 79.

In some embodiments, a targeting moiety comprises a heavy chain CDR1 sequence of GYTFTNYY (SEQ ID NO: 80). In some embodiments, a targeting moiety comprises a heavy chain CDR2 sequence of INPSNGGT (SEQ ID NO: 81). In some embodiments, a targeting moiety comprises a heavy chain CDR3 sequence of ARRDYRFDMGFDY (SEQ ID NO: 82). In some embodiments, a targeting moiety comprises a HCDR1 of SEQ ID NO: 80, a HCDR2 of SEQ ID NO: 81, and a HCDR3 of SEQ ID NO: 82.

In some embodiments, a targeting moiety comprises a light chain variable region of

(SEQ ID NO: 83)
EIVLTQSPATLSLSPGERATLSCRASKGVSTSGYSYLHWYQQKPGQAPRLL
IYLASYLESGVPARFSGSGSGTDFTLTISSLEPEDFAVYYCQHSRDLPLTF
GGGTKVEIKTSENLYFQ.

In some embodiments, a targeting moiety comprises an amino acid sequence having at least about 80% sequence identity to SEQ ID NO: 83. In some embodiments, a targeting moiety comprises an amino acid sequence having at least about 85% sequence identity to SEQ ID NO: 83. In some embodiments, a targeting moiety comprises an amino acid sequence having at least about 90% sequence identity to SEQ ID NO: 83. In some embodiments, a targeting moiety comprises an amino acid sequence having at least about 92% sequence identity to SEQ ID NO: 83. In some embodiments, a targeting moiety comprises an amino acid sequence having at least about 95% sequence identity to SEQ ID NO: 83. In some embodiments, a targeting moiety comprises an amino acid sequence having at least about 96% sequence identity to SEQ ID NO: 83. In some embodiments, a targeting moiety comprises an amino acid sequence having at least about 97% sequence identity to SEQ ID NO: 83. In some embodiments, a targeting moiety comprises an amino acid sequence having at least about 98% sequence identity to SEQ ID NO: 83. In some embodiments, a targeting moiety comprises an amino acid sequence having at least about 99% sequence identity to SEQ ID NO: 83.

In some embodiments, a targeting moiety comprises a light chain of

(SEQ ID NO: 84)
EIVLTQSPATLSLSPGERATLSCRASKGVSTSGYSYLHWYQQKPGQAPRLL
IYLASYLESGVPARFSGSGSGTDFTLTISSLEPEDFAVYYCQHSRDLPLTF
GGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQG
LSSPVTKSFNRGEC.

In some embodiments, a targeting moiety comprises an amino acid sequence having at least about 80% sequence identity to SEQ ID NO: 84. In some embodiments, a targeting moiety comprises an amino acid sequence having at least about 85% sequence identity to SEQ ID NO: 84. In some embodiments, a targeting moiety comprises an amino acid sequence having at least about 90% sequence identity SEQ ID NO: 84. In some embodiments, a targeting moiety comprises an amino acid sequence having at least about 92% sequence identity to SEQ ID NO: 84. In some embodiments, a targeting moiety comprises an amino acid sequence having at least about 95% sequence identity to SEQ ID NO: 84. In some embodiments, a targeting moiety comprises an amino acid sequence having at least about 96% sequence identity to SEQ ID NO: 84. In some embodiments, a targeting moiety comprises an amino acid sequence having at least about 97% sequence identity to SEQ ID NO: 84. In some embodiments, a targeting moiety comprises an amino acid sequence having at least about 98% sequence identity to SEQ ID NO: 84. In some embodiments, a targeting moiety comprises an amino acid sequence having at least about 99% sequence identity to SEQ ID NO: 84.

In some embodiments, a targeting moiety comprises a light chain CDR1 sequence of KGVSTSGYSY (SEQ ID NO: 85). In some embodiments, a targeting moiety comprises a light chain CDR2 sequence of LAS (SEQ ID NO: 86). In some embodiments, a targeting moiety comprises a light chain CDR3 sequence of QHSRDLPLT (SEQ ID NO: 87). In some embodiments, a targeting moiety comprises a LCDR1 of SEQ ID NO: 85, a LCDR2 of SEQ ID NO: 86, and a LCDR3 of SEQ ID NO: 87.

In some embodiments, a targeting moiety comprises a HCDR1 of SEQ ID NO: 80, a HCDR2 of SEQ ID NO: 81, a HCDR3 of SEQ ID NO: 82, a LCDR1 of SEQ ID NO: 85, a LCDR2 of SEQ ID NO: 86, and a LCDR3 of SEQ ID NO: 87.

In some embodiments, a targeting moiety comprises a heavy chain variable region of

(SEQ ID NO: 213)
QVQLVESGGGVVQPGRSLRLDCKASGITFSNSGMHWVRQAPGKGLEWVAVI
WYDGSKRYYADSVKGRFTISRDNSKNTLFLQMNSLRAEDTAVYYCATNDDY
WGQGTLVTVSS.

In some embodiments, a targeting moiety comprises an amino acid sequence having at least about 80% sequence identity to SEQ ID NO: 213. In some embodiments, a targeting moiety comprises an amino acid sequence having at least about 85% sequence identity to SEQ ID NO: 213. In some embodiments, a targeting moiety comprises an amino acid sequence having at least about 90% sequence identity to SEQ ID NO: 213. In some embodiments, a targeting moiety comprises an amino acid sequence having at least about 92% sequence identity to SEQ ID NO: 213. In some embodiments, a targeting moiety comprises an amino acid sequence having at least about 95% sequence identity to SEQ ID NO: 213. In some embodiments, a targeting moiety comprises an amino acid sequence having at least about 96% sequence identity to SEQ ID NO: 213. In some embodiments, a targeting moiety comprises an amino acid sequence having at least about 97% sequence identity to SEQ ID NO: 213. In some embodiments, a targeting moiety comprises an amino acid sequence having at least about 98% sequence identity to SEQ ID NO: 213. In some embodiments, a targeting moiety comprises an amino acid sequence having at least about 99% sequence identity to SEQ ID NO: 213.

In some embodiments, a targeting moiety comprises a heavy chain of

(SEQ ID NO: 211)
QVQLVESGGGVVQPGRSLRLDCKASGITFSNSGMHWVRQAPGKGLEWVAVI
WYDGSKRYYADSVKGRFTISRDNSKNTLFLQMNSLRAEDTAVYYCATNDDY
WGQGTLVTVSSASTKGPSVFPLAPCSRSTSESTAALGCLVKDYFPEPVTVS
WNSGALTSGVHTFPAVLQSSGLYSLSSVVTVPSSSLGTKTYTCNVDHKPSN
TKVDKRVESKYGPPCPPCPAPEFLGGPSVFLFPPKPKDTLMISRTPEVTCV
VVDVSQEDPEVQFNWYVDGVEVHNAKTKPREEQFNSTYRVVSVLTVLHQDW
LNGKEYKCKVSNKGLPSSIEKTISKAKGQPREPQVYTLPPSQEEMTKNQVS
LTCLVKGFYPSDIAVEWESNGQPENNYKTTPPVLDSDGSFFLYSRLTVDKS
RWQEGNVFSCSVMHEALHNHYTQKSLSLSLGK.

In some embodiments, a targeting moiety comprises an amino acid sequence having at least about 80% sequence identity to SEQ ID NO: 211. In some embodiments, a targeting moiety comprises an amino acid sequence having at least about 85% sequence identity to SEQ ID NO: 211. In some embodiments, a targeting moiety comprises an amino acid sequence having at least about 90% sequence identity to SEQ ID NO: 211. In some embodiments, a targeting moiety comprises an amino acid sequence having at least about 92% sequence identity to SEQ ID NO: 211. In some embodiments, a targeting moiety comprises an amino acid sequence having at least about 95% sequence identity to SEQ ID NO: 211. In some embodiments, a targeting moiety comprises an amino acid sequence having at least about 96% sequence identity to SEQ ID NO: 211. In some embodiments, a targeting moiety comprises an amino acid sequence having at least about 97% sequence identity to SEQ ID NO: 211. In some embodiments, a targeting moiety comprises an amino acid sequence having at least about 98% sequence identity to SEQ ID NO: 211. In some embodiments, a targeting moiety comprises an amino acid sequence having at least about 99% sequence identity to SEQ ID NO: 211.

In some embodiments, a targeting moiety comprises a heavy chain of

(SEQ ID NO: 214)
QVQLVESGGGVVQPGRSLRLDCKASGITFSNSGMHWVRQAPGKGLEWVAVI
WYDGSKRYYADSVKGRFTISRDNSKNTLFLQMNSLRAEDTAVYYCATNDDY
WGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSN
TKVDKKVEPKSC

In some embodiments, a targeting moiety comprises an amino acid sequence having at least about 80% sequence identity to SEQ ID NO: 214. In some embodiments, a targeting moiety comprises an amino acid sequence having at least about 85% sequence identity to SEQ ID NO: 214. In some embodiments, a targeting moiety comprises an amino acid sequence having at least about 90% sequence identity to SEQ ID NO: 214. In some embodiments, a targeting moiety comprises an amino acid sequence having at least about 92% sequence identity to SEQ ID NO: 214. In some embodiments, a targeting moiety comprises an amino acid sequence having at least about 95% sequence identity to SEQ ID NO: 214. In some embodiments, a targeting moiety comprises an amino acid sequence having at least about 96% sequence identity to SEQ ID NO: 214. In some embodiments, a targeting moiety comprises an amino acid sequence having at least about 97% sequence identity to SEQ ID NO: 214. In some embodiments, a targeting moiety comprises an amino acid sequence having at least about 98% sequence identity to SEQ ID NO: 214. In some embodiments, a targeting moiety comprises an amino acid sequence having at least about 99% sequence identity to SEQ ID NO: 214.

In some embodiments, a targeting moiety comprises a heavy chain CDR1 sequence of GITFSNSG (SEQ ID NO: 216). In some embodiments, a targeting moiety comprises a heavy chain CDR2 sequence of VIWYDGSKRYYADSVKG (SEQ ID NO: 217). In some embodiments, a targeting moiety comprises a heavy chain CDR3 sequence of ATNDDY (SEQ ID NO: 218). In some embodiments, a targeting moiety comprises a HCDR1 of SEQ ID NO: 216, a HCDR2 of SEQ ID NO: 217, and a HCDR3 of SEQ ID NO: 218.

In some embodiments, a targeting moiety comprises a light chain of

(SEQ ID NO: 212)
EIVLTQSPATLSLSPGERATLSCRASQSVSSYLAWYQQKPGQAPRLLIYDA
SNRATGIPARFSGSGSGTDFTLTISSLEPEDFAVYYCQQSSNWPRTFGQGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGE.

In some embodiments, a targeting moiety comprises an amino acid sequence having at least about 80% sequence identity to SEQ ID NO: 212. In some embodiments, a targeting moiety comprises an amino acid sequence having at least about 85% sequence identity to SEQ ID NO: 212. In some embodiments, a targeting moiety comprises an amino acid sequence having at least about 90% sequence identity to SEQ ID NO: 212. In some embodiments, a targeting moiety comprises an amino acid sequence having at least about 92% sequence identity to SEQ ID NO: 212. In some embodiments, a targeting moiety comprises an amino acid sequence having at least about 95% sequence identity to SEQ ID NO: 212. In some embodiments, a targeting moiety comprises an amino acid sequence having at least about 96% sequence identity to SEQ ID NO: 212. In some embodiments, a targeting moiety comprises an amino acid sequence having at least about 97% sequence identity to SEQ ID NO: 212. In some embodiments, a targeting moiety comprises an amino acid sequence having at least about 98% sequence identity to SEQ ID NO: 212. In some embodiments, a targeting moiety comprises an amino acid sequence having at least about 99% sequence identity to SEQ ID NO: 212.

In some embodiments, a targeting moiety comprises a light chain variable region of

(SEQ ID NO: 215)
EIVLTQSPATLSLSPGERATLSCRASQSVSSYLAWYQQKPGQAPRLLIYDA
SNRATGIPARFSGSGSGTDFTLTISSLEPEDFAVYYCQQSSNWPRTFGQGT
KVEIK.

In some embodiments, a targeting moiety comprises an amino acid sequence having at least about 80% sequence identity to SEQ ID NO: 215. In some embodiments, a targeting moiety comprises an amino acid sequence having at least about 85% sequence identity to SEQ ID NO: 215. In some embodiments, a targeting moiety comprises an amino acid sequence having at least about 90% sequence identity SEQ ID NO: 215. In some embodiments, a targeting moiety comprises an amino acid sequence having at least about 92% sequence identity to SEQ ID NO: 215. In some embodiments, a targeting moiety comprises an amino acid sequence having at least about 95% sequence identity to SEQ ID NO: 215. In some embodiments, a targeting moiety comprises an amino acid sequence having at least about 96% sequence identity to SEQ ID NO: 215. In some embodiments, a targeting moiety comprises an amino acid sequence having at least about 97% sequence identity to SEQ ID NO: 215. In some embodiments, a targeting moiety comprises an amino acid sequence having at least about 98% sequence identity to SEQ ID NO: 215. In some embodiments, a targeting moiety comprises an amino acid sequence having at least about 99% sequence identity to SEQ ID NO: 215.

In some embodiments, a targeting moiety comprises a light chain CDR1 sequence of QSVSSY (SEQ ID NO: 219). In some embodiments, a targeting moiety comprises a light chain CDR2 sequence of DAS (SEQ ID NO: 220). In some embodiments, a targeting moiety comprises a light chain CDR3 sequence of QQSSNWPRT (SEQ ID NO: 221). In some embodiments, a targeting moiety comprises a LCDR1 of SEQ ID NO: 219, a LCDR2 of SEQ ID NO: 220, and a LCDR3 of SEQ ID NO: 221.

In some embodiments, a targeting moiety comprises a HCDR1 of SEQ ID NO: 216, a HCDR2 of SEQ ID NO: 217, a HCDR3 of SEQ ID NO: 218, a LCDR1 of SEQ ID NO: 219, a LCDR2 of SEQ ID NO: 220, and a LCDR3 of SEQ ID NO: 221.

In some embodiments, a targeting domain is fused to an Fc polypeptide. In some embodiments, the C-terminus of a targeting domain is fused to the N-terminus of an Fc polypeptide. In some embodiments, the heavy chain of a Fab is fused to an Fc polypeptide. In some embodiments, the C-terminus of the heavy chain of a Fab is fused to the N-terminus of an Fc polypeptide. In some embodiments, an Fc polypeptide comprises a cleavage site.

Exemplary Targeted and Masked Cytokines

In one aspect, the present invention provides, among other things, a targeted masked cytokine comprising a cytokine, a masking moiety, a targeting moiety, and an engineered cleavable Fc domain comprising a protease cleavage site, wherein the engineered Fc domain is fused to the cytokine moiety or the masking moiety such that the masking moiety binds to the cytokine moiety and upon cleavage of the tumor-associated protease cleavage site on the engineered Fc domain, the cytokine moiety is released from the masking moiety.

In some embodiments, a targeted masked cytokine comprises a first polypeptide comprising a heavy chain of a targeting moiety, a non-cleavable Fc polypeptide, and a cytokine in N-to-C terminus orientation, a second polypeptide comprising a heavy chain of the targeting moiety, an engineered cleavable Fc polypeptide, and a masking moiety in N-to-C terminus orientation, and a third polypeptide comprising a light chain of the targeting moiety that associates with the heavy chain of the targeting moiety.

In some embodiments, a targeted masked cytokine comprises a first polypeptide comprising a heavy chain of a targeting moiety, an engineered cleavable Fc polypeptide, and a cytokine in N-to-C terminus orientation, a second polypeptide comprising a heavy chain of the targeting moiety, a non-cleavable Fc polypeptide, and a masking moiety in N-to-C terminus orientation, and a third polypeptide comprising a light chain of the targeting moiety that associates with the heavy chain of the targeting moiety.

In some embodiments, a targeted masked cytokine comprises a first polypeptide comprising a scFv targeting moiety (e.g., heavy chain variable region fused to a light chain variable region (VH-VL or VL-VH)), a non-cleavable Fc polypeptide linked to a cytokine via a non-cleavable linker in N-to-C terminus orientation, and a second polypeptide comprising a scFv targeting moiety (e.g., heavy chain variable region fused to a light chain variable region (VH-VL or VL-VH)), a engineered cleavable Fc polypeptide linked to a masking moiety via a non-cleavable linker in N-to-C terminus orientation.

In some embodiments, a targeted masked cytokine comprises a first polypeptide comprising a scFv targeting moiety (e.g., heavy chain variable region fused to a light chain variable region (VH-VL or VL-VH)), an engineered cleavable Fc polypeptide linked to a cytokine via a non-cleavable linker in N-to-C terminus orientation, and a second polypeptide comprising a scFv targeting moiety (e.g., heavy chain variable region fused to a light chain variable region (VH-VL or VL-VH)), a non-cleavable Fc polypeptide linked to a masking moiety via a non-cleavable linker in N-to-C terminus orientation.

In some embodiment, a targeted masked cytokine in accordance with the invention comprises:
a) a cytokine (optionally comprising one or more amino acid substitutions);
b) a masking moiety (e.g., a single-chain antibody that specifically binds the cytokine);
c) a targeting moiety (e.g., antibody, or antigen-binding fragment thereof, that specifically binds an antigen expressed on the surface of a target cell);
d) a first Fc polypeptide; and
e) a second Fc polypeptide engineered to comprise a protease cleavage site;
   wherein:
i. the first Fc polypeptide is fused to the cytokine moiety and the second Fc polypeptide is fused to the masking moiety such that the masking moiety binds to the cytokine moiety and, upon cleavage of the cleavage site, the cytokine moiety is released from the masking moiety; and
ii. the first Fc polypeptide comprises mutations to form a hole and the second Fc polypeptide comprises mutations to form a knob, or the first Fc polypeptide comprises mutations to form a knob and the second Fc polypeptide comprises mutations to form a hole.

In some embodiments, the mutations to form a hole are amino acid substitutions Y349C, T366S, L368A and Y407V. In some embodiments, the mutations to form a hole are amino acid substitutions Y349C, T366S, L368A, Y407V, and N297A.

In some embodiments, the mutations to form a knob are amino acid substitutions S354C and T366W. In some embodiments, the mutations to form a knob are amino acid substitutions S354C, T366W and N297A.

In some embodiments, the first Fc polypeptide has the amino acid sequence of SEQ ID NO: 11, and the second Fc polypeptide has the amino acid sequence of SEQ ID NO: 5 engineered to comprise a protease cleavage site.

In some embodiments, the C-terminus of the first Fc polypeptide is fused to the cytokine via a first linker. In some embodiments, the first linker is a non-cleavable linker rich in amino acid residues G, S and P. In some embodiments, the first linker only includes amino acid residues selected from the group consisting of G, S and P. In some embodiments, the first linker includes a ‘GS’ repeat. In some embodiments, the first linker includes an N-terminal ‘P’ residue. In some embodiments, the first linker has or comprises the amino acid sequence of SEQ ID NO: 89.

In some embodiments, the C-terminus of the second Fc polypeptide is fused to the masking moiety via a second linker. In some embodiments, the second linker is a non-cleavable linker comprising the amino acid sequence GGS. In some embodiments, the second linker includes [(G),S], where n=4 or 5. In some embodiments, the second linker has or comprises the amino acid sequence of SEQ ID NO: 140.

In some embodiments, the masking moiety is a single-chain Fv (scFv) or a single domain antibody (e.g., VHH) that specifically binds the cytokine.

In some embodiments, the C-terminus of the targeting moiety is fused to the first Fc polypeptide and/or the second Fc polypeptide. In some embodiments, the C-terminus of the targeting moiety is fused to the first Fc polypeptide and/or the second Fc polypeptide without a linker.

In some embodiments, the target cell is a tumor cell. In some embodiments, the target cell is an immune cell. In some embodiments, the targeting moiety binds an antigen expressed on the surface of the immune cell, such as PD-1, PD-L1 or CTLA-4. In some embodiments, the targeting moiety is an antibody, or antigen-binding fragment thereof (e.g., a Fab), that specifically binds PD-1, PD-L1 or CTLA-4.

In some embodiments, the targeting moiety is an antigen-binding fragment such as a Fab. In some embodiments, the targeting moiety is a single-chain antibody such as a single-chain Fv (scFv) or a single domain antibody (e.g., VHH).

In some embodiments, the targeting moiety comprises a heavy chain (HC) fragment lacking an Fc domain. In some embodiments, the C-terminus of the HC fragment is fused to N-terminus of each of the first Fc polypeptide and the second Fc polypeptide. In some embodiments, the targeting moiety is formed by the HC fragment binding to a light chain (LC).

In some embodiments, the targeting moiety is an anti-PD1 Fab. In some embodiments, the HC fragment has the amino acid sequence of SEQ ID NO: 78 and the LC has the amino acid sequence of SEQ ID NO: 84.

In some embodiments, the second Fc polypeptide is engineered by substitution of amino acids in the CH3 domain to comprise the protease cleavage site. In some embodiments, the second Fc polypeptide is engineered by substitution of one or more amino acids at positions 438 to 447. In some embodiments, the second Fc polypeptide is engineered by substitution of four or more (e.g., five, six, seven, eight or nine) amino acids. In some embodiment, the second Fc polypeptide comprises one set of substitutions listed in Table 11. In some embodiment, the second Fc polypeptide comprises one set of substitutions listed in Table 11a. In some embodiment, the second Fc polypeptide comprises one set of substitutions listed in Table 1b. In some embodiment, the second Fc polypeptide comprises one set of substitutions listed in Table 11c.

In some embodiments, the protease cleavage site is cleaved by a tumor-associated protease (e.g., MMP2, MMP3, or MMP9). In some embodiments, the protease cleavage site comprises PLGL, MPY (e.g., MPYDLYHP), APAG (e.g., APAGLIVPYN) or PAN (e.g., PANLVAPDP).

In some embodiments, the protease cleavage site is cleaved by cathepsin B. In some embodiments, the protease cleavage site is cleaved by matriptase.

In some embodiments, the cytokine is IL-2, IL-12, or IL-15. In some embodiments, the cytokine comprises one or more (e.g., one, two, three, four or five) amino acid substitutions, for example, to increases the affinity of the cytokine for its receptor.

In some embodiments, the cytokine is IL-2 comprising the amino acid substitution C125A. In some embodiments, the cytokine is IL-2 comprising the amino acid substitutions R38A, F42A, Y45A, E62A, and C125A.

In some embodiments, the cytokine has the amino acid sequence of SEQ ID NO: 13 or 14, and the masking moiety has the amino acid sequence of SEQ ID NO: 121.

In some embodiments, the first and second Fc polypeptides are IgG1. In some embodiments, the first and second Fc polypeptides are IgG4.

In some embodiments, the Fc polypeptide comprising the knob mutations comprises an CH3 domain that comprises an IgG3 sequence. In some embodiments, the Fc polypeptide comprising the knob mutations further comprises RF mutations (435R/436F).

TABLE 12a
Exemplary Constructs for Targeted Masked Cytokine

| | | Masking | Targeting | First/Second | First/Second |
|---|---|---|---|---|---|
| Construct | Cytokine | Moiety | Moiety | Fc polypeptide | Linker |
| #1 | IL-2 with | N/A | Anti-PD-1 Fab | Fc knob (SEQ ID | SEQ ID |
| | substitution | | HC | NO: 11) | NO: 89 |
| | C125A | | (SEQ ID NO: | | |
| | (SEQ ID | | 78) and LC | | |
| | NO: 13) | | (SEQ ID NO: | | |
| | | | 84) | | |
| | N/A | Anti- IL-2 | Anti-PD-1 Fab | Fc hole + protease | SEQ ID |
| | | VHH | HC | site (SEQ ID NO: 5 | NO: 140 |
| | | (SEQ ID NO: | (SEQ ID NO: | engineered to include | |
| | | 121) | 78) and LC | PLGL) | |
| | | | (SEQ ID NO: | | |
| | | | 84) | | |
| #2 | IL-2 with | N/A | Anti-PD-1 Fab | Fc knob (SEQ ID | SEQ ID |
| | substitutions | | HC | NO: 11) | NO: 89 |
| | R38A, F42A, | | (SEQ ID NO: | | |
| | Y45A, E62A, | | 78) and LC | | |
| | C125A | | (SEQ ID NO: | | |
| | (SEQ ID | | 84) | | |
| | NO: 14) | | | | |
| | N/A | Anti- IL-2 | Anti-PD-1 Fab | Fc hole + protease | SEQ ID |
| | | VHH | HC | site (SEQ ID NO: 5 | NO: 140 |
| | | (SEQ ID NO: | (SEQ ID NO: | engineered to include | |
| | | 121) | 78) and LC | PLGL) | |
| | | | (SEQ ID NO: | | |
| | | | 84) | | |
| #3 | IL-2 with | N/A | Anti-PD-1 Fab | Fc knob (SEQ ID | SEQ ID |
| | substitutions | | HC | NO: 11) | NO: 89 |
| | R38A, F42A, | | (SEQ ID NO: | | |
| | Y45A, E62A, | | 78) and LC | | |
| | C125A | | (SEQ ID NO: | | |
| | (SEQ ID | | 84) | | |
| | NO: 14) | | | | |
| | N/A | Anti- IL-2 | Anti-PD-1 Fab | Fc hole + protease | SEQ ID |
| | | VHH | HC | site (SEQ ID NO: 5 | NO: 140 |
| | | (SEQ ID NO: | (SEQ ID NO: | engineered to include | |
| | | 121) | 78) and LC | MPYDLYHP) | |
| | | | (SEQ ID NO: | | |
| | | | 84) | | |
| #4 | IL-2 with | N/A | Anti-PD-1 Fab | Fc knob (SEQ ID | SEQ ID |
| | substitutions | | HC | NO: 11) | NO: 89 |
| | R38A, F42A, | | (SEQ ID NO: | | |
| | Y45A, E62A, | | 78) and LC | | |
| | C125A | | (SEQ ID NO: | | |
| | (SEQ ID | | 84) | | |
| | NO: 14) | | | | |
| | N/A | Anti- IL-2 | Anti-PD-1 Fab | Fc hole + protease | SEQ ID |
| | | VHH | HC | site (SEQ ID NO: 5 | NO: 140 |
| | | (SEQ ID NO: | (SEQ ID NO: | engineered to include | |
| | | 121) | 78) and LC | APAGLIVPYN) | |
| | | | (SEQ ID NO: | | |
| | | | 84) | | |
| #5 | IL-2 with | N/A | Anti-PD-1 Fab | Fc knob (SEQ ID | SEQ ID |
| | substitutions | | HC | NO: 11) | NO: 89 |
| | R38A, F42A, | | (SEQ ID NO: | | |
| | Y45A, E62A, | | 78) and LC | | |
| | C125A | | (SEQ ID NO: | | |
| | (SEQ ID | | 84) | | |
| | NO: 14) | | | | |
| | N/A | Anti- IL-2 | Anti-PD-1 Fab | Fc hole + protease | G + SEQ ID |
| | | VHH | HC | site (SEQ ID NO: 5 | NO: 140 |
| | | (SEQ ID NO: | (SEQ ID NO: | engineered to include | |
| | | 121) | 78) and LC | PANLVAPDP) | |
| | | | (SEQ ID NO: | | |
| | | | 84) | | |
| #6 | IL-2 with | N/A | Anti-PD-1 Fab | Fc knob (SEQ ID | SEQ ID |
| | substitutions | | HC | NO: 11) | NO: 89 |
| | R38A, F42A, | | (SEQ ID NO: | | |
| | Y45A, E62A, | | 78) and LC | | |
| | C125A | | (SEQ ID NO: | | |
| | (SEQ ID | | 84) | | |
| | NO: 14) | | | | |
| | N/A | Anti- IL-2 | Anti-PD-1 Fab | Fc hole + protease | SEQ ID |
| | | VHH | HC | site (SEQ ID NO: 5 | NO: 140 |
| | | (SEQ ID NO: | (SEQ ID NO: | engineered to include | |
| | | 121) | 78) and LC | GGPLGL) | |
| | | | (SEQ ID NO: | | |
| | | | 84) | | |
| #7 | IL-2 with | N/A | Anti-PD-1 Fab | Fc knob (SEQ ID | SEQ ID |
| | substitutions | | HC | NO: 11) | NO: 89 |
| | R38A, F42A, | | (SEQ ID NO: | | |
| | Y45A, E62A, | | 78) and LC | | |
| | C125A | | (SEQ ID NO: | | |
| | (SEQ ID | | 84) | | |
| | NO: 14) | | | | |
| | N/A | Anti- IL-2 | Anti-PD-1 Fab | Fc hole + protease | SEQ ID |
| | | VHH | HC | site (SEQ ID NO: 5 | NO: 140 |
| | | (SEQ ID NO: | (SEQ ID NO: | engineered to include | |
| | | 121) | 78) and LC | GGPLGL) | |
| | | | (SEQ ID NO: | | |
| | | | 84) | | |

Other Masked or Targeted Therapeutic Molecule

A cleavable carrier or an engineered cleavable Fe domain of the present invention is adaptable and applicable beyond targeted or masked cytokines. For example, a cleavable carrier or an engineered cleavable Fc domain of the present invention can be fused to any other therapeutically active domain.

In accordance with the present disclosure, the engineered cleavable Fc domain comprising a tumor-associated protease cleavage site can be fused to a masked therapeutically active domain comprising a therapeutically active domain and a masking moiety. Alternatively, the engineered cleavable Fc domain comprising a tumor-associated protease cleavage site can be fused to a targeted therapeutically active domain comprising a therapeutically active domain, a masking moiety, and a targeting moiety. Upon the cleavage of the Fc domain, e.g., at the protease cleavage site, the masked and targeted therapeutically active molecules are released and become active at the site of disease, and are able to specifically target a cell of interest for effective treatment of various diseases without causing undesired side effects.

In some embodiments, a therapeutically active domain is linked to an engineered cleavable Fc domain comprising a tumor-associated protease cleavage site, via a non-cleavable linker. In some embodiments, a therapeutically active domain is linked to an Fc domain without the engineered cleavable site via a non-cleavable linker.

In some embodiments, a therapeutically active domain is a cell engager. In some embodiments, a therapeutically active domain is a T cell engager. In some embodiments, a therapeutically active domain is a bispecific T cell engager (BiTE). In some embodiments, a therapeutically active domain is a NK cell engager. In some embodiments, a cell engager is an anti-CD3 antibody or antigen-binding fragment thereof. In some embodiments, a cell engager is an anti-CD16 antibody or antigen-binding fragment thereof. In some embodiments, a cell engager is an anti-NKG2D antibody or antigen-binding fragment thereof. In some embodiments, a cell engager is an anti-CD16/NKG2D bispecific antibody or antigen-binding fragment thereof. In some embodiments, a cell engager is an anti-CD3/CD33 bispecific antibody or antigen-binding fragment thereof. In some embodiments, a cell engager is an anti-CD3/CD19 bispecific antibody or antigen-binding fragment thereof. In some embodiments, a cell engager is an anti-CD56 antibody or antigen-binding fragment thereof. In some embodiments, a cell engager is an anti-CD4 antibody or antigen-binding fragment thereof. In some embodiments, a cell engager is an anti-CD8 antibody or antigen-binding fragment thereof. In some embodiments, a cell engager is an anti-CD25 antibody or antigen-binding fragment thereof. In some embodiments, a cell engager is an anti-CD127 antibody or antigen-binding fragment thereof. In some embodiments, a cell engager is an anti-FoxP3 antibody or antigen-binding fragment thereof. In some embodiments, a cell engager is an anti-CD161 antibody or antigen-binding fragment thereof. In some embodiments, a cell engager is an anti-CD94 antibody or antigen-binding fragment thereof. In some embodiments, a cell engager is an anti-CD57 antibody or antigen-binding fragment thereof. In some embodiments, a cell engager is an anti-NKp46 antibody or antigen-binding fragment thereof. In some embodiments, a cell engager is an anti-NKp30 antibody or antigen-binding fragment thereof.

In some embodiments, a therapeutically active domain is co-stimulatory domain. In some embodiments. In some embodiments, a therapeutically active domain is an agonistic antibody. In some embodiments, a therapeutically active domain is an antagonistic antibody. In some embodiments, a therapeutically active domain is a CD28. In some embodiments, a therapeutically active domain is B7. In some embodiments, a therapeutically active domain is ICOS. In some embodiments, a therapeutically active domain is CD226. In some embodiments, a therapeutically active domain is 41BB. In some embodiments, a therapeutically active domain is OX40. In some embodiments, a therapeutically active domain is CD27. In some embodiments, a therapeutically active domain is GITR. In some embodiments, a therapeutically active domain is HVEM. In some embodiments, a therapeutically active domain is CD40. In some embodiments, a therapeutically active domain is BAFFR. In some embodiments, a therapeutically active domain is BAFF. In some embodiments, a therapeutically active domain is TNF. In some embodiments, a therapeutically active domain is TNF receptor. In some embodiments, a therapeutically active domain is CTLA-4. In some embodiments, a therapeutically active domain is PD-1. In some embodiments, a therapeutically active domain is CD30. In some embodiments, a therapeutically active domain is CD40L. In some embodiments, a therapeutically active domain is TIM-1. In some embodiments, a therapeutically active domain is TIM-2. In some embodiments, a therapeutically active domain is TIM-3. In some embodiments, a therapeutically active domain is CD2. In some embodiments, a therapeutically active domain is CD137. In some embodiments, a therapeutically active domain is 2B4.

In some embodiments, a therapeutically active domain is an anti-CD28 antibody. In some embodiments, a therapeutically active domain is an anti-B7 antibody. In some embodiments, a therapeutically active domain is an anti-ICOS antibody. In some embodiments, a therapeutically active domain is an anti-CD226 antibody. In some embodiments, a therapeutically active domain is an anti-41BB antibody. In some embodiments, a therapeutically active domain is an anti-OX40 antibody. In some embodiments, a therapeutically active domain is an anti-CD27 antibody. In some embodiments, a therapeutically active domain is an anti-GITR antibody. In some embodiments, a therapeutically active domain is an anti-HVEM antibody. In some embodiments, a therapeutically active domain is an anti-CD40 antibody. In some embodiments, a therapeutically active domain is an anti-BAFFR antibody. In some embodiments, a therapeutically active domain is an anti-BAFF antibody. In some embodiments, a therapeutically active domain is an anti-TNF antibody. In some embodiments, a therapeutically active domain is an anti-TNFR antibody. In some embodiments, a therapeutically active domain is an anti-CTLA-4 antibody. In some embodiments, a therapeutically active domain is an anti-PD-1 antibody. In some embodiments, a therapeutically active domain is an anti-CD30 antibody. In some embodiments, a therapeutically active domain is an anti-CD40L antibody. In some embodiments, a therapeutically active domain is an anti-TIM-1 antibody. In some embodiments, a therapeutically active domain is an anti-TIM-2 antibody. In some embodiments, a therapeutically active domain is an anti-TIM-3 antibody. In some embodiments, a therapeutically active domain is an anti-CD2 antibody. In some embodiments, a therapeutically active domain is an anti-CD137 antibody. In some embodiments, a therapeutically active domain is an anti-2B4 antibody.

In some embodiments, a masking moiety is linked to an engineered cleavable Fc domain via a non-cleavable linker. In some embodiments, a targeting moiety is linked to an engineered Fc domain with or without non-cleavable linker. In some embodiments, a masking moiety binds a therapeutically active domain. In some embodiments, a masking moiety inhibits the activity of a therapeutically active domain.

In some embodiments, a therapeutically active domain is linked to an engineered cleavable Fc domain comprising a tumor-associated protease cleavage site, via a non-cleavable linker and a masking moiety is linked to an Fc domain without a cleavage site via a non-cleavable linker. In some embodiments, a masking domain is linked to an engineered cleavable Fc domain comprising a tumor-associated protease cleavage site, via a non-cleavable linker and a therapeutically active domain is linked to an Fc domain without a cleavage site via a non-cleavable linker.

In one aspect, the present invention provides, among other things, a masked therapeutically active molecule comprising a therapeutically active domain, a masking moiety, and an engineered Fc domain comprising an engineered Fc domain comprising a tumor-associated protease cleavage site; wherein the engineered Fc domain is fused to the therapeutically active domain or the masking moiety such that the masking moiety binds to the therapeutically active domain, and the therapeutically active domain is released from the masking moiety upon cleavage of the tumor-associated protease cleavage site on the engineered Fc domain.

In one aspect, the present invention provides, among other things, a masked cell engager comprising a cell engager, a masking moiety, and an engineered Fc domain comprising an engineered Fc domain comprising a tumor-associated protease cleavage site; wherein the engineered Fc domain is fused to the cell engager or the masking moiety such that the masking moiety binds to the cell engager, and the cell engager is released from the masking moiety upon cleavage of the tumor-associated protease cleavage site on the engineered Fc domain.

In one aspect, the present invention provides, among other things, a masked co-stimulatory molecule comprising a co-stimulatory domain, a masking moiety, and an engineered Fc domain comprising an engineered Fc domain comprising a tumor-associated protease cleavage site; wherein the engineered Fc domain is fused to the co-stimulatory domain or the masking moiety such that the masking moiety binds to the co-stimulatory domain, and the co-stimulatory domain is released from the masking moiety upon cleavage of the tumor-associated protease cleavage site on the engineered Fc domain.

In some embodiments, a masked therapeutically active molecule further comprises a targeting moiety. In some embodiments, a masked cell engager further comprises a targeting moiety. In some embodiments, a masked co-stimulatory molecule further comprises a targeting moiety.

In some embodiments, a targeting moiety binds a tumor-associated antigen. In some embodiments, a targeting moiety is an antibody or an antigen binding fragment that binds a tumor-associated antigen. In some embodiments, a targeting moiety is an bispecific antibody or an antigen binding fragment that binds a tumor-associated antigen. In some embodiments, a targeting moiety is an anti-alpha-fetoprotein (AFP) antibody or a fragment thereof. In some embodiments, a targeting moiety is an anti-B2M antibody or a fragment thereof. In some embodiments, a targeting moiety is an anti-beta-human chorionic gonadotropin (beta-hCG) antibody or a fragment thereof. In some embodiments, a targeting moiety is an anti-CD117 antibody or a fragment thereof. In some embodiments, a targeting moiety is an anti-CD19 antibody or a fragment thereof. In some embodiments, a targeting moiety is an anti-CD20 antibody or a fragment thereof. In some embodiments, a targeting moiety is an anti-CD22 antibody or a fragment thereof. In some embodiments, a targeting moiety is an anti-CD25 antibody or a fragment thereof. In some embodiments, a targeting moiety is an anti-CD30 antibody or a fragment thereof. In some embodiments, a targeting moiety is an anti-CD33 antibody or a fragment thereof. In some embodiments, a targeting moiety is an anti-CD151 antibody or a fragment thereof. In some embodiments, a targeting moiety is an anti-MUC-1 antibody or a fragment thereof.

Linkers

In accordance with the present disclosure, the moieties of the masked cytokines and/or targeted cytokines are fused to generate fusion products. In some embodiments, the moieties of the masked cytokines and/or targeted cytokines are fused directly to each other. For example, the engineered Fc domain is directly fused to the cytokine moiety or the masking moiety.

In other embodiments, the moieties of the masked cytokines and/or targeted cytokines are linked through a linker moiety, such as a peptide linker. In some embodiments, the linker is non-cleavable. In other embodiments, the linker is cleavable.

Provided herein are linkers for use in a targeted cytokine or cleavage product thereof. A linker as provided herein refers to a peptide of two more amino acids that is used to link two functional components together in the targeted cytokines described herein.

The targeted cytokine comprises a first linker and a second linker, where at least the first linker or the second linker comprises a proteolytically cleavable peptide.

In some embodiments, a first Fc polypeptide is linked to a cytokine or a variant thereof through a first linker. In some embodiments, a second Fc polypeptide is linked to a masking moiety through a second linker. In some embodiments, a first Fc polypeptide is linked to a cytokine or a variant thereof through a cleavable linker. In some embodiments, a second Fc polypeptide is linked to a masking moiety through a cleavable linker. In some embodiments, a first Fc polypeptide is linked to a cytokine or a variant thereof through a cleavable linker and a second Fc polypeptide is linked to a masking moiety through a non-cleavable linker. In some embodiments, a first Fc polypeptide is linked to a cytokine or a variant thereof through a non-cleavable linker and a second Fc polypeptide is linked to a masking moiety through a cleavable linker. In some embodiments, a first Fc polypeptide is linked to a cytokine or a variant thereof through a cleavable linker and a second Fc polypeptide is linked to a masking moiety through a cleavable linker. In some embodiments, a first Fc polypeptide is linked to a cytokine or a variant thereof through a non-cleavable linker and a second Fc polypeptide is linked to a masking moiety through a non-cleavable linker.

Non-Cleavable Linker

In some embodiments, the non-cleavable linker is between 2 and 25 amino acids in length. In some embodiments, the non-cleavable linker is between 3 and 21 amino acids in length. In some embodiments, the non-cleavable linker is between 3 and 18 amino acids in length. In some embodiments, the non-cleavable linker is between 5 and 18 amino acids in length. In some embodiments, the non-cleavable linker is between 3 and 8 amino acids in length. In some embodiments, the non-cleavable linker is between 4 and 6 amino acids in length.

In some embodiments, the non-cleavable linker is 15 amino acids in length. In some embodiments, the non-cleavable linker is 16 amino acids in length. In some embodiments, the non-cleavable linker is 17 amino acids in length. In some embodiments, the non-cleavable linker is 18 amino acids in length. In some embodiments, the non-cleavable linker is 19 amino acids in length. In some embodiments, the non-cleavable linker is 20 amino acids in length.

In some embodiments, the non-cleavable linker is rich in amino acid residues G, S and P. In some embodiments, the non-cleavable linker only includes amino acid residue types selected from the group consisting of G, S and P. In some embodiments, the non-cleavable linker includes a ‘GS’ repeat. In some embodiments, the non-cleavable linker includes an N′ terminal ‘P’ residue.

In some embodiments, the non-cleavable linker comprises an amino acid sequence as shown in SEQ ID NO: 88 (PGSGS).

In some embodiments, the non-cleavable linker comprises an amino acid sequence as shown in SEQ ID NO: 89 (GGSSPPGGGSSGGGSGP).

In some embodiments, the non-cleavable linker comprises an amino acid sequence GGS.

In some embodiments, the non-cleavable linker includes [(G)ₙS], where n=4 or 5.

In some embodiments, the non-cleavable linker comprises an amino acid sequence as shown in SEQ ID NO: 43 (GGGGS).

In some embodiments, the non-cleavable linker comprises an amino acid sequence as shown in SEQ ID NO: 90 (GGGGSGGGGS).

In some embodiments, the non-cleavable linker comprises an amino acid sequence as shown in SEQ ID NO: 91 (GGSGGGSGGGGGS).

In some embodiments, the non-cleavable linker comprises an amino acid sequence as shown in SEQ ID NO: 92 (GGSGGSGGSGGSGGSSGP).

In some embodiments, the non-cleavable linker comprises an amino acid sequence as shown in SEQ ID NO: 93 (PGGSGP).

In some embodiments, the non-cleavable linker comprises an amino acid sequence as shown in SEQ ID NO: 94 (GGSPG).

In some embodiments, the non-cleavable linker comprises an amino acid sequence as shown in SEQ ID NO: 120 (GGSSPPGGG).

In some embodiments, wherein the second linker comprises a proteolytically cleavable peptide such that the second linker is a proteolytically cleavable linker and the first linker does not comprise a proteolytically cleavable peptide such that the first linker is a non-proteolytically cleavable linker, the non-cleavable linker is between 3 and 18 amino acids in length. In some embodiments, the non-cleavable linker comprises an amino acid sequence as shown in SEQ ID NO: 43 (GGGGS). In some embodiments, the non-cleavable linker comprises an amino acid sequence as shown in SEQ ID NO: 90 (GGGGSGGGGS). In some embodiments, the non-cleavable linker comprises an amino acid sequence as shown in SEQ ID NO: 91(GGSGGGSGGGGGS). In some embodiments, the non-cleavable linker comprises an amino acid sequence as shown in SEQ ID NO: 92 (GGSGGSGGSGGSGGSSGP). In some embodiments, the non-cleavable linker comprises an amino acid sequence as shown in SEQ ID NO: 93(PGGSGP). In some embodiments, the non-cleavable linker comprises an amino acid sequence as shown in SEQ ID NO: 94 (GGSPG).

In some embodiments, it is desirable for the first and second polypeptide chains to be of the same or a similar length to facilitate the first half life extension domain associating with the second half life extension domain and the masking moiety masking the cytokine or functional fragment thereof in the assembled construct. As such where the masking moiety is a shorter amino acid sequence than the cytokine or functional fragment thereof, the difference in length may be compensated fully or in part by using a longer linker L1.

### III. Polynucleotides, Vectors and Host Cells

Provided in the present disclosure also include polynucleotides, vectors and host cells for the engineered cleavable Fc domain and cytokines described herein.

In some embodiments, the nucleotide sequence encoding the engineered cleavable Fc domain is provided. The nucleotide sequence may be codon optimized. In some embodiments, the nucleotide sequence comprises at least one nucleotide modification. In other embodiments, the nucleotide sequence is unmodified.

In some embodiments, a polynucleotide encoding the present cleavable Fc domain comprising a tumor-associated cleavage site is provided. In some embodiments, a polynucleotide that encodes the cleavable Fc domain linked cytokine (masked or targeted cytokine) is provided. The polynucleotide may be produced using any recombinant technologies known in the art. The polynucleotide may be codon optimized. In some embodiments, the polynucleotide comprises at least one nucleotide modification. In some embodiments, the nucleotides of the polynucleotide are un-modified.

In some embodiments, vectors comprising a polynucleotide encoding a cleavable Fc domain comprising a tumor-associated cleavage site or a cytokine of the present disclosure are provided. Vectors include, for example, plasmids, cosmids, viral vectors (such as adenoviruses (“Ad”), adeno-associated viruses (AAV), lentiviruses, and retroviruses), liposomes and other lipid-containing complexes, and other macromolecular complexes capable of mediating delivery of a polynucleotide to a host cell. Other vectors include those described by Chen et al; BioTechniques, 34: 167-171 (2003). A large variety of such vectors are also known in the art and are generally available.

In some embodiments, a host cell comprising a polynucleotide encoding a cleavable Fc domain comprising a tumor-associated cleavage site or a cytokine of the present disclosure is provided. Methods of using the host cell to produce the cleavable Fc domain comprising a tumor-associated cleavage site or the present cytokine are provided as well. As used herein, a “host cell” refers to a cell which is capable of protein expression and optionally protein secretion. For the host cell to express a polypeptide, a nucleotide sequence encoding the polypeptide is present or introduced in the cell. Host cells provided can be prokaryotes or eukaryotes. Examples of eukaryotic cells include, but are not limited to, vertebrate cells, nammalian cells. human cells, animal cells, invertebrate cells, plan cells, nemnatodal cells, insect cells, sten cells, fungal cells or yeast cells. In some embodiments, the host cell is a mammalian cell, a recombinant cell or an engineered cell.

### IV. Methods for Production

Some embodiments of the methods and comnpositions provided herein relate to methods for producing a therapeutic molecule such as targeted masked cytokines. The targeted masked cytokines as described herein can be produced and manufactured using any known technology in the art. The targeted masked cytokines and/or constructs may be produced using any mammalian expression system.

Mammalian cells are commonly used for producing recombinant proteins. The cells are be derived from human, rat, mouse and other mammals. Commonly used mammalian cell lines include but are not limited to, HEK cells, CHO cells, recombinant CHO cells, BHK cells, NSO cells, SP2/O-Agl4 cells, HT-1080 cells, PER.C6 cells, CAP (CEVEC's Amniocyte Production), Hela cells, and HuH-7 (Human hepatoma) cells. The cell line can be used for transient gene expression or as stable cell line for stable expression.

In some embodiments, the cells are Chinese Hamster ovary (CHO) cells. The CHO cells can be recombinant CHO cell lines, e.g., CHO-K1, CHO DUX and CHO DG44.

In some embodiments, the cells are Human Embryo Kidney (HEK) cells or variants thereof (e.g., HKB11).

The host cells are transformed to express a nucleic acid or vector encoding a targeted masked cytokine described herein. Where appropriate, the engineered host cells can be cultured in conventional nutrient media.

In other embodiments, the targeted masked cytokines may be produced using non-mammalian expression systems, such as baculovirus expression systems, bacterial systems, yeast cells, insect cell lines and plant cells. The insect cell lines include but are not limited to s Sf9, Sf21 and BTI 5B1-4. E. coli cells are commonly used bacteria to express a recombinant protein.

In some embodiments, the masked cytokines and/or constructs may be produced using a cell-free protein express system. Cell-free expression systems can use mRNA transcribed from a DNA construct comprising a promoter operably linked to a nucleic acid encoding the polypeptide or fragment thereof.

Pharmaceutical Compositions

In one aspect of the present disclosure, compositions and pharmaceutical compositions comprising the present engineered cleavable Fc domain, the masked cytokine and/or the targeted cytokine are provided. The pharmaceutical composition of the invention can comprise one or more pharmaceutically acceptable carriers, excipients and/or diluents. The composition comprises a therapeutically effective amount of the engineered cleavable Fc domain, the masked cytokine, or the targeted cytokine, and a pharmaceutically acceptable carrier, excipient and/or diluent, including but not limited to, saline, buffered saline, protein stabilizer, solvent, dextrose, water, glycerol, ethanol, and combinations thereof. The compositions are formulated and dosed in a fashion consistent with standard medical practice.

In some embodiments, the present composition is formulated that is suitable the mode of administration. As non-limiting examples, the pharmaceutical composition is formulated for administration in a manner including oral, buccal, nasal, rectal, parenteral, intraperitoneal, intradermal, transdermal, subcutaneous, intravenous, intra-arterial, intracardiac, intraventricular, intracranial, intratracheal, and intrathecal administration, etc., or otherwise by implantation or inhalation. Thus, the present compositions can be formulated into preparations in solid, semi-solid, liquid or gaseous forms, such as tablets, capsules, powders, granules, ointments, solutions, suppositories, enemas, injections, inhalants and aerosols. The following methods and excipients are merely exemplary and are in no way limiting.

In some embodiments, the present compositions are formulated for oral administration, in the form of solutions, suspensions, tablets, pills, granules, capsules, sustained release formulations, oral rinses, or powders. In some embodiments, the present compositions are formulated for parenteral delivery. In other embodiments, the present compositions are formulated in aerosol formulation to be administered via inhalation.

In some embodiments, the pharmaceutical composition comprises one or more additional therapeutic agents.

In some embodiments, the present disclosure also provides a pharmaceutical pack or kit comprising the engineered cleavable Fc domain, the masked cytokine and/or the targeted cytokine.

### V. Methods of Use

In another aspect, the present disclosure provides methods of use of cytokines described herein. In some embodiments, provided includes a method for treating or preventing a disease in a subject in need; the method comprises administering to the subject an effective amount of any masked or targeted cytokine described herein or compositions thereof. In some embodiments, the subject (e.g., a human patient) has been diagnosed with cancer or is at risk of developing cancer. As used herein, “cancer” refers to all types of cancer or neoplasm or malignant tumor found in mammals, including, but not limited to: leukaemia, lymphomas, melanomas, carcinomas and sarcomas. Examples of cancers are cancer of the brain, breast, pancreas, cervix, colon, head & neck, kidney, lung, non-small cell lung, melanoma, mesothelioma, ovary, sarcoma, stomach, uterus and Medulloblastoma.

In some embodiments, cytokines and compositions described in the present disclosure are used for treatment of neoplastic disease; the treatment is capable of invoking one or more effects such as inhibition, to some extent, of tumor growth, including slowing down and/or complete growth arrest; reduction in the number of tumor cells; maintaining tumor size and/or reduction in tumor size; inhibition (e.g., reduction, (slowing down and/or complete prevention of tumor cell infiltration into peripheral organs; inhibition (e.g., reduction, slowing down and/or complete prevention of metastasis; enhancement of anti-tumor immune response, which may result in maintaining tumor size, reducing tumor size, slowing the growth of a tumor, reducing, slowing or preventing invasion, and/or reducing, slowing or preventing metastasis; and/or relief, to some extent, of one or more symptoms associated with the disorder.

In some embodiments, cytokines and compositions described in the present disclosure are used for treatment of an inflammatory disease.

In some embodiments, cytokines and compositions described in the present disclosure are used for treatment of an autoimmune disease, including but not limited to, type 1 diabetes, rheumatoid arthritis, psoriasis, multiple sclerosis, systemic lupus erythematosus (SLE), autoimmune vasculitis, pernicious anaemia and inflammatory bowel disease.

In some embodiments, methods are provided for treating or preventing a disease in a subject comprising administering to the subject an effective amount of any targeted cytokine described herein or compositions thereof, wherein the targeted cytokine is activated upon cleavage of the protease cleavage site within the Fc domain by a protease. In some embodiments, the targeted cytokine is activated at a tumor microenvironment by a tumor-associated protease. The targeted cytokine is therapeutically active after it has cleaved. Thus, in some embodiments, the active agent is the cleavage product.

For the prevention or treatment of disease, the appropriate dosage of an active agent will depend on the type of disease to be treated, as defined herein, the severity and course of the disease, whether the agent is administered for preventive or therapeutic purposes, previous therapy, the subject's clinical history and response to the agent, and the discretion of the attending physician. The agent is suitably administered to the subject at one time or over a series of treatments.

In some embodiments of the methods described herein, an interval between administrations of a targeted cytokine described herein is about one week or longer. In some embodiments of the methods described herein, an interval between administrations of a targeted cytokine described herein is about two days or longer, about three days or longer, about four days or longer, about five days or longer, or about six days or longer. In some embodiments of the methods described herein, an interval between administrations of a targeted cytokine described herein is about one week or longer, about two weeks or longer, about three weeks or longer, or about four weeks or longer. In some embodiments of the methods described herein, an interval between administrations of a targeted cytokine described herein is about one month or longer, about two months or longer, or about three months or longer. As used herein, an interval between administrations refers to the time period between one administration of the targeted cytokine and the next administration of the targeted cytokine. As used herein, an interval of about one month includes four weeks. In some embodiments, the treatment includes multiple administrations of the targeted cytokine, wherein the interval between administrations may vary. For example, in some embodiments, the interval between the first administration and the second administration is about one week, and the intervals between the subsequent administrations are about two weeks. In some embodiments, the interval between the first administration and the second administration is about two days, three days, four days, or five days, or six days, and the intervals between the subsequent administrations are about one week.

In some embodiments, the targeted cytokine is administered on multiple occasions over a period of time, from days, weeks to months. The dosage that is administered to the subject on multiple occasions can, in some embodiments, be the same dosage for each administration, or, in some embodiments, the targeted cytokine can be administered to the subject at two or more different dosages. For example, in some embodiments, a targeted cytokine is initially administered at one dosage on one or more occasions and is later administered at a second dosage on one or more occasions beginning at a later time point.

In some embodiments, a targeted polypeptide described herein is administered at a flat dose. In some embodiments, a targeted polypeptide described herein is administered to a subject at a dosage from about 25 mg to about 500 mg per dose. In some embodiments, the targeted polypeptide is administered to a subject at a dosage of about 25 mg to about 50 mg, about 50 mg to about 75 mg, about 75 mg to about 100 mg, about 100 mg to about 125 mg, about 125 mg to about 150 mg, about 150 mg to about 175 mg, about 175 mg to about 200 mg, about 200 mg to about 225 mg, about 225 mg to about 250 mg, about 250 mg to about 275 mg, about 275 mg to about 300 mg, about 300 mg to about 325 mg, about 325 mg to about 350 mg, about 350 mg to about 375 mg, about 375 mg to about 400 mg, about 400 mg to about 425 mg, about 425 mt to about 450 mg, about 450 mg, to about 475 mg, or about 475 mg to about 500 mg per dose.

In some embodiments, a masked cytokine and/or a targeted cytokine described herein is administered to a subject at a dosage based on the subject's weight or body surface area (BSA). Depending on the type and severity of the disease, about 1 μg/kg to 15 mg/kg (e.g. 0.1 mg/kg-10 mg/kg) of targeted polypeptide can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the targeted polypeptide would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. In some embodiments, a targeted polypeptide described herein is administered to a subject at a dosage from about 0.1 mg/kg to about 10 mg/kg or about 1.0 mg/kg to about 10 mg/kg. In some embodiments, a targeted polypeptide described herein is administered to a subject at a dosage of about any of 0.1 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, or 10.0 mg/kg. In some embodiments, a targeted polypeptide described herein is administered to a subject at a dosage of about or at least about 0.1 mg/kg, about or at least about 0.5 mg/kg, about or at least about 1.0 mg/kg, about or at least about 1.5 mg/kg, about or at least about 2.0 mg/kg, about or at least about 2.5 mg/kg, about or at least about 3.0 mg/kg, about or at least about 3.5 mg/kg, about or at least about 4.0 mg/kg, about or at least about 4.5 mg/kg, about or at least about 5.0 mg/kg, about or at least about 5.5 mg/kg, about or at least about 6.0 mg/kg, about or at least about 6.5 mg/kg, about or at least about 7.0 mg/kg, about or at least about 7.5 mg/kg, about or at least about 8.0 mg/kg, about or at least about 8.5 mg/kg, about or at least about 9.0 mg/kg, about or at least about 9.5 mg/kg, about or at least about 10.0 mg/kg, about or at least about 15.0 mg/kg, about or at least about 20 mg/kg, about or at least about 30 mg/kg, about or at least about 40 mg/kg, about or at least about 50 mg/kg, about or at least about 60 mg/kg, about or at least about 70 mg/kg, about or at least about 80 mg/kg, about or at least about 90 mg/kg, or about or at least about 100 mg/kg. Any of the dosing frequencies described above may be used.

In some embodiments, provided herein is a method of treatment or prevention of a cancer by administration of any targeted cytokines or compositions described herein. In some embodiments, provided herein is a method of treatment or prevention of a cancer by administration of any cytokine or composition described herein in combination with an anti-cancer agent. The anti-cancer agent can be any agent capable of reducing cancer growth, interfering with cancer cell replication, directly or indirectly killing cancer cells, reducing metastasis, reducing tumor blood supply, or reducing cell survival. In some embodiments, the anti-cancer agent is selected from the group consisting of a PD-1 inhibitor, an EGFR inhibitor, a HER2 inhibitor, a VEGFR inhibitor, a CTLA-4 inhibitor, a BTLA inhibitor, a B7H4 inhibitor, a B7H3 inhibitor, a CSFIR inhibitor, an HVEM inhibitor, a CD27 inhibitor, a KIR inhibitor, an NKG2A inhibitor, an NKG2D agonist, a TWEAK inhibitor, an ALK inhibitor, a CD52 targeting antibody, a CCR4 targeting antibody, a PD-L1 inhibitor, a KIT inhibitor, a PDGFR inhibitor, a BAFF Inhibitor, an HD AC inhibitor, a VEGF ligand inhibitor, a CD19 targeting molecule, a FOFR1 targeting molecule, a DFF3 targeting molecule, a DKK1 targeting molecule, a MUC1 targeting molecule, a MUG 16 targeting molecule, a PSMA targeting molecule, an MSFN targeting molecule, an NY-ESO-1 targeting molecule, a B7H3 targeting molecule, a B7H4 targeting molecule, a BCMA targeting molecule, a CD29 targeting molecule, a CD151 targeting molecule, a CD 123 targeting molecule, a CD33 targeting molecule, a CD37 targeting molecule, a CDH19 targeting molecule, a CEA targeting molecule, a Claudin 18.2 targeting molecule, a CFEC12A targeting molecule, an EGFRVIII targeting molecule, an EPCAM targeting molecule, an EPHA2 targeting molecule, an FCRH5 targeting molecule, an FLT3 targeting molecule, a GD2 targeting molecule, a glypican 3 targeting molecule, a gpA33 targeting molecule, a GPRC5D targeting molecule, an IL-23R targeting molecule, an IL-1RAP targeting molecule, a MCSP targeting molecule, a RON targeting molecule, a ROR1 targeting molecule, a STEAP2 targeting molecule, a TfR targeting molecule, a CD166 targeting molecule, a TPBG targeting molecule, a TROP2 targeting molecule, a proteasome inhibitor, an ABE inhibitor, a CD30 inhibitor, a FLT3 inhibitor, a MET inhibitor, a RET inhibitor, an IL-1β inhibitor, a MEK inhibitor, a ROS1 inhibitor, a BRAE inhibitor, a CD38 inhibitor, a RANKE inhibitor, a B4GALNTi inhibitor, a SLAMF7 inhibitor, an IDH2 inhibitor, an mTOR inhibitor, a CD20 targeting antibody, a BTK inhibitor, a PI3K inhibitor, a FLT3 inhibitor, a PARP inhibitor, a CDK4 inhibitor, a CDK6 inhibitor, an EGFR inhibitor, a RAF inhibitor, a JAK1 inhibitor, a JAK2 inhibitor, a JAK3 inhibitor, an IL-6 inhibitor, a IL-17 inhibitor, a Smoothened inhibitor, an IL-6R inhibitor, a BCL2 inhibitor, a PTCH inhibitor, a PIGF inhibitor, a TGFB inhibitor, a CD28 agonist, a CD3 agonist, CD40 agonist, a GITR agonist, a 0X40 agonist, a VISTA agonist, a CD137 agonist, a LAG3 inhibitor, a TIM3 inhibitor, a TIGIT inhibitor, and an IL-2R inhibitor.

In some embodiments, provided herein is a method of treatment or prevention of a cancer by administration of any cytokine described herein in combination with an anti-inflammatory agent. The anti-inflammatory agent can be any agent capable of preventing, counteracting, inhibiting, or otherwise reducing inflammation.

In some embodiments, the anti-inflammatory agent is a cyclooxygenase (COX) inhibitor. The COX inhibitor can be any agent that inhibits the activity of COX-1 and/or COX-2. In some embodiments, the COX inhibitor selectively inhibits COX-1 (i.e., the COX inhibitor inhibits the activity of COX-1 more than it inhibits the activity of COX-2). In some embodiments, the COX inhibitor selectively inhibits COX-2 (i.e., the COX inhibitor inhibits the activity of COX-2 more than it inhibits the activity of COX-1). In some embodiments, the COX inhibitor inhibits both COX-1 and COX-2.

In some embodiments, the COX inhibitor is a selective COX-1 inhibitor and is selected from the group consisting of SC-560, FR122047, P6, mofezolac, TFAP, flurbiprofen, and ketoprofen. In some embodiments, the COX inhibitor is a selective COX-2 inhibitor and is selected from the group consisting of celecoxib, rofecoxib, meloxicam, piroxicam, deracoxib, parecoxib, valdecoxib, etoricoxib, a chromene derivative, a chroman derivative, N-(2-cyclohexyloxynitrophenyl) methane sulfonamide, parecoxib, lumiracoxib, RS 57067, T-614, BMS-347070, JTE-522, S-2474, SVT-2016, CT-3, ABT-963, SC-58125, nimesulide, flosulide, NS-398, L-745337, RWJ-63556, L-784512, darbufelone, CS-502, LAS-34475, LAS-34555, S-33516, diclofenac, mefenamic acid, and SD-8381. In some embodiments, the COX inhibitor is selected from the group consisting of ibuprofen, naproxen, ketorolac, indomethacin, aspirin, naproxen, tolmetin, piroxicam, and meclofenamate. In some embodiments, the COX inhibitor is selected from the group consisting of SC-560, FR122047, P6, mofezolac, TFAP, flurbiprofen, ketoprofen, celecoxib, rofecoxib, meloxicam, piroxicam, deracoxib, parecoxib, valdecoxib, etoricoxib, a chromene derivative, a chroman derivative, N-(2-cyclohexyloxynitrophenyl) methane sulfonamide, parecoxib, lumiracoxib, RS 57067, T-614, BMS-347070, JTE-522, S-2474, SVT-2016, CT-3, ABT-963, SC-58125, nimesulide, flosulide, NS-398, L-745337, RWJ-63556, L-784512, darbufelone, CS-502, LAS-34475, LAS-34555, S-33516, diclofenac, mefenamic acid, SD-8381, ibuprofen, naproxen, ketorolac, indomethacin, aspirin, naproxen, tolmetin, piroxicam, and meclofenamate.

In some embodiments, the anti-inflammatory agent is an NF-KB inhibitor. The NF-KB inhibitor can be any agent that inhibits the activity of the NF-KB pathway. In some embodiments, the NF-KB inhibitor is selected from the group consisting of an IKK complex inhibitor, an IKB degradation inhibitor, an NF-KB nuclear translocation inhibitor, a p65 acetylation inhibitor, an NF-KB DNA binding inhibitor, an NF-KB transactivation inhibitor, and a p53 induction inhibitor.

In some embodiments, the IKK complex inhibitor is selected from the group consisting of TPCA-1, NF-KB Activation Inhibitor VI (BOT-64), BMS-345541, amlexanox, SC-514 (GK-01140), IMD-0354, and IKK-16. In some embodiments, the IKB degradation inhibitor is selected from the group consisting of BAY-11-7082, MG-115, MG-132, lactacystin, epoxomicin, parthenolide, carfilzomib, and MLN-4924 (pevonedistat). In some embodiments, the NF-KB nuclear translocation inhibitor is selected from the group consisting of JSH-23 and rolipram. In some embodiments, the p65 acetylation inhibitor is selected from the group consisting of gallic acid and anacardic acid. In some embodiments, the NF-KB DNA binding inhibitor is selected from the group consisting of GYY-4137, p-XSC, CV-3988, and prostaglandin E2 (PGE2). In some embodiments, the NF-KB transactivation inhibitor is selected from the group consisting of LY-294002, wortmannin, and mesalamine. In some embodiments, the p53 induction inhibitor is selected from the group consisting of quinacrine and flavopiridol. In some embodiments, the NF-KB inhibitor is selected from the group consisting of TPCA-1, NF-KB Activation Inhibitor VI (BOT-64), BMS-345541, amlexanox, SC-514 (GK-01140), IMD-0354, IKK-16, BAY-11-7082, MG-115, MG-132, lactacystin, epoxomicin, parthenolide, carfilzomib, MLN-4924 (pevonedistat), JSH-23 rolipram, gallic acid, anacardic acid, GYY-4137, p-XSC, CV-3988, prostaglandin E2 (PGE2), LY-294002, wortmannin, mesalamine, quinacrine, and flavopiridol.

In some embodiments, provided herein is a method of treatment or prevention of a cancer by administration of any cytokine or composition described herein in combination with an anti-¬cancer therapeutic protein. The anti-cancer therapeutic protein can be any therapeutic protein capable of reducing cancer growth, interfering with cancer cell replication, directly or indirectly killing cancer cells, reducing metastasis, reducing tumor blood supply, or reducing cell survival. Exemplary anti-cancer therapeutic proteins may come in the form of an antibody or fragment thereof, an antibody derivative, a bispecific antibody, a chimeric antigen receptor (CAR) T cell, a fusion protein, or a bispecific T-cell engager (BiTE). In some embodiments, provided herein is a method of treatment or prevention of a cancer by administration of any targeted cytokine or composition described herein in combination with CAR-NK (Natural Killer) cells.

Additionally, the present disclosure provides methods of use of the engineered cleavable Fc domain comprising a protease cleavage site. The cleavability of the engineered Fc domain provides a universal applicability. In some embodiment, the present engineered cleavable Fc domain may be linked to any therapeutic agent. The method comprises fusing the Fc domain with an engineered protease cleavage site to a molecule using a suitable linker wherein the fusion does not affect the cleavage of the Fc domain and wherein the cleavage of the Fc domain releases the fused molecule.

### EXAMPLES

While certain compounds, compositions and methods of the present invention have been described with specificity in accordance with certain embodiments, the following examples serve only to illustrate the compounds of the invention and are not intended to limit the same.

### Example 1. Universal Cleavable Fc Platform

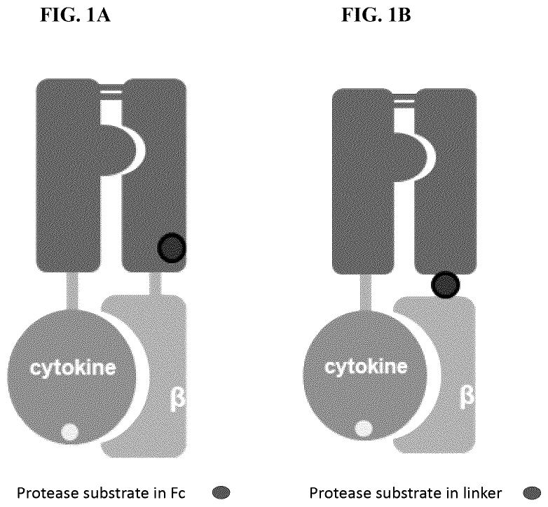

A universal cleavable Fc platform offers a universal design architecture for in vivo delivery of a cytokine. An exemplary schematics of universal cleavable Fc platform is shown in FIG. 1A. In a universal cleavable Fc platform, one or more protease substrates are disposed at the Fc domain, and the Fc domain is cleavable upon interaction with suitable proteases. As the protease substrates are disposed at the Fc domain, a universal cleavable Fc platform allows fusion of different kinds of cytokines and masking moieties to the Fc domain in a fashion similar to a “plug and play” type system which is not possible in a conventional platform. In a conventional platform, one or more protease substrates are disposed either between the Fc domain and the masking moiety or between the Fc domain and the cytokine. As a result, a conventional platform requires designing of a new and specific linker for every cytokine and masking moiety, and for their combination. An exemplary schematics of a convention platform is shown in FIG. 1B. In some embodiments, the masking moiety can be a cytokine receptor, an scFv, VHH, or any antibody binding fragment that binds to the cytokine. In the convention platform shown in FIG. 1B, a protease substrate is disposed between the Fe domain and the masking moiety.

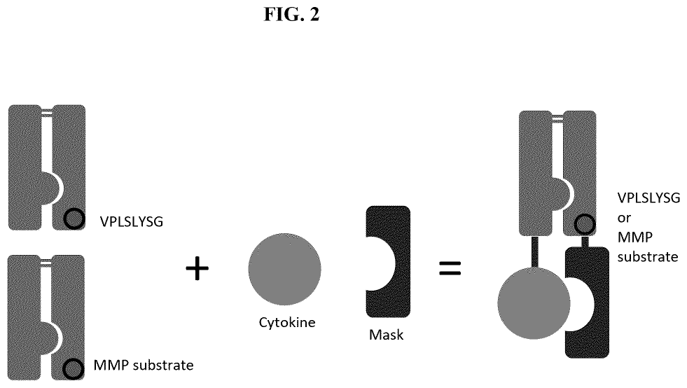

FIG. 2 illustrates two exemplary universal cleavable Fc platforms displaying two different protease substrates (VPLSLYSG (SEQ ID NO: 95) and MMP7-specific substrate), and either of which can be fused with any cytokine and/or a masking moiety demonstrating the universal applicability or versatility of such platform. The universal cleavable Fc platform can be highly optimized using protein display to identify novel protease substrates which can even be selected for protease specificity with the right selection.

### Example 2. Strategies for Incorporating a Protease Substrate in a Human Fc (hFc) for Designing a Universal Cleavable Fc Platform

Described herein are some of the ways or strategies that are used to incorporate a protease substrate in an hFc domain. One strategy includes mutating a known protease site in an hFc domain to a desired protease substrate. For example, one strategy includes replacing 358-LTKNQVSL-365 (SEQ ID NO: 96) and/or 418-QQGNVFSC-425 (SEQ ID NO: 97) in an hFc with VPLSLYSG (SEQ ID NO: 95).

Another strategy includes finding a sequence in hFc that is closest to a publicly known substrate, and mutating that sequence to the publicly known protease substrate. A list of publicly known protease substrates is provided below in Table 1. These protease substrates shown in Table 1 look similar (within three amino acids) to a motif on hFc. Most of these mutations in the hFc does not destabilizes the hFc domain as has been confirmed by computational modeling. The strategy optionally also involves mixing multiple substrates to take advantage of sequence similarity of substrates and stabilization. Another strategy also includes mutating PLGL into a CH3 C-terminus.

TABLE 1
Creation of protease substrates in human Fc domain at positions with similar
motifs

| Cleavage site | Cleavage site Substrate | | |
|---|---|---|---|
| (Substrate) | (coding sequence) | Human Fc sequence | Protease |
| IHVTLKSL (SEQ | attcatgtgaccctgaaaagcctg (SEQ | 434-NHYTQKSL-441 (SEQ ID | MMP2 |
| ID NO: 99) | ID NO: 111) | NO: 98) | |
| NSYTIKGL (SEQ | aacagctataccattaaaggcctg (SEQ | 434-NHYTQKSL-441 (SEQ ID | MMP9 |
| ID NO: 100) | ID NO: 112) | NO: 98) | |
| SNESLSLS (SEQ | agcaacgaaagcctgagcctgagc (SEQ | 437-TQKSLSLS-444 (SEQ ID | MMP2 |
| ID NO: 102) | ID NO: 113) | NO: 101) | |
| QVSSSLSP (SEQ | caggtgagcagcagcctgagcccg (SEQ | 438-QKSLSLSP-445 (SEQ ID NO: | MMP3 |
| ID NO: 104) | ID NO: 114) | 103) | |
| PTSTSLSP (SEQ | ccgaccagcaccagcctgagcccg (SEQ | 438-QKSLSLSP-445 (SEQ ID | MMP9 |
| ID NO: 105) | ID NO: 115) | NO: 103) | |
| ESLSLSEE (SEQ | gaaagcctgagcctgagcgaagaa (SEQ | 439-KSLSLSPG-446 (SEQ ID | MMP2 |
| ID NO: 107) | ID NO: 116) | NO: 106) | |
| ASLSLAPV (SEQ | gcgagcctgagcctggcgccggtg (SEQ | 439-KSLSLSPG-446 (SEQ ID | MMP2 |
| ID NO: 108) | ID NO: 117) | NO: 106) | |

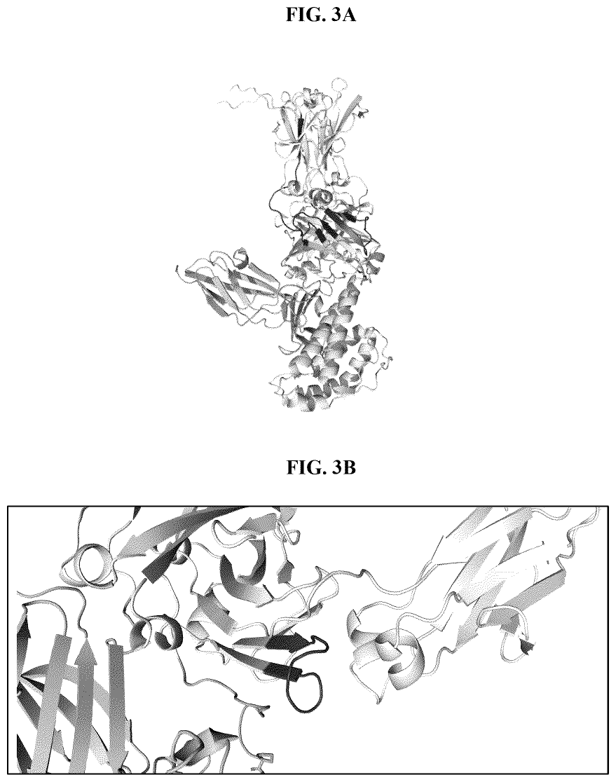

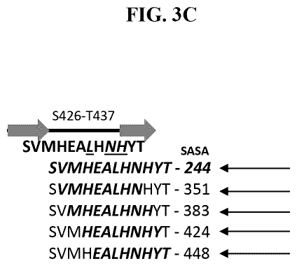

Another strategy includes mutation of surface exposed beta strands of hFc to a known substrate i.e., publicly known protease substrate or applicant's proprietary protease substrate. This strategy includes grafting protease sites onto hFc beta strands as well as onto loop regions of hFc as illustrated in FIGS. 3A and 3B. The strategy involves selecting regions of hFc that are solvent exposed to allow for proteolysis and adding a protease site onto it. Thus, the protease site gets in the way of Protein A binding and serves as a functional equivalent of RF mutations (which prevent excess knob impurities). A list of protease sites where protease substrates are grafted are provided in FIG. 3C.

### Example 3. Preparation of Protein Constructs to Test the Viability of Universal Cleavable Fc Platform

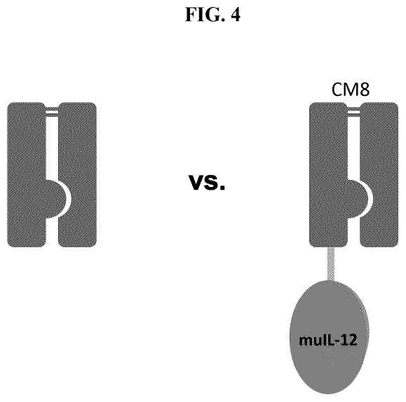

Described below in Table 2 are twenty-three protein constructs that were prepared to test the viability of universal cleavable Fc platform in vitro. All of the twenty-three protein constructs are knob-in-hole Fc's with the knob-chain Fc (SEQ ID NO: 10) modified to contain the protease sites, and an unchanged hole-chain (SEQ ID NO: 5). An unmodified knob-in-hole Fc construct was used as the control construct #1. Table 2 provides protease site (protease substrate) and location the protease site corresponding to each construct. The protease site in each construct is disposed to either at CH3 domains or in the linker of CH2 and CH3 domains. Construct 1 does not include any protease site, and serves as a negative control. As fusing any protein to the C-terminus of the knob-chain allows for easy visualization of cleavage at any site on the knob chain Fc, a mutated IL-12 (muIL-12) was fused to the C-terminal of the knob chain of each protein construct as illustrated in FIG. 4. These constructs were produced in parallel using rapid prototyping using 3 mL cultures in 24 deep-well plates. To obtain sufficient amount of protein for in vitro testing, a parental fusion protein (CM8) without engineered protease cleavage sites in the Fc domain that expresses at a high level was used as scaffold to maximize the protein expression. The parental fusion protein (CM8) acts as another control.

TABLE 2
Protein constructs incorporating different
protease substrates

| | | | Number of Fc |
|---|---|---|---|
| | | | residues |
| | | Location of | remaining |
| | Protease Site | Protease | post- |
| Construct | (Substrate) | Site | cleavage |
| 1 | None | None | N/A |
| 2 | VPLSLYSG | CH2-CH3 | 104 |
| | | Linker | |
| 3 | VPLSLYSG | CH3 | 65 |
| 4 | VPLSLYSG | CH3 | 64 |
| 5 | VPLSLYSG | CH3 | 59 |
| 6 | VPLSLYSG | CH3 | 57 |
| 7 | VPLSLYSG | CH3 | 56 |
| 8 | VPLSLYSG | CH3 | 16 |
| 9 | VPLSLYSG | CH3 | 15 |
| 10 | VPLSLYSG | CH3 | 14 |
| 11 | VPLSLYSG | CH3 | 13 |
| 12 | VPLSLYSG | CH3 | 12 |
| 13 | IHVTLKSL (MMP2) | CH3 | 9 |
| 14 | NSYTIKGL (MMP9) | CH3 | 9 |
| 15 | SQESLSLS (MMP2) | CH3 | 7 |
| 16 | QVSSSLSP (MMP3) | CH3 | 6 |
| 17 | PTSTSLSP (MMP9) | CH3 | 5 |
| 18 | ESLSLSEE (MMP2) | CH3 | 4 |
| 19 | ASLSLAPV (MMP2) | CH3 | 4 |
| 20 | PLGL | CH3 | 5 |
| 21 | VPLSLYSG | CH3 | 4 |
| 22 | VPLSLYSG | CH3 | 85 |
| 23 | VPLSLYSG | CH3 | 25 |

### Example 4. Production of Universal Cleavable Fc Molecules

Plasmids encoding the constructs (shown in Table 2) were transfected into expi293F cells using 24 deep-well transfection platform for 5 days and were subsequently purified. All 23 molecules expressed well, and sufficient material was purified, as shown below in Table 3, for in vitro testing.

TABLE 3
Production of universal cleavable Fc molecules

| | | Protein Ex. | Elution | Post Protein A Yield |
|---|---|---|---|---|
| Protein | Elution A280 | Co. | (mg/mL) | (μg) |
| 1 | 0.96 | 1.293 | 0.7425 | 163.34 |
| 2 | 1.15 | 1.306 | 0.8806 | 193.72 |
| 3 | 0.65 | 1.257 | 0.5171 | 113.76 |
| 4 | 0.72 | 1.256 | 0.5732 | 126.11 |
| 5 | 0.69 | 1.305 | 0.5287 | 116.32 |
| 6 | 0.64 | 1.293 | 0.4950 | 108.89 |
| 7 | 0.76 | 1.294 | 0.5873 | 129.21 |
| 8 | 0.71 | 1.307 | 0.5432 | 119.51 |
| 9 | 0.84 | 1.307 | 0.6427 | 141.39 |
| 10 | 0.76 | 1.308 | 0.5810 | 127.83 |
| 11 | 0.72 | 1.295 | 0.5560 | 122.32 |
| 12 | 0.69 | 1.294 | 0.5332 | 117.31 |
| 13 | 0.84 | 1.28 | 0.6563 | 144.38 |
| 14 | 0.64 | 1.294 | 0.4946 | 108.81 |
| 15 | 0.84 | 1.293 | 0.6497 | 142.92 |
| 16 | 0.85 | 1.293 | 0.6574 | 144.62 |
| 17 | 0.87 | 1.293 | 0.6729 | 148.03 |
| 18 | 1.26 | 1.291 | 0.9760 | 214.72 |
| 19 | 1.41 | 1.293 | 1.0905 | 239.91 |
| 20 | 1.2 | 1.293 | 0.9281 | 204.18 |
| 21 | 1.41 | 1.305 | 1.0805 | 237.70 |
| 22 | 0.71 | 1.307 | 0.5432 | 119.51 |
| 23 | 0.67 | 1.306 | 0.5130 | 112.86 |
| CM8 | 1.2 | 1.292 | 0.9288 | 204.33 |

### Example 5. Testing of Universal Cleavable Fc Molecules for In Vitro Protease Cleavage by MMPs

Universal cleavable Fc molecules were tested for in vitro protease cleavage by MMPs. MMP10 was activated with APMA (4-aminophenylmercuric acetate) and was incubated with each of the universal cleavable Fc molecule overnight at a ratio of 10 μg of protein to 800 ng of MMP10.

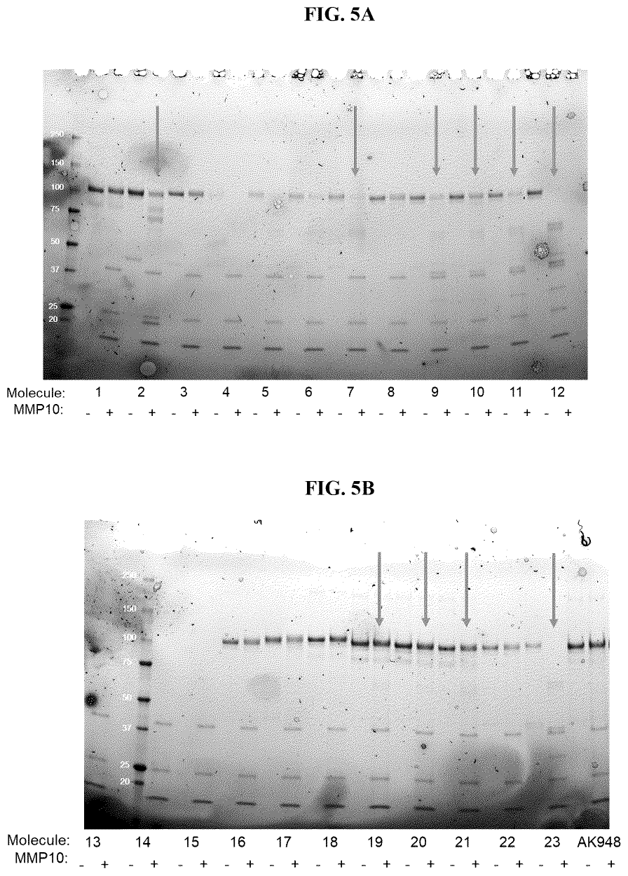

SDS-PAGE images, shown as FIGS. 5A and 5B, indicate cleavage of several constructs—full cleavage of constructs 12 and 23, and partial cleavage of constructs 7, 9, 10, 11, 19, 20, and 21. MMP10 could cleave 10 out of 23 constructs tested, and all of the constructs with VPLSLYSG substrates except 19.

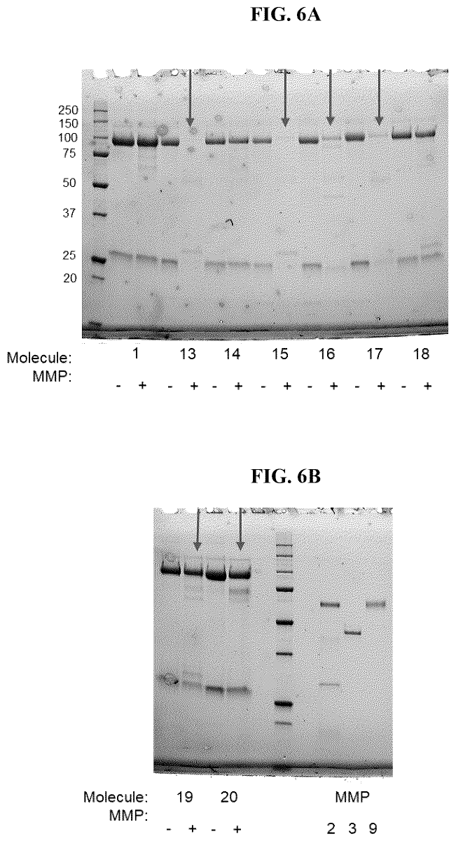

MMP2, MMP3, or MMP9 were also used to test constructs 13-19 in a similar manner as described for MMP10. SDS-PAGE images, as shown in FIGS. 6A and 6B indicate cleavage for several constructs: nearly full cleavage of constructs 13, 15, 16, and 17; and partial cleavage of constructs 19 and 20. Table 4 provides summary of the cleavability of each construct by an activated protease—MMP2, MMP3, MMP9, or MMP10. Table 4 confirms that 12-14 out of the total 23 molecules demonstrated either partial or full cleavage by a protease.

TABLE 4
Universal cleavable Fc molecules and feasibility of their cleavage by proteases

| | | Location of | Number of Fc | | Protease |
|---|---|---|---|---|---|
| Construct | Substrate | Protease Site | residues | Cleavage | Used |
| 1 | None | None | N/A | No | |
| 2 | VPLSLYSG | CH2-CH3 Linker | 104 | Partial | MMP10 |
| 3 | VPLSLYSG | CH3 | 65 | No | MMP10 |
| 4 | VPLSLYSG | CH3 | 64 | No | MMP10 |
| 5 | VPLSLYSG | CH3 | 59 | No | MMP10 |
| 6 | VPLSLYSG | CH3 | 57 | No | MMP10 |
| 7 | VPLSLYSG | CH3 | 56 | Partial | MMP10 |
| 8 | VPLSLYSG | CH3 | 16 | No | MMP10 |
| 9 | VPLSLYSG | CH3 | 15 | Partial | MMP10 |
| 10 | VPLSLYSG | CH3 | 14 | Partial | MMP10 |
| 11 | VPLSLYSG | CH3 | 13 | Partial | MMP10 |
| 12 | VPLSLYSG | CH3 | 12 | Strong | MMP10 |
| 13 | IHVTLKSL | CH3 | 9 | Strong | MMP2 |
| | (MMP2) | | | | |
| 14 | NSYTIKGL | CH3 | 9 | No | MMP9 |
| | (MMP9) | | | | |
| 15 | SNESLSLS | CH3 | 7 | Strong | MMP2 |
| | (MMP2) | | | | |
| 16 | QVSSSLSP | CH3 | 6 | Strong | MMP3 |
| | (MMP3) | | | | |
| 17 | PTSTSLSP | CH3 | 5 | Strong | MMP9 |
| | (MMP9) | | | | |
| 18 | ESLSLSEE | CH3 | 4 | No | MMP2 |
| | (MMP2) | | | | |
| 19 | ASLSLAPV | CH3 | 4 | Mild | MMP2 |
| | (MMP2) | | | | |
| 20 | PLGL | CH3 | 5 | Mild | MMP10/MMP9 |
| 21 | VPLSLYSG | CH3 | 4 | Mild | MMP10 |
| 22 | VPLSLYSG | CH3 | 85 | No | MMP10 |
| 23 | VPLSLYSG | CH3 | 25 | Strong | MMP10 |

### Example 6. Masking and Restoration of IL-2 Potency of Cleavable Fc Domain by Protease Cleavage

This example illustrates that various cleavage substrates can be incorporated into the Fc region to create cleavable Fc domains, and the cleavage substrates can be successfully cleaved by proteases, restoring the potency of a cytokine incorporated in the universal cleavable molecules.

Various cleavage substrates were incorporated in o the Fc domain as shown in Table 5. In this particular experiment, IL-2 cytokine (SEQ ID NO: 14) was fused to the first Fc polypeptide (SEQ ID NO: 10), and a masking moiety of an anti-IL-2 scFv (SEQ ID NO: 142) was incorporated in to the second Fc polypeptide (SEQ ID NO: 5) that contains a cleavage substrate site as shown in Table 5. CM1, CM2, and CM3 were used as controls. CM1 has a scFv as a masking moiety and does not have a cleavage site. CM2 does not have a masking moiety, and therefore is “unmasked.” CM3 has a cleavage substrate in the linker connecting the second Fc domain to the masking moiety of CD122 (IL-2Rβ3).

TABLE 5
Exemplary Cleavable Fc Domains with various cleavage substrates

| | | | Cleavage Substrate |
|---|---|---|---|
| | Cleavage | | location in SEQ ID |
| Construct | Substrate | Fc Mutations | NO: 5 |
| UCM1 | VPLSLYSG | Q438V, K439P, S440L, L441S, S442L, | 438-VPLSLYSG-445 |
| | | L443Y, P445G | |
| UCM2 | VPLSLYSG | K439V, S440P, S444Y, P445S | 439-VPLSLYSG-446 |
| UCM3 | VPLSLYSG | S440V, L441P, S442L, L443S, S444L, | 440-VPLSLYSG-447 |
| | | P445Y, G446S | |
| UCM4 | MPYDLYHP | Q438M, K439P, S440Y, L441D, S442L, | 438-MPYDLYHP- |
| | | L443Y, S444H | 445 |
| UCM5 | MPYDLYHP | K439M, S440P, L441Y, S442D, S444Y, | 439-MPYDLYHP- |
| | | P445H, G446P | 446 |
| UCM6 | MPYDLYHP | S440M, L441P, S442Y, L443D, S444L, | 440-MPYDLYHP- |
| | | P445Y, G446H, G447P | 447 |
| UCM7 | RAAAVKSP | Q438R, K439A, S440A, L441A, S442V, | 438-RAAAVKSP- |
| | | L443K | 445 |
| UCM8 | RAAAVKSP | K439R, S440A, L441A, S442A, L443V, | 439-RAAAVKSP- |
| | | S444K, P445S, G446P | 446 |
| UCM9 | RAAAVKSP | S440R, L441A, S442A, L443A, S444V, | 440-RAAAVKSP- |
| | | P445K, G446S, G447P | 447 |
| UCM10 | RPLALWRS | K439R, S440P, S442A, S444W, P445R, | 439-RPLALWRS-446 |
| | | G446S | |
| UCM11 | APAGLIVPYN | Q438A, K439P, S440A, L441G, S442L, | 438-APAGLIVPYN- |
| | | L443I, S444V, G446Y, G447N | 447 |
| UCM12 | PVSLRSGS | K439P, S440V, L441S, S442L, L443R, | 439-PVSLRSGS-446 |
| | | P445G, G446S | |
| UCM13 | PANLVAPDP | Q438P, K439A, S440N, S442V, L443A, | 438-PANLVAPDP- |
| | | S444P, P445D, G446P | 446 |
| UCM14 | TQKPLGLS | S440P, S442G | 437-TQKPLGLS-444 |
| UCM15 | PLGL | K439G, S440P, S442G | 440-PLGL-443 |
| UCM16 | PLGL | L441P, S442L, L443G, S444L | 441-PLGL-444 |
| UCM17 | PLGL | S440G, L441P, S442L, L443G, S444L | 441-PLGL-444 |
| UCM18 | PLGL | S442P, S444G, P445L | 442-PLGL-445 |
| UCM19 | PLGL | S444P, P445L, G447L | 444-PLGL-447 |
| UCM20 | PLGL | L443G, S444P, P445L, G447L | 444-PLGL-447 |
| UCM21 | PLGL | L443G, S444P, P445L, G447L | 444-PLGL-447 |
| CM1 | Non-cleavable | — | — |
| CM2 | Cleavage site in | — | — |
| | the linker | | |
| | (Unmasked) | | |
| CM3 | Cleavage site in | — | — |
| | the linker | | |

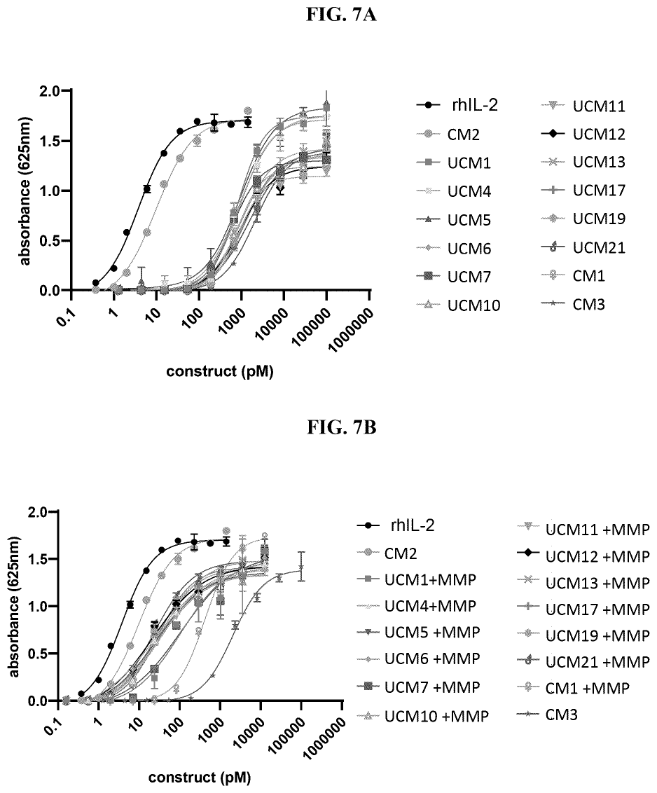

The prepared exemplary constructs were tested in cell-based reporter assay (HEK Blue IL-2 assay) to determine % calculated active cytokine and allows for determination of EC50 values. As shown in FIG. 7A, all molecules were effectively masked except recombinant human IL-2 (rhIL-2) and CM2 (control), which does not contain a masking moiety. Notably, all molecules were masked above 60-fold as compared to CM2 (Table 6). Next, MMP was added to cleave the masking moiety. As shown in FIG. 7B, molecules incorporating cleavage substrate in the Fc region demonstrated an increase in potency after proteolytic activation. EC50 values and fold masking values in Table 6 also demonstrate that upon cleavage, IL-2 activity is restored.

TABLE 6
EC50 values as determined by the HEK Blue
IL-2 assay, with and without MMP protease

| | Intact molecule | | +MMP molecule | |
|---|---|---|---|---|
| | EC50 | Fold Masking | EC50 | Fold Masking |
| Construct | (pM) | CM2 | (pM) | CM2 |
| rhIL-2 | 3.762 | | | |
| CM2 | 9.755 | | | |
| UCM1 | 813.4 | 83.4 | 104.7 | 10.7 |
| UCM4 | 938.4 | 96.2 | 33.93 | 3.5 |
| UCM5 | 925.5 | 94.9 | 27.08 | 2.8 |
| UCM6 | 910 | 93.3 | 23.08 | 2.4 |
| UCM7 | 602 | 61.7 | 107.3 | 11.0 |
| UCM10 | 796.1 | 81.6 | 28.26 | 2.9 |
| UCM11 | 845.2 | 86.6 | 28.68 | 2.9 |
| UCM12 | 925.8 | 94.9 | 21.35 | 2.2 |
| UCM13 | 1006 | 103.1 | 34.12 | 3.5 |
| UCM17 | 1219 | 125.0 | 29.43 | 3.0 |
| UCM19 | 1172 | 120.1 | 25.95 | 2.7 |
| UCM21 | 1587 | 162.7 | 22.78 | 2.3 |
| CM1 | 1135 | 116.4 | 421.9 | 43.2 |
| CM3 | 2054 | 210.6 | | |

### Example 7. Cleavage of Masked and Targeted Cytokines with Cleavable Fc Domains by Various MMPs

Matrix Metalloproteinases (MMPs), such as MMP2, MMP7, and MMP9 are expressed in tumor microenvironment. This example illustrates that cleavage substrate incorporated into the universal cleavable Fc constructs are efficiently cleaved by various MMPs.

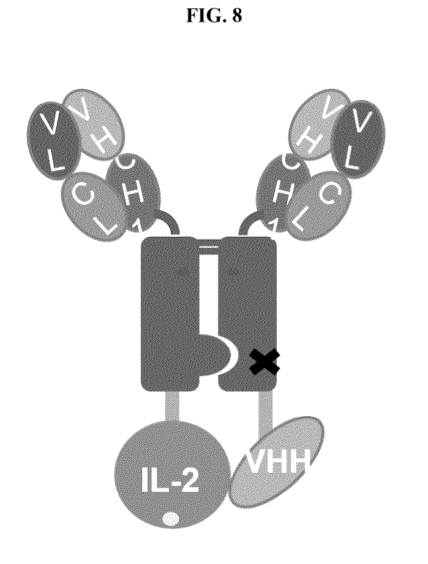

In this particular experiment, masked and targeted IL-2 cytokines containing engineered cleavable Fe domains represented by FIG. 8 were prepared. These constructs contain a targeting moiety that binds to PD-1 (SEQ ID NOs: 77 and 83), and an anti-IL-2 VHH (SEQ ID NO: 121) as a masking moiety. The IL-2 cytokine (either SEQ ID NO: 13 or 14) was fused to the first Fc polypeptide (SEQ ID NO: 10), and the masking moiety was incorporated in to the second Fc polypeptide (SEQ ID NO: 5) modified to contain a cleavage substrate site (PLGL).

The catalytic efficiency (k_{cat}/Kₘ) of MMPs (R&D Systems) to cleave each drug molecule was assessed by cleavage time-course assay.

All the MMPs were activated at 370 C before cleavage reactions, following the manufacturer's instruction. The activation and cleavage reaction were conducted in the same buffer containing 50 mM Tris-HCl pH7.5, 150 mM NaCl, 10 mM CaCl₂, 0.05% Brij35 for MMP-2, MMP-7, and MMP-9.

To initiate a cleavage reaction, 20 nM active MMP was added to 2 μM drug molecule in a 90 μL solution and incubated at 370 C. Aliquots (16 μL) were removed and quenched by 4 μL 250 mM EDTA at 0, 30, 60, 150, 1360 minutes. The percentage of un-cleaved drug in the samples was determined by non-reducing CE-SDS technology (PerkinElmer) and used to calculate for % product=100−(un-cleaved drug %). Cleavage time course was fitted in GraphPad Prism using one-phase association model:

% Product=(Plateau−% Product_{0 min})*(1−exp(−K*time))

The derived first-order constant K was then divided by MMP concentration to yield catalytic efficiency.

As shown in Table 7, the engineered cleavable Fc molecules were efficiently cleaved by various MMPs.

TABLE 7
Cleavage Efficiency of MMPs on engineered cleavable Fc molecules

| | Cleavage | | Est. k_{cat}/K_{M} (M⁻¹S⁻¹) | | | | | |
|---|---|---|---|---|---|---|---|---|
| Construct | Substrate | IL-2 Mutations | MMP-2 | | MMP-7 | | MMP9 | |
| UCM22 | PLGL | C125A | 2.0E+04 | 2.1E+04 | 1.8E+03 | 2.1E+03 | 1.8E+04 | 1.9E+04 |
| | | (SEQ ID NO: | | | | | | |
| | | 13) | | | | | | |
| UCM23 | PLGL | R38A, F42A, | 1.8E+04 | 1.8E+04 | 2.0E+03 | 2.1E+03 | 1.8E+04 | 1.8E+04 |
| | | Y45A, E62A, | | | | | | |
| | | C125A | | | | | | |
| | | (SEQ ID NO: | | | | | | |
| | | 14) | | | | | | |

### Example 8. PD-1/PD-L1 Blockade of Targeted Cytokines with Cleavable Fc Domains

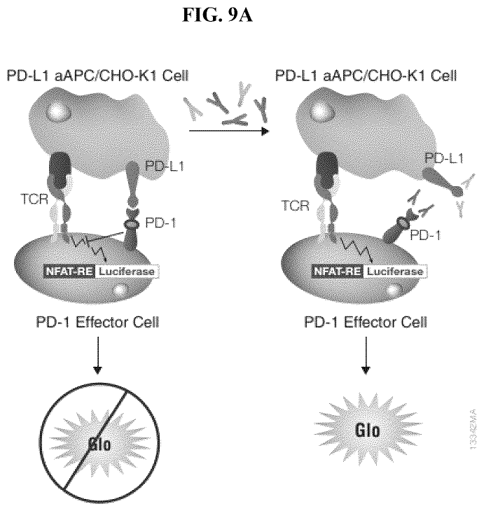

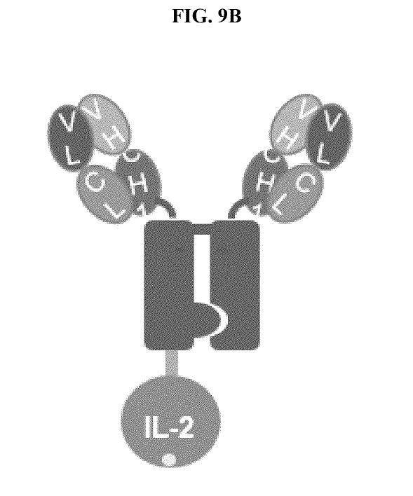

The PD-1/PD-L1 Blockade Bioassay is a biologically relevant MOA-based assay that can be used to measure the potency and stability of antibodies and other biologics designed to block the PD-1/PD-L1 interaction. When the two cell types are co-cultured, the PD-1/PD-L1 interaction inhibits TCR signaling and NFAT-mediated luciferase activity, as shown in FIG. 9A. Addition of an antibody that blocks either PD-1 or PD-L1 releases the inhibitory signal and results in TCR signaling and NFAT-mediated luciferase activity. Fold Induction can be calculated as shown in below formula:

$$Fold Induction = \frac{RLU ( Antibody - Background )}{RLU ( No antibody contorl - Background )}$$

In this particular experiment, seven universal cleavable Fc molecules, UCM22, UCM23, UCM25, UCM26, UCM 27, UCM 28, and UCM29, each containing an engineered cleavable Fc domain, IL-2 cytokine, VHH masking moiety, and PD-1 Fab as a targeting moiety, were prepared (Tables 8a and 8b). CM4 and CM6 are “unmasked” molecules as represented in FIG. 9B.

TABLE 8a
Exemplary Engineered Cleavable Fc domains

| Con- | Cleavage | | Cleavage Substrate |
|---|---|---|---|
| struct | Substrate | Fc Mutations | location |
| UCM22 | PLGL | S444P, P445L, G447L | 444-PLGL-447 |
| UCM23 | PLGL | S444P, P445L, G447L | 444-PLGL-447 |
| UCM25 | MPY | S440M, L441P, S442Y, | 440-MPYDLYHP-447 |
| | | L443D, S444L, P445Y, | |
| | | G446H, G447P | |
| UCM26 | APAG | Q438A, K439P, S440A, | 438-APAGLIVPYN-447 |
| | | L441G, S442L, L443I, | |
| | | S444V, G446Y, G447N | |
| UCM27 | PAN | Q438P, K439A, S440N, | 438-PANLVAPDP-446 |
| | | S442V, L443A, S444P, | |
| | | P445D, G446P | |
| UCM28 | PLGL | S442G, L443G, S444P, | 442-GG-443 & 444- |
| | | P445L, G447L | PLGL-447 |
| UCM29 | PLGL | S444P, P445L, G447L | 444-PLGL-447 |

TABLE 8b
Targeted Universal cleavable Fc molecules

| | | Masking | PD-1 Targeting | | |
|---|---|---|---|---|---|
| Construct | IL-2 | Moiety | Moiety | Fc | Linker |
| UCM22 | C125A | N/A | Fab | Fc knob (SEQ ID | SEQ ID |
| | (SEQ ID NO: 13) | | (SEQ ID NOs: | NO: 11) | NO: 89 |
| | | | 78 and 84) | | |
| | N/A | VHH | Fab | Fc hole + protease | SEQ ID |
| | | (SEQ ID NO: | (SEQ ID NOs: | site (SEQ ID NO: | NO: 140 |
| | | 121) | 78 and 84) | 5 mutated to | |
| | | | | include PLGL) | |
| UCM23 | R38A, F42A, | N/A | Fab | Fc knob (SEQ ID | SEQ ID |
| | Y45A, E62A, | | (SEQ ID NOs: | NO: 11) | NO: 89 |
| | C125A | | 78 and 84) | | |
| | (SEQ ID NO: 14) | | | | |
| | N/A | VHH | Fab | Fc hole + protease | SEQ ID |
| | | (SEQ ID NO: | (SEQ ID NOs: | site (SEQ ID NO: | NO: 140 |
| | | 121) | 78 and 84) | 5 mutated to | |
| | | | | include PLGL) | |
| UCM25 | R38A, F42A, | N/A | Fab | Fc knob (SEQ ID | SEQ ID |
| | Y45A, E62A, | | (SEQ ID NOs: | NO: 11) | NO: 89 |
| | C125A | | 78 and 84) | | |
| | (SEQ ID NO: 14) | | | | |
| | N/A | VHH | Fab | Fc hole + protease | SEQ ID |
| | | (SEQ ID NO: | (SEQ ID NOs: | site (SEQ ID NO: | NO: 140 |
| | | 121) | 78 and 84) | 5 mutated to | |
| | | | | include | |
| | | | | MPYDLYHP) | |
| UCM26 | R38A, F42A, | N/A | Fab | Fc knob (SEQ ID | SEQ ID |
| | Y45A, E62A, | | (SEQ ID NOs: | NO: 11) | NO: 89 |
| | C125A | | 78 and 84) | | |
| | (SEQ ID NO: 14) | | | | |
| | N/A | VHH | Fab | Fc hole + protease | SEQ ID |
| | | (SEQ ID NO: | (SEQ ID NOs: | site (SEQ ID NO: | NO: 140 |
| | | 121) | 78 and 84) | 5 mutated to | |
| | | | | include | |
| | | | | APAGLIVPYN) | |
| UCM27 | R38A, F42A, | N/A | Fab | Fc knob (SEQ ID | SEQ ID |
| | Y45A, E62A, | | (SEQ ID NOs: | NO: 11) | NO: 89 |
| | C125A | | 78 and 84) | | |
| | (SEQ ID NO: 14) | | | | |
| | N/A | VHH | Fab | Fc hole + protease | G + SEQ ID |
| | | (SEQ ID NO: | (SEQ ID NOs: | site (SEQ ID NO: | NO: 140 |
| | | 121) | 78 and 84) | 5 mutated to | |
| | | | | include | |
| | | | | PANLVAPDP) | |
| UCM28 | R38A, F42A, | N/A | Fab | Fc knob (SEQ ID | SEQ ID |
| | Y45A, E62A, | | (SEQ ID NOs: | NO: 11) | NO: 89 |
| | C125A | | 78 and 84) | | |
| | (SEQ ID NO: 14) | | | | |
| | N/A | VHH | Fab | Fc hole + protease | SEQ ID |
| | | (SEQ ID NO: | (SEQ ID NOs: | site (SEQ ID NO: | NO: 140 |
| | | 121) | 78 and 84) | 5 mutated to | |
| | | | | include GGPLGL) | |
| UCM29 | R38A, F42A, | N/A | Fab | Fc knob (SEQ ID | SEQ ID |
| | Y45A, E62A, | | (SEQ ID NOs: | NO: 11) | NO: 89 |
| | C125A | | 78 and 84) | | |
| | (SEQ ID NO: 14) | | | | |
| | N/A | VHH | Fab | Fc hole + protease | SEQ ID |
| | | (SEQ ID NO: | (SEQ ID NOs: | site (SEQ ID NO: | NO: 140 |
| | | 121) | 78 and 84) | 5 mutated to | |
| | | | | include GGPLGL) | |
| CM4 | C125A | | Fab | | |
| | (SEQ ID NO: 13) | | (SEQ ID NOs: | | |
| | | | 78 and 84) | | |
| CM6 | R38A, F42A, | | Fab | | |
| | Y45A, E62A, | | (SEQ ID NOs: | | |
| | C125A | | 78 and 84) | | |
| | (SEQ ID NO: 14) | | | | |

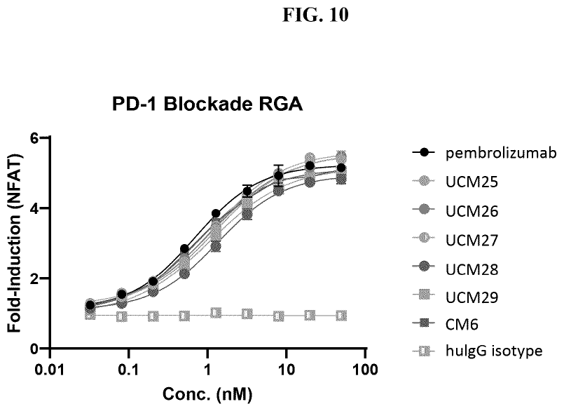

As shown in FIG. 10 and Table 9, all molecules showed comparable PD-1 blockade efficacy as compared to pembrolizumab (KEYTRUDA®), a FDA-approved anti-PD-1 antibody for cancer treatment.

TABLE 9
EC50 values of Constructs used in Example 8

| Construct | EC50 (nM) |
|---|---|
| UCM22 | 0.928 |
| UCM23 | 0.844 |
| UCM25 | 1.278 |
| UCM26 | 1.145 |
| UCM27 | 1.201 |
| UCM28 | 1.360 |
| UCM29 | 1.088 |
| CM4 | 0.586 |
| CM6 | 0.779 |
| Pembrolizumab | 0.700 |

### Example 9. Masking and Restoration of IL-2 Potency of Targeted Cytokines with Cleavable Fc Domains by Protease Cleavage

This example illustrates that the targeted and masked cytokines comprising an engineered cleavable Fe domain are effectively masked and the cytokine activity is restored upon proteolytic cleavage of the masking moiety.

The engineered cleavable Fc molecules containing a targeting moiety prepared in Example 8 were tested in cell-based reporter assay (HEK Blue IL-2 assay) to determine % calculated active cytokine. As shown in FIGS. 11A-11B, all molecules were effectively masked and IL-2 activity was restored to a level similar to the unmasked CM6 molecule upon proteolytic cleavage by an MMP. The corresponding EC50 values are shown in Table 10.

TABLE 10
EC50 values of Constructs used in Example 9

| Construct | EC50 (pM) without MMP | EC50 (pM) with MMP |
|---|---|---|
| UCM22 | 212.6 | 1.4 |
| UCM23 | 957 | 3.6 |
| UCM25 | 1656 | 24.8 |
| UCM26 | 1274 | 6.3 |
| UCM27 | 4161 | 5.7 |
| UCM28 | 2129 | 6.1 |
| UCM29 | 1381 | 6.4 |
| CM4 | 1.7 | — |
| (unmasked control) | | |
| CM6 | 8.4 | — |
| (unmasked control) | | |

### Example 10. In Vivo Efficacy of Targeted and Masked Cytokines with Cleavable Fc Domains

This example illustrates the targeted and masked cytokines containing a cleavable Fe domain are efficacious in vivo. In this particular study, human PD-1 transgenic mice bearing murine tumors were used. The efficacy of UCM23 were assessed against CM6 (unmasked) and pembrolizumab controls.

Tumor cell lines were grown and maintained at 37° C. and 5% CO₂ upon reaching 50-70% confluence, cells were passaged for a total of two passages, prior to in vivo implantation. Cells were harvested using TrypLE Express, re-suspended in PBS, and 0.5-1×10⁶ cells in 100 μL PBS were subcutaneously implanted into the right flank of female B-hPD1 mice.

In vivo mouse assessments of universal cleavable molecules were performed in C57BL/6-Pdcd1tm1(PDCD1) Bcgen/Bcgen transgenic mice purchased from Biocytogen (Beijing, China) and were 8-12 weeks old at the start of the study. C57BL/6 mice were engineered to express human PD-1 exon 2 (encoding the extracellular domain of PD-1) in place of the mouse counterpart (term B-hPD-1). Tumor cell were injected subcutaneously into the right flank of each mouse, and tumor volume was calculated twice weekly (Length*(Width\hat{ }2)/2) using dial calipers. For the efficacy studies, mice were randomized into treatment groups (N=8 mice each) when the mean tumor volume reached approximately 100-150 mm³. For the tumor pharmacodynamics (PD) studies, mice were randomized into treatment groups (N=5 mice each) when tumor volume reached approximately 200-400 mm³. Dosing for Efficacy or Pharmacodynamics (PD) was initiated following randomization on the same day, Day 0. Tumor volume and body weights were recorded two times per week for the duration of the study.

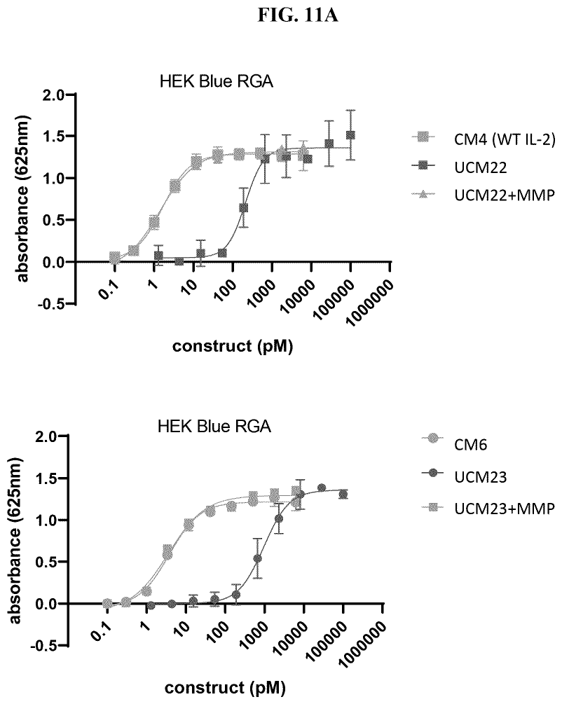

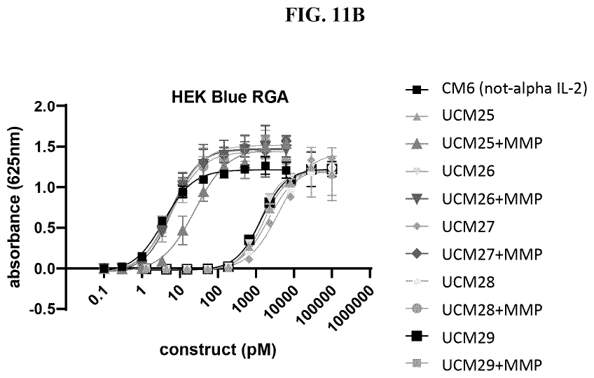

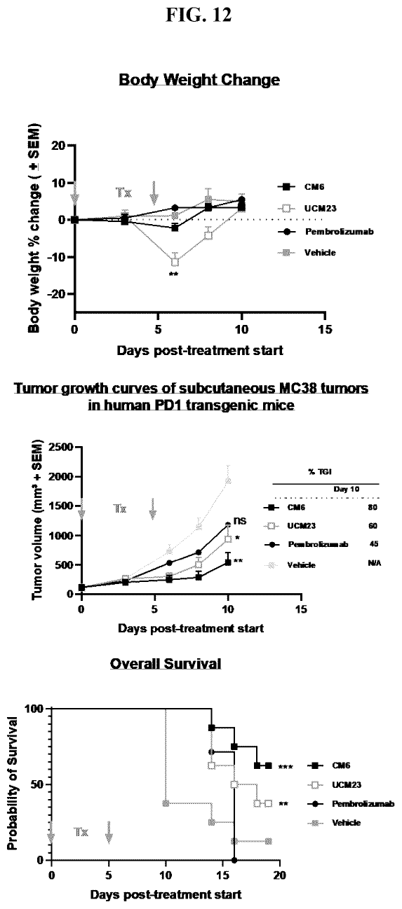

The results are shown in FIG. 12. Overall, the data shows that the masking moiety in the targeted cytokine with an engineered cleavable Fc domain is effectively cleaved in vivo, activating the IL-2 activity. The activated targeted cytokine was effective in inhibiting tumor growth as compared to vehicle and pembrolizumab. Additionally, overall survival probability of the treated mice increased.

### Example 11. Cleavage Substrates in G-Strand of the Fc Domain does not Disrupt Protein a Binding

Protein A-based affinity chromatography is the most commonly used capture step when purifying antibodies and Fc-fusion proteins. The example illustrates that introduction of cleavage substrates in the G-strand of the Fc domain (e.g., residues 438-447 by EU numbering) does not disrupt protein A binding. The constructs illustrated in Table 5 (as shown in Example 6) were transfected into cells. After culture, masked cytokines were purified by protein A. The molecules were purified effectively by Protein A with PhyNexus tips on Tecan. For each construct, the proteins recovered after Protein A purification range of 46 ug-130 ug per 3 mL supernatant, in which the recovery ranges from 46-88%. More than 70% purity is shown by analytical-SEC. The Protein A purification results are shown in Table 12b below.

TABLE 12b
Production of universal cleavable molecules
with cleavable substrate in G-strand

| | Aggregated | ProA Titer | Post ProA Yield from 3 mL |
|---|---|---|---|
| Construct | MW | (ug/mL) | (ug) |
| UCM1 | 95502.6 | Too Low | 69.61 |
| UCM2 | 95573.68 | 59.5 | 95.78 |
| UCM3 | 95644.8 | 29.4 | 78.42 |
| UCM4 | 95702.82 | 26.4 | 57.91 |
| UCM5 | 95773.9 | 37.8 | 70.27 |
| UCM6 | 95845.02 | 29.7 | 54.32 |
| UCM7 | 95466.57 | 57.7 | 89.18 |
| UCM8 | 95537.65 | 45.8 | 70.39 |
| UCM9 | 95608.77 | 55.3 | 77.99 |
| UCM10 | 95736.9 | 71.5 | 104.17 |
| UCM11 | 95567.71 | 20.6 | 46.03 |
| UCM12 | 95540.61 | 33.2 | 67.88 |
| UCM13 | 95503.58 | 35.2 | 59.08 |
| UCM14 | 95506.64 | 76.5 | 113.13 |
| USM15 | 95435.52 | 36.8 | 62.81 |
| UCM16 | 95506.64 | 29.8 | 79.19 |
| UCM17 | 95476.62 | 34.7 | 65.32 |
| UCM18 | 95522.68 | 77.2 | 114.39 |
| UCM19 | 95608.8 | 85.1 | 134.59 |
| UCM20 | 95552.7 | 65. | 124.44 |
| UCM21 | 95522.68 | 73.6 | 126.96 |
| CM7 | 67085.96 | 80.6 | 104.13 |
| CM1 | 95526.62 | 74.6 | 130.73 |

These results indicate that incorporation of various cleavage substrates into the G-strand of the Fc domain does not impact binding of the cleavable Fc molecules to Protein A.

### Example 12. Introduction of Cleavage Substrates in F-Strand or FG-Loop of the Fc Domain

Protein A-based affinity chromatography is the most commonly used capture step when purifying antibodies and Fc-fusion proteins. The example illustrates various cleavage substrates can be incorporated into the F-strand (e.g., residues 416-425 by EU numbering) or FG-loop of the Fc domain (e.g., residues 426-437 by EU numbering) and masked cytokines comprising the cleavable Fc domains can be successfully expressed and purified. Additionally, the impact of incorporation of cleavage substrates into the F-strand or the FG-loop of the Fc domain on Protein A binding was tested.

Various cleavage substrates were incorporated into different subareas in the Fc domain as shown in Table 13. In this particular experiment, IL-2 cytokine (SEQ ID NO: 14) was fused to the first Fc polypeptide (SEQ ID NO: 10), and a masking moiety of an anti-IL-2 scFv (SEQ ID NO: 142) was incorporated in to the second Fc polypeptide (SEQ ID NO: 5) that contains a cleavage substrate site as shown in Table 13. The first Fc polynucleotide comprises RF mutation to prevent protein A binding. Each construct includes a his-tag. CM3, CM7 and CM8 were used as controls. CM3 has a cleavage substrate in the linker connecting the second Fc domain to the masking moiety of CD122 (IL-2Rβ).

TABLE 13
Exemplary masked cytokines with cleavable Fc Domains with various cleavage
substrates

| | Protease | | Cleavage Substrate | Cleavage |
|---|---|---|---|---|
| | cleavage | | location in SEQ ID NO: | Substrate |
| Construct | substrate | Fc Mutations | 5 | location in Fc |
| UCM30 | VPLSLYSG | C367S, Q418V, Q419P, G420L, | 418-VPLSLYSG-425 | F-Strand |
| | | N421S, V422L, F423Y, C425G | | |
| UCM31 | RAAAVKSP | C367S, Q418R, Q419A, G420A, | 418-RAAAVKSP-425 | |
| | | N421A, F423K, C425P | | |
| UCM32 | MPYDLYHP | C367S, Q418M, Q419P, G420Y, | 418-MPYDLYHP-425 | |
| | | N421D, V422L, F423Y, S424H, | | |
| | | C425P | | |
| UCM33 | VPLSLYSG | C367S, W417V, Q418P, Q419L, | 417-VPLSLYSG-424 | |
| | | G420S, N421L, V422Y, F423S, | | |
| | | S424G, C425G | | |
| UCM34 | VPLSLYSG | C367S, R416V, W417P, Q418L, | 416-VPLSLYSG-423 | |
| | | Q419S, G420L, N421Y, V422S, | | |
| | | F423G, C425G | | |
| UCM35 | ISSGLLSGRS | C367S, R416I, W417S, Q418S, | 416-ISSGLLSGRS-425 | |
| | | Q419G, G420L, N421L, V422S, | | |
| | | F423G, S424R, C425S | | |
| UCM36 | RPLALWRS | C367S, Q418R, Q419P, G420L, | 418-RPLALWRS-425 | |
| | | N421A, V422L, F423W, S424R, | | |
| | | C425S | | |
| UCM37 | TQKPLGLS | C367S, Q418T, G420K, N421P, | 418-TQKPLGLS-425 | |
| | | V422L, F423G, S424L, C425S | | |
| UCM38 | APAGLIVPYN | C367S, R416A, W417P, Q418A, | 416-APAGLIVPYN-425 | |
| | | Q419G, G420L, N421I, F423P, | | |
| | | S424Y, C425N | | |
| UCM39 | PVSLRSGS | C367S, Q418P, Q419V, G420S, | 418-PVSLRSGS-425 | |
| | | N421L, V422R, F423S, S424G, | | |
| | | C425S | | |
| UCM40 | PANLVAPDP | C367S, W417P, Q418A, Q419N, | 417-PANLVAPDP-425 | |
| | | G420L, N421V, V422A, F423P, | | |
| | | S424D, C425P | | |
| UCM41 | PLGL | C367S, N421P, V422L, F423G, | 421-PLGL-424 | |
| | | S424L, C425G | | |
| UCM42 | PLGL | C367S, G420P, N421L, V422G, | 420-PLGL-423 | |
| | | F423L, C425G | | |
| UCM43 | PLGL | C367S, Q419P, G420L, N421G, | 419-PLGL-422 | |
| | | V422L, C425G | | |
| UCM44 | VPLSLYSG | C367S, S375C, P396C, Q418V, | 418-VPLSLYSG-425 | |
| | | Q419P, G420L, N421S, V422L, | | |
| | | F423Y, C425G | | |
| UCM45 | VPLSLYSG | C367S, Q418V, Q419P, G420L, | 418-VPLSLYSG-425 | |
| | | N421S, V422L, F423Y, C425G, | | |
| | | L432C, T437C | | |
| UCM46 | VPLSLYSG | S408C, Q418V, Q419P, G420L, | 418-VPLSLYSG-425 | |
| | | N421S, V422L, F423Y, C425G | | |
| UCM47 | VPLSLYSG | C367S, V379C, S408C, Q418V, | 418-VPLSLYSG-425 | |
| | | Q419P, G420L, N421S, V422L, | | |
| | | F423Y, C425G | | |
| UCM48 | VPLSLYSG | C367S, W381C, L410C, Q418V, | 418-VPLSLYSG-425 | |
| | | Q419P, G420L, N421S, V422L, | | |
| | | F423Y, C425G | | |
| UCM49 | VPLSLYSG | C367S, K370C, F405C, Q418V, | 418-VPLSLYSG-425 | |
| | | Q419P, G420L, N421S, V422L, | | |
| | | F423Y, C425G | | |
| UCM50 | VPLSLYSG | C367S, G371C, S403C, Q418V, | 419-VPLSLYSG-426 | |
| | | Q419P, G420L, N421S, V422L, | | |
| | | F423Y, C425G | | |
| UCM51 | VPLSLYSG | E430V, A431P, H433S, N434L, | 430-VPLSLYSG-437 | FG-loop |
| | | H435Y, Y436S, T437G, | | |
| UCM52 | RAAAVKSP | E430R, L432A, H433A, N434V, | 430-RAAAVKSP-437 | |
| | | H435K, Y436S, T437P | | |
| UCM53 | MPYDLYHP | E430M, A431P, L432Y, H433D, | 430-MPYDLYHP-437 | |
| | | N434L, H435Y, Y436H, T437P | | |
| UCM54 | VPLSLYSG | 428-ins-VP, H429L, E430S, | 428-ins-VPLSLYSG-434 | |
| | | A431L, L432Y, H433S, N434G | | |
| UCM55 | RAAAVKSP | 428-ins-RA, H429A, E430A, | 428-ins-RAAAVKSP-434 | |
| | | A431V, L432K, H433S, N434P | | |
| UCM56 | MPYDLYHP | 428-ins-MP, H429Y, E430D, | 428-ins-MPYDLYHP-434 | |
| | | A431L, L432Y, N434P | | |
| UCM57 | VPLSLYSG | 428-ins-VPL, H429S, E430L, | 428-ins-VPLSLYSG-433 | |
| | | A431Y, L432S, H433G | | |
| UCM58 | RAAAVKSP | 428-ins-RAA, H429A, E430V, | 428-ins-RAAAVKSP-433 | |
| | | A431K, L432S, H433P | | |
| UCM59 | MPYDLYHP | 428-ins-MPY, H429D, E430L, | 428-ins-MPYDLYHP-433 | |
| | | A431Y, L432H, H433P | | |
| UCM60 | ISSGLLSGRS | 428-ins-I, H429S, E430S, | 428-ins-ISSGLLSGRS- | |
| | | A431G, H433L, N434S, H435G, | 437 | |
| | | Y436R, T437S | | |
| UCM61 | RPLALWRS | E430R, A431P, H433A, N434L, | 430-RPLALWRS-437 | |
| | | H435W, Y436R, T437S | | |
| UCM62 | TQKPLGLS | E430T, A431Q, L432K, H433P, | 430-TQKPLGLS-437 | |
| | | N434L, H435G, Y436L, T437S | | |
| UCM63 | APAGLIVPYN | 428-ins-A, H429P, E430A, | 428-ins-APAGLIVPYN- | |
| | | A431G, H433I, N434V, H435P, | 437 | |
| | | T437N | | |
| UCM64 | PVSLRSGS | E430P, A431V, L432S, H433L, | 430-PVSLRSGS-437 | |
| | | N434R, H435S, Y436G, T437S | | |
| UCM65 | PANLVAPDP | H429P, E430A, A431N, H433V, | 429-PANLVAPDP-437 | |
| | | N434A, H435P, Y436D, T437P | | |
| UCM66 | PLGL | N434P, H435L, Y436G, T437L | 434-PLGL-437 | |
| UCM67 | PLGL | H433P, N434L, H435G, Y436L | 433-PLGL-436 | |
| UCM68 | PLGL | L432P, H433L, N434G, H435L | 432-PLGL-435 | |
| UCM69 | PLGL | A431P, H433G, N434L | 431-PLGL-434 | |
| UCM70 | PLGL | E430G, A431P, H433G, N434L | 431-PLGL-434 | |
| UCM71 | PLGL | E430P, A431L, L432G, H433L | 430-PLGL-433 | |
| UCM72 | PLGL | H429G, E430P, A431L, L432G, | 430-PLGL-433 | |
| | | H433L | | |
| UCM73 | PLGL | 428-ins-G, H429P, E430L, | 429-PLGL-432 | |
| | | A431G, H433G | | |
| UCM74 | PLGL | 428-ins-GGP, H429L, E430G, | 428-ins-PLGL-431 | |
| | | A431L, L432G | | |

Expressing cells were transfected with each construct in table 13 and cultured. 10 ul of cell culture supernatants were analyzed by SDS-PAGE. Universal cleavable Fc molecules showed expression in cell culture supernatants by SDS-PAGE (data not shown).

Protein A binding was not detected for UCMs 30-74 as measured by Protein A biosensors on octet, whereas protein A binding was detected for control molecules (Table 14). Additionally, protein A binding was detected and measured for masked cytokines comprising cleavage substrates in the G-strand of the Fc region (e.g., UCM1, UCM4-UCM7, UCM1-UCM13, UCM17, UCM 19 and UCM21), as shown in Table 15.

TABLE 14
Protein A binding detection as measured by octet
titers for masked cytokines comprising cleavage
substrates in F-strand or FG-loop of the Fc domain

| Construct | Titer (ug/mL) | Construct | Titer (ug/mL) |
|---|---|---|---|
| UCM30 | Too Low | UCM51 | Too Low |
| UCM31 | Too Low | UCM52 | Too Low |
| UCM32 | Too Low | UCM53 | Too Low |
| UCM33 | Too Low | UCM54 | Too Low |
| UCM34 | Too Low | UCM55 | Too Low |
| UCM35 | Too Low | UCM56 | Too Low |
| UCM36 | 11 | UCM57 | Too Low |
| UCM37 | Too Low | UCM58 | Too Low |
| UCM38 | Too Low | UCM59 | Too Low |
| UCM39 | Too Low | UCM60 | Too Low |
| UCM40 | Too Low | UCM61 | Too Low |
| UCM41 | Too Low | UCM62 | Too Low |
| UCM42 | Too Low | UCM63 | Too Low |
| UCM43 | Too Low | UCM64 | Too Low |
| UCM44 | Too Low | UCM65 | Too Low |
| UCM45 | Too Low | UCM66 | Too Low |
| UCM46 | Too Low | UCM67 | Too Low |
| UCM47 | Too Low | UCM68 | Too Low |
| UCM48 | Too Low | UCM69 | Too Low |
| UCM49 | Too Low | UCM70 | Too Low |
| UCM50 | Too Low | UCM71 | Too Low |
| CM3 | 82 | UCM72 | Too Low |
| CM7 | 96 | UCM73 | Too Low |
| CM8 | 59 | UCM74 | Too Low |

Protein A binding detection was measured by octet titers for masked cytokines comprising cleavage substrates in G-strand of the Fc domain.

TABLE 15
Octet Titers of cleavable Fc molecules by Protein A Biosensors

| Construct | Titer (ug/mL) |
|---|---|
| UCM1 | 6.785824139 |
| UCM4 | 14 |
| UCM5 | 39.90694793 |
| UCM6 | 27.44277728 |
| UCM7 | 31.11711478 |
| UCM10 | 78.79653084 |
| UCM11 | 17.81561533 |
| UCM12 | 33.75930883 |
| UCM13 | 44 |
| UCM17 | 30.4839787 |
| UCM19 | 43.10996128 |
| UCM21 | 75.15864034 |
| CM1 | 88.06487571 |

These results suggest that incorporation of cleavage substrates into certain subareas in the Fc region impacts protein A binding of cleavable Fc molecules. In particular, the incorporation of cleavage substrates into F-strand or FG-loop (e.g., residues 416-425 or 426-437 by EU numbering, respectively) of the Fc domain impacts protein A binding.

To further confirm the impact of incorporation of cleavage substrates in F-strand or FG-loop on protein A binding, UCMs 30-74 were purified via Ni-NTA Robo-columns and concentrated to a concentration between 50-500 ug/ml. Then, the concentrated UCM molecules were tested again using ProA biosensors. Proteins from constructs CM1 and UCM19 were used as positive controls as those molecules have shown binding to ProA biosensors. All molecules were normalized to 200 ug/mL concentration in 1×PBS and assessed for Protein A binding using Octet with ProA biosensors. As shown in Table 16, all molecules with cleavage substrates incorporated into the FG-loop (e.g., residues 426-437 by EU numbering) in the Fc region are not detected by ProA biosensors, suggesting diminished or complete loss of Protein A binding. Among the molecules with cleavage substrates incorporated into the F-strand (e.g., residues 416-425 by EU numbering) in the Fc region, only molecules from 3 constructs (UCM32, UCM36 and UCM45) show some binding to ProA biosensors, while the rest of 18 constructs are not detected using ProA biosensors, suggesting diminished or complete loss of Protein A binding in these molecules (Table 16).

TABLE 16
Cleavable Fc molecules show no quantitation by ProA Biosensors

| | ProA Titer | | ProA Titer |
|---|---|---|---|
| Construct | (ug/mL) | Construct | (ug/mL) |
| UCM30 | Too Low | UCM51 | 0 |
| UCM31 | Too Low | UCM52 | 0 |
| UCM32 | 38.88437621 | UCM53 | Too Low |
| UCM33 | Too Low | UCM54 | Too Low |
| UCM34 | Too Low | UCM55 | 0 |
| UCM35 | Too Low | UCM56 | 0 |
| UCM36 | 37.85229167 | UCM57 | Too Low |
| UCM37 | Too Low | UCM58 | 0 |
| UCM38 | Too Low | UCM59 | 0 |
| UCM39 | Too Low | UCM60 | 0 |
| UCM40 | Too Low | UCM61 | Too Low |
| UCM41 | Too Low | UCM62 | Too Low |
| UCM42 | Too Low | UCM63 | Too Low |
| UCM43 | Too Low | UCM64 | Too Low |
| UCM44 | Too Low | UCM65 | Too Low |
| UCM45 | 41.84808534 | UCM66 | Too Low |
| UCM46 | Too Low | UCM67 | Too Low |
| UCM47 | Too Low | UCM68 | Too Low |
| UCM48 | Too Low | UCM69 | Too Low |
| UCM49 | Too Low | UCM70 | Too Low |
| UCM50 | Too Low | UCM71 | Too Low |
| | | UCM72 | 0 |
| UCM19 | 90.33900385 | UCM73 | Too Low |
| CM1 | 56.69958283 | UCM74 | 0 |

### Example 13. In Vitro Cleavage of Exemplary Masked Cytokines with Universal Cleavable Fc Molecules

This example shows that the 13 exemplary masked molecule constructs comprising a cleavage substrate in the G-strand of Fc domain are efficiently cleaved by various MMPs.

Kinetic study was performed to determine the cleavage efficiency of the masked cytokines listed in Table 17 by MMP7, MMP8, MMP9, MMP10, and MMP14. The following steps were performed: 1) Cleavage of 2 μM substrate by 20 nM pre-activated MMP in TNCB buffer (or with 5 μM ZnCl₂ for MMP14); and 2) Reaction quenched by 50 mM EDTA at 4 time-points (30′, 60′, 150′, 1360′). 3) Each reaction was repeated twice.

To calculate kinetics (kcaₜ/Km), a single-phase association curve fits the equation was used:

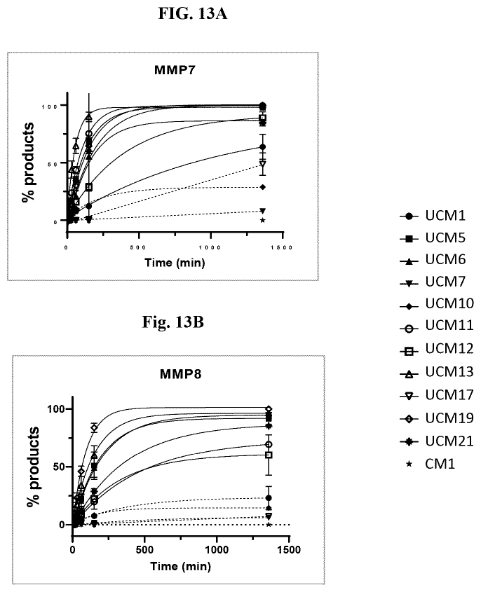

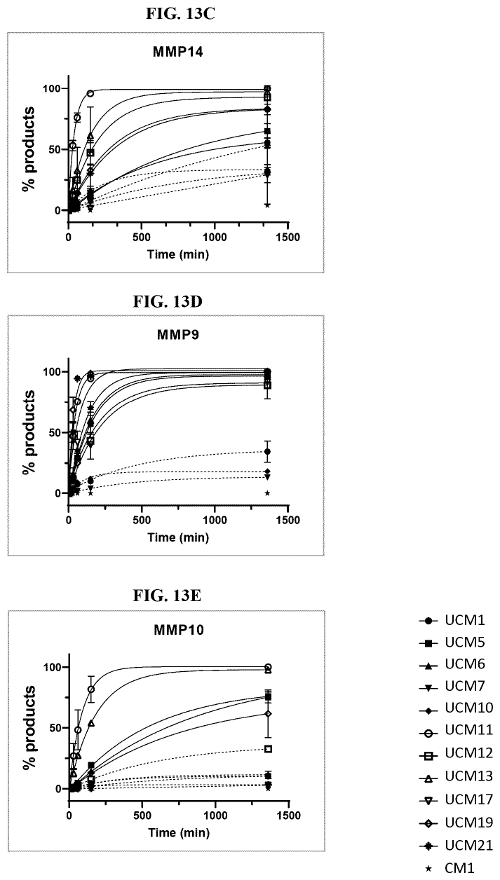

Y=Y0+(Plateau−Y0)*(1−exp(−K*t))
where Y is products % at each time point.
   k_{cat}/Kₘ is calculated by K/[MMP]. The association curve for each MMP is shown in FIG. 13A to FIG. 13E. The k_{cat}/Km values are shown in Table 17.

TABLE 17
Kinetics of cleavable Fc molecules

| | Substrates in the G- | Est. k_{cat}/K_{M}(M⁻¹s⁻¹) | | | | |
|---|---|---|---|---|---|---|
| Construct | strand | MMP-7 | MMP-8 | MMP-9 | MMP-10 | MMP-14 |
| UCM1 | VPLSLYSG | 8.0E+02^{‡} | <1.0E+01 | <1.0E+01 | <1.0E+01 | 1.3E+03^{‡} |
| UCM5 | MPYDLYHP | 6.4E+03^{‡} | 4.4E+03 | 5.0E+03 | 1.5E+03 | 1.0E+03^{‡} |
| UCM6 | MPYDLYHP | 4.3E+03^{‡} | 4.0E+03 | 6.7E+03 | 9.3E+02 | <1.0E+01 |
| UCM7 | RAAAVKSP | <1.0E+01 | <1.0E+01 | <1.0E+01 | <1.0E+01 | <1.0E+01 |
| UCM10 | RPLALWRS | <1.0E+01 | <1.0E+01 | <1.0E+01 | <1.0E+01 | <1.0E+01 |
| UCM11 | APAGLIVPYN | 7.9E+03^{‡} | 1.7E+03 | 1.9E+04 | 9.2E+03 | 2.1E+04^{‡} |
| UCM12 | PVSLRSGS | 2.1E+03^{‡} | 2.5E+03 | 3.7E+03 | <1.0E+01 | 4.0E+03^{‡} |
| UCM13 | PANLVAPDP | 1.5E+04^{‡} | 5.6E+03 | 5.2E+03 | 4.5E+03 | 5.6E+03^{‡} |
| UCM17 | PLGL | <1.0E+01 | <1.0E+01 | 4.0E+03 | <1.0E+01 | <1.0E+01 |
| UCM19 | PLGL | 5.5E+03 | 9.0E+03 | 1.0E+04 | 1.1E+03 | 2.7E+03 |
| UCM21 | PLGL | 5.2E+03 | 2.2E+03 | 2.5E+04 | <1.0E+01 | 2.5E+03 |
| CM1 | Uncleavable | <1.0E+01 | <1.0E+01 | <1.0E+01 | <1.0E+01 | <1.0E+01 |

Next, the thermal stabilities of the cleaved products were measured using Differential Scanning Fluorimetry (DSF) assay. The results indicate most molecules show little to no change in the thermal stability of the Fc domain post MMP cleavage. Similarly, no significant changes in the thermal stability of IL2 and scFv were detected by DSF.

Intact Mass Spectrometry (iMS) Analysis of In Vitro MMP Cleavage of Masked Molecules

Additionally, mass spectrometry analysis was used to confirm correct cleavage, including primary sequence confirmation, MMP cleavage sequence location and evaluation of specificity.

The samples were first treated with MMP2, MMP7 or MMP9 (as shown in Table 19). iMS with reduced conditions was performed to validate the primary sequences of all molecules and to identify the site of MMP protease cleavage in the treated samples by searching for specific N- and C-term masses within the cleavage site region of the sequence.

The results show that the MMPs specifically cleaved most of the masked molecules at the desired location. primary sequence of all control and MMP molecules were validated by intact mass analysis.

### Example 14. Scale-Up Preparations of Universal Cleavable Fc Molecules and Characterization

This example illustrates that the masked cytokines comprising cleavable Fc domain can be produced at high yield with high purity. In this particular example, the masked cytokines were reformatted to remove the his-tag at the end of the Fc polypeptide, as shown in Table 19. Notably, all constructs could be produced with final yield of between 21 mg to 77 mg and purity of greater than 97%.

TABLE 19
Exemplary UCM constructs used in this study

| Construct | | Corresponding construct |
|---|---|---|
| (without his-tag) | Substrates in Fc | (with his-tag) |
| UCM75 | 438-VPLSLYSG-445 | UCM1 |
| UCM76 | 439-MPYDLYHP-446 | UCM5 |
| UCM77 | 438-RAAAVKSP-445 | UCM7 |
| UCM78 | 439-RPLALWRS-446 | UCM10 |
| UCM79 | 438-APAGLIVPYN-447 | UCM11 |
| UCM80 | 439PVSLRSGS446 | UCM12 |
| UCM81 | 438PANLVAPDP446 | UCM13 |
| UCM82 | 441PLGL444 | UCM17 |
| UCM83 | 444PLGL447 | UCM19 |
| UCM84 | Uncleavable | CM1 |

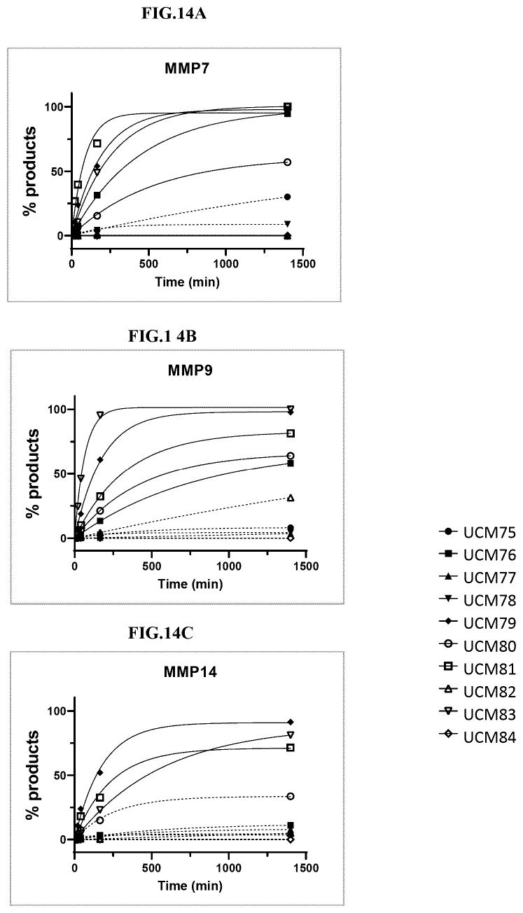

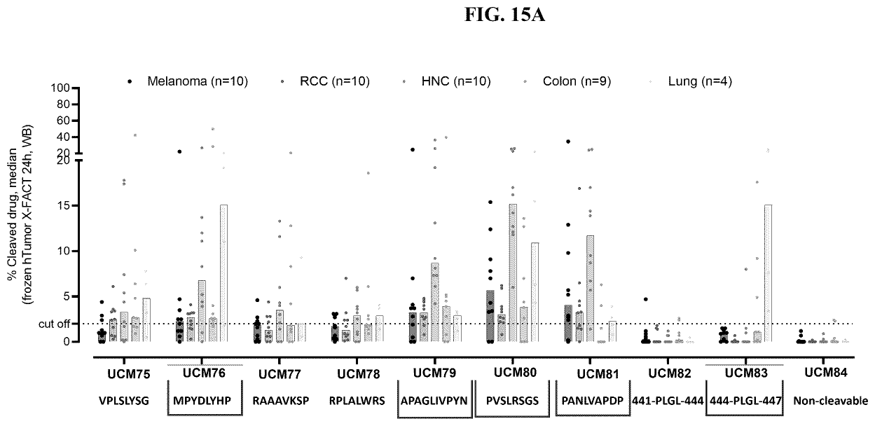

The cleavage kinetics of 10 UCM molecules by MMP 7, MMP9, and MMP14 were measured as described in Example 13. The association curve for each MMP is shown in FIG. 14A to FIG. 14C. The k_{cat}/Km values are shown in Table 20. Most masked cytokines were efficiently cleaved by at least two MMPs.

TABLE 20
Kinetics of cleavable Fc molecules from scale-up preparation

| Con- | | Est. k_{cat}/K_{M} (M⁻¹s⁻¹) | | |
|---|---|---|---|---|
| struct | Fc substrate | MMP-7 | MMP-9 | MMP-14 |
| UCM75 | 438-VPLSLYSG-445 | <1.0E+01 | <1.0E+01 | <1.0E+01 |
| UCM76 | 439-MPYDLYHP-446 | 1.9E+03^{‡} | 1.1E+03 | <1.0E+01 |
| UCM77 | 438-RAAAVKSP-445 | <1.0E+01 | <1.0E+01 | <1.0E+01 |
| UCM78 | 439-RPLALWRS-446 | <1.0E+01 | <1.0E+01 | <1.0E+01 |
| UCM79 | 438-APAGLIVPYN-447 | 4.4E+03^{‡} | 4.7E+03 | 4.7E+03 |
| UCM80 | 439-PVSLRSGS-446 | 1.6E+03^{‡} | 2.0E+03 | <1.0E+01 |
| UCM81 | 438-PANLVAPDP-446 | 9.6E+03^{‡} | 2.5E+03 | 3.6E+03 |
| UCM82 | 441-PLGL-444 | <1.0E+01 | <1.0E+01 | <1.0E+01 |
| UCM83 | 444-PLGL-447 | 3.2E+03 | 1.3E+04 | 1.5E+03 |
| UCM84 | Non Cleavable | <1.0E+01 | <1.0E+01 | <1.0E+01 |

### Example 15: Ex Vivo Cleavage of Masked Cytokines with Cleavable Fc Domains

This example illustrates that masked cytokines comprising cleavable Fc domains can be cleaved by human tumors but not by plasma.

Frozen cells extracted from fresh tumor tissues (X-FACT assay) and human plasma, were used for ex vivo cleavage of masked cytokines with cleavable Fc domains. Cells from five different indications: Melanoma, NSCLC, H&N, RCC, and Colon cancers were used for the cleavage assay. 10 molecules were incubated 24 hours with frozen tumor cells from 5 indications: Melanoma, NSCLC, H&N, RCC, Colon, or incubated in in human plasma derived from healthy donors and patients with Melanoma, NSCLC, H&N, and Colon tumors.

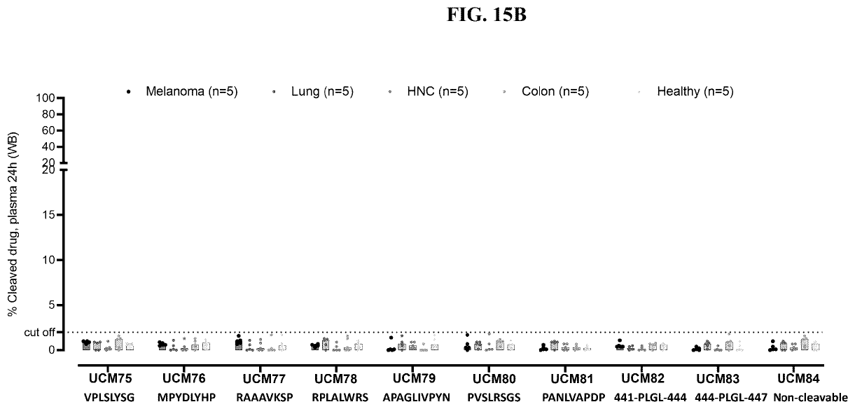

The cleavage results were analyzed by western blotting. All masked cytokines were cleaved by human tumors ex vivo, as shown in FIG. 15A. On the other hand, the masked cytokines were not cleaved by human plasma, indicating specific cleavage by tumors as desired. (FIG. 15B).

### Example 16: FcRn Binding of Masked Cytokines with Cleavable Fc Domain

This example illustrates that the incorporation of the cleavage site into the Fe does not disrupt its binding to human FcRn. Surface Plasmon Resonance (SPR) protein binding assay was used to measure FcRn binding. As shown in Table 21, the masked cytokines comprising cleavable domains bind to FcRn similar to Ipilimumab (positive control), and they are expected to show normal FcRn binding in vivo.

TABLE 21
FcRn Binding by SPR

| | Ligand | | | | | |
|---|---|---|---|---|---|---|
| | Level | | KD | Rmax | Chi² | offset |
| Ligand | (RU) | Sample | (nM) | (RU) | (RU²) | (RU) |
| hFcRn | 115.6 | UCM75 | 1490* | 162.1 | 0.93 | 6.061 |
| hFcRn | 112.7 | UCM76 | 551 | 112.5 | 0.104 | 30.75 |
| hFcRn | 111.9 | UCM77 | 312 | 104.9 | 0.88 | 21.06 |
| hFcRn | 110.1 | UCM78 | 181 | 108.1 | 4.48 | 21.63 |
| hFcRn | 111 | UCM79 | 1310* | 147.1 | 0.94 | 15.98 |
| hFcRn | 110.5 | UCM80 | 565 | 110.2 | 0.00358 | 17.92 |
| hFcRn | 109.6 | UCM81 | 1100* | 134.8 | 2.45 | 25.56 |
| hFcRn | 109 | UCM82 | 294 | 104 | 3.37 | 14.2 |
| hFcRn | 109.2 | UCM83 | 359 | 104.5 | 0.722 | 26.06 |
| hFcRn | 110.6 | UCM84 | 235 | 103.9 | 8.23 | 13.09 |
| hFcRn | 113.7 | Ipilimumab | 518 | 204.5 | 4.36 | 70.07 |

### Example 17: Cleavable Fc Molecules with Non-MMP Substrates

This example illustrates preparation of cleavable Fc molecules with cleavable substrates recognized by proteases other than MMP, for example, by Cathepsin B (CatB) (encoded by CTSB) or Matriptase (encoded by ST14). As shown in Table 22, 35 constructs (UCM85-UCM119) include cleavable substrate sites for Cathepsin B and 35 constructs (UCM120-UCM154) include cleavable substrate sites for matriptase. The substrate sequences and locations in the Fc region are shown in Table 22. The molecules were purified using Protein A.

TABLE 22
Cleavable Fc molecules with substrates for CatB and Matriptase

| | | Octet | Post-ProA | Purity After |
|---|---|---|---|---|
| | Protease | Titer | Yield | Post ProA |
| Construct | Cleavage Site | (mg/L) | (mg) | (%) |
| UCM85 | 440-RSKYLATA-447 | 64.1 | 1.36 | 74.58 |
| UCM86 | 440-GRPRHQGV-447 | 29 | 0.82 | 77.32 |
| UCM87 | 444-GLFG-447 | 92.2 | 1.79 | 71.39 |
| UCM88 | 444-GFLG-447 | 106.3 | 2.56 | 80.16 |
| UCM89 | 441-AGRRAAK-447 | 75 | 1.58 | 74.67 |
| UCM90 | 443-FRLWA-447 | 97.1 | 1.98 | 75.03 |
| UCM91 | 443-FRLWS-447 | 84.9 | 1.77 | 75.22 |
| UCM92 | 440-NFFGVGGE-447 | 89.8 | 2.29 | 77.31 |
| UCM93 | 440-PMKRLTLA-447 | 88.1 | 1.76 | 70.91 |
| UCM94 | 438-PMKRLTLA-445 | 19.4 | 1.13 | 68.69 |
| UCM95 | 436-PMKRLTLA-443 | 33 | 0.97 | 71.28 |
| UCM96 | 440-FPLATYAP-447 | 48 | 1.39 | 73.66 |
| UCM97 | 440-FLVGGASL-447 | 98.4 | 1.97 | 72.16 |
| UCM98 | 440-KPMQFLGD-447 | 36.7 | 1.33 | 73.63 |
| UCM99 | 440-GIVRAKGV-447 | 93.7 | 2.00 | 69.98 |
| UCM100 | 440-ALFKSSFP-447 | 99.3 | 2.16 | 72.35 |
| UCM101 | 440-SGRRSPGG-447 | 55.3 | 1.13 | 69.9 |
| UCM102 | 440-SLGRRPGG-447 | 88.1 | 2.04 | 71.52 |
| UCM103 | 440-SLSGRRGG-447 | 94.8 | 2.21 | 71.3 |
| UCM104 | 440-SLSLGRRG-447 | 91.2 | 2.14 | 75.39 |
| UCM105 | 440-SLSLSGRR-447 | 105.7 | 2.25 | 70.65 |
| UCM106 | 442-GGPRRL-447 | 90 | 1.82 | 69.67 |
| UCM107 | 442-GGPLRL-447 | 96.6 | 2.01 | 70.96 |
| UCM108 | 442-GGPKLL-447 | 85.5 | 1.84 | 76.6 |
| UCM109 | 442-GGPRNL-447 | 92.2 | 1.66 | 69.8 |
| UCM110 | 442-GGPRML-447 | 99 | 1.94 | 69.65 |
| UCM111 | 440-EHLRSPGG-447 | 34.2 | 1.53 | 71.5 |
| UCM112 | 440-FRSGVPGG-447 | 46.3 | 1.20 | 71.23 |
| UCM113 | 440-SLLLRTGN-447 | 75.9 | 1.53 | 73.09 |
| UCM114 | 440-AGLRSPGG-447 | 43.6 | 1.29 | 70.69 |
| UCM115 | 440-SLFRSAGP-447 | 100.6 | 1.99 | 70.85 |
| UCM116 | 440-SLFRAPGP-447 | 99.8 | 1.96 | 71.57 |
| UCM117 | 440-WLFRSPLG-447 | 90.2 | 1.99 | 76.17 |
| UCM118 | 440-SRLRSPQG-447 | 34.9 | 1.02 | 74.52 |
| UCM119 | 440-SLVLSGRR-447 | 90.8 | 1.75 | 71.87 |
| UCM120 | 440-KQLRHMRG-447 | 33 | 1.12 | 74.18 |
| UCM121 | 440-LSGRSDNH-447 | 70.8 | 1.42 | 68.27 |
| UCM122 | 444-LSGK-447 | 84.9 | 1.89 | 76.22 |
| UCM123 | 444-LSGR-447 | 92.5 | 1.98 | 72.67 |
| UCM124 | 440-RQARVVGG-447 | 60.5 | 1.42 | 69.8 |
| UCM125 | 440-RQRRVVGG-447 | 58.4 | 1.21 | 65.95 |
| UCM126 | 438-RQRRVVGG-445 | 55.5 | 0.96 | 68.49 |
| UCM127 | 436-RQRRVVGG-443 | 35.6 | 0.98 | 71.52 |
| UCM128 | 440-RQYRVVGG-447 | 55.8 | 1.19 | 72.27 |
| UCM129 | 440-SKGRSLIG-447 | 36.5 | 0.94 | 71.47 |
| UCM130 | 440-PRFKIIGG-447 | 34.2 | 1.02 | 75.16 |
| UCM131 | 440-KQLRVVNG-447 | 31.7 | 1.06 | 75.46 |
| UCM132 | 440-IQPRITGG-447 | Too Low | −0.03 | NA |
| UCM133 | 440-KQSRKFVP-447 | 62.1 | 1.33 | 74.12 |
| UCM134 | 440-GRQSRAGG-447 | 33.5 | 0.84 | 77.47 |
| UCM135 | 440-SGRSSPGG-447 | 53.3 | 1.08 | 73.14 |
| UCM136 | 440-SSGRSPGG-447 | 64 | 1.40 | 72.99 |
| UCM137 | 440-SLSGRSGG-447 | 84.7 | 1.70 | 73.95 |
| UCM138 | 440-SLSSGRSG-447 | 89.4 | 1.91 | 74.85 |
| UCM139 | 440-SLSLSGRS-447 | 86.3 | 1.85 | 74.52 |
| UCM140 | 441-PLRLSRA-447 | Too Low | 0.10 | 87.59 |
| UCM141 | 441-PLKLSRA-447 | 33.2 | 1.07 | 74.42 |
| UCM142 | 440-PLGLSGRS-447 | 86.3 | 1.81 | 70.88 |
| UCM143 | 440-PLGLRSRA-447 | 81.7 | 1.76 | 74.72 |
| UCM144 | 440-PLGLKSRA-447 | 83.4 | 1.74 | 73.26 |
| UCM145 | 442-RGSRAG-447 | 86.5 | 1.83 | 75.82 |
| UCM146 | 442-RLSRGK-447 | 87.1 | 1.92 | 76.38 |
| UCM147 | 442-RGSRGG-447 | 87.2 | 1.86 | 77.01 |
| UCM148 | 442-KGSRAG-447 | 89.5 | 2.00 | 75.18 |
| UCM149 | 442-KLSRGK-447 | 75.5 | 1.55 | 75.81 |
| UCM150 | 442-GRSRAG-447 | 84 | 1.80 | 76.17 |
| UCM151 | 442-LRSRGK-447 | 65 | 1.34 | 78.75 |
| UCM152 | 442-GRSRGG-447 | 79.4 | 1.67 | 75.79 |
| UCM153 | 442-GKSRAG-447 | 67.1 | 1.59 | 75.39 |
| UCM154 | 442-LKSRGK-447 | 75.6 | 1.59 | 76.81 |
| XM719 | Control Molecule | 32.3 | 0.66 | 81.41 |
| AK1119 | Control Molecule | 41.6 | 0.93 | 93.98 |

Next, kinetics study was performed to evaluate the protease cleavage of the masked cytokines with Fc domains comprising CatB or matriptase cleavage substrate. The results are shown in Tables 23-24. Most masked are effectively cleaved by CatB or matriptase.

TABLE 23
Cleavage Kinetics of Masked Cytokines by Matriptase

| | | | CTSB Est. | ST14 Est. | MMP9 Est. |
|---|---|---|---|---|---|
| Construct | Protease Site | Target protease(s) | kcat/Km | kcat/Km | kcat/Km |
| UCM129 | SKGRSLIG | matriptase | <1.0E+01 | 1.2E+04 | <1.0E+01 |
| UCM134 | GRQSRAGG | matriptase | <1.0E+01 | 1.6E+04 | <1.0E+01 |
| UCM143 | PLGLRSRA | matriptase, MMP | <1.0E+01 | 1.0E+03 | <1.0E+01 |
| UCM126 | RQRRVVGG | matriptase | <1.0E+01 | 1.9E+03 | <1.0E+01 |
| UCM135 | SGRSSPGG | matriptase | <1.0E+01 | 1.1E+03 | <1.0E+01 |
| UCM151 | LRSRGK | matriptase | <1.0E+01 | 1.1E+04 | <1.0E+01 |
| UCM146 | RLSRGK | matriptase | 2.3E+03 | 1.6E+04 | <1.0E+01 |
| UCM154 | LKSRGK | matriptase | <1.0E+01 | <1.0E+01 | <1.0E+01 |
| UCM149 | KLSRGK | matriptase | <1.0E+01 | 1.2E+04 | <1.0E+01 |
| UCM141 | PLKLSRA | matriptase, MMP | <1.0E+01 | 9.4E+02 | <1.0E+01 |
| UCM133 | KQSRKFVP | matriptase | <1.0E+01 | 3.6E+03 | <1.0E+01 |
| UCM144 | PLGLKSRA | matriptase, MMP | <1.0E+01 | 5.0E+02 | <1.0E+01 |
| UCM142 | PLGLSGRS | matriptase, MMP | <1.0E+01 | 3.0E+02 | <1.0E+01 |
| UCM124 | RQARVVGG | matriptase | 2.6E+03 | 2.6E+02 | <1.0E+01 |

TABLE 24
Cleavage Kinetics of Masked Cytokines by Cathepsin B

| | | | CTSB Est. | ST14 Est. | MMP9 Est. |
|---|---|---|---|---|---|
| Construct | Protease Site | Target protease(s) | kcat/Km | kcat/Km | kcat/Km |
| UCM108 | GGPKLL | CatB, MMP, matriptase | 4.5E+03 | <1.0E+01 | <1.0E+01 |
| UCM93 | PMKRLTLA | CatB | 5.8E+03 | <1.0E+01 | <1.0E+01 |
| UCM107 | GGPLRL | CatB, MMP, matriptase | 2.7E+03 | <1.0E+01 | <1.0E+01 |
| UCM106 | GGPRRL | CatB, MMP, matriptase | 4.5E+03 | <1.0E+01 | <1.0E+01 |
| UCM110 | GGPRML | CatB, MMP, matriptase | 2.4E+03 | <1.0E+01 | <1.0E+01 |
| UCM99 | GIVRAKGV | CatB | 2.1E+03 | <1.0E+01 | <1.0E+01 |
| UCM86 | GRPRHQGV | CatB, X-PSD | 2.3E+03 | 1.6E+03 | <1.0E+01 |
| UCM117 | WLFRSPLG | CatB, K, L, S | <1.0E+01 | <1.0E+01 | <1.0E+01 |
| UCM119 | SLVLSGRR | CatB, K, L, S | <1.0E+01 | 4.4E+02 | <1.0E+01 |
| UCM85 | RSKYLATA | CatB, X-PSD | 2.2E+03 | <1.0E+01 | <1.0E+01 |
| UCM97 | FLVGGASL | CatB | <1.0E+01 | <1.0E+01 | <1.0E+01 |
| UCM109 | GGPRNL | CatB, MMP, matriptase | <1.0E+01 | <1.0E+01 | 3.5E+03 |
| UCM105 | SLSLSGRR | CatB | <1.0E+01 | 1.3E+03 | <1.0E+01 |
| UCM100 | ALFKSSFP | CatL | <1.0E+01 | <1.0E+01 | <1.0E+01 |

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. An engineered Fc domain comprising one or more amino acid substitutions as compared to a wild-type Fc domain, wherein the engineered Fc domain comprises a protease cleavage site.

2. (canceled)

3. The engineered Fc domain of claim 1, wherein the protease cleavage site is a tumor associated protease cleavage site.

4. (canceled)

5. An engineered Fc domain comprising one or more amino acid substitutions in the hinge region, in the CH2 domain, in the CH3 domain, in the CH2-CH3 linker, and/or positions 436-447 or 426-437 according to EU numbering, such that the engineered Fc domain comprises a protease cleavage site.

6. The engineered Fc domain of claim 5, wherein the engineered Fc domain comprising one or more amino acid substitutions in the G-strand, FG-loop or F-stand of the CH3 domain.

7-8. (canceled)

9. The engineered Fc domain of claim 1, wherein the one or more amino acid substitutions are located in positions 416-425 according to EU numbering.

10. The engineered Fc domain of claim 9, wherein the one or more substitutions reduce or eliminate binding of the engineered Fc domain to protein A.

11-17. (canceled)

18. The engineered Fc domain of claim 1, wherein the protease cleavage site comprises any one of SEQ ID NOs: 95, 97, 99-100, 102, 104, 105, 107-110, 119, 122, 123, 135-139, 143-208, and 209.

19. The engineered Fc domain of claim 1, wherein the engineered Fc domain comprises any one of amino acid substitution sets listed in Table 8a, Table 5, Table 11, Table 11a, Table 11b, or Table c.

20-24. (canceled)

25. A masked cytokine comprising:
a cytokine moiety,
a masking moiety, and
an engineered Fc domain comprising a tumor-associated protease cleavage site;
wherein the engineered Fc domain is fused to the cytokine moiety or the masking moiety such that the masking moiety binds to the cytokine moiety and upon cleavage of the tumor-associated protease cleavage site on the engineered Fc domain, the cytokine moiety is released from the masking moiety.

26. The masked cytokine of claim 25, wherein the engineered Fc domain is fused to the cytokine moiety or the masking moiety via a non-cleavable linker.

27. (canceled)

28. A masked therapeutically active molecule comprising:
a therapeutically active domain,
a masking moiety, and
an engineered Fc domain comprising a tumor-associated protease cleavage site;
wherein the engineered Fc domain is fused to the therapeutically active domain or the masking moiety such that the masking moiety binds to the therapeutically active domain and upon cleavage of the tumor-associated protease cleavage site on the engineered Fc domain, the therapeutically active domain is released from the masking moiety.

29-30. (canceled)

31. The masked therapeutically active molecule of claim 28, wherein the therapeutically active domain is a cell engager, or a co-stimulatory domain.

32. A masked cytokine comprising:
a cytokine moiety,
a masking moiety, and
a carrier moiety comprising an engineered tumor-associated protease cleavage site;
wherein the carrier moiety is fused to the cytokine moiety and/or the masking moiety such that the masking moiety binds to the cytokine moiety and upon cleavage of the engineered tumor-associated protease cleavage site, the cytokine moiety is activated.

33. (canceled)

34. The masked cytokine of claim 32, wherein the carrier moiety is a half-life extension domain selected from albumin, transferrin, or a tissue factor.

35. The masked cytokine of claim 32, wherein the carrier moiety is selected from immunoglobulin, Fab, scFv, VHH, nanobody, VH, VL, single-domain Ab immunoglobulin kappa constant region (CK) and immunoglobulin lambda constant region (CL).

36-116. (canceled)

117. A nucleic acid encoding the engineered Fc domain of claim 1.

118-119. (canceled)

120. A method of producing a masked cytokine comprising culturing a host cell comprising the nucleic acid of claim 117 under a condition that produces the targeted cytokine.

121. (canceled)

122. A pharmaceutical composition or a kit comprising the engineered Fc domain of claim 1.

123. (canceled)

124. A method of treating or preventing a neoplastic disease in a subject, the method comprising administering to the subject an effective amount of the engineered Fc domain of claim 1.

125. A method of treating or preventing an inflammatory or autoimmune disease in a subject, the method comprising administering to the subject an effective amount of the engineered Fc domain of claim 1.

126-129. (canceled)
